SEQUENCE LISTING (12 sequences)

SEQ ID NO: 1 - DNA, 30 bp, Artificial Sequence
  Description of Artificial Sequence Synthetic primer
tctgttgccatcccaagacaacattgatgg

SEQ ID NO: 2 - DNA, 20 bp, Artificial Sequence
  Description of Artificial Sequence Synthetic primer
aaatctctggaggaggttgg

SEQ ID NO: 3 - DNA, 20 bp, Artificial Sequence
  Description of Artificial Sequence Synthetic primer
caaggccaaggagaaggtta

SEQ ID NO: 4 - DNA, 20 bp, Artificial Sequence
  Description of Artificial Sequence Synthetic primer
tttgaagttctcgggagtga

SEQ ID NO: 5 - DNA, 20 bp, Artificial Sequence
  Description of Artificial Sequence Synthetic primer
ccagaagccaatgcacctat

SEQ ID NO: 6 - DNA, 20 bp, Artificial Sequence
  Description of Artificial Sequence Synthetic primer
agcaataacagctcccacca

SEQ ID NO: 7 - DNA, 20 bp, Artificial Sequence
  Description of Artificial Sequence Synthetic primer
attctggcctccagttacca

SEQ ID NO: 8 - DNA, 20 bp, Artificial Sequence
  Description of Artificial Sequence Synthetic primer
ggcttcattcttggtgcttc

SEQ ID NO: 9 - DNA, 20 bp, Artificial Sequence
  Description of Artificial Sequence Synthetic primer
aacaacccttccacagatgc

SEQ ID NO: 10 - DNA, 20 bp, Artificial Sequence
  Description of Artificial Sequence Synthetic primer
tctccattaagggcgcatag

SEQ ID NO: 11 - DNA, 20 bp, Artificial Sequence
  Description of Artificial Sequence Synthetic primer
cttccgcaagttcacctacc

SEQ ID NO: 12 - DNA, 20 bp, Artificial Sequence
  Description of Artificial Sequence Synthetic primer
tacttgagggggatgaatcg

(19) United States
(12) Patent Application Publication
(10) Pub. No.: US 2014/0302042 A1
(43) Pub. Date: Oct. 9, 2014

(54) METHODS OF PREDICTING PROGNOSIS IN CANCER

(71) Applicants: Lynda Chin, Houston, TX (US); Kenneth L. Scott, Sugar Land, TX (US); Papia Ghosh, Boston, MA (US); Kunal Rai, Jamaica Plain, MA (US); Chengyin Min, Brookline, MA (US)

(72) Inventors: Lynda Chin, Houston, TX (US); Kenneth L. Scott, Sugar Land, TX (US); Papia Ghosh, Boston, MA (US); Kunal Rai, Jamaica Plain, MA (US); Chengyin Min, Brookline, MA (US)

(73) Assignee: DANA-FARBER CANCER INSTITUTE, INC., Boston, MA (US)

(21) Appl. No.: 14/130,145

(22) PCT Filed: Jun. 29, 2012
(86) PCT No.: PCT/US2012/045120

§ 371 Date: Jun. 23, 2014

Related U.S. Application Data
(60) Provisional application No. 61/504,033, filed on Jul. 1, 2011.

(51) Int. Cl. C12Q 1/68; G01N 33/50; G01N 33/574

(52) U.S. Cl. CPC C12Q 1/6886; G01N 33/5748; G01N 33/5011
USPC 424/139.1; 506/9; 506/2; 506/16; 506/18; 514/44 A; 424/174.1; 435/6.13

(57) ABSTRACT

A set of biomarkers (e.g., genes and gene products) that can accurately inform about the risk of cancer progression and recurrence, as well as methods of their use are disclosed.

### CROSS REFERENCES TO OTHER APPLICATIONS

This application claims priority from U.S. Provisional Application 61/504,033, filed Jul. 1, 2011. The disclosure of that application is incorporated by reference herein in its entirety.

### FIELD OF THE INVENTION

This invention relates to using biomarker panels to predict prognosis in cancer patients.

### BACKGROUND OF THE INVENTION

Metastasis is the cardinal feature of most lethal solid tumors and represents a complex multi-step biological process driven by an ensemble of genetic or epigenetic alterations that confer a tumor cell the ability to bypass local control and invade through surrounding matrix, survive transit in vasculature or lymphatics, ultimately colonize on foreign soil and grow (Gupta et al., Cell 127, 679-695 (2006)). It is the general consensus that such metastasis-conferring genetic events can be acquired stochastically as tumor grows and expands; indeed, total tumor burden is a positive predictor of metastatic risk. On the other hand, mounting evidence has promoted the thesis that some tumors may be endowed (or not) from the earliest stages with the capacity to metastasize. That some tumors are “hard-wired” for metastasis early in their life history is supported by clinical observation of widely varying outcomes among tumors of the equivalent early stage (i.e., similar tumor burden). Correspondingly, it has been shown that transcriptomic state of a metastasis is more similar to its matched primary than to other metastasis (Perou et al., Nature 406, 747-752 (2000)). In addition, it has been demonstrated that wholesale genomic aberrations in a cancer genome occurs early at the transition from benign to malignant stage (Chin et al., Nat Genet 36, 984-988 (2004)); Rudolph et al., Nat Genet. 28, 155-159 (2001)). However, it remains unknown what genes are involved in driving malignancy and what genes can provide reliable prognosis in cancer development.

### SUMMARY OF THE INVENTION

The present invention relates in part to the discovery that certain biological markers, such as proteins, nucleic acids, polymorphisms, metabolites, and other analytes, as well as certain physiological conditions and states, can accurately inform the risk of cancer progression and recurrence, as well as methods of their use. These biomarkers provide prognostic value for human cancer patients.

The invention provides a method for predicting prognosis of a cancer patient. In this method, one obtains a tissue sample from the patient, and measures the levels of two or more biomarkers in the sample or determines the nucleotide or amino acid sequence of one or more biomarkers in the sample, wherein the measured levels, or a mutation in the determined sequence as compared to a reference sequence, is indicative of the prognosis of the cancer patient. In some embodiments, the levels of two, three, four, five, six, seven, eight, nine, ten, fifteen, sixteen, seventeen, eighteen, nineteen, twenty, thirty, forty, fifty or more biomarkers are measured. In some embodiments, the nucleotide or amino acid sequence of one, two, three, four, five, six, seven, eight, nine, ten, fifteen, sixteen, seventeen, eighteen, nineteen, twenty, thirty, forty, fifty or more biomarkers are determined. In some embodiments, at least one of the selected biomarkers, i.e., the biomarkers being measured or sequenced, is associated with anoikis resistance. In these embodiments, the biomarkers may be selected from the group consisting of HNRPR, CDC20, PRIM2A, HRSP12, ENY2, TMEM141, RECQL, STK3, MX2, CDCA1, CEP68, SPBC25, HCAP-G, CDC25C, ANLN, GRID 1, PRIM1, DUT, RRAD, BIRC5, KNTC2, and PGEA1. In some embodiments, at least one of the selected biomarkers is associated with invasion, in these embodiments, the biomarkers may be selected from the group consisting of: 1) ACP5, FSCN1, HOXA1, HSF1, NDC80, VSIG4, NCAPH, ASF1B, MTHFD2, RNF2, SPAG5, ANLN, DEPDC1, HMGB1, ITGB3BP, MCM7, UBE2C, and UCHL5; or 2) ACP5, ANLN, ASF1B, BRRN1, BUB1, CDC2, CENPM, DEPDC1, ELTD1, EXT1, FSCN1, HCAP-G, HMGB1, HMGB2, HOXA1, HSF1, ITGB3BP, KIF20A, KIF2C, KNTC2, MCM7, MTHFD2, NASP, PLVAP, PTP4A3, RNF2, SPAG5, TGM2, UBE2C, UCHL5, and VSIG4. In some embodiments, at least one of the selected biomarkers is associated with tumorigenesis. In these embodiments, the biomarkers may be selected from the group consisting of ACP5, FSCN1, HOXA1, HSF1, NDC80, VSIG4, BRRN1, RNF2, UCHL5, HNRPR, PRIM2A, HRSP12, ENY2, and MX2. In some embodiments, the selected biomarkers comprise at least one of the biomarkers associated with invasion, at least one of the biomarkers associated with anoikis resistance, and at lease one of the biomarkers associated with tumorigenesis. In alternative embodiments, the biomarkers may be selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25CDC25C, GRID1, PRIM1, DUT, RRAD, BIRC5, and PGEA1. See, for example, Table 12 for two-biomarker combinations. In some embodiments, the prognosis may be that the patient is at a low risk of having metastatic cancer or recurrence of cancer. In other embodiments, the prognosis may be that the patient is at a high risk of having metastatic cancer or recurrence of cancer. In these embodiments, the patient may have melanoma, breast cancer, prostate cancer, or colon cancer.

The invention also provides a method for analyzing a tissue sample from a cancer patient. In this method, one obtains the tissue sample from the patient, measures the levels of two or more biomarkers in the sample or determines the nucleotide or amino acid sequence of one or more biomarkers in the sample, wherein the biomarkers are selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID 1, PRIM1, DUT, RRAD, BIRC5, and PGEA1. See, for example, Table 12 for two-biomarker combinations.

This invention additionally provides a method for identifying a cancer patient in need of adjuvant therapy. In this method, one obtains a tissue sample from the patient, measures the levels of two or more biomarkers in the sample or determines the nucleotide or amino acid sequence of one or more biomarkers in the sample selected from the group consisting of FSCN1, KIF2C, DEPDC1, ACP5, ANLN, ASF1B, BRRN1, BUB1CDC2, CENPM, ELTD1, EXT1, HCAP-G, HMGB1, HMGB2, HOXA1, HSF1, ITGB3BP, KIF20A, KNTC2. MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1, wherein the measured levels, or a mutation in the determined sequence as compared to a reference sequence, indicates that the patient is in need of adjuvant therapy. See, for example, Table 12 for two-biomarker combinations. For example, the adjuvant therapy may be selected from the group consisting of radiation therapy, chemotherapy, immunotherapy, hormone therapy, and targeted therapy. In some embodiments, the targeted therapy targets another component of a signaling pathway in which one or more of the selected biomarkers is a component. In alternative embodiments, the targeted therapy targets one or more of the selected biomarkers.

This invention also provides a further method for treating a cancer patient. In this method, one measures the levels of two or more biomarkers, or determines the nucleotide or amino acid sequence of one or more biomarkers, in a tissue sample from the patient, wherein the biomarkers are selected from the group consisting of FSCN1, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3MX2, CDCA1, CEP68, SPBC25, CDC25C, GRID1, PRIM1, DUT, RRAD, BIRC5, and PGEA1, and treats the patient with adjuvant therapy if the measured levels, or a mutation in the determined sequence as compared to a reference sequence, indicates that the patient is at a high risk of having metastatic cancer or recurrence of cancer. In some embodiments, the adjuvant therapy is an experimental therapy. See, for example, Table 12 for two-biomarker combinations.

This invention additionally provides a method for monitoring the progression of a tumor in a patient. In this method, one obtains a tumor tissue sample from the patient; and measures the levels of two or more biomarkers in the sample or determines the nucleotide or amino acid sequence of one or more biomarkers in the sample, wherein the biomarkers are selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1, and wherein the measured levels, or a mutation in the determined sequence as compared to a reference sequence, is indicative of the progression of the tumor in the patient. See, for example, Table 12 for two-biomarker combinations.

This invention further provides a method for identifying a cancer patient in need of a sentinel lymph node biopsy. In this method, one measures the levels of two or more biomarkers in the sample or determines the nucleotide or amino acid sequence of one or more biomarkers in the sample, wherein the biomarkers are selected from the group consisting of FSCN1, KIF2C, DEPDC1, ACP5; ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, RRAD, BIRC5, and PGEA1, and performs sentinel lymph node biopsy on the patient if the measured levels, or a mutation in the determined sequence as compared to a reference sequence, indicates that the patient is at a high risk of having metastatic cancer or recurrence of cancer. The invention conversely provides a method for identifying a cancer patient not in need of a sentinel lymph node biopsy. In this method, one measures the levels of two or more biomarkers in the sample or determines the nucleotide or amino acid sequence of one or more biomarkers in the sample, wherein the biomarkers are selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, CDC20, PRIM2A, HRSP12, ENY2, TMEM141RECQL, STK3, MX2, CDCA1, CEP68, SPBC25, CDC25C, GRID1, PRIM1, DUT, RRAD, BIRC5, and PGEA1, and does not perform sentinel lymph node biopsy on the patient if the measured levels, or a mutation in the determined sequence as compared to a reference sequence, indicates that the patient is at a low risk of having metastatic cancer or recurrence of cancer. See, for example, Table 12 for two-biomarker combinations.

In some embodiments, the selected biomarkers comprise one or more of ACP5, FSCN1, HOXA1, HSF1, NDC80, and VSIG4. Moreover, the selected biomarkers may further comprise one or more of ASF1B, MTHFD2, RNF2, and SPAG5. In some embodiments, the selected biomarkers comprise one or more of: 1) HNRPR, CDC20, PRIM2A, HRSP12, ENY2, TMEM141, RECQL, STK3, and MX2; or 2) ACP5, FSCN1, HOXA1, HSF1, NDC80, VSIG4, NCAPH, ASF1B, MTHFD2, RNF2, SPAG5, ANLN, DEPDC1, HMGB1, ITGB3BP, MCM7, UBE2C, and UCHL5; or 3) HNRPR, CDC20, PRIM2A, HRSP12, ENY2, TMEM141RECQL, STK3, MX2, CDCA1, CEP68, SPBC25, HCAP-G, CDC25C, ANLN, GRID1, DUT, RRAD, BIRC5, KNTC2, and PGEA1; or 4) ACP5, FSCN1, HOXA1, HSF1, NDC80, VSIG4, NCAPH, ASF1B, MTHFD2, RNF2, SPAG5, ANLN, DEPDC1, HMGB1, ITGB3BP, MCM7, UBE2C, and UCHL5; and at least one or more of HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA1.

In some embodiments, the levels of the selected biomarkers are determined based on the DNA copy number alteration. In these embodiments, the DNA copy number alteration of the selected biomarker indicates DNA gain or loss. In some embodiments, the nucleotide sequence or amino acid sequence of the selected biomarkers is determined by sequencing. For example, the nucleotide sequence may be determined by a polymerase chain reaction (PCR)-based assay, genotyping, sequencing by hybridization, reversible terminator sequencing, pyrosequencing, or sequencing by oligonucleotide ligation and detection. The amino acid sequence may be determined by mass spectrometry, immunoassay, or chromatography. In some embodiments, the RNA transcript levels of the selected biomarkers are measured. In certain embodiments, the RNA transcript levels may be determined by microarray, quantitative RT-PCR, sequencing, nCounter® multiparameter quantitative detection assay (NanoString), branched DNA assay (e.g., Panomics QuantiGene® Plex technology), or quantitative nuclease protection assay (e.g., Highthroughput Genomics qNPA™), nCounter® system is developed by NanoString Technology. It is based on direct multiplexed measurement of gene expression and capable of providing high levels of precision and sensitivity (<1 copy per cell) (see 72.5.117.165/applications/technology/). In particular, the nCounter® assay uses molecular “barcodes” and single molecule imaging to detect and count hundreds of unique transcripts in a single reaction. Panomics QuantiGene® Plex technology can also be used to assess the RNA expression of biomarkers this invention. The QuantiGene® platform is based on the branched DNA technology, a sandwich nucleic acid hybridization assay that provides a unique approach for RNA detection and quantification by amplifying the reporter signal rather than the sequence (Flagella et al., Analytical Biochemistry 352(1):50-60 (2006)). It can reliably measure quantitatively RNA expression in fresh, frozen or formalin-fixed, paraffin-embedded (FFPE) tissue homogenates (Knudsen et al., Journal of Molecular Diagnostics 10(2): 170-175 (2008)). In some embodiments, the protein levels of the selected biomarkers are measured. In certain embodiments, the protein levels may be measured, for example, by antibodies, immunohistochemistry or immunofluorescence. In these embodiments, the protein levels may be measured in subcellular compartments, for example, by measuring the protein levels of biomarkers in the nucleus relative to the protein levels of the biomarkers in the cytoplasm. In some embodiments, the protein levels of biomarkers may be measured in the nucleus and/or in the cytoplasm.

In some embodiments, the levels of the biomarkers may be measured separately. Alternatively, the levels of the biomarkers may be measured in a multiplex reaction.

In some embodiments, the noncancerous cells are excluded from the tissue sample. In some embodiments, the tissue sample is a solid tissue sample, a bodily fluid sample, or circulating tumor cells. In some embodiments, the bodily fluid sample may be blood, plasma, urine, saliva, lymph fluid, cerebrospinal fluid (CSF), synovial fluid, cystic fluid, ascites, pleural effusion, interstitial fluid, or ocular fluid. In some embodiments, the solid tissue sample may be a formalin-fixed paraffin embedded tissue sample, a snap-frozen tissue sample, an ethanol-fixed tissue sample, a tissue sample fixed with an organic solvent, a tissue sample fixed with plastic or epoxy, a cross-linked tissue sample, surgically removed tumor tissue, or a biopsy sample. In some embodiments, the tissue sample is a cancerous tissue sample. In some embodiments, the cancerous tissue is melanoma, prostate cancer, breast cancer, or colon cancer tissue.

In some embodiments, at least one standard parameter associated with the cancer is measured in addition to the measured levels (or determined sequences) of the selected biomarkers. The at least one standard parameter may be, for example, tumor stage, tumor grade, tumor size, tumor visual characteristics, tumor location, tumor growth, lymph node status, tumor thickness (Breslow score), ulceration, age of onset, PSA level, or Gleason score.

The invention provides a kit for measuring the levels of two or more biomarkers selected from the group consisting of FSCN1KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1. See, for example, Table 12 for two-biomarker combinations. The kit comprises reagents for specifically measuring the levels of the selected biomarkers. The invention also provides a kit for determining the nucleotide or amino acid sequence of one or more biomarkers in the sample selected from the group consisting of: FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1. See, for example, Table 12 for two-biomarker combinations. The kit comprises reagents for specifically determining the sequences of the selected biomarkers. In some embodiments, the reagents are nucleic acid molecules. In these embodiments, the nucleic acid molecules are PCR primers or hybridizing probes. In alternative embodiments, the reagents are antibodies.

The invention also provides a method for predicting prognosis of a cancer patient, comprising measuring the level of ACP5 or determines the nucleotide or amino acid sequence of ACP5 in a tissue sample from the patient, wherein the measured level of ACP5, or a mutation in the determined sequence of ACP5 as compared to a reference sequence of ACP5, is indicative of the prognosis of the cancer patient. In some embodiments, the level of the phosphatase activity of ACP5 is measured. In some embodiments, one or more biomarkers in addition to ACP5 are selected for measuring the levels or determining the nucleotide or amino acid sequence. These biomarkers may be selected from the group consisting of: 1) ANLN, ASF1B, BRRN1, BUB1, CDC2, CENPM, DEPDC1, ELTD1, EXT1, FSCN1, HCAP-G, HMGB1, HMGB2, HOXA1, HSF1, ITGB3BP, KIF20A, KIF2C, KNTC2, MCM7, MTHFD2, NASP, PLVAP, PTP4A3, RNF2, SPAG5, TGM2, UBE2C, UCHL5, and VSIG4; or 2) HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA1. In some embodiments, the prognosis is that the patient is at a low risk of having metastatic cancer or recurrence of cancer. Alternatively, the prognosis is that the patient is at a high risk of having metastatic cancer or recurrence of cancer.

This invention also provides a method for treating a cancer patient in need thereof. In this method, one measures the level of a biomarker selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1. PRIM1, DUT, RRAD, BIRC5, and PGEA1, and administers an agent that modulates the level of the selected biomarker. In some embodiments, the administered agent may be a small molecule modulator. In some embodiments, the administered agent may be a small molecule inhibitor. In some embodiments, the administered agent may be, for example, siRNA or an antibody. In one embodiment, the selected biomarker is ACP5. In some embodiments, the administered agent may inhibit the catalytic activity, for example, phosphatase activity of ACP5, or inhibit the secretion of ACP5 or the secreted ACP5. In some embodiments, the administered agent may cause a conformational change of ACP5, thereby preventing its biological activity or function. In some embodiments, the administered agent may cause disruption of the interaction between ACP5 and a substrate of ACP5. In some embodiments, the administered agent may target one or more residues of ACP5, for example, the histidine residue at position 111, the histidine residue at position 214, and the aspartic acid residue at position 265 of ACP5. Alternatively, the selected biomarker may be RNF2, UCHL5, HOXA1, UBE2C, FSCN1, HSF1, NDC80, VSIG4, BRRN1, HNRPR, PRIM2A, HRSP12, ENY2, or MX2.

This invention also provides a method of identifying a compound capable of reducing the risk of cancer recurrence or development of metastatic cancer. In this method, one provides a cell expressing a biomarker selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1, contacts the cell with a candidate compound, and determines whether the candidate compound alters the expression or activity of the selected biomarker, whereby the alteration observed in the presence of the compound indicates that the compound is capable of reducing the risk of cancer recurrence or development of metastatic cancer. In one embodiment, the selected biomarker is ACP5. In this embodiment, the identified compound inhibits the phosphatase activity or secretion of ACP5. In another embodiment, the selected biomarker is RNF2. In another embodiment, the selected biomarker is UCHL5. See, for example, Table 12 for two-biomarker combinations.

This invention also provides a method of identifying a compound capable of treating cancer. In this method, one provides a cell expressing a biomarker selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1, contacts the cell with a candidate compound, and determines whether the candidate compound alters the expression or activity of the selected biomarker, whereby the alteration observed in the presence of the compound indicates that the compound is capable of treating cancer. In one embodiment, the selected biomarker is ACP5. In this embodiment, the identified compound inhibits the phosphatase activity or secretion of ACP5. In another embodiment, the selected biomarker is RNF2. In another embodiment, the selected biomarker is UCHL5. See, for example, Table 12 for two-biomarker combinations.

This invention also provides a method of identifying a compound capable of reducing the risk of cancer occurrence or development of cancer. In this method, one provides a cell expressing a biomarker selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1 contacts the cell with a candidate compound, and determines whether the candidate compound alters the expression or activity of the selected biomarker, whereby the alteration observed in the presence of the compound indicates that the compound is capable of reducing the risk of cancer occurrence or development of cancer. In one embodiment, the selected biomarker is ACP5. In some embodiments, the identified compound may inhibit the catalytic activity, for example, phosphatase activity of ACP5, or inhibit the secretion of ACP5 or the secreted ACP5. In some embodiments, the identified compound may cause a conformational change of ACP5, thereby preventing its biological activity or function. In some embodiments, the identified compound may cause disruption of the interaction between ACP5 and a substrate of ACP5. In some embodiments, the identified compound may target one or more residues of ACP5, for example, the histidine residue at position 111, the histidine residue at position 214, and the aspartic acid residue at position 265 of ACP5. Alternatively, the selected biomarker may be RNF2, UCHL5, HOXA1, UBE2C, FSCN1, HSF1, NDC80, VSIG4, BRRN1, HNRPR, PRIM2A, HRSP12, ENY2, or MX2. See, for example, Table 12 for two-biomarker combinations.

Other features and advantages of the invention will be apparent from and encompassed by the following detailed description and claims.

### BRIEF DESCRIPTION OF THE DRAWINGS

FIGS. 1A-1H. Melanocyte-specific MET expression promotes formation of cutaneous metastatic melanoma. (A) Melanocytes were harvested from the indicated animals (Ink4a/Arf^{-/-}, Tet-Met and iMet) and adapted to culture for total RNA extraction following treatment with or without doxycycline (Dox). Expression of MET (Tg MET) was assayed by RT-PCR using transgene-specific primers. R15, ribosomal protein R15 internal control; −RT, no reverse transcriptase PCR control. (B) RT-qPCR was performed to analyze HGF expression in MET-induced primary melanomas (T1-T6). Tumor expression data is normalized to expression in two Ink4a/Arf^{-/-} melanocyte cell lines (Error bars indicate +/−SD). (C-D) Immunohistochemical staining of total c-Met and phosphorylated c-Met in a MET-induced primary melanoma. Scale bar=100 μm (top) and 50 μm (bottom). (E-H) H&E stained sections of a primary cutaneous spindle cell melanoma in the dorsal skin of an iMet transgenic mouse induced with doxycycline and distal metastases residing in lymph node, adrenal gland and lung. Scale bar=50 μm (primary tumor) and 100 μm (metastases).

FIGS. 2A-2D. Multi-dimensional genomic analyses and low-complexity functional genetic screen for cell invasion. (A) Schematic illustrating the integrative cross-species oncogenomics comparison. See also FIGS. 100A-10C, Table 3. (B) Flowchart depicting the low-complexity genetic screen for invasion and validation processes. (C) Histogram of 18 pro-invasion genes satisfying sequencing, expression and secondary screen verification efforts. GFP=negative control; TNTC=Too numerous to count. (D) Shown are representative invasion chamber images for HMEL468 cells stably expressing HOXA1 and ACP5. Scale bar=1.6 mm. See also Table 11.

FIGS. 3A-3E. Assessment of oncogenic activity by pro-invasion genes. (A-B) 1205Lu melanoma cells expressing non-targeting control (shGFP; NT) or individual shRNAs against ACP5 (−2 and −4) were assayed for effects on anchorage-independent growth in soft agar. Representative images and immunoblots depict colony formation and ACP5 protein knockdown, respectively. P value calculated by two-tailed t-test. (C) Kaplan-Meier tumor-free survival analysis for xenograft assays in Ncr-Nude mice using non-tumorigenic HMEL468 cells (1×10⁶ cells/injection site) stably expressing GFP or ACP5 (n=10 each). P value calculated by log-rank test. (D) M619 melanoma cells expressing non-targeting control (GFP) or individual shRNAs against the indicated candidates were assayed for effects on anchorage-independent growth in soft agar as in (A-B). See FIGS. 12A-12D for additional data using C918 melanoma cells and complementary knockdown verification data. (E) Kaplan-Meier tumor-free survival analysis for xenograft assays in Ncr-Nude mice using non-tumorigenic HMEL468 cells stably expressing the indicated genes. Log-rank calculated P values for individual candidates indicated at right of plot. Error bars indicate +/−SD. See FIGS. 45A and 45B for representative H&E staining of tumor sections.

FIGS. 4A-4K. In vivo metastasis studies. (A-C) Representative H&E stained sections showing lung and lymph node metastases in athymic mice (2/5) harboring orthotopic tumors generated from 1205Lu melanoma cells expressing ACP5. No metastases (0/5 animals) were detected in the GFP-expressing control cohort. Scale bar=200 μm. (D-E) Orthotopic fat pad metastasis assay using GFP-positive non-metastatic murine breast adenocarcinoma cells (NB008; 2×10⁴ cells/injection site) stably expressing vector control or ACP5. Shown are endpoint primary tumor size (top) and Kaplan-Meier metastasis-free survival analysis (bottom). P values calculated by two-tailed t-test (top) and log-rank (bottom). (F-K) Representative images of GFP-positive lung metastases and H&E stained sections of infiltrated lung from the ACP5 cohort. Scale bar=5.5 mm (left 2 panels) 300 μm (right 4 H&E panels).

FIGS. 5A-5F. ACP5 expression on melanoma tissue microarrays. (A) Box plot demonstrating the distribution of ACP5 cytoplasmic scores for primary (n=182) and metastatic (n=325) lesions on the Yale Melanoma Outcome Annotated TMA (YTMA59). P value calculated by mixed model ANOVA. Error bars indicate data within 1.5 interquartile range of the mean. (B) Primary tumors from (A) were divided into quartiles based on cytoplasmic expression of ACP5 thereby indicating a trend towards prolonged survival for the lowest expressing group (see also FIG. 13). Shown are the top 3 quartiles (red) compared to the first quartile (green), revealing a significantly shorter melanoma specific survival for the high expressers of ACP5 versus low expressers. P value calculated by log-rank test. (C—F) Representative staining of ACP5 (red) across histospot tumor specimens on YTMA59. S100/GP100 (green) defines tumor and nonnuclear compartments, and DAPI (blue) defines the nuclear compartments. Scale bar=100 μm.

FIGS. 6A-6F. ACP5 expression modulates phosphorylation status of adhesion molecules. (A-D) WM115 (top) and 1205Lu (bottom) cells over-expressing ACP5 or treated with shRNA targeting ACP5 (shACP5), respectively. Vec=vector control-expressing cells; shNT=non-targeting shRNA. Scale bar=10 μm (top) and 5 μm (bottom). (E) WM115 cells expressing empty vector (EV) or ACP5 were grown on plates coated with or without Matrigel and Fibronectin, and resulting protein lysates were immunoblotted with the indicated antibodies. See also FIG. 14. (F) Protein lysates extracted from WM115 and HMEL468 cells were immunoprecipitated (IP) with antibodies against focal adhesion kinase (FAK or F) and paxillin (PAX or P) for immunoblotting with the indicating antibodies. Tyrosine phosphorylation (pTyr) is determined by anti-pTyr immunoblot analysis.

FIGS. 7A-7G. Kaplan-Meier survival curves in breast cancer cohorts. (A-F) K-means clustering analysis based on the 18-gene pro-invasion oncogene (top) and Mammaprint® (bottom) signature using three independent cohorts of early-staged breast cancers: NKI metastasis-free survival (MFS)(van de Vijver et al., 2002); NCI recurrence-free survival (RFS) (Sotiriou et al., J Natl Cancer Inst 98, 262-272 (2006)); and Stockholm RFS (Pawitan et al., Breast Cancer Res 7, R953-964 (2005)). P values calculated by log-rank test. (G) Comparison of the 18-gene signature performance with the Mammaprint® (Agendia, Huntington Beach, Calif.) prognostic signature using the patient cohorts specified in (A-F). HR=Hazard ratio; C═C statistics.

FIG. 8. Table 1 summarizes the result of invasion validation and progression-correlated expression analysis concerning the 18 pro-invasion genes.

FIGS. 9A-9D. Melanocyte-specific MET expression promotes formation of cutaneous melanoma. (A) Primary tumors (T1-T6) were harvested from iMet animals on doxycycline and assessed for expression of the melanocytic markers Tyrosinase, TRP1 and Dct by RT-PCR using gene-specific primers. XB2, mouse keratinocyte cell line; B 16F10, mouse melanoma cell line; R15, ribosomal protein R15 internal control; −RT, no reverse transcriptase PCR control. (B) Melanocyte-specific immunohistochemical staining of S100 in a MET-induced primary melanoma. t, tumor; f, folicule; fm, folicular melanocytes; a, adipocytes. (C-D) HRAS* and iMet tumor cells (5×10⁵) were injected in the tail vein of athymic mice and followed for formation of lung nodules, a correlate of metastatic seeding. Left panel: H&E stained section of nodule-free lung tissue harvested from animals tail vein injected with an HRAS* melanoma cell line (0/4 mice); Right panel: H&E stained section of nodule-infiltrated lung tissue harvested animals tail vein-injected with the MET-driven BC014 cell line (iMet) (3/4 mice). t, tumor.

FIGS. 10A-100C. IPA enrichment analysis and low-complexity genetic screen for pro-invasion genes. (A-B) Ingenuity Pathway Analysis of differentially expressed genes between iHRAS* and iMet mouse melanomas (1597 probe sets, top) and cross-species integrated gene list (360 filtered gene list, bottom) were compared to 9 randomly drawn gene sets of equal size. Top 4 significant functional classifications are shown. (C) HMEL468 melanocytes were transduced with individual pro-metastasis candidate cDNA virus, followed by loading onto 96-well transwell invasion assay plates (BD Bioscience). Invasiveness was measured via florescence-mediated quantitation and values were normalized to empty vector control. Candidate cDNAs driving invasion 2× standard deviations from the GFP controls in two independent screening efforts were considered primary screen hits (n=45).

FIGS. 11A-11N. Candidates exhibit progression-correlated expression in malignant melanoma. (A-L) Representative staining (red) for the indicated candidates across nevi, primary and metastasis (Met) tumor specimens (histospots 46. 3 and 29, respectively) on the Yale Melanoma Progression Tissue Microarray (YTMA98; see Table 1). S100/GP100 (green) defines tumor and nonnuclear compartments. Informative cores were assessed for AQUA® scores for ACP5 and HSF1 staining in the cytoplasmic and nuclear cellular compartments, respectively. (M-N) Box plots demonstrate the distribution of AQUA scores. Significance calculated by mixed model ANOVA. Original magnification=20×.

FIGS. 12A-12D. Invasion genes are required for maintaining anchorage-independent growth. (A-B) M619 (A) and C918 (B) melanoma cells expressing non-targeting control (shGFP) or individual shRNAs against the indicated genes were assayed for effects on anchorage-independent growth in soft agar. Note that (A) is the full panel of growth assays including those representatives shown in FIG. 3B. (C-D) shRNA knockdown verification by RT-qPCR analysis for M619 (C) and C918 (D) plotted as percent knockdown relative to GFP. Error bars indicate +/−s.d.

FIG. 13. Kaplan-Meier survival curve of melanoma specific mortality. Primary melanomas on the Yale Melanoma Outcome Annotated TMA (YTMA59) were divided into quartiles based on cytoplasmic expression of ACP5 thereby indicating a trend towards prolonged survival for the lowest expressing group. The top 3 quartiles were subsequently combined and compared to the first quartile, revealing a significantly shorter melanoma specific survival for the high expressers of ACP5 (see FIG. 5B).

FIG. 14. ACP5 modulated phosphorylation of paxillin. Ectopic expression of ACP5 in WM115 and HMEL468 cells leads to reduced site-specific (Tyr118) phosphorylation of paxillin (PAX).

FIG. 15. Table 2 summarizes tumor incidence in the experimental mouse colony and impact of wounding on tumorigenesis (see FIGS. 1A-1H).

FIGS. 16A-16B. An improved acid phosphatase assay was used to measure the phosphatase activity of ACP5 in cell lysates (A) and conditioned medium (B). Molybdate was used as an acid phosphatase inhibitor. The increased phosphatase activity of ACP5 was inhibited by increased concentrations of molybdate. 293T cells were transfected with GFP/pLenti6 (GFP/pL6) and ACP5/pLenti6 (ACP5/pL6) lentiviral vectors using Lipofectamine™ 2000 for 48 h. Cell lysates and conditioned medium were collected and subjected to the acid phosphatase activity assay.

FIG. 17. The same acid phosphatase assay in FIGS. 16A-16B was used to measure the phosphatase activity of a recombinant human ACP5 purchased from R&D systems. The increased phosphatase activity of the recombinant ACP5 was also inhibited by increased concentrations of molybdate.

FIG. 18. The effect of molybdate as an acid phosphatase inhibitor was compared to imidazole, an alkaline phosphatase inhibitor. The increased activity of ACP5 was inhibited by molybdate, but not by imidazole. HMEL cells stably expressing GFP and ACP5 were generated using lentiviral infection. Cell lysates were prepared and 1 μg lysates were subjected to acid phosphatase activity assay in the presence of increased concentrations of molybdate and imidazole.

FIGS. 19A-19C. Generation of ACP5 mutants. (A-B) Three single amino acid mutants H111A, H214A and D265A and a deletion mutant (deleting the signal peptide) (referred as “−sp”) were generated based on the structural information of rat ACP5 protein (A). The mutants were generated using Quikchange™ site-directed mutagenesis kit (Strategen). (C) 293T cells were transfected as described in FIGS. 16A-16B and cell lysates and medium were collected and subjected to immunoblotting with antibody against ACP5. 293T cells transfected with GFP, LacZ, ACP5 and the four mutants (H111A, H214A, D265A and −sp) were cultured and immunoblotted. 37 Kd corresponds to full-length 5a isoform of ACP5 and 23 Kd corresponds to fragments of 5b isoform of ACP5 under reduced condition.

FIGS. 20A-20C. Phosphatase activity assay and invasion assay of ACP5 mutants in HMEL cells stably expressing ACP5 mutants. HMEL stable cells were generated using lentiviral infection and selection with Blasticidine. (A) Phosphatase activity of ACP5 mutants—H111A, H214A, D265A and −sp (deleting the signal peptide). The H111A and H214A mutants almost completely lost the phosphatase activity as compared to wild type ACP5. The D265A mutant retained −40% of the phosphatase activity as compared to wild type ACP5. The −sp mutant, similar to the H111A mutant, almost completely lost the phosphatase activity. (B) Wild type ACP5 significantly induced invasion of HMEL cells, as compared to H111A, H214A and D265 mutants, in the Boyden chamber invasion assay. (C) Wild type ACP5 significantly increased invasion of HMEL cells, while −sp mutant had no effect. Y-axis is average cell number invaded through the filter.

FIGS. 21A-21C. A fluorescence staining assay using ELF97 phosphatase substrate (Invitrogen) was also tested to measure the phosphatase activity of ACP5 mutant. HMEL stable cell lines expressing ACP5 wild type, ACP5-sp mutant and LacZ were grown on cover-slip for 48 h. ELF97 was used as phosphatase substrate to visualize phosphatase activity according to manufacturer's directions. The nucleus was counterstained with propidium iodide.

FIGS. 22A-22H. Wild-type ACP5 induced invasion of pMEL/BRAF cells as compared to GFP controls. The H111A mutant had no effect on invasion. The results indicate that the phosphatase activity of ACP5 is required for its function in melanoma cell invasion. (A-F) pMEL/BRAF stable cells expressing GFP, ACP5 wild-type and ACP5 H111A mutant were generated using lentiviral infection as described above and subjected to Boyden chamber invasion assay. (G) Immunoblotting of cell lysates. (H) Comparison of phosphatase activity of pMEL/BRAF cells expressing GFP, wild-type ACP5 and the H111A mutant. pMEL/BRAF stable cells expressing ACP5 and its mutants were generated similar to the above-described HMEL stable cells expression ACP5 and its mutants. Cells were subjected to Boyden chamber invasion assay for 24 h as described in FIG. 20B-20C.

FIGS. 23A-23F. Wild-type ACP5 induced invasion of WM115 cells as compared to GFP controls. The H111A mutant had no effect on invasion. The results indicate that the phosphatase activity of ACP5 is required for its function in melanoma cell invasion. Stable WM115 stable cells expressing GFP, ACP5 wild-type and ACP5 H111A mutant were generated using lentiviral infection as described above and subjected to Boyden chamber invasion assay for 24 h.

FIGS. 24A-24C. ACP5 drives in vivo metastasis to lung and lymph node and ACP5 phosphatase activity is required for its function in melanoma metastasis. (A-B) An in vivo metastasis assay was performed to examine the association between the phosphatase activity of ACP5 and its function in metastasis. Stable cell lines (1205Lu) expressing GFP, wild-type ACP5, ACP5 H111A mutant were generated through lentiviral infection. Cells were then injected subcutaneously into the right flank of nude mice at 1×10⁶ cells/site (5 mice per group). Mice were monitored for tumor growth and later sacrificed when tumors reached 2 cm in one dimension. Metastasis was confirmed by H&E. (C) Two out of the five mice in the group injected with cells expressing wild-type ACP5 had lung metastasis. Metastasis was not observed in those mice injected with cells expressing GFP control or the H111A mutant. These results confirm that the phosphatase activity of ACP5 is required for its function in metastasis.

FIGS. 25A-25D. H&E staining confirms metastasis in lung and lymph node for one of the mice injected with cells expressing wild-type ACP5 (Mice 3).

FIGS. 26A-26B. H&E staining confirms metastasis in lung for one of the mice injected with cells expressing wild-type ACP5 (Mice 5).

FIGS. 27A-27B. Two additional in vivo metastasis assays were performed using pMEL/NRAS (A) and iNRAS (B) cell lines. pMEL/NRAS cells were transduced with GFP/pLenti6.3, ACP5/pLenti6.3 and H111A/pLenti6.3. iNRAS mouse cells were transduced with RFP/pHAGE, ACP5/pHAGE and H111A/pHAGE. Stable cells were injected subcutaneously into the right flank of nude mice at 1×10⁶ cells/site (5 mice per group). Tumor size was measured and volume calculated on day 55 and day 21 for pMEL/NRAS and iNRAS, respectively. The expression of ACP5 promoted primary tumor growth and this effect is dependent on the phosphatase activity of ACP5. The results are consistent with the previous findings in 1205Lu cell lines.

FIGS. 28A-28C. Cross-species oncogenomic analysis and in vitro anoikis resistance screen. (A) depicts the overall strategy for identifying pro-metastatic determinants: integration of murine expression data with human a-CGH data identified 298 up-regulated genes of which all available ORFs were pursued in the in vitro and in vivo analysis. (B) depicts the work-flow of in vitro anoikis resistant gene screen. (C) In vitro anoikis screen results. The positive control, TrkB, which has been shown to be an anoikis resistance gene, and its ligand, BDNF, confer increased survival to RIE compared to empty vector. Shown are representative results of two independent passes of the screens.

FIG. 29. Table 4 summarizes the results of in vitro anoikis resistance screen. Nine genes hit on two independent passes of the screen. Among those nine genes, seven had greater than two standard deviations from the mean.

FIGS. 30A-30H. Anoikis resistance genes promote in vivo metastasis from a subcutaneous injection. (A) Kaplan-Meier curve showing tumor-free survival of anoikis resistance genes in vivo (2 cm²). Hoxa1 and TrkB were also included. (B) Kaplan-Meier curve of metastasis free survival. Three genes (HNRPR, ENY2, and MX2) promote metastasis to the lymph node or lung in vivo. (C) In vivo metastasis results including the time of initial tumor formation and end-point detection of metastasis for the tested genes. (D-F) H&E staining of GFP and HNRPR in lung showing metastatic nodules. (G-H) Immunoblotting analysis of HNRPR expression in injected cells and HNRPR mRNA expression of in vivo samples.

FIGS. 31A-31C. Eny 2, as one of the top nine anoikis resistance genes in Table 4, demonstrates a positive correlation with tumor progression (p<0.05; p value was calculated by a two-tailed Student's t-test.). Eny 2 also exhibits significant over-expression in various melanoma metastatic data-sets including the Riker data-set (FIG. 31A; Riker et al., BMC Med Genomics 1, 13 (2008)), and a comparison of primary and metastatic melanoma (Kabbarah et al., PLoS One 5, e10770 (2010)).

(FIG. 31B), and the Talantov data-set (FIG. 31C; Talantov et al., Clin Cancer Res. 11, 7234-7242 (2005))

FIGS. 32A-32B. HNRPR, as one of the top nine anoikis resistance genes in Table 4, exhibits significant (p<0.05) over-expression in the Riker melanoma data-set (FIG. 32A) and the Talantov melanoma data-set (FIG. 32B). P value was calculated using a two-tailed Student's t-test.

FIG. 32C. RECQL is one of the top nine anoikis resistance genes in Table 4. RECQL shows significant (p<0.05) over-expression in the Riker melanoma data-set. P-value was calculated using a t-test.

FIGS. 33A-33E. Individual anoikis resistant genes show correlation with survival in various tumor types, indicating that anoikis resistance genes have relevance in non-melanoma data-sets. (A) Summary of Kaplan-Meier survival analysis of the top nine anoikis resistance genes in Table 4. Analysis was done by K-means clustering. Shown are those that had a Hazard Ratio >1 and p<0.05 (Hazard Ratio was calculated by univariate Cox proportional hazard regression model; p value was estimated from log-rank test). Results are written as HR/WP. GBM=glioblastoma; OV=ovarian; BR=breast; PR=prostate; OS=over=all survival; MFS=metastasis free survival; RFS=recurrence free survival; BCR=biochemical recurrence; TCGA=The Cancer Genome Atlas; Data-sets: Vijver et al., N Engl J Med 347, 1999-2000 (2002); Wang et al., Lancet 365, 671-679 (2005); Pawitan et al. Breast Cancer Res 7, R953-964 (2005); and Glinsky et al., J. Clin. Invest. 113, 913-923 (2004). (B-C) Increased expression of Eny2 and HNRPR in other cancers (Oncomine). (D-E) Survival curves of the top nine anoikis resistance genes in Table 4 and the remaining thirteen anoikis resistance genes in breast cancer metastasis free survival.

FIGS. 34A-34E. The top nine anoikis resistance genes from Table 4 promote cell proliferation and soft agar colony formation. (A) Eny2 and HNRPR promote cell proliferation of 1205Lu. Crystal violet staining was read at an absorbance of OD540. (B-C) HNRPR and ENY2 significantly increased colony formation in 1205Lu (p<0.05). (D-E) STK3, CDC20, PRIM2A, HNRPR and RECQL all increased colony formation in WM239A relative to GFP control.

FIGS. 35A-35H. Eny2 reduces apoptosis in non-adherent conditions and promotes soft agar colony formation. (A-B) Eny2 over-expression increases survival of rat intestinal epithelial cells in non-adherent conditions as measured by an ATP assay. (C-F) Eny2 over-expression reduces apoptosis of rat intestinal epithelial cells in non-adherent conditions as measured by Annexin/PI. (G-H) Eny2 over-expression promotes soft agar colony formation in Mewo, a cell line with low Eny2 levels.

FIGS. 36A-36F. Functional studies of Eny2 indicate that Eny2 may regulate histone ubiquitination dependant on the SAGA-DUB complex. (A) Eny2 is involved in various complexes that control histone ubiquitination (SAGA-DUB), mRNP formation (THO) and mRNA localization to the nuclear pore (AMEX) (Kopytova et al., Cell Cycle 9, 479-481 (2010)). (B) 1205Lu cells stably over-expressing Eny2 were transduced with two independent pLKO.1 shRNA against USP22. One of the shRNAs, i.e., shRNA A4, resulted in greater loss of USP22 and further rescued ENY2-mediated decrease in H2BUb. Therefore, shRNA-mediated loss of USP22, which is the catalytic member of the SAGA-DUB complex, inhibits Eny2 over-expression-mediated decrease in H2BUb levels in 1205Lu cells. (C) Over-expression of Eny2 in an additional melanoma cell line, WM115, also reduces H2BUb. (D) Increased invasion of 1205Lu in a Boyden Invasion Chamber by Eny2 over-expression is reduced with shRNA silencing of USP22 (shRNA A4). (E) Loss of Eny2 in 1205Lu lung metastatic cells stably over-expressing Eny2 increases H2BUb. (F) USP22 expression is increased in more progressive samples in the Talantov melanoma data-set. P-value is calculated using a t-test. These results indicate that Eny2 regulates H2Bub in some melanoma cells lines and this regulation may be dependent on the catalytic subunit of the SAGA-DUB complex, USP22. Furthermore, Eny2 promotion of invasion may also be dependent on USP22. Eny2 is necessary for inhibition of H2BUb in cells derived from metastatic lung nodules stably expressing Eny2.

FIGS. 37A-37K. HNRPR over-expression increases survival in non-adherent conditions. (A-B) HNRPR over-expression increases survival of rat intestinal epithelial cells in non-adherent conditions. Shown are associated Western blotting results. (C—F) HNRPR over-expression reduces apoptosis of rat intestinal epithelial cells in non-adherent conditions (Annexin/PI). (G-H) shRNA-mediated loss of HNRPR in 501MeI decreases 501MeI cell proliferation and survival in non-adherent conditions. Loss of HNRPR in Mewo also has no effect on survival (data not shown). (1-K) HNRPR over-expression increases survival of 1205Lu in non-adherent conditions and increases Akt (S473).

FIGS. 38A-38E. Expression of MX2 increases survival and reduces apoptosis of rat intestinal epithelial cells in non-adherent conditions.

FIG. 39. Summary histogram of fold-increase in invasive activity relative to control for the 31 pro-invasion genes.

FIGS. 40A-40B. Assessment of HOXA1 oncogenic activity. (A) WM115 melanoma cells expressing either empty vector (EV; left) or HOXA1 (right) were plated in soft-agar to assess anchorage independent growth. (B) quantitative measurement of the assay (n=6 wells each).

FIGS. 41A-41B. UBE2C exhibits higher expression in melanomas versus nevi and cooperatively transforms primary fibroblasts. (A) QuantiGene® analysis of RNA expression of UBE2C in a cohort of Spitz nevi and melanoma FFPE specimen. (B) Primary Ink4a/Arf-deficient MEFs were transfected with the indicated vectors expressing HRASV12, MYC and UBE2C. Vec=LacZ vector control; bars indicate ±S.D.

FIGS. 42A-42F. RNF2 is shown to be oncogenic. RNF2 promotes anchorage-independent growth and tumor formation of immortalized primary melanocytes in nude mice. (A-C) Representative images (A-B) and colony count (C) for soft agar colony formation assay of HMEL-GFP and HMEL-RNF2 cells. (D-E) Representative pictures of mice injected with HMEL-GFP and HMEL-RNF2 cells. (F) Kaplan-Meier curve of tumor free survival for mice injected with HMEL-GFP and HMEL-RNF2 cells.

FIGS. 43A-43G. RNF2 induces invasion and is required for lung seeding. (A-D) Crystal violet stained invasive cells pictured after invasion from a Boyden Chamber assay in HMEL and WM115 cells over-expressing GFP (A and C) and RNF2 (B and D). The results indicate that RNF2 promotes invasiveness of immortalized primary melanocytes and melanoma cells suggesting its role in metastasis process. (E-G) Bright light microscope images of lungs of mice injected with GFP-expressing cells as indicated to assess lung nodule formation. The results indicate that RNF2 is essential for lung seeding of pro-invasive melanocytes establishing its requirement for metastasis process.

FIGS. 44A-44D. UCHL5 induces invasion and metastasis. (A-B) Crystal violet stained invasive cells pictured after invasion from a Boyden Chamber assay of WM115 cells over-expressing GFP or UCHL5. UCHL5 promotes invasiveness of melanoma cells suggesting its role in metastasis process. (C-D) Pictures (2×) of H&E stained lung of mice injected with WM115-GFP cells or WM115-UCHL5 cells. Arrows indicate tumor cells/nodules. UCHL5 over-expression leads to lung metastasis from subcutaneous site suggesting UCHL5 is sufficient to impart metastatic properties to non-metastatic melanoma cells.

FIGS. 45A-45B. Representative H&E staining of tumor sections for xenograft assays in Ncr-Nude mice using non-tumorigenic HMEL468 cells stably expressing the indicated genes as in FIG. 3E.

FIGS. 46A-46B. HOXA1 drives distal metastasis from primary tumors. Mammary fat pad metastasis assay using GFP-positive non-metastatic murine breast adenocarcinoma cells (NB008; 2×10⁴ cells/injection site) stably expressing vector control or HOXA1. Shown are representative images of GFP-positive lung metastases.

### DETAILED DESCRIPTION

We posit that the genetic determinants or biomarkers of a tumor's metastatic potential are pre-existing in early stage primary malignancies, and such determinants are functionally active in the very processes responsible for metastatic dissemination. Therefore, such metastasis determinants or biomarkers are not only potential therapeutic targets but also determinants of aggressiveness of the cancerous disease; hence the metastatic determinants are also prognostic determinants. In particular, we have discovered that a biomarker panel comprising one or more members from the group consisting of: FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1 (Table 6) are useful in providing molecular, evidence-based reliable prognosis about cancer patients.

As described below, the inventors of the present invention utilized two genetically engineered mouse models with contrasting metastatic potential and further adopted a comparative oncogenomics-guided function-based strategy to identify genes/proteins that are associated with invasion, anoikis resistance, and/or tumorigenesis. These identified genes or gene products can be used, either alone or in combination, as biomarkers for predicting prognosis in cancer with high sensitivity and specificity, and as therapeutic targets for cancer treatment.

### Biomarkers and Biomarker Panels

The inventors of the present invention have identified fifty biomarkers that are associated with invasion, anoikis resistance, and/or tumorigenesis: FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A. HRSP12. ENY2, TMEM141, RECQL, STK3. MX2, CDCA1, CEP68, SPBC25, CDC25C, GRID1, PRIM1, DUT, RRAD, BIRC5, and PGEA1 (see Table 6). As used herein, the term “biomarker” or “marker” refers to an analyte (e.g., a nucleic acid, peptide, protein, or metabolite) whose biological characteristics (e.g., amount, activity level, sequence, activation (e.g., phosphorylation) state) can be used as an indicator for a physiological condition, such as a disease condition. We have discovered that the levels (e.g., expression or activity), or the presence (or absence) of mutations (e.g., mutations that affect activity of the biomarker, such as substitutions, deletions, or insertion mutations) or polymorphisms, or the DNA copy numbers (e.g., gain or loss) of one or more of these biomarkers can be used in prognosis of cancer as well as in many clinical applications as described below.

The inventors have also discovered that a biomarker panel that can be used in the methods of the present invention may comprise: 1) one or more biomarkers associated with invasion and one or more biomarkers associated with anoikis resistance; or 2) one or more biomarkers associated with tumorigenesis and one or more biomarkers associated with anoikis resistance; or 3) one or more biomarkers associated with invasion and one or more biomarkers associated with tumorigenesis; or 4) one or more biomarkers associated with invasion, one or more biomarkers associated with anoikis resistance, one or more biomarkers associated with invasion, and one or more biomarkers associated with tumorigenesis. A biomarker panel that comprises multiple biomarkers that are associated with different pathways involved in metastasis or cancer recurrence can achieve high sensitivity and specificity of cancer prognosis.

Biomarker panels of the present invention can be constructed with one or more of the biomarkers described herein. For example, a biomarker panel that can be used in the methods of the present invention may comprise one or more biomarkers selected from FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20. PRIM2A. HRSP12. ENY2, TMEM141, RECQL, STK3. MX2, CDCA1, CEP68, SPBC25, CDC25C, GRID1, PRIM1. DUT, RRAD, BIRC5, and PGEA1. In some embodiments, at least two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five or fifty biomarkers are selected to constitute the panel. See, for example, Table 12 for two-biomarker combinations.

In certain embodiments, to construct a biomarker panel tailored to provide a particular piece of prognostic information, one can use one or more algorithms or models that prioritize the candidate biomarkers as well as train the optimal formula to combine the results from multiple biomarkers for a panel. By way of example, one may use linear or non-linear equations and statistical classification analyses to determine the relationship between levels of the biomarkers detected in a training cohort and the cohort's known clinical outcome (e.g., survival at a given time point). Examples of algorithms or models that can be used to construct biomarker panels include, without limitation, structural and syntactic statistical classification algorithms, methods of risk index construction, utilizing pattern recognition features, cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Many of these techniques are useful either when combined with a biomarker selection technique, such as forward selection, backwards selection, or stepwise selection, complete enumeration of all potential panels of a given size, genetic algorithms, or when they may themselves include biomarker selection methodologies. These may also be coupled with information criteria, such as Akaike's Information Criterion (AIC) or Bayes Information Criterion (BIC), in order to quantify the tradeoff between additional biomarkers and model improvement, and to aid minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art.

The performance (e.g., predictive power) and thus, usefulness of biomarker panels may be assessed in multiple ways. For example, the sensitivity, the specificity, positive predictive value (or rate), and negative predictive value (or rate) of the panel may be considered. These parameters can be calculated according to algorithms or equations known in the art. For example, “sensitivity” can be calculated by TP/(TP+FN) or the true positive fraction of disease subjects. “Specificity” can be calculated by TN/(TN+FP) or the true negative fraction of non-disease or normal subjects. “TN” is true negative, which for a disease state test means classifying a non-disease or normal subject correctly. “TP” is true positive, which for a disease state test means correctly classifying a disease subject. “FN” is false negative, which for a disease state test means classifying a disease subject incorrectly as non-disease or normal. “FP” is false positive, which for a disease state test means classifying a normal subject incorrectly as having disease. “Positive predictive value” or “PPV” is calculated by TP/(TP+FP) or the true positive fraction of all positive test results. It is inherently impacted by the prevalence of the disease and pre-test probability of the population intended to be tested. “Negative predictive value” or “NPV” is calculated by TN/(TN+FN) or the true negative fraction of all negative test results. It also is inherently impacted by the prevalence of the disease and pre-test probability of the population intended to be tested.

Among the fifty biomarkers in Table 6, thirty-one biomarker are identified as being associated with invasion: ACP5, ANLN, ASF1B, BRRN1, BUB1, CDC2, CENPM, DEPDC1, ELTD1, EXT1, FSCN1, HCAP-G, HMGB1, HMGB2, HOXA1, HSF1, ITGB3BP, KIF20A, KIF2C, KNTC2, MCM7, MTHFD2, NASP, PLVAP, PTP4A3, RNF2, SPAG5, TGM2, UBE2C, UCHL5, and VSIG4. In some embodiments, a biomarker panel that can be used in the methods of the invention may comprise one or more biomarkers selected from these thirty-one biomarkers. In some embodiments, a biomarker panel that can be used in the methods of the invention may comprise one more biomarkers selected from ACP5, FSCN1, HOXA1, HSF1, NDC80, VSIG4, NCAPH, ASF1B, MTHFD2, RNF2, SPAG5, ANLN, DEPDC1, HMGB1, ITGB3BP, MCM7, UBE2C, and UCHL5. In some embodiments, a biomarker panel that can be used in the methods of the invention comprise one or more biomarkers selected from ACP5, FSCN1, HOXA1, HSF1, NDC80, and VSIG4. In some embodiments, a biomarker panel that can be used in the methods of the invention comprise one or more biomarkers selected from ACP5, FSCN1, HOXA1, HSF1, NDC80, and VSIG4 and further comprise one or more biomarkers selected from ASF1B, MTHFD2, RNF2, and SPAG5.

Among the fifty biomarkers in Table 6, twenty-two biomarker are identified as being associated with anoikis resistance: HNRPR, CDC20, PRIM2A, HRSP12, ENY2, TMEM141, RECQL, STK3, MX2, CDCA1, CEP68, SPBC25, HCAP-G, CDC25C. ANLN, GRID1. PRIM1, DUT, RRAD. BIRC5, KNTC2, and PGEA1. In some embodiments, a biomarker panel that can be used in the methods of the invention may comprise one or more biomarkers selected from these twenty-two biomarkers. In some embodiments, a biomarker panel that can be used in the methods of the invention may comprise one or more biomarkers selected from HNRPR, CDC20. PRIM2A, HRSP12. ENY2, TMEM141, RECQL, STK3, and MX2.

Among the fifty biomarkers in Table 6, fourteen biomarker are identified as being associated with tumorigenesis: ACP5, FSCN1, HOXA1, HSF1, NDC80, VSIG4, BRRN1, RNF2, UCHL5, HNRPR, PRIM2A, HRSP12, ENY2, and MX2. In some embodiments, a biomarker panel that can be used in the methods of the invention may comprise one or more biomarkers selected from these fourteen biomarkers.

In certain embodiments, a biomarker panel that can be used in the methods of the invention may comprise one or more biomarkers selected from the identified invasion-associated biomarkers (ACP5, ANLN, ASFB, BRRN1, BUB1, CDC2, CENPM, DEPDC1 ELTD1, EXT1, FSCN1, HCAP-G, HMGB1, HMGB2, HOXA1, HSF1, ITGB3BP, KIF20A, KIF2C, KNTC2, MCM7, MTHFD2, NASP, PLVAP, PTP4A3. RNF2, SPAG5, TGM2, UBE2C, UCHL5, and VSIG4) and one or more biomarkers selected from the identified anoikis resistance-associated biomarkers (HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA1).

In certain embodiments, a biomarker panel that can be used in the methods of the invention may comprise one or more biomarkers selected from the identified tumorigenesis-associated biomarkers (ACP5, FSCN1, HOXA1, HSF1, NDC80, VSIG4, BRRN1, RNF2, UCHL5, HNRPR, PRIM2A, HRSP12, ENY2, and MX2) and one or more biomarkers selected from the identified invasion-associated biomarkers (ACP5, ANLN, ASF1B. BRRN1, BUB1, CDC2, CENPM, DEPDC1, ELTD1, EXT1, FSCN1, HCAP-G, HMGB1, HMGB2, HOXA1, HSF1, ITGB3BP, KIF20A, KIF2C, KNTC2, MCM7, MTHFD2, NASP, PLVAP, PTP4A3, RNF2, SPAG5, TGM2, UBE2C, UCHL5, and VSIG4).

In certain embodiments, a biomarker panel that can be used in the methods of the invention may comprise one or more biomarkers selected from the identified tumorigenesis-associated biomarkers (ACP5, FSCN1, HOXA1, HSF1, NDC80, VSIG4, BRRN1, RNF2, UCHL5, HNRPR, PRIM2A, HRSP12, ENY2, and MX2) and one or more biomarkers selected from the identified anoikis resistance-associated biomarkers (HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA1).

In certain embodiments, a biomarker panel that can be used in the methods of the invention may comprise one or more biomarkers selected from the identified tumorigenesis-associated biomarkers (ACP5, FSCN1, HOXA1 HSF1, NDC80, VSIG4, BRRN1, RNF2, UCHL5, HNRPR, PRIM2A, HRSP12, ENY2, and MX2); one or more biomarkers selected from the identified anoikis resistance-associated biomarkers (HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA 1); and one or more invasion-associated biomarkers (ACP5, ANLN, ASF1B, BRRN1, BUB1, CDC2, CENPM, DEPDC1, ELTD1, EXT1, FSCN1, HCAP-G, HMGB1, HMGB2, HOXA1, HSF1, ITGB3BP, KIF20A, KIF2C, KNTC2, MCM7, MTHFD2, NASP, PLVAP, PTP4A3, RNF2, SPAG5, TGM2, UBE2C, UCHL5, and VSIG4).

In certain embodiments, a biomarker panel of the present invention may further comprise one or more of the 360 biomarkers listed in Table 1.

In certain embodiments, the biomarker panel of the present invention may be modified by replacing one or more of the selected biomarkers with one or more new biomarkers. The new substitute biomarker(s) may be involved in the same or similar biological process or pathway as the existing biomarker. In some embodiments, the existing biomarker and its substitute biomarker are both associated with anoikis resistance or invasion or tumorigenesis. In some embodiments, the existing biomarker and its substitute biomarker are both involved in a PTEN pathway, PI3K pathway, Ras pathway, mTOR pathway or other signaling pathways. The modified biomarker panel may maintain the same or similar sensitivity and/or specificity as the previous biomarker panel. In some embodiments, the modified biomarker panel may produce higher sensitivity and/or specificity than the previous biomarker panel.

### Measurement of Biomarkers

The biomarkers of this invention can be measured in various forms. For example, one may measure the gene copy numbers (e.g., DNA gain or loss) of the biomarkers. Alternatively, one may measure the RNA transcript levels of the biomarkers. One also may measure DNA methylation states or DNA acetylation states of the biomarkers. Or one may measure the protein activity (e.g., phosphatase activity or enzymatic activity) or level of the biomarkers. In some embodiments, one may determine the presence or absence of a mutation or polymorphism in the nucleotide (or amino acid) sequence of the biomarker(s).

At the nucleic acid level, biomarkers may be measured by electrophoresis, Northern and Southern blot analyses, in situ hybridization (e.g., single or multiplex nucleic acid in situ hybridization technology such as Advanced Cell Diagnostic's RNAscope technology), RNAse protection assays, and microarrays (e.g., Illumina BeadArray™ technology; Beads Array for Detection of Gene Expression (BADGE)). Biomarkers may also be measured by polymerase chain reaction (PCR)-based assays, e.g., quantitative PCR, real-time PCR, quantitative real-time PCR (qRT-PCR), and reverse transcriptase PCR (RT-PCR). Other amplification-based methods include, for example, transcript-mediated amplification (TMA), strand displacement amplification (SDA), nucleic acid sequence based amplification (NASBA), and signal amplification methods such as bDNA. Nucleic acid biomarkers also may be measured by sequencing-based techniques such as, for example, serial analysis of gene expression (SAGE), RNA-Seq, and high-throughput sequencing technologies (e.g., massively parallel sequencing), and Sequenom MassARRAY® technology. Nucleic acid biomarkers also may be measured by, for example, NanoString nCounter, and high coverage expression profiling (HiCEP).

At the protein level, biomarkers may be measured in whole cells and/or in subcellular compartments (e.g., nucleus, cytoplasm and cell membrane). Exemplary methods include, without limitation, immunoassays such as immunohistochemistry assays (IHC), immunofluorescence assays (IF), enzyme-linked immunosorbent assays (ELISA), immunoradiometric assays, and immunoenzymatic assays. In immunoassays, one may use, for example, antibodies that bind to a biomarker or a fragment thereof. The antibodies may be monoclonal, polyclonal, chimeric, or humanized. One may also use antigen-binding fragments of a whole antibody, such as single chain antibodies, Fv fragments, Fab fragments, Fab′ fragments, F(ab′)₂ fragments, Fd fragments, single chain Fv molecules (scFv), bispecific single chain Fv dimers, diabodies, domain-deleted antibodies, single domain antibodies, and/or an oligoclonal mixture of two or more specific monoclonal antibodies. Other methods to measure biomarkers at the protein level include, for example, chromatography, mass spectrometry, Luminex xMAP Technology, microfluidic chip-based assays, surface plasmon resonance, sequencing, Western blot analysis, aptamer binding, molecular imprints, or a combination thereof. To determine whole cell and/or subcellular levels of a biomarker, one may also use methods such as AQUA® (see, e.g., U.S. Pat. Nos. 7,219,016, and 7,709,222; Camp et al., Nature Medicine, 8(11): 1323-27 (2002)), and Definiens TissueStudio™ (see, e.g., U.S. Pat. Nos. 7,873,223, 7,801,361, 7,467,159, and 7,146,380, and Baatz et al., Comb Chem High Throughput Screen, 12(9):908-16 (2009)).

For biomarker proteins known to have enzymatic activity, their levels can be measured through their activities. Such assays include, without limitation, kinase assays, phosphatase assays, and reductase assays, among many others. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

The nucleotide or amino acid sequences of the biomarkers may be determined by any methods known in the art to detect genotypes, single nucleotide polymorphisms, gene mutations, gene copy numbers, DNA methylation states, or DNA acetylation states.

Exemplary methods include, but are not limited to, polymerase chain reaction (PCR) analysis, sequencing analysis, electrophoretic analysis, restriction fragment length polymorphism (RFLP) analysis, Northern blot analysis, quantitative PCR, reverse-transcriptase-PCR analysis (RT-PCR), co-amplification at lower denaturation temperature-PCR (COLD-PCR), multiplex PCR, allele-specific oligonucleotide hybridization analysis, comparative genomic hybridization, heteroduplex mobility assay (HMA), single strand conformational polymorphism (SSCP), denaturing gradient gel electrophisis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, Hpall tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfite-driven conversion of non-methylated cytosine to uracil, co-amplification at lower denaturation temperature-PCR (COLD-PCR), multiplex PCR, methyl-binding PCR analysis, or a combination thereof.

In some embodiments, post-translational modifications of a biomarker may be relevant to cancer prognosis. Such modifications include, without limitation, phosphorylation (e.g., tyrosine, serine, or threonine phosphorylation), glycosylation (e.g., N-linked, O-linked, C-linked), acylation, acetylation, ubiquitination, deacetylation, alkylation, methylation, amidation, biotinylation, gamma-carboxylation, glutamylation, glycyation, hydroxylation, covalent attachment of heme moiety, iodination, isoprenylation, lipoylation, prenylation, GPI anchor formation, myristoylation, farnesylation, geranylgeranylation, covalent attachment of nucleotides or derivatives thereof, ADP-ribosylation, flavin attachment, oxidation, palmitoylation, pegylation, covalent attachment of phosphatidylinositol, phosphopantetheinylation, polysialylation, pyroglutamate formation, racemization of proline by prolyl isomerase, tRNA-mediation addition of amino acids such as arginylation, sulfation, the addition of a sulfate group to a tyrosine, or selenoylation of the biomarker. Such modification may be detected, for example, by antibodies specific for the modifications, or by mass spectrometry (e.g., MALDI-TOF).

### Sample Sources

The skilled worker would appreciate that a sample that be used in the methods of the present invention for measuring the levels or determining sequences of a biomarker or a biomarker panel can be any sample useful for this purpose, such as a cancerous tissue sample or a bodily fluid sample comprising circulating tumor cells. In some embodiments, the noncancerous cells are excluded from the tissue sample. In some embodiments, the tissue sample is a solid tissue sample, a bodily fluid sample, or circulating tumor cells. In some embodiments, the tissue sample is a cancerous tissue sample. In some embodiments, the cancerous tissue is melanoma, prostate cancer, breast cancer, or colon cancer tissue. Examples of a biological sample that can be used in this invention include, without limitation, cancerous tissue samples, blood cells, tumor cells, lymphoma cells, epithelia cells, endothelial cells, stem cells, progenitor cells, mesenchymal cells, osteoblast cells, osteocytes, hematopoietic stem cells, foam cells, adipose cells, transcervical cells, cardiocytes, fibrocytes, cancer stem cells, myocytes, cells from kidney, cells from gastrointestinal tract, cells from lung, cells from reproductive organs, cells from central nervous system, hepatic cells, cells from spleen, cells from thymus, cells from thyroid, cells from an endocrine gland, cells from parathyroid, cells from pituitary, cells from adrenal gland, cells from islets of Langerhans, cells from pancreas, cells from hypothalamus, cells from prostate tissues, cells from breast tissues, cells from circulating retinal cells, ophthalmic cells, auditory cells, epidermal cells, cells from the urinary tract, blood, urine, stool, saliva, lymph fluid, cerebrospinal fluid, synovial fluid, cystic fluid, ascites, pleural effusion, interstitial fluid, or ocular fluid. The sample may be circulating cells or non-circulating cells (e.g., biopsied sample).

In some embodiments, the solid tissue sample may be a formalin-fixed paraffin embedded tissue sample, a snap-frozen tissue sample, an ethanol-fixed tissue sample, a tissue sample fixed with an organic solvent, a tissue sample fixed with plastic or epoxy, a cross-linked tissue sample, surgically removed tumor tissue, or a biopsy sample (e.g., a core biopsy, an excisional tissue biopsy, or an incisional tissue biopsy).

### Clinical Applications of Biomarkers and Biomarker Panels

By measuring the levels (e.g., expression or activity) of the biomarkers described herein in a sample from a cancer patient, one can reliably predict survival of the patient at a given time point. The levels can used to predict prognosis, such as low or high risk of having metastatic cancer or recurrence of cancer. As used herein, the term “prognosis” refers to the prediction of the likely outcome of a disease. For example, prognosis of cancer may refer to the prediction, within a given period, of how the cancer will progress, or the likelihood of cancer recurrence or metastasis, or the likelihood or risk of death attributable to cancer. In various embodiments, the given period of time may be at least six months, one year, two years, three years, five years, eight years, ten years, fifteen years or longer.

The levels of the biomarkers described herein also can be used to analyze a tissue sample taken from the patient for diagnostic uses, such as staging (e.g., stage I, II, III, or IV) cancer. The levels of the biomarkers also can be used to monitor the progression of a tumor in a patient. The levels also can be used to monitor efficacy of a cancer therapy (e.g., surgery, radiation therapy, or chemotherapy) independent of, or in addition to, traditional, established risk assessment procedures.

The levels of the biomarkers described herein also can be used to identify a patient in need of adjuvant therapy. As used herein, the term “adjuvant therapy” refers to a therapy given in conjunction with surgery. Examples of adjuvant therapy that can be used in the present invention include, without limitation, radiation therapy, chemotherapy, immunotherapy, hormone therapy, experimental therapy (e.g., as part of a clinical trial), neo-adjuvant therapy (therapy administered prior to the primary therapy), and targeted therapy. As used herein, the term “targeted therapy” refers to using a biologics or agent or compound to inhibit or enhance the function of molecular target, or a signaling pathway associated therewith, in cancer cells. Targeted therapy associated with methods of this invention may include therapy that targets one or more biomarkers described herein and/or a component of the signaling pathway associated with one or more of the biomarkers.

The levels of the biomarkers also can be used to select a treatment regimen for a cancer patient. For example, if the measured levels of the biomarkers indicate that a patient is at a high risk of having metastatic cancer or recurrence of cancer, the patient may need adjuvant therapy. The biomarkers can further help select an appropriate adjuvant therapy. For example, one can measure the levels of the biomarkers from a patient before and after the proposed adjuvant therapy and compare the two measurements. An observed difference between the two measurements may indicate that the proposed adjuvant therapy is suitable for the patient. If no significant difference is identified between the two treatments, the proposed adjuvant therapy may not be suitable for the patient.

The levels of the biomarkers described herein also can be used to guide further diagnostic tests. For example, the levels can be used to identify if a patient is in need of a sentinel lymph node biopsy. If the measured levels of the biomarkers indicate that a patient is at a high risk of having metastatic cancer or recurrence of cancer, the patient may need a sentinel lymph node biopsy. By contrast, if the measured levels of the biomarkers indicate that a patient is at a low risk of having metastatic cancer or recurrence of cancer, the patient may not need a sentinel lymph node biopsy.

By determining if the sequences (e.g., nucleotide or amino acid) of the biomarkers described herein in a sample from a cancer patient comprise a mutation or mutations (e.g., presence of a mutation compared to a wild-type or reference sequence associated with high risk of metastatic cancer or recurrence of cancer), one also can reliably predict survival of the patient at a given time point. For example, the presence or absence of the mutation(s) can used to predict prognosis (e.g., low or high risk of having metastatic cancer or recurrence of cancer).

The presence or absence of the mutation(s) of the biomarkers described herein also can be used to analyze a tissue sample taken from the patient for diagnostic uses, such as staging (e.g., stage I, II, III, or IV) cancer. The presence or absence of the mutation(s) of the biomarkers also can be used to monitor the progression of a tumor in a patient. The presence or absence of the mutation(s) also can be used to monitor efficacy of a cancer therapy (e.g., surgery, radiation therapy, or chemotherapy) independent of, or in addition to, traditional, established risk assessment procedures.

The presence or absence of the mutation(s) of the biomarkers described herein also can be used to identify a patient in need of adjuvant therapy

The presence or absence of the mutation(s) of the biomarkers also can be used to select a treatment regimen for a cancer patient. For example, if the presence of the mutation(s) in the biomarkers indicates that a patient is at a high risk of having metastatic cancer or recurrence of cancer, the patient may need adjuvant therapy. The biomarkers can further help select an appropriate adjuvant therapy. For example, one can detect the presence or absence of the mutation(s) of the biomarkers from a patient before and after the proposed adjuvant therapy and compare the two measurements. An observed difference between the two measurements may indicate that the proposed adjuvant therapy is suitable for the patient. If no significant difference is identified between the two treatments, the proposed adjuvant therapy may not be suitable for the patient.

The presence or absence of the mutation(s) of the biomarkers described herein also can be used to guide further diagnostic tests. For example, the presence or absence of the mutation(s) can be used to identify if a patient is in need of a sentinel lymph node biopsy. If the presence of the mutation(s) in the biomarkers indicates that a patient is at a high risk of having metastatic cancer or recurrence of cancer, the patient may need a sentinel lymph node biopsy. By contrast, if the presence of the mutation(s) in of the biomarkers indicates that a patient is at a low risk of having metastatic cancer or recurrence of cancer, the patient may not need a sentinel lymph node biopsy.

### ACP5

The inventors have identified ACP5, a tartrate-resistant acid phosphatase, as a pro-invasion oncogenic biomarker that can confer enhanced metastasis risk in vivo and also carry prognostic significance in patients diagnosed with primary melanomas (see Example 3 described below). The inventors have also discovered that the tumorigenesis and metastasis of melanoma requires the phosphatase activity of ACP5 (see Example 3 described below). The present invention provides new diagnostic methods and therapies by targeting the phosphatase activity of ACP5 to treat melanoma and other types of cancer (e.g., neutralizing antibodies and/or chemical inhibitors).

In one aspect, the invention provides a biomarker panel that can be used in the present invention comprising ACP5. For example, the invention provides a method for predicting prognosis of a cancer patient, comprising measuring the level of ACP5 (e.g., expression or activity) or determining the nucleotide or amino acid sequence of ACP5 in a sample from the patient (e.g., a cancerous tissue sample). The measured level of ACP5, or the presence (or absence) of a mutation in the determined sequence of ACP5 as compared to a reference sequence of ACP5, is indicative of the prognosis of the cancer patient. In some embodiments, the method of the invention measures the level of the catalytic activity or phosphatase activity of ACP5. The biomarker panel also may further comprise measuring the levels or determining the nucleotide or amino acid sequences of one or more other biomarkers described herein, such as one or more biomarkers selected from the group consisting of ANLN, ASF1B. BRRN1, BUB1, CDC2, CENPM, DEPDC1, ELTD1. EXT1, FSCN1, HCAP-G, HMGB1, HMGB2, HOXA1, HSF1, ITGB3BP, KIF20A, KIF2C, KNTC2, MCM7, MTHFD2, NASP, PLVAP, PTP4A3, RNF2, SPAG5, TGM2, UBE2C, UCHL5, and VSIG4 or one or more biomarkers selected from the group consisting of HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA1.

In another aspect, the invention provides a method for treating a cancer patient in need thereof by administering an agent that modulates the level (e.g., expression or activity) of ACP5. In some embodiments, the administered agent (compound, drug, or biologics) may cause a conformational change of ACP5, thus preventing the biological activity of ACP5 (e.g., phosphatase activity). In some embodiments, the administered agent (compound, drug, or biologics) may cause disruption of the interaction between ACP5 and a substrate of ACP5. In some embodiments, the administered agent (compound, drug, or biologics) may target one or more residues in ACP5 that are associated with the phosphatase activity of ACP5. For example, His111, His214 and Asp265 are known to be important for the phosphatase activity of ACP5 based on the available structural information or a rat ACP5 protein. In some embodiments, the administered agent (compound, drug, or biologics) can inhibit the secretion of ACP5 or the activity of the secreted ACP5. Examples of the agents that can be used to modulate the level of ACP5 include, without limitation, chemical inhibitors, acid phosphatase inhibitors (e.g., molybdate), or antibodies.

### Therapeutic Application of Biomarkers and Biomarker Panels

Biomarkers or biomarker panels of the present invention also have therapeutic applications in treating cancer or reducing the risk of cancer recurrence or development of cancer (e.g., metastatic cancer). In one aspect, biomarkers or biomarkers panels of the present invention can be used to aid identification of potential therapeutic agents (e.g., compounds, drugs, or biologics) that are capable of treating cancer or reducing the risk of cancer recurrence or development of cancer (e.g., metastatic cancer). For example, a cell expressing a biomarker or biomarker panel described herein can be contacted with a candidate compound. It is then determined that whether the candidate compound alters the expression or activity of the biomarker or biomarker panel. The alteration observed in the presence of the candidate compound indicates that the compound is capable of reducing the risk of cancer occurrence or development of cancer (e.g., metastatic cancer) or capable of treating cancer. If the expression or activity level of the biomarker is known to be up-regulated in patients at a high risk of having metastatic cancer or cancer recurrence, the candidate compound that is capable of down-regulating the expression or activity level of the biomarker can have potential therapeutic applications. If the expression or activity level of the biomarker is known to be down-regulated in patients at a high risk of having metastatic cancer or cancer recurrence, the candidate compound that is capable of up-regulating the expression or activity level of the biomarker can have potential therapeutic applications.

In some embodiments, the biomarker panel may comprise one or more biomarker selected from FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4. HNRPR. CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1. In some embodiments, the biomarker panel may comprise one or more biomarker selected from FSCN1, HOXA1, HSF1, NDC80, VSIG4, BRRN1, HNRPR, PRIM2A, HRSP12, ENY2, or MX2.

In some embodiments, the biomarker panel that can be used for identifying therapeutic compounds comprise ACP5. The inventors have identified ACP5 as a pro-invasion tumorigenic biomarker and its phosphatase activity is required for metastasis or tumorigenesis. Accordingly, if a candidate compound that is capable of inhibiting the biological activity (e.g., phosphatase activity) or reducing the expression level (e.g., inhibiting secretion) of ACP5, such compound may be a potential therapeutic compound for cancer (e.g., melanoma). The candidate compound may cause a conformation change of ACP5, or disrupt the interaction between ACP5 and a substrate of ACP5, or inhibit the secreting of ACP5. The candidate compound may target one or more residues of in ACP5 that are associated with the phosphatase activity of ACP5. For example, His111, His214 and Asp265 are known to be important for the phosphatase activity of ACP5 based on the available structural information or a rat ACP5 protein.

In one embodiment, the biomarker panel that can be used for identifying therapeutic compounds comprise RNF2. In another embodiment, the biomarker panel that can be used for identifying therapeutic compounds comprise UCHL5.

### Kits

The levels of the biomarkers in a panel may be measured using a kit with detection reagents that specifically detect and quantify the biomarkers. The detection reagents may have been detectably labeled, or the kit provides labeling reagents for conjugation to the detection reagents. The kit may comprise an array of detection reagents, e.g., antibodies and/or oligonucleotides that can bind to biomarker proteins (or fragments thereof) or nucleic acids, respectively. In some embodiments, the biomarkers are proteins and the kit contains antibodies that bind to the biomarkers. In other embodiments, the biomarkers are nucleic acids and the kit contains oligonucleotides or aptamers that bind to the biomarkers. In some embodiments, the oligonucleotides may be fragments of the biomarker genes. For example the oligonucleotides can be 200, 150, 100, 50, 25, or fewer nucleotides in length.

A kit also may contain in separate containers a nucleic acid or antibody (alone, or already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, quantum dots, luciferase, and radiolabels, among others. Instructions (e.g., written, tape, VCR, CD-ROM, and/or DVD) for carrying out the assay may be included in the kit.

The biomarker detection reagents provided in a kit can be immobilized on a solid matrix such as a porous strip to form at least one biomarker detection site. The measurement or detection region of the porous strip may include a plurality of sites containing, for example, a nucleic acid or antibody, and may optionally contain sites for negative and/or positive controls. Alternatively, control sites can be located on a separate strip from the test strip. Optionally, the different detection sites may contain different amounts of biomarker detection reagents, e.g., a higher amount in the first detection site and lesser amounts in subsequent sites. Upon the addition of test sample, the number of sites displaying a detectable signal may provide a quantitative indication of the amount or level of biomarkers present in the sample. The detection sites may be configured in any suitably detectable shape and can be in the shape of a bar or dot spanning the width of a test strip.

In some embodiments, a kit comprises a nucleic acid substrate array comprising one or more nucleic acid sequences that specifically identify one or more biomarker nucleic acid sequences. In certain embodiments, the substrate array can be on a solid substrate (for example, a “chip” such as a microarray chip (see, e.g., U.S. Pat. No. 5,744,305)). Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen. Carlsbad, Calif.). In alternative embodiments, a kit comprises an antibody substrate array comprising one or more antibodies that specifically identify one or more biomarker proteins (e.g., an array for performing an immunoassay such as an ELISA assay or AQUA®).

### Additional Prognostic Factors

The biomarker panels of this invention may be used in conjunction with additional biomarkers, clinical parameters, or traditional laboratory risk factors known to be present or associated with the clinical outcome of interest. In some embodiments, the biomarker panels, when used in conjunction with an additional prognostic factor, achieves better performance (e.g., higher sensitivity or specificity) in cancer prognosis. Clinical parameters or traditional laboratory risk factors for tumor metastasis may include, for example, tumor stage, tumor grade, tumor size, tumor visual characteristics, tumor location, tumor growth, lymph node status, histology, tumor thickness (Breslow score), ulceration, proliferative index, tumor-infiltrating lymphocytes, age of onset, PSA level, or Gleason score. Other traditional laboratory risk factors for tumor metastasis are known to those skilled in the art.

The biomarker panels of the present invention provide useful prognostic information about a variety of cancers, including, for example, carcinomas (e.g., malignant tumors derived from epithelial cells such as, for example, common forms of breast, prostate, lung, and colon cancer), sarcomas (e.g., malignant tumors derived from connective tissue or mesenchymal cells), lymphomas and leukemias (i.e., malignancies derived from hematopoietic cells), germ cell tumors (i.e., tumors derived from totipotent cells). Specific examples of these cancers include, without limitation, cancers of: breast, skin, bone, prostate, ovaries, uterus, cervix, liver, lung, brain, spine, larynx, gallbladder, pancreas, rectum, parathyroid, thyroid, adrenal gland, immune system, head and neck, colon, stomach, bronchi, and kidneys.

Further details of the invention will be described in the following non-limiting Examples. It should be understood that these examples, while indicating preferred embodiments of the invention, are given by way of illustration only, and should not be construed as limiting the appended embodiments. From the present disclosure and these examples, one skilled in the art can ascertain certain characteristics of this invention, and without departing from the spirit and scope thereof, can make various changes and modifications of the invention to adapt it to various usages and conditions.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and embodiments, the word “comprise,” or variations such as “comprises” or “comprising” will be understood to imply the inclusion of a stated integer or group of integers but not the exclusion of any other integer or group of integers. The materials, methods, and examples are illustrative only and not intended to be limiting.

The following examples are meant to illustrate the methods and materials of the present invention. Suitable modifications and adaptations of the described conditions and parameters normally encountered in the art which are obvious to those skilled in the art are within the spirit and scope of the present invention.

The following materials and methods were used in the experiments described in the Examples below.

Genetically Engineered Mouse (GEM) Models for Melanoma, Comparative Data Analyses and In vivo Tumor Assays: All mice were bred and maintained under defined conditions at the Dana-Farber Cancer Institute (DFCI), and all procedures were approved by the Animal Care and Use Committee of DFCI and conformed to the legal mandates and national guidelines for the care and maintenance of laboratory animals. The tetracycline-inducible MET-driven mouse (iMet) model (Tyr-rtTA;Tet-Met;Ink4a/Arf−/−) was constructed similar to the iHRAS* model (Tyr-rtTA;Tet-HRASa^{V12G};Ink4a/Arf^{-/-}) described in Chin et al., Nature 400, 468-472 (1999). Mice were sacrificed according to institute guidelines and organs were fixed in 10% buffered formalin and paraffin embedded. Tissue sections were stained with H&E to enable classification of the lesions and detection of tumor metastasis. For detection of c-Met protein, tumor sections were immunostained with total c-Met and phospho c-Met (Tyr1349) antibodies (Cell Signaling Technology). iMet tumors were additionally immunostained with S100 antibody (Sigma). RNA from cutaneous melanomas derived from iMet or iHRAS* models were profiled on Affymetrix Gene Chips and resultant transcriptomes were compared using Significance Analysis of Microarray (SAM 2.0) to generate a phenotype-based (metastatic capable or not) differentially expressed gene list. Cross-species triangulation to human gene expression and copy number aberrations was based on ortholog mapping.

For xenograft tumorigenicity studies, HMEL468 cells were transduced with pLenti6/V5 DEST-generated virus for stable expression of GFP (control) or the indicated genes. Following selection with blasticidin (Invitrogen; 5 μg/ml) for 5-7 days, 1.0×10⁶ cells [prepared in Hanks Balanced Salts (HBS) at 1:1 with Matrigel] were injected subcutaneously into the right flank of NCr-Nude (Taconic) mice. Two-tailed t-test calculations were performed using Prism 4 (Graphpad). In vivo metastasis assays were performed by 1) orthotopic skin tumor assays using 1205Lu cells stably-expressing GFP (control) or ACP5 and 2) orthotopic mammary fad pad assays using non-metastatic NB008 adenocarcinoma cells stably-expressing vector (control) or ACP5.

Cell Culture:

HMEL468 primed melanocytes were a subclone of PMEL/hTERT/CDK4(R24C)/p53DD/BRAF^{V600E} cells as described (Garraway et al., Nature 436, 117-122 (2005)). The non-metastatic NB008 cell line was established from a spontaneous tumor isolated from the breast of a G4 52-week old female mTerc−/−, p53+/− mouse. GFP-mTerc was re-introduced into the resulting cell line by lentiviral transduction prior to use in these studies. The WM115 melanoma cell line was obtained from the Wistar Institute, and the 1205Lu melanoma cell line was obtained from the American Type Culture Collection. M619 and C918 melanoma lines have been described in Maniotis et al., Am J Pathol 155, 739-752 (1999). All cell lines were propagated at 37° C. and 5% CO₂ in humidified atmosphere in RPMI 1640 medium supplemented with 10% FBS.

Invasion Screen and Transwell Invasion Assays:

The low complexity genetic screen for cell invasion was performed using Tert-immortalized melanocytes HMEL468 in 96-well modified Boyden chambers coated with Matrigel (96-well tumor invasion plates; BD Bioscience) following the manufacture's recommendations. Invaded cells were detected with labeling using 4 uM Calcein AM (BD Bioscience) and measured by fluorescence at 494/517 nm (Abs/Em) after 20 hrs incubation at 37° C. and 5% CO₂. Positive-scoring candidates were identified as those scoring 2× standard deviations from the vector control. Validation assays for cell invasion were performed in standard 24-well invasion chambers containing Matrigel (BD Bioscience) following the manufacture's recommendations. Following 18-20 hrs incubation at 37° C. and 5% CO₂, chambers were fixed in 10% formalin, stained with crystal violet for manual counting or by pixel quantitation with Adobe Photoshop (Adobe). Data was normalized to input cells to control for differences in cell number (loading control).

Automated Quantitative Analysis (AQUA®):

Uses of human tissues in this study are approved by the Yale institutional IRB, HIC protocol number 9500008219 including consent and waived consent. AQUA® analysis and the Yale Melanoma Arrays and tissue microarray construction have been described in Camp et al., Nat Med 8, 1323-1327 (2002); and Gould Rothberg et al., J Clin Oncol 27, 5772-5780 (2009). Arrays were stained with the following antibodies: monoclonal anti-Fascin 1 diluted 1:500 (clone 55K2, Santa Cruz Biotechnology, Inc.), polyclonal anti-HOXA1 diluted 1:50 (BO1P, Abnova), polyclonal anti-HSF1 diluted 1:2500 (AO1, Abnova), monoclonal anti-NDC80 diluted 1:50 (clone 1A10, Abnova), monoclonal anti-ACP5 diluted 1:100 (clone 26E5, Abcam), polyclonal anti-NCAPH diluted 1:750 (Bethyl Laboratories, Inc.), and polyclonal anti-VSIG4 diluted 1:1000 (ab56037, Abcam).

Anchorage Independent Growth Assays:

Soft-agar colony formation assays were performed on 6-well plates in triplicate for cells transduced with pLKO-shGFP (Open Biosystems) or shRNA (Bill Hahn, DFCI/Broad Institute; available via Open Biosystems) hairpins targeting the indicated genes. Cells were selected for 5 days with 2.5 μg/μl puromycin, and 1×10⁴ cells were mixed thoroughly in cell growth medium containing 0.4% SeaKem LE agarose (Fisher) in RPMI+10% FBS, followed by plating onto bottom agarose prepared with 0.65% agarose in RPMI+10% FBS. Each well was allowed to solidify and subsequently covered in 1 ml RPMI+10% FBS+P/S, which was refreshed every 4 days. Colonies were stained with 0.05% (wt/vol) iodonitrotetrazolium chloride (Sigma) and scanned at 1200 dpi using a flatbed scanner, followed by counting and two-tailed t-test calculation using Prism 4 (Graphpad). Verification of knockdown was achieved by qRT-PCR using gene-specific primer sets (SABiosciences).

Co-Immunoprecipitation and Immunoblotting:

For immunoprecipitation studies, lysates were prepared in NP-40 buffer (20 mMTris-HCl, pH 8.0, 150 mMNaCl, 2 mM EDTA, 1% NP40) containing 1 mM PMSF, 1× Protease Inhibitor Cocktail (Roche) and 1× Phosphatase inhibitor (Calbiochem) for immunoprecipitation. Anti-Paxillin (Abeam) or anti-FAK (Santa Cruz) antibody was added to cell lysates for 2 hr at 4° C. with rocking, followed by incubation overnight with protein G sepharose (Roche) at 4° C. with rocking. Immunoprecipitates were washed 3× for 10 min with lysis buffer, eluted by the addition of SDS loading buffer after centrifugation and resolved on NuPAGE 4-12% Bis-Tris gels (Invitrogen) for immunoblotting on PVDF (Millipore). The following antibodies were used for immunoblotting following the manufacture's recommendations: anti-FAK (Santa Cruz); anti-FAK (Tyr397; Cell Signaling); anti-Paxillin (Abeam); anti-Paxillin (Tyr118; Cell Signaling); anti-Vinculin (Santa Cruz); anti-V5 (for ACP5 detection; Invitrogen) and anti-phospho-tyrosine (Millipore).

Cell Imaging:

Single-plane phase image was collected on a Nikon Ti with a 40× Plan-Apochromatic phase objective NA 0.95 and a Clara camera using Andor iQ software (Andor Technology). Time lapse phase images were collected on a Nikon TE2000-E with a 10× phase objective and an OrcaER camera (Hamamatsu) at the Dana-Farber Cancer Institute Confocal and Light Microscopy Core. Shutters, stage position, and camera were controlled by NIS-Elements software (Nikon, Melville, N.Y.). Images were collected every 2 minutes at 6-12 stage positions for 20 hours.

Breast Cancer Prognostic Studies:

Expression patterns of the 18 candidate pre-invasion oncogenes and MammaPrint® 70-gene signature were used for Kaplan-Meier survival analyses of the indicated breast cancer datasets by K-means clustering using the survival package in R.

Accession Numbers:

Expression array data for the iMet and iRas tumors generated by these studies have been deposited into the GEO database with accession GSE29074.

Inducible, Melanocyte-Specific MET Expression in Transgenic Mice.

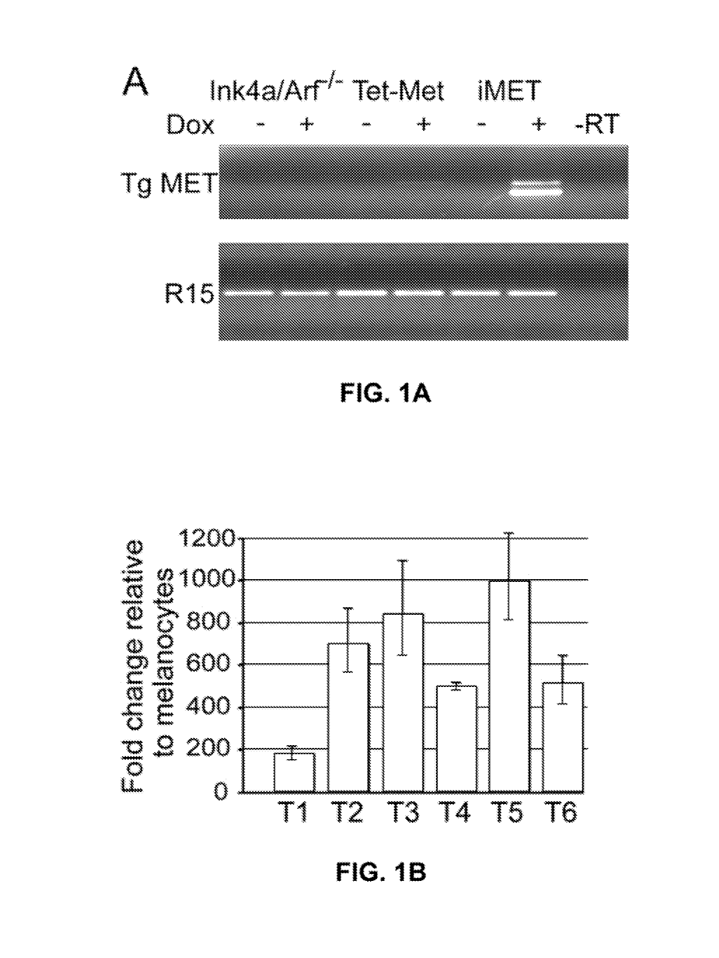

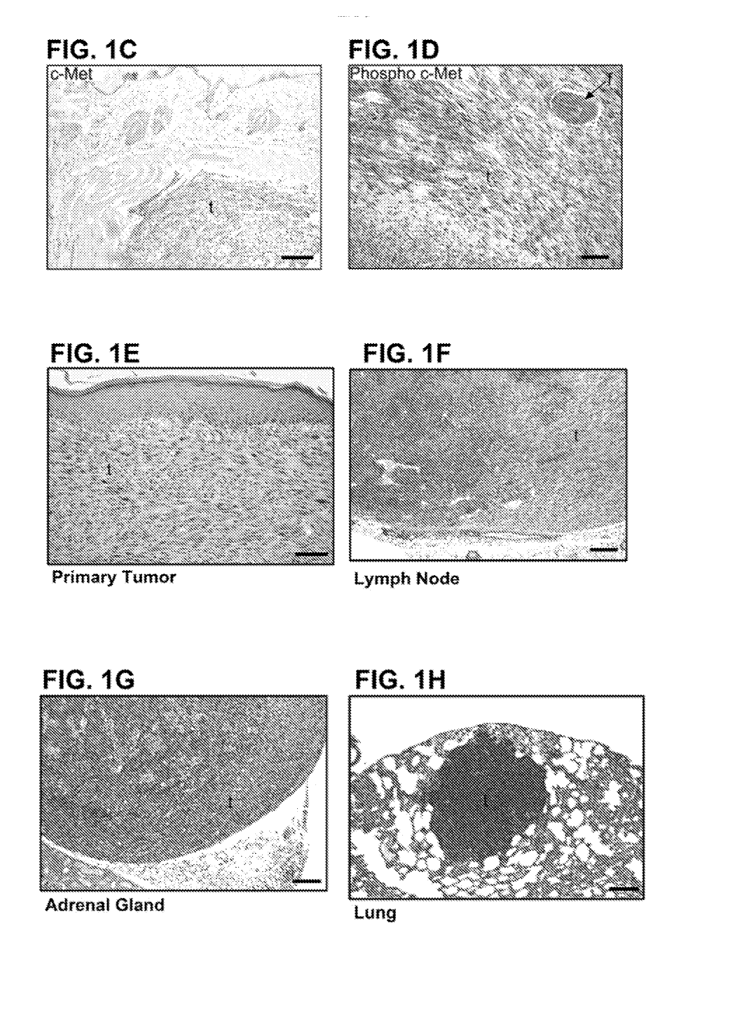

In order to engineer the inducible Met transgene, the reverse tetracycline transactivator, Tet promoter and the tyrosinase enhancer/promoter transgene were used as described in Chin et al., Genes Dev 11, 2822-2834 (1997); Chin et al., Nature 400, 468-472 (1999); and Ganss et al., EMBO J. 13, 3083-3093 (1994). Mouse c-Met cDNA (a gift from George F Vande Woude, Grand Rapids, Mich.) was cloned under the control of a Tet promoter similar to as described in Chin et al., Nature 400, 468-472 (1999). Multiple transgene founder lines were generated at the expected frequency. Tet-Met transgenic animals were subsequently crossed to transgenic allele carrying the reverse tetracycline transactivator under the control of tyrosinase gene promoter-enhancer elements (designated Tyr-rtTA) (Gossen et al., Science 268, 1766-1769 (1995)). Given the frequency and demonstrated relevance of INK4a/Arf deletions in melanoma (Hussussian et al., Nat Genet. 8, 15-21 (1994); Kamb et al., Science 264, 436-440 (1994)), animals were intercrossed with INK4a/Arf null mice to generate cohorts of single and double transgenic mice (designated iMet) that were deficient for INK4a/ARF. To verify doxycycline induced expression of the MET transgene, melanocytes were harvested from Ink4a/Arf−/−, Tet-Met, and iMet animals and cultured in the presence or absence of doxycycline. Semi-quantitative RT-PCR analysis specific for the MET transgene confirms expression in only those melanocytes generated from iMet animals on doxycycline and not from Ink4a/Arf^{-/-} and Tet-Met control animals (FIG. 1A).

A cohort consisting of 63 single (Tyr-rtTA or Tet-Met) and double (iMet) transgenic mice (Table 2) were administered doxycycline in drinking water upon weaning and monitored for melanoma formation. While no single transgenic animals in the presence or absence of doxycycline developed tumors, two of 30 iMet animals on doxycycline formed spontaneous melanomas. In addition to spontaneous melanoma, it was observed that other tumor types associated with germline INK4a/ARF mutations (Serrano et al., Cell 85, 27-37 (1996)). Many of these tumors, consisting primarily of lymphomas, materialized early leading to mortality and therefore deterred detection of additional melanomas.

Hepatocyte growth factor (HGF), the activating ligand for MET, is up-regulated during wound healing responses (Michalopoulos et al., Proc Natl Acad Sci USA 90, 8817-8821 (1997)); therefore, a subset of animals were dorsally wounded by skin biopsy and monitored the cohort for melanoma. Following wounding, six out of eight iMet mice on doxycycline formed melanomas with an average latency of 12 weeks. These data suggest that recruitment of HGF through the process of wound healing is required for tumor initiation in the iMet transgenic animals.

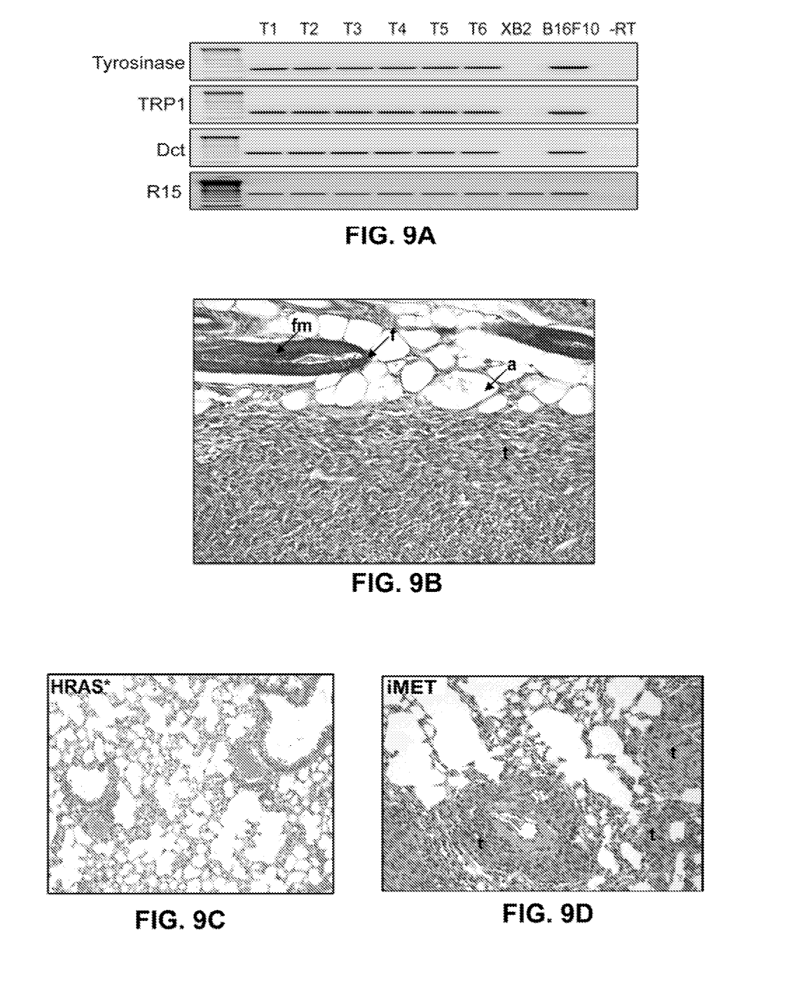

In order to verify the melanocytic origin of the six tumors isolated from the iMet animals, expression of the melanocytic markers Tyrosinase, TRP1 and Dct were assayed using RNA collected from tumor specimens (FIG. 9A). Similar to the well-characterized B16F10 melanoma tumor cell line, all six Met-induced tumors express the melanocytic markers. In contrast, these markers are not expressed in the XB2 keratinocytic cell line as expected. S100 immunohistochemistry revealed positivity in tumor cells (FIG. 9B) further indicating melanocytic tumor origin.

The melanomas developed in wounded iMet animals initiated as lesions at the biopsy site and later expanded as plaque-like tumors with alopecia. Zones of progression to malignancy were apparent by the emergence of local vertical thickenings that developed into melanomas with ulceration through the epidermis (data not shown) similar to the phenotype observed in the wound-induced melanoma GEM characterized by Mintz and Silvers (Mintz and Silvers, 1993). Histological analysis of the primary melanomas revealed a dermal spindle and epithelioid cell malignant neoplasm. Cytological atypia was moderate and numerous mitotic figures were present. Immunohistochemical analysis revealed Met over-expression in tumors but not in normal surrounding skin structures, and activation of c-Met was determined by positive immune-staining with a phospho-specific MET antibody (FIGS. 1C-1D). The level of HGF expression in the six MET induced tumors isolated from the iMet animals were assayed (FIG. 1E). RT-PCR analyses demonstrate a higher level of HGF expression in all six tumor samples compared to non-transformed Ink4a/Arf melanocytes. A full histological survey was performed in four of the six advanced tumor-bearing mice and detected micrometastases in three animals. Primary tumors metastasized mainly to lymph nodes and fewer cases to adrenal glands and lung (FIGS. 1E-1H and 9C-9D). Notably, the histologic features of the metastatic lesions and the primary tumors were indistinguishable on light microscopy of hematoxylin and eosin-stained sections.

Gene Expression Profiling and Data Analyses.

Met- and HRAS*-driven mouse tumor RNAs were labeled and hybridized to Affymetrix GeneChip Mouse Genome 430 2.0 Arrays by the Dana-Farber Cancer Institute Microarray Core Facility according to the manufacturer's protocol. Expression data was processed using the R/bioconductor package (www.bioconductor.org). Briefly, the background correction method was MAS (v4.5), normalization method was constant, expression value summary method was median polish (RMA). P/M/A call method was MAS5. Probe sets with at least 2 present calls among all 12 tumor samples (16,434 probe sets) were selected for further differential expression analyses between six iMet tumors versus six iHRas tumors. Significance Analysis of Microarray (SAM 2.0; www-stat.stanford.edu/˜tibs/SAM/) was used for differential expression analysis (Tusher et al., Proc Natl Acad Sci USA 98, 5116-5121 (2001)). Two-class unpaired sample analysis was performed, followed by filtering for minimum 2-fold change and delta value adjustment so that the false discovery rate would be less than 0.05. The Ingenuity Pathways Analysis program (www.ingenuity.com/index.html) was used to further analyze the cellular functions and pathways that were significantly regulated in metastatic melanoma.

Comparison of Mouse Gene Expression and Human Expression/Array-CGH Data.

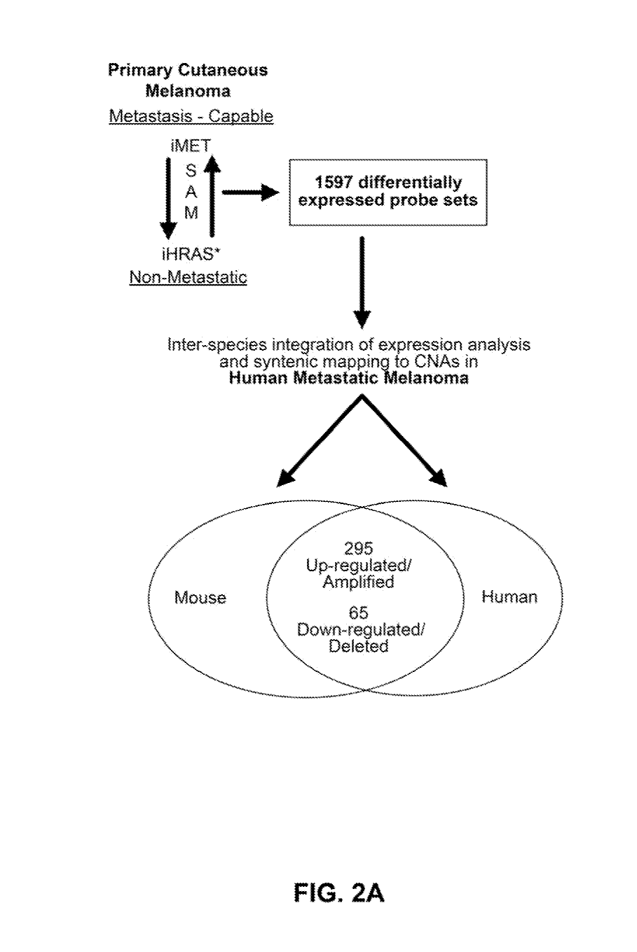

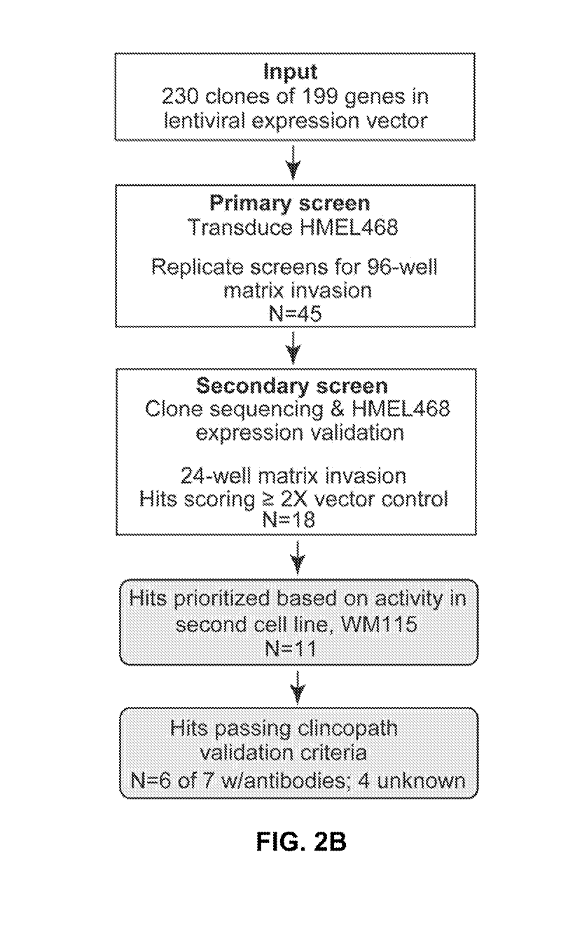

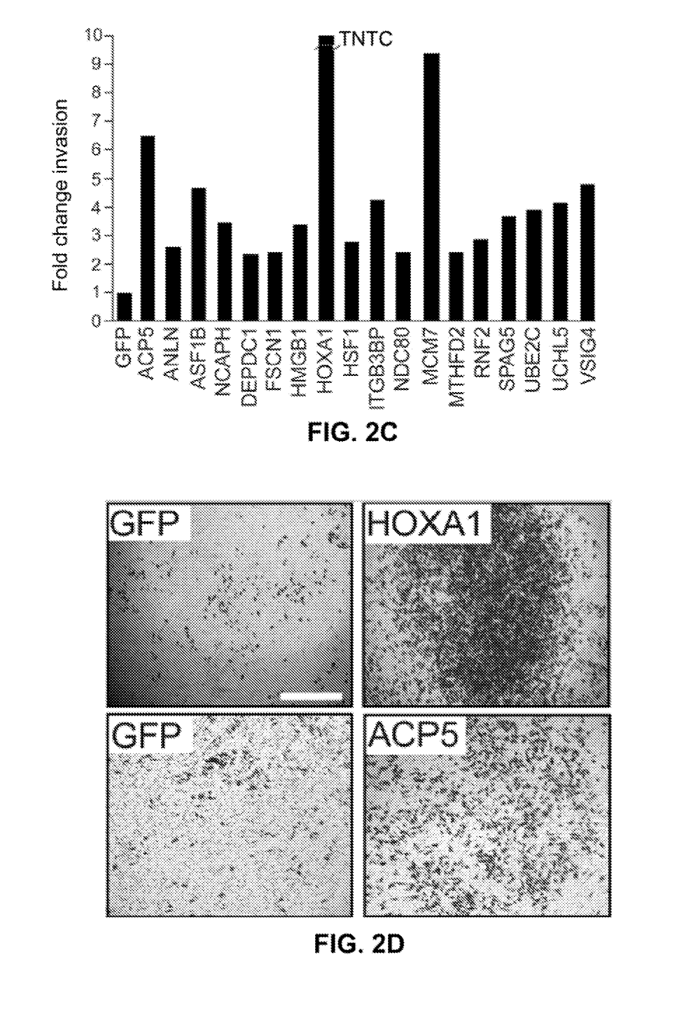

Non-redundant, differentially-expressed probe sets obtained from the expression analysis of mouse tumors (described above) were mapped to human orthologs (using NCBI Homologene database) that showed 1) statistically significant (≧2-fold) expression in human melanoma specimens (Kabbarah et al., PLoS One 5, e10770 (2010)) and/or 2) are present in copy number aberrations in human metastatic melanoma identified by array-CGH (GSE7606). This comparative oncogenomic analysis led to a list of 360-genes comprised of 295 up-regulated/amplified and 65 down-regulated/deleted candidates (see FIG. 2A and Table 3).

DNA Constructs and Low-Complexity Library.

For the low complexity cDNA library, 230 cDNAs representing 199 genes of the 295 up-regulated/amplified genes described in Table 3 were obtained from the ORFeome collection (Dana-Farber Cancer Institute) and transferred to pLcnti6/V5 DEST (Invitrogen) via Gateway recombination following the manufacture's recommendations. The 20 candidate cDNAs scoring in the invasion screen were sequence and expression verified, and homogenous clone preparations of the validated 18 genes (listed below in Table 7) were used for all invasion and tumor validation studies using virus prepared following the Invitrogen's recommendations.

TABLE 7

| | Gene | CDNA | BC | |
|---|---|---|---|---|
| Gene | ID | length | Number | Notes |
| ACP5 | 54 | 978 | BC025414 | |
| ANLN | 54443 | 1218 | BC034692 | Variant; Transcription |
| | | | | start at nucleotide 2158 |
| ASF1B | 55723 | 609 | BC036521 | |
| DEPDC1 | 55635 | 1584 | BC065304 | |
| FASCIN | 6624 | 1482 | BC000521 | |
| HMGB1 | 3146 | 648 | BC003378 | |
| HOXA1 | 3198 | 1008 | BC032547 | |
| HSF1 | 3297 | 1590 | BC014638 | |
| ITGB3BP | 23421 | 534 | BC009929 | |
| MCM7 | 4176 | 2160 | BC013375 | |
| MTHFD2 | 10797 | 747 | BC015062 | |
| NCAPH | 23397 | 2226 | BC024211 | |
| NDC80 | 10403 | 1929 | BC035617 | |
| RNF2 | 6045 | 1011 | BC012583 | |
| SPAG5 | 10615 | 3582 | BC000322 | Contains a one nucleotide |
| | | | | deletion at C-term that |
| | | | | shifts out-of-frame with |
| | | | | V5 tag in pLent6 V5/DEST; |
| | | | | alters last four residues |
| | | | | (EFLS* > LNF*) |
| UBE2C | 11065 | 540 | BC016292 | |
| UCHL5 | 51377 | 987 | BC015521 | |
| VSIG4 | 11326 | 1200 | BC010525 | |

96-Well Viral Production, Transduction and Transwell Invasion Assays.

Approximately 3×10⁴ 293T cells were seeded in 100 μl per each well in 96-well flat bottom plates 24 hrs prior to transfection (˜90% confluent) in DMEM+10% FBS. For each well transfection, 150 ng viral backbone and 110 ng lentiviral packaging vectors were diluted to 15 μl using Opti-MEM (Invitrogen). The resulting vector mix was combined with 15 μl Opti-MEM containing 0.6 μl Liptofectamine-2000 (Invitrogen), incubated RT for 20 min and added to the 100 μl media covering the 293T cells. The media was replaced with DMEM+10% FBS+1% penicillin/streptomycin approximately 10 hrs post-transfection, and 4 viral supernatant collections were taken starting at 36 hrs post transfection and combined. 150 μl viral supernatant containing 8 ug/ml polybrene was added to target cells (HMEL468) that were seeded into 96-well flat bottom plates 24 hrs prior to infection (70-80% confluent). Cells were infected twice and allowed to recover in RPMI+10% FBS+P/S for 24 hours following the second infection, after which cells were trypsized and applied to 96-well tumor invasion plates coated with Matrigel (BD Bioscience) following the manufacture's recommendations. Invaded cells were detected with labeling using 4 uM Calcein AM (BD Bioscience) and measured by fluorescence at 494/517 nm (Abs/Em). Positive-scoring candidates were identified as those scoring 2× standard deviations from the vector control.

For standard 24-well transwell invasion assays, Matrigel coated chambers (BD Biosciences) were utilized to assess invasiveness following the manufacture's suggestions. Briefly, cells were trypsinized, rinsed twice with PBS, resuspended in serum-free RPMI 1640 media, and seeded at 7.5×10⁴ cells/well for HMEL468 and 5.0×10 for WM115. Chambers were seeded in triplicate or quadruplicate and placed in 10% serum-containing media as a chemo-attractant as well as in cell culture plates in duplicate as input controls. Following 18-20 hrs incubation, chambers were fixed in 10% formalin, stained with crystal violet for manual counting or by pixel quantitation with Adobe Photoshop (Adobe). Data was normalized to input cells to control for differences in cell number (loading control).

Gene Expression Real-time Quantitative PCR.

For analyses of gene expression, total RNA was isolated from primary cutaneous melanomas or from cultured cells using Trizol (Invitrogen) according to manufacturer's protocol. Total RNA was treated with RQ1 DNAse (Promega) and 1 μg total RNA was used for reverse transcription reaction using Superscript II polymerase (Invitrogen) primed with oligo(dT). Coding regions were amplified by PCR or quantitative real time PCR using SYBR Green (Applied Biosystems) on an Mx3000P real-time PCR system (Stratagene), and the comparative cycle threshold method was used to quantify mRNA copy number. For the iMet GEM-related studies Ribosomal protein R15 was used as an internal expression control.

TABLE 8
Primer sequences

| | |
|---|---|
| c-Met: | 5′-TCTGTTGCCATCCCAAGACAACATTGATGG |
| | 5′-AAATCTCTGGAGGAGGTTGG |
| HGF | 5′-CAAGGCCAAGGAGAAGGTTA |
| | 5′-TTTGAAGTTCTCGGGAGTGA |
| Tyr | 5′-CCAGAAGCCAATGCACCTAT |
| | 5′-AGCAATAACAGCTCCCACCA |
| TRP1 | 5′-ATTCTGGCCTCCAGTTACCA |
| | 5′-GGCTTCATTCTTGGTGCTTC |
| DCT: | 5′-AACAACCCTTCCACAGATGC |
| | 5′-TCTCCATTAAGGGCGCATAG |
| R15 | 5′-CTTCCGCAAGTTCACCTACC |
| | reverse-TACTTGAGGGGGATGAATCG |

For RNAi knockdown verification, RNA expression levels were normalized to human GAPDH. GAPDH and gene-specific primer sets were purchased from SABiosciences.

Histological Analysis and Immunohistochemical Staining.

Mice were sacrificed according to institute guidelines and organs were fixed in 10% buffered formalin and paraffin embedded. Tissue sections were stained with H&E to enable classification of the lesions and detection of tumor metastasis. For detection of c-Met protein, tumor sections were immunostained with total c-Met and phospho c-Met (Tyr1349) antibodies (Cell Signaling Technology). iMet tumors were additionally immunostained with S100 antibody (Sigma).

TMA-IHC and Automated Quantitative Analysis (AQUA®).

Patient characteristics for the Yale Melanoma Discovery Array and tissue microarray construction have been described in Gould Rothberg et al., J Clin Oncol 27, 5772-5780 (2009). The Yale Melanoma Progression Array was constructed by the Yale University Tissue Microarray Facility and included single 0.6 mm cores from 20 benign nevi, 20 vertical growth phase primary melanomas and 20 metastases, the latter representing lesions from subcutaneous, lymph node and visceral sites. TMAs were deparaffinized with xylene, rehydrated and antigen-retrieved by pressure cooking for 15 min in citrate buffer (pH=6). Slides were pre-incubated with 0.3% bovine serum albumin (BSA) in 0.1M tris-buffered saline (TBS, pH=8) for 30 min at RT. Melanoma TMAs were then incubated overnight with a cocktail of either a rabbit polyclonal anti-S100 antibody diluted 1:100 (Z0311, Dako), rabbit polyclonal anti-GP100 diluted 1:25 (ab27435, Abcam) and a mouse target antibody including the monoclonal anti-Fascin 1 diluted 1:500 (clone 55K2, Santa Cruz Biotechnology, Inc.), polyclonal anti-HOXA1 diluted 1:50 (BO1P, Abnova), polyclonal anti-HSF1 diluted 1:2500 (AO1, Abnova), monoclonal anti-KNTC2 (NDC80) diluted (clone 1A10, Abnova), monoclonal anti-ACP5 diluted (clone 26E5, Abcam), or a mouse monoclonal S100 antibody diluted 1:100 (15E2E2, BioGenex) and a rabbit target antibody including the polyclonal antiBRRN1 (NCAPH) diluted 1:750 (Bethyl Laboratories, Inc.), polyclonal anti-VSIG4 diluted 1:1000 (ab56037, Abcam). This was followed by a 1 hr incubation with Alexa 546-conjugated goat anti-mouse secondary antibody (A11003, Molecular Probes) diluted 1:100 in rabbit EnVision reagent (K4003, Dako) and Alexa 546-conjugated goat anti-rabbit secondary antibody (A11010, Molecular Probes) diluted 1:100 in mouse EnVision reagent (K4001, Dako) for mouse and rabbit target antibodies respectively. Cyanine 5 (Cy5) directly conjugated to tyramide (FP1117, Perkin-Elmer) at a 1:50 dilution was used as the fluorescent chromagen for target detection. Prolong mounting medium (ProLong Gold, P36931, Molecular Probes) containing 4′,6-Diamidino-2-phenylindole (DAPI) was used to identify nuclei. Serial sections of a small control slide of 30 melanoma specimens and 10 normal controls were stained alongside to assess reproducibility and a negative control in which the primary antibody was omitted, were used for each immunostaining run.

Automated Quantitative Analysis (AQUA®) quantifies protein expression within specific subcellular compartments and has been described in Camp et al., Nat Med 8, 1323-1327 (2002). In brief, a series of high resolution monochromatic in- and out-of-focus images were obtained for each histospot using the signal from the DAPI, S100 (GP100)-Alexa 546 and the target-Cy5 channel by the PM-2000 microscope. Stromal and non-stromal elements are distinguished from tumor by creating a tumor “mask” from the S100 (GP100) signal. The binary tumor mask (each pixel being either “on” or “off”) was based on an intensity threshold set upon visual inspection of each histospot. The cytoplasmic compartment is subsequently generated from subtracting the DAPI based nuclear compartment from the tumor mask. AQUA® scores of the proteins of interest in each subcellular compartment (total tumor mask, nuclear, and cytoplasmic) were calculated by dividing the signal intensity (scored on a scale from 0-255) by the area of the specific compartment.

For statistical analysis, histospots containing less than 0.17 mm² of tumor were excluded from analysis. The AQUA® scores were averaged for individuals with multiple histospots on any array before analysis. Ratios of Cytoplasmic:Nuclear AQUA® Scores were compared following log transformation. Bivariate comparisons between target scores and clinicopathologic variables were assessed using ANOVA analysis. For ACP5, survival curves were calculated using the Kaplan-Meier product-limit method and significance determined by the Mantel-Cox log-rank statistic. All statistical analyses were done using Statview 5.0 (SAS Institute).

Anchorage Independent Growth.

Soft-agar assays were performed on 6-well plates in triplicate for cells transduced with pLKO-shGFP (Open Biosystems) or each of the following shRNA (Bill Hahn, DFCI/Broad Institute; available via Open Biosystems) hairpins targeting the indicated genes (Table 9). (see www.broadinstitute.org/mai/public/gene/search for additional clone details).

TABLE 9

| | | | | Clone |
|---|---|---|---|---|
| Gene | Gene | | | Desig- |
| Symbol | ID | Clone ID | Clone Name | nation |
| ACP5 | 54 | TRCN0000050566 | NM_001611.2- | ACP5-2 |
| | | | 1015s1c1 | |
| ACP5 | 54 | TRCN0000050564 | NM_001611.2- | ACP5-4 |
| | | | 276s1c1 | |
| FSCN1 | 6624 | TRCN0000123041 | NM_003088.2- | FSCN1-1 |
| | | | 1112s1c1 | |
| FSCN1 | 6624 | TRCN0000123039 | NM_003088.2- | FSCN1-3 |
| | | | 1699s1c1 | |
| HOXA1 | 3198 | TRCN0000015030 | NM_005522.3- | HOXA1-1 |
| | | | 149s1c1 | |
| HOXA1 | 3198 | TRCN0000015028 | NM_005522.3- | HOXA1-3 |
| | | | 1866s1c1 | |
| HSF1 | 3297 | TRCN0000007484 | NM_005526.1- | HSF1-4 |
| | | | 1312s1c1 | |
| HSF1 | 3297 | TRCN0000007483 | NM_005526.1- | HSF1-5 |
| | | | 331s1c1 | |
| NDC80* | 10403 | TRCN0000107942 | NM_006101.1- | NDC80-3 |
| | | | 302s1c1 | |
| VSIG4 | 11326 | TRCN0000137889 | NM_007268.1- | VSIG4-2 |
| | | | 1516s1c1 | |
| VSIG4 | 11326 | TRCN0000137643 | NM_007268.1- | VSIG4-4 |
| | | | 450s1c1 | |

Following transduction following the manufacturer's protocol and selection for 5 days with 2.5 μg/μl puromycin, 1×10⁴ cells were mixed thoroughly in cell growth medium containing 0.4% SeaKem LE agarose (Fisher) in RPMI+10% FBS, followed by plating onto bottom agarose prepared with 0.65% agarose in RPMI+10% FBS. Each well was allowed to solidify and subsequently covered in 1 ml RPMI+10% FBS+P/S, which was refreshed every 4 days. Colonies were stained with 0.05% (wt/vol) iodonitrotetrazolium chloride (Sigma) and scanned at 1200 dpi using a flatbed scanner, followed by counting and two-tailed t-test calculation using Prism 4 (Graphpad). Verification of knockdown was achieved by qRT-PCR (described above) and immunoblotting with candidate-specific antibodies where available.

In Vivo Metastasis.

For the 1205Lu melanoma model, cells were transduced with pLenti6.3/V5 DEST-generated lentivirus. Cell lines stably expressing GFP (control) or ACP5 were generated by selection with blastidicin (5 μg/ml) for 4 days following viral transduction. 1.0×10⁶ cells suspended in 200 μl HBSS were injected subcutaneously into the right flank of NCr-Nude (Taconic) mice (n=5). Tumor growth was monitored over time and mice were sacrificed based on tumor burden (largest dimension ≦2 cm) in accordance with the PI's IACUC-approved animal protocol. Organs were screened for metastasis by H&E.

For the orthotopic fat pad model, 2.5×10⁴ cells were injected in a 20 microliter volume with Matrigel (1:1) in the right inguinal fat pad of female hosts. Mice were closely monitored and sacrificed as described above for metastasis screening by use of UV light (for expression of GFP) and H&E. The NB008 cell line used in this study was established from a spontaneous tumor isolated from the breast of a G4 52-week old female mTerc−/−, p53+/− mouse. mTerc was re-introduced into the resulting cell line by lentiviral transduction.

### Example 1

### Identification and Characterization of Biomarkers Associated Invasion and Tumorigenesis

This example adopts a comparative oncogenomics-guided function-based strategy involving (i) comparison of global transcriptomes of two genetically engineered mouse models with contrasting metastatic potential, (ii) genomic and transcriptomic profiles of human melanoma, (iii) functional genetic screen for enhancers of cell invasion and (iv) evidence of expression selection in human melanoma tissues. This integrated effort identified a set of genes that are potently pro-invasive and oncogenic. These genes can be used as biomarkers for predicting prognosis in cancer.

Early-stage melanoma is often cured by surgical excision, yet some cases without clinical evidence of dissemination recur with lethal metastatic disease despite successful surgical removal of the primary tumor. Elucidation of the molecular basis underlying such aggressive biology has been a longstanding focus, with the goal of identifying prognostic biomarkers and rational therapeutics for high-risk patients diagnosed with early-stage disease who are in need of further treatment in adjuvant setting. This example teaches how genetically engineered mouse models, cross-species cancer genomics knowledge, and functional screens can be exploited and integrated to identify robust pro-invasion drivers of metastasis that are also bona fide oncogenes.

Cancers are highly heterogeneous on both the genomic and cellular levels such that similarly staged early disease can exhibit radically different clinical outcomes—from cure following surgical removal of the primary tumor to death within months of diagnosis due to widespread metastasis. Metastasis is responsible for the majority of cancer-related mortality and involves multiple interrelated steps by which primary tumor cells spread to establish cancerous lesions at distant sites (Gupta et al., Cell 127, 679-695 (2006)). To become metastatic, tumor cells acquire a number of biological capabilities to overcome barriers of dissemination and distant growth such as invasion, anoikis resistance, extravasation, colonization and growth in new microenvironments. Each of these biological attributes can be conferred by genetic or epigenetic events observed in tumors (Hanahan et al., Cell 144, 646-674 (2011)), supporting the thesis that biological heterogeneity of cancers, including metastatic potential, is dictated by underlying genomic alterations.

While significant data exists in support of a classical model of stepwise accumulation of genetic events which endow increasing malignant potential, the identification of extensive genome rearrangements in early stage cancers (driven in part by telomere crisis) (Rudolph et al., Nat Genet. 28, 155-159 (2001); Chin et al., Nat Genet. 36, 984-988 (2004)) raise the possibility that some tumors may acquire genomic alterations with significant metastatic potential early in their evolution. Such tumors would inherently carry higher risk of metastasis despite early diagnoses. This deterministic model is consistent with the finding that transcriptomic profiles of primary tumors share striking resemblance with their metastatic lesions (Perou et al., Nature 406, 747-752 (2000)), and gene expression patterns of the primary bulk tumor can predict the likelihood of recurrence or metastatic spread, e.g. MammaPrint® and OncotypeDx® (van't Veer et al., Nature 415, 530-536 (2002); Paik et al., N Engl J Med 351, 2817-2826 (2004)). Furthermore, the prognostic significance of these gene expression signatures supports the view that information on metastatic propensity is encoded in the bulk of the primary tumor (van't Veer et al., Nature 415, 530-536 (2002); van de Vijver et al., N Engl J Med 347, 1999-2009 (2002); Ramaswamy et al., Nat Genet. 33, 49-54 (2003)).

Therefore, pro-metastatic genetic alterations acquired early at primary tumor stage might themselves be classical oncogenes and tumor suppressor genes which can confer a selective growth advantage during tumorigenesis, and if so, such genes would be subject to recurrent genomic alterations in cancer (i.e., amplification and loss). The present invention has identified a number of such pro-metastasis oncogenes. These pro-metastasis oncogenes therefore can be used as both prognostic markers as well as therapeutic targets for inherently aggressive early stage cancers. The present invention has used melanoma as a disease model and systematically identified a number of putative metastasis driving genes which also confer transforming oncogenic activity in early stage cancers. The existence of such genes has further validated the concept of ‘oncogenic driver of metastasis’ or ‘metastasis oncogenes’.

Evolutionarily-Conserved, Differentially Expressed Genes with Metastatic Potential

In view of the enormous genomic complexity of human melanoma and the less than complete certainty surrounding occult metastatic disease in any given human patient two extensively characterized genetically engineered mouse (GEM) models of human melanoma with completely distinct metastatic profiles were used as extreme cases for comparison. The selected melanoma models are (i) the HRAS^{V12G}-driven mouse melanoma model (Tvr-rtTA; Tet-HRAS^{V12G};Ink4a/Arf^{-/-}, hereafter “iHRAS*”) (Chin et al., Nature 400, 468-472 (1999)), and (ii) a Met-driven GEM model (Tyr-rtTA; Tet-Met;Ink4a/Arf^{-/-}, hereafter “iMet”). Briefly, following a similar engineering strategy used for the iHRAS model, the iMet model is constructed with an inducible Met transgene (Tet-Met) by placing murine c-Met cDNA downstream of a reverse tetracycline-responsive promoter element as described previously (Ganss et al., EMBO J. 13, 3083-3093 (1994); Chin et al., Genes Dev 11, 2822-2834 (1997): Chin et al., Nature 400, 468-472 (1999)). Tet-Met transgenic animals were subsequently bred with transgenic mice carrying the reverse tetracycline transactivator under the control of tyrosinase gene promoter-enhancer elements (designated Tyr-rtTA) (Gossen et al., Science 268, 1766-1769 (1995)). Given the frequency and demonstrated relevance of INK4a/Arf deletions in melanoma (Hussussian et al., Nat Genet. 8, 15-21 (1994); Kamb et al., Science 264, 436-440 (1994)), these compound transgenic alleles were further intercrossed onto an INK4a/Arf null background to generate cohorts of single and double transgenic mice (designated iMet) deficient for INK4a/ARF whose melanocytes express Met upon induction with doxycycline (FIG. 1A).

iMet mice develop melanomas at sites of skin wounding with an average latency of 12 weeks (Table 2). These lesions are positive for prototypical melanocyte markers and express phospho-Met receptor and its ligand hepatocyte growth factor (HGF) (FIGS. 1B-1D and FIGS. 9A-9B). These iMet melanomas uniformly metastasize to lymph nodes and show occasional dissemination to the adrenal glands and lung parenchyma, which are common sites for metastases in human melanoma (FIGS. 1E-1H). In sharp contrast, the iHRAS* melanoma model develops aggressive cutaneous melanomas which do not metastasize (Chin et al., Genes Dev 11, 2822-2834 (1997); Chin et al., Nature 400, 468-472 (1999)). Consistent with the contrasting metastatic potential of iMet and iHRAS* primary tumors, only iMet melanoma-derived cell lines were able to seed and grow to large macroscopic lesions in tail-vein experimental metastasis assays (FIGS. 9C-9D).

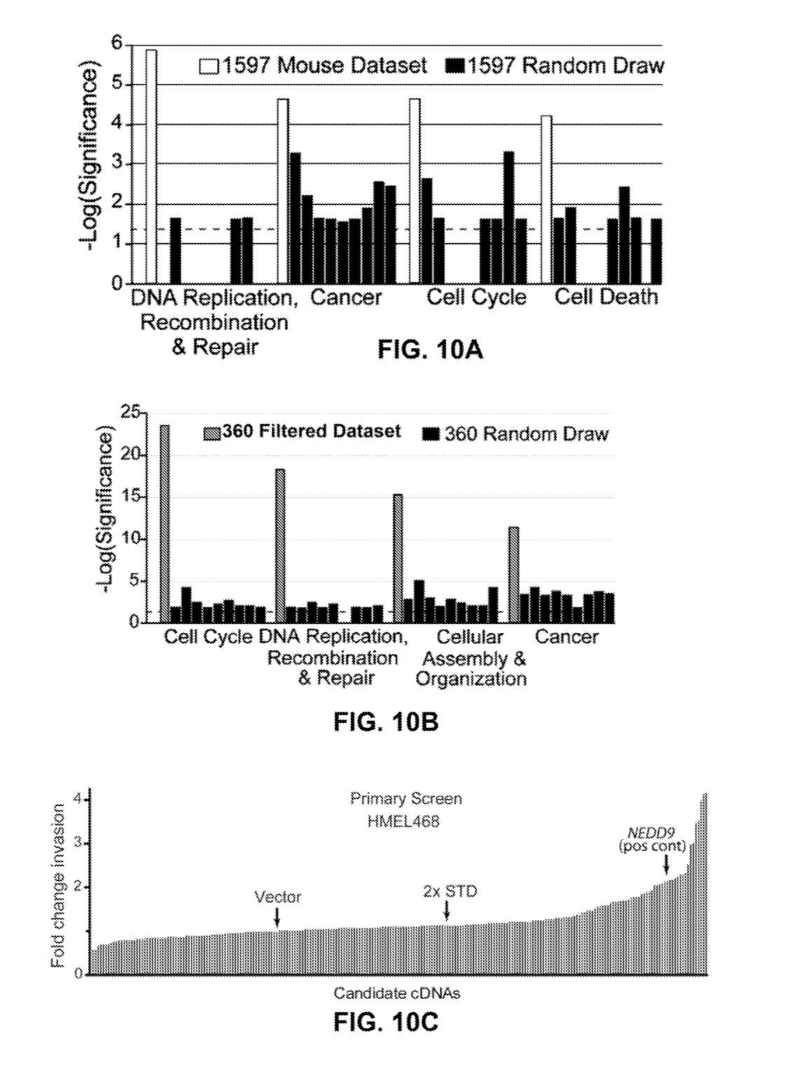

Using these two GEM models as “extreme cases”, the transcriptomic profiles of primary cutaneous melanomas from iHRAS* and iMet models were compared to define 1597 gene probe sets with ≧2-fold differential expression at a false discovery rate <0.05. This list of differentially expressed genes was next intersected with genes residing in recurrent copy number aberrations (CNAs) in human metastatic melanoma (GEO accession #GSE7606) and/or genes exhibiting significant differential expression between primary and metastatic melanomas in human (Kabbarah et al., PLoS One 5, e10770 (2010)). This comparative oncogenomics analysis led to a list of 360-genes comprised of 295 up-regulated/amplified and 65 down-regulated/deleted candidates (FIG. 2A; Table 3), representing differentially expressed genes in primary melanoma that are correlated with metastatic potential. Compared with the 1597 probe set, this cross-species intersected list of 360 genes was significantly more enriched for cancer-relevant functional networks based on Ingenuity Pathway Analysis (IPA; FIGS. 10A-10B).

Identification and Functional Characterization of Biomarkers Associated with Invasion and Tumorigenesis

From the above cross-species triangulated gene list for metastatic potential, functionally active metastasis drivers in primary melanomas were identified following the experimental outline in FIG. 2B. In particular, a genetic screen was designed to screen for genes present in such primary melanoma that have pro-invasive active. These genes can be potentially metastasis drivers in such primary drivers because the ability of primary melanoma cells to invade downward into the dermis and subcutis is significantly correlated with metastasis, and a primary melanoma with pro-invasive genetic events is more likely to metastasize early. In particular, the 295 up-regulated genes selected by the screen were further investigated using a gain-of-function screening design given their possible therapeutic potential. The human ORFeome collection (horfdb.dfci.harvard.edu/) contained 230 open reading frame (ORF) cDNAs corresponding to 199 of the 295 unique up-regulated/amplified candidates (Table 11), which were then transferred to a lentiviral expression system for transduction into HMEL468 (PMEL/hTERT/CDK4(R24C)/p53DD), a TERT-immortalized primary human melanocyte line engineered with BRAFV600^{E} mutation (Garraway et al., Nature 436, 117-122 (2005)). For the primary screen, a 96-well transwell invasion assay with fluorometric readout was utilized to measure the ability of candidate genes to enhance migration and invasion of HMEL468 through Matrigel (BD Biosciences), which simulates extracellular matrix. Lentiviral expression vectors encoding GFP and NEDD9 (Kim et al., Cell 125, 1269-1281 (2006); O'Neill et al., Cancer Res 67, 8975-8979 (2007); Sanz-Moreno et al., Cell 135, 510-523 (2008); Izumchenko et al., Cancer Res 69, 7198-7206 (2009)) were used as negative and positive controls, respectively. The primary screen was performed in duplicate, and 45 candidates that reproducibly scored two standard deviations from the GFP control were considered as primary screen hits (FIG. 10C; Table 3). Secondary validation of these 45 candidate genes was performed by assaying their invasive ability in standard 24-well Matrigel invasion chambers with parallel sequencing and expression verification, yielding 18 genes (Table 5) possessing >2-fold enhancement of invasion compared to the GFP control (FIGS. 2C-2D and Table 1). As a frame of reference, the positive control pro-metastasis gene, NEDD9, enhanced invasion by 1.5-fold in this system and has been shown to be required for cell movement (Sanz-Moreno et al., Cell 135, 510-523 (2008)) and in vivo metastasis of breast cancers (Izumchenko et al., Cancer Res 69, 7198-7206 (2009)).

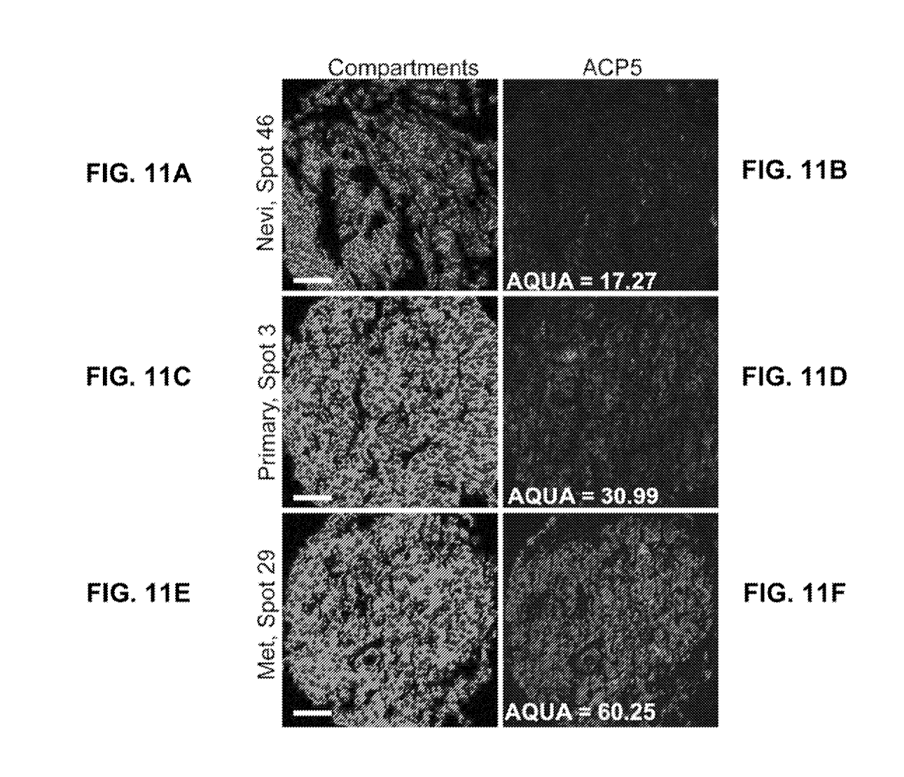

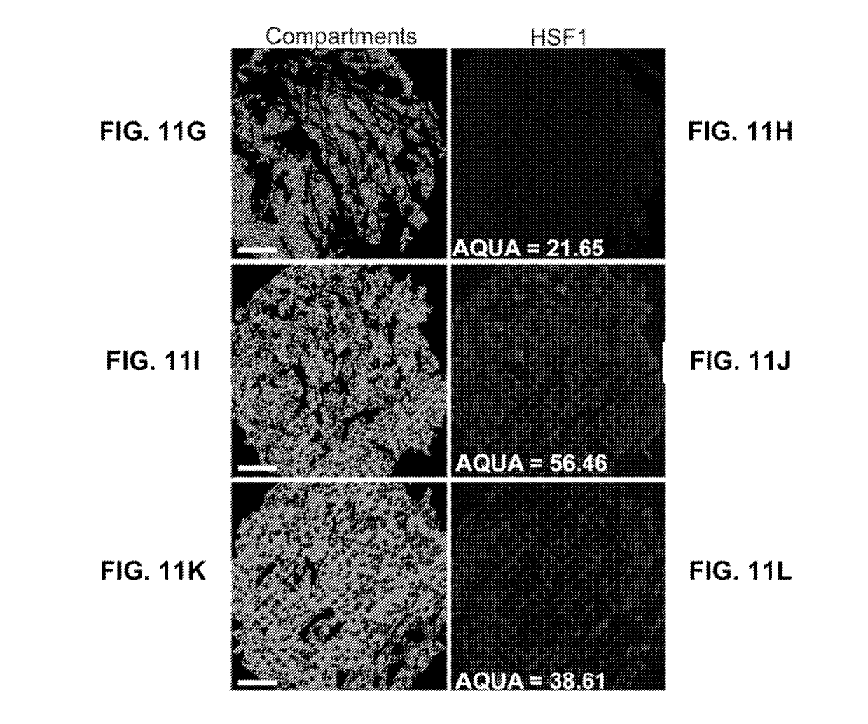

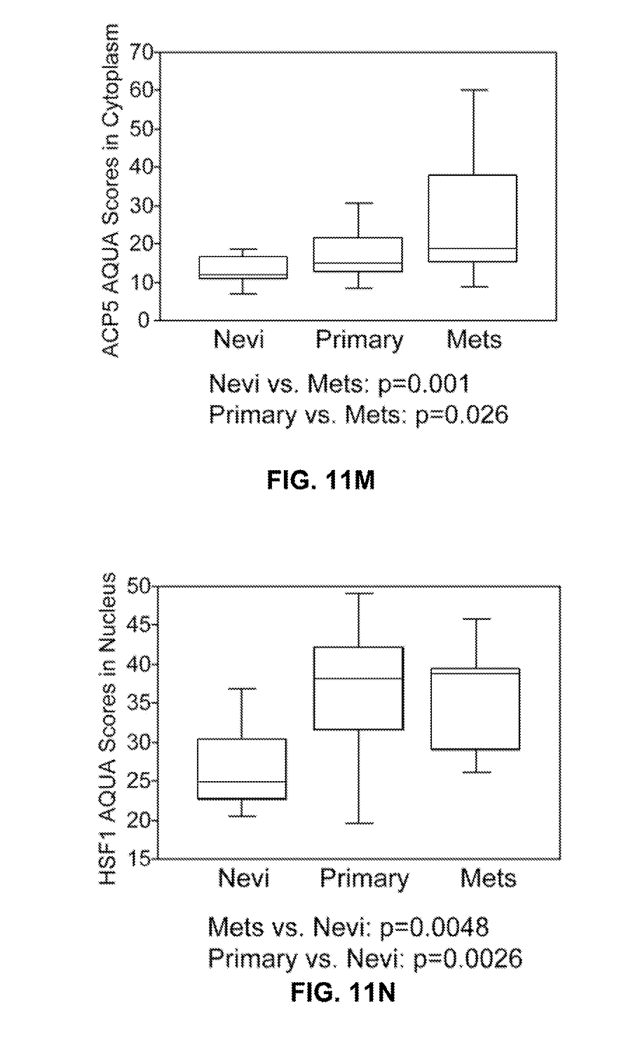

To prioritize downstream validation efforts, the 18 candidates were next assayed for ability to confer a 2-fold increase of invasion in a second melanoma cell system, WM115. This identified 11 robust pro-invasion genes (Table 1). The expression patterns of these pro-invasion genes were further investigated in human melanocytic lesions for evidence of human relevance, specifically increasing expression from benign to malignant and/or from primary to metastasis lesions as criteria for clinicopathological validation. To this end, commercially available antibodies were screened. Among those antibodies, 7 antibodies for 7 of the 11 genes were successfully qualified and optimized for quantitative immunofluorescence staining on formalin-fixed paraffin-embedded tissue. Using the AQUA® platform (Camp et al., Nat Med 8, 1323-1327 (2002)), protein expression levels were quantitated on the Yale Melanoma Progression Tissue Microarray (YTMA98) containing 20 specimens each of benign nevi, primary melanoma and melanoma metastases. As summarized in Table 1, six of seven pro-invasion genes (ACP5, FSCN1, HOXA1, HSF1, NDC80, and VSIG4) showed significantly higher expression across the benign-to-malignant and/or primary-to-metastasis transitions in human (Table 1 and FIGS. 11A-11N), qualifying them as validated pro-invasion genes in human melanomas.

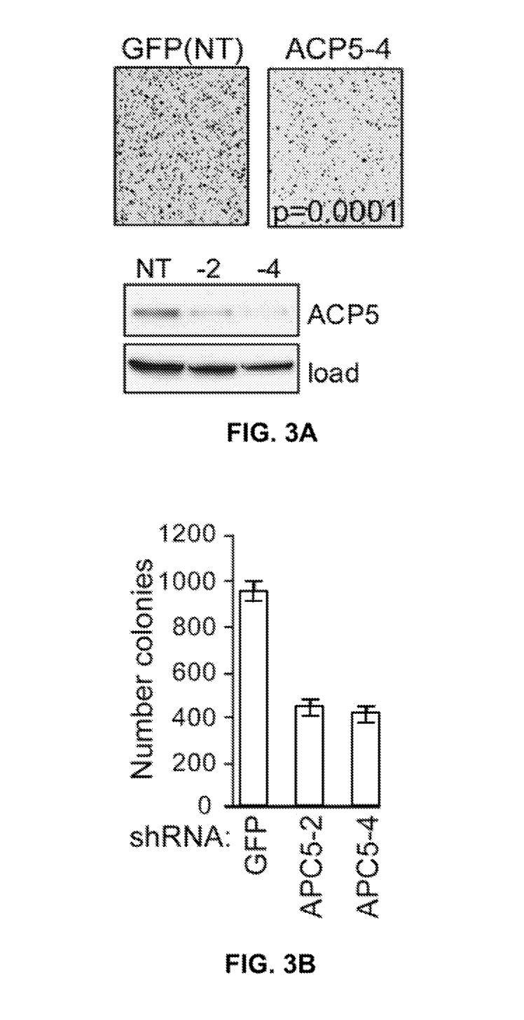

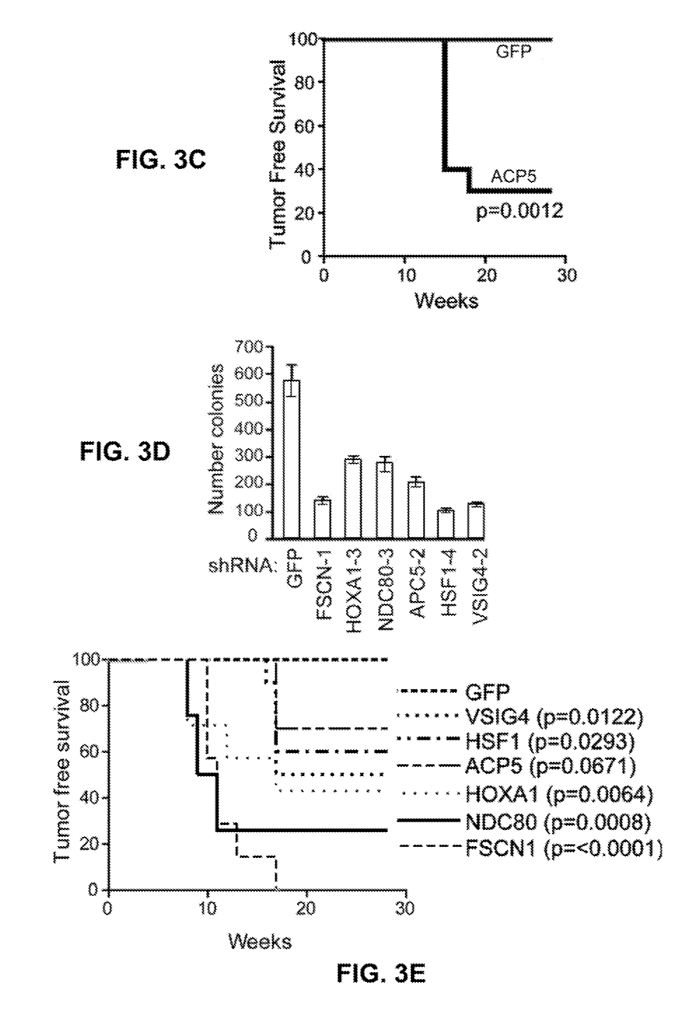

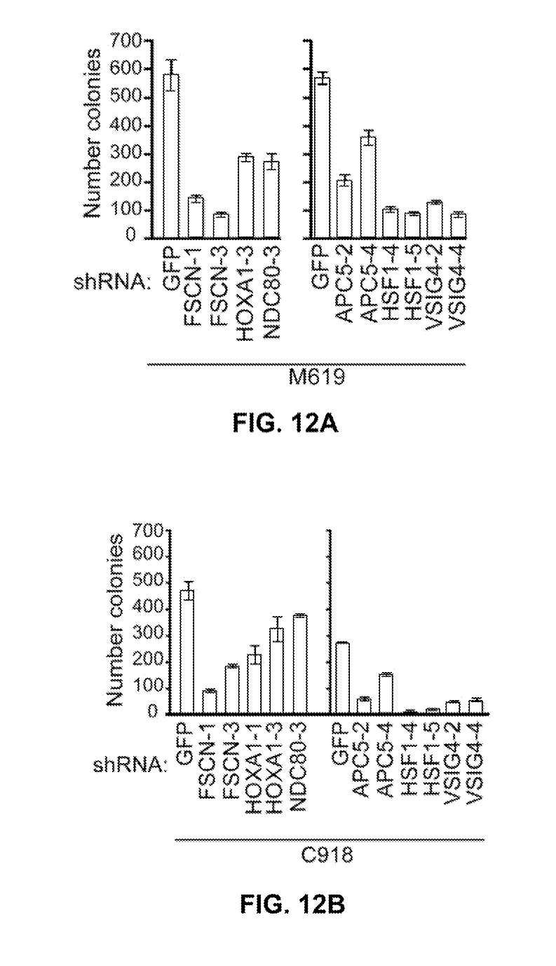

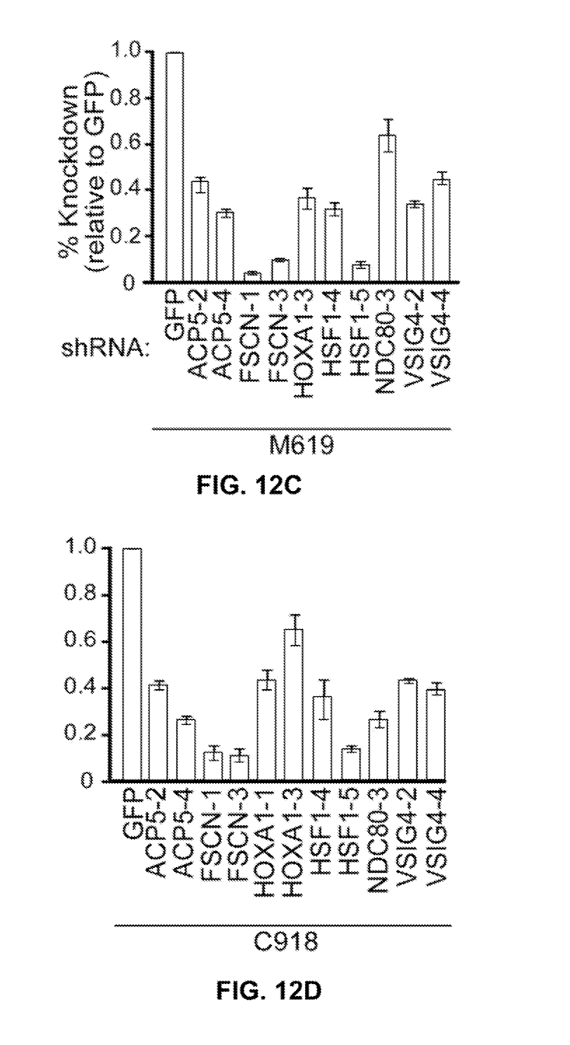

The acquisition of metastasis drivers in some early stage tumors might reflect their roles as bona fide oncogenes that could provide a proliferative advantage to the emergent primary tumors as speculated by Bernards and Weinberg (Bernards et al., Nature 418, 823 (2002)). The oncogenic potential of the 6 validated pro-invasion genes were further examined by assaying their requirement in maintaining the tumorigenic phenotype of established human melanoma cells in vitro and their ability to transform immortalized human melanocytes in vivo. For example, using anchorage independent growth as a surrogate for tumorigenic phenotype, depletion of ACP5 using two independent shRNAs in the human melanoma cell line 1205Lu resulted in a 56% reduction in soft agar colony formation (p=0.0001, FIGS. 3A-3B). Conversely, HMEL468 melanocytes (1×10⁶ cells/injection) stably expressing ACP5 became robustly tumorigenic when subcutaneously implanted into the right flank of athymic nude mice (p=0.0012, FIG. 3C). Importantly, extending these assays to the remaining 5 pro-invasion genes, it was found that knockdown of all 6 in M619 and C918 human melanoma cells significantly decreased colony formation when compared with non-targeting (shGFP) shRNA (FIG. 3D and FIGS. 12A-12D). Similarly, mice injected with HMEL468 cells over-expressing each of the 6 genes succumbed to tumor formation in vivo, compared to none of the animals injected with GFP control HMEL468 cells after 30 weeks of observation (FIG. 3E). Together, these complementary loss- and gain-of-function studies proved unequivocally that all 6 of these pro-invasion genes are oncogenic. These results are particularly striking finding given that transforming activity of these genes was not screened for in the course of their identification.

From the initial cross-species differentially-expressed list of 199 genes enlisted into the functional screen for cell invasion, 18 candidate metastasis oncogenes were identified. Of these, 7 candidates were prioritized for multi-level functional and clinicopathological validation, 6 were confirmed as potent pro-invasion oncogenes, capable of robust transforming and invasive activities in immortalized non-transformed human melanocytes, whose expressions are positively selected for in human melanomas during transformation or progression. Of the 6 validated metastasis oncogenes, most are not known or implicated in metastasis although some have been linked to cancer. For example, HSF1 (Heat Shock Factor 1) is a regulator of cell transformation and in vivo tumorigenesis (Dai et al., Cell 130, 1005-1018 (2007)), and HSF1-deficient cells exhibit markedly impaired migration and MAP kinase signaling (O'Callaghan-Sunol et al., Cell Cycle 5, 1431-1437 (2006)). In a transgenic mouse model with over-expression of NDC80, a component of the spindle checkpoint, tumor development was reported in multiple organs (Diaz-Rodriguez et al., Proc Natl Acad Sci USA 105, 16719-16724 (2008)), and depletion of NDC80 impairs tumor growth (Gurzov et al., Gene Ther 13, 1-7 (2006)). HOXA1 (Homeobox Transcription factor 1) has oncogenic activity in breast models (Zhang et al., J Biol Chem 278, 7580-7590 (2003)) and is up-regulated in multiple cancers including breast, squamous cell carcinoma and melanoma (Chariot et al., Biochem Biophys Res Commun 222, 292-297 (1996); Maeda et al., Int J Cancer 114, 436-441 (2005); Abe et al., Oncol Rep 15, 797-802 (2006)). VSIG4 (V-set and immunoglobulin domain containing 4) is a cell surface protein whose expression is mainly restricted to macrophages where it functions as a potent T-cell inhibitor (Vogt et al., J Clin Invest 116, 2817-2826 (2006); Xu et al., Immunol Lett 128, 46-50 (2010)). Based on its significantly higher expression in aggressive breast and ovarian tissues compared to benign tissues, ACP5 expression has been suggested to represent a progression marker (Honig et al., BMC Cancer 6, 199 (2006); Adams et al., Cell Biol Int 31, 191-195 (2007)), consistent with the data provided here in melanoma.

Metastasis Oncogenes are Non-Lineage Specific

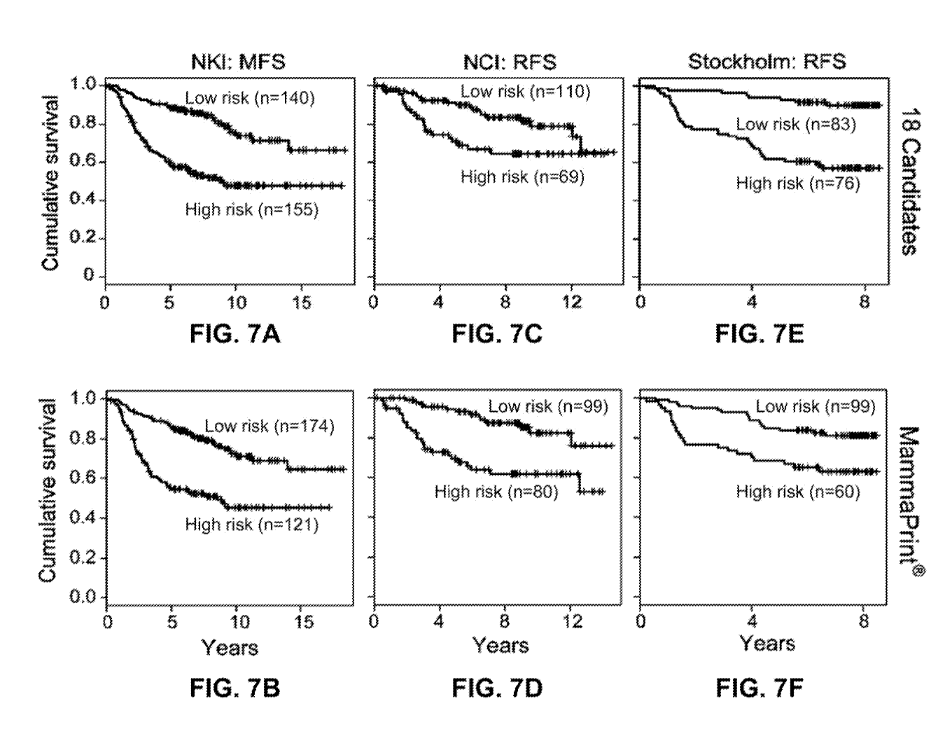

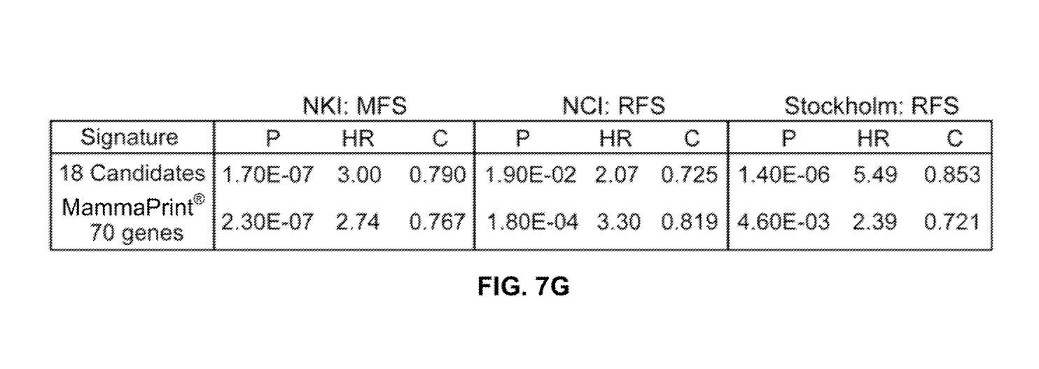

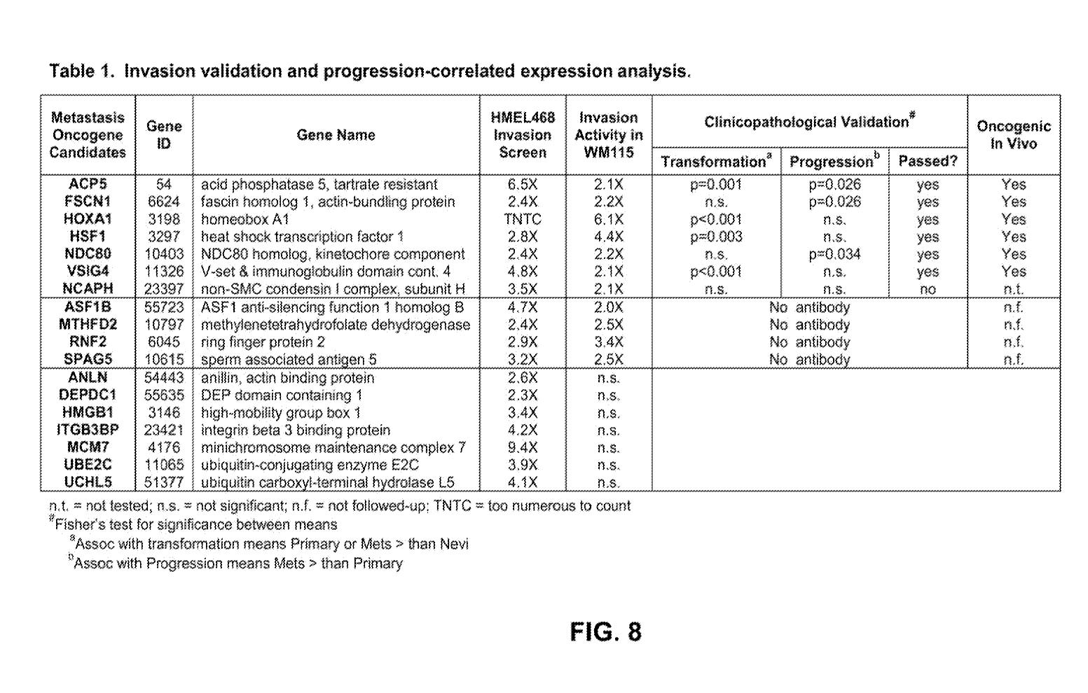

The majority of pro-invasion genes identified from the integrated functional genetic screen of the present invention have not been linked to metastasis. The prognostic relevance of these pro-invasion genes in other tumor types were further examined using RNA expression. Breast cancer was focused on based on the availability of 3 independent cohorts of transcriptome datasets on Stage I/II breast adenocarcinomas with outcome (recurrence or metastasis-free survival) annotation (van de Vijver et al., N Engl J Med 347, 1999-2009 (2002); Pawitan et al., Breast Cancer Res 7, R953-964 2005); Sotiriou et al., J Natl Cancer Inst 98, 262-272 (2006)). As summarized in FIGS. 7A-7F, expression levels of the 18 pro-invasion genes were able to stratify patients by K-mean clustering into two subgroups with significant differences in metastasis-free or recurrence-free survival by Kaplan-Meier survival analysis in all 3 independent datasets. Moreover, by C-statistics, these 18 genes were comparable to the 70-genes in the FDA-approved Mammaprint® (Agendia, Huntington Beach, Calif.) in their ability to prognosticate recurrence or metastasis (FIG. 7G). These data are remarkable in light of the fact that these genes were discovered in melanoma. Such cross-tumor prognostic significance reinforces the human relevance and highlights the power of this integrative functional genomics approach for discovery of metastasis oncogenes that can function across different tumor types.

In this example, well-defined GEM models, comparative oncogenomics, and functional genomics were employed to identify genes capable of driving invasion and transformation in early-staged melanomas. The genomic and biological homogeneity of GEM tumors and filtering power of cross-species comparisons proved highly effective in generating a shorter, more biologically significant list of genes enriched for cancer- and metastasis-relevant networks than either human or mouse datasets alone. Subsequent functional screen and stringent validation efforts identified high priority drivers of invasion—the key biological process that correlates with metastatic potential in melanoma. Finally, although oncogenic activity was not screened for, it is remarkable that every one of the 6 pro-invasion genes is robustly transforming in vivo, a finding that supports the hypothesis that drivers of metastasis in early-staged primary tumors also serve as professional oncogenes promoting tumorigenesis.

The majority of cancer-related deaths result from metastases. With the improvement of early detection capability by serum biomarkers and imaging advances, an increasing number of cancer cases will be diagnosed and surgically resected prior to apparent metastatic spread, leading to better overall survival relative to high-stage disease. At the same time, it is long-recognized that equivalent low-stage cancers are clinically heterogeneous with a subset exhibiting high-risk behavior, recurring with metastatic spread in the years ahead. The precise identification of such high-risk cases would enable more aggressive management in adjuvant setting, while avoiding unnecessary treatment in those patients cured by surgical intervention alone. Therefore, there is a growing need for the development of molecular-based prognostic biomarkers that can stratify risk for metastasis in the early-stage cancer population which constitutes an increasing proportion of cancer diagnoses each year. Transcriptomic and genomic characterization of human cancers supports the presence of molecular signals resident in primary tumors that can predict risk for metastasis. The development of MammaPrint® and OncotypeDx® has provided a strong measure of clinical proof of this concept. In comparison to the predominantly statistical correlative analyses from which these signatures were derived, the approach used in this example focuses on discovery of functional drivers of the metastatic process that are also oncogenic in early-stage cancers. Given their functional nature, the mechanism-of-action through which these pro-invasion oncogenes drive metastasis are expected to inform evidence-based therapeutic decisions in the adjuvant setting, in addition to themselves being rational points for therapeutic intervention. In this regard, the convergence of targeted therapeutics for melanoma (such as the selective BRAF inhibitor) and identification of pro-invasion oncogenes as prognostic biomarkers (such as ACP5) will be able to stratify a molecularly high-risked subpopulation among early-stage primary melanoma patients for clinical investigation aimed to explore the efficacy of these new therapies in the prevention of recurrence and metastasis.

### Example 2

### Identification and Functional Characterization of Biomarkers Associated with Anoikis Resistance

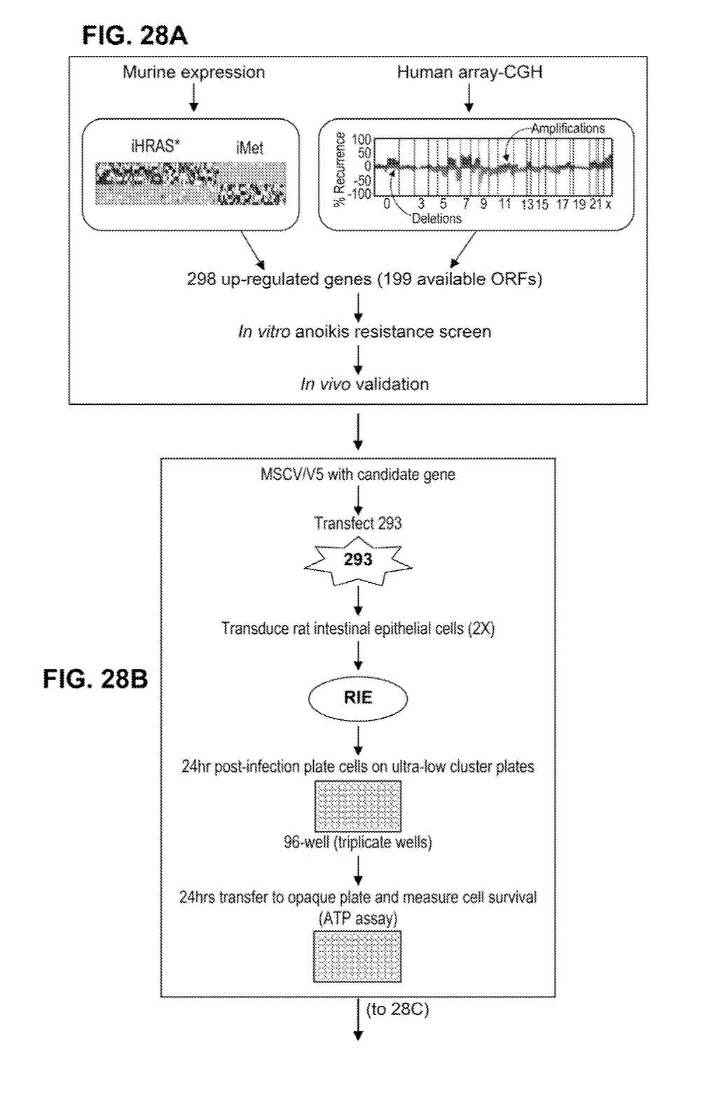

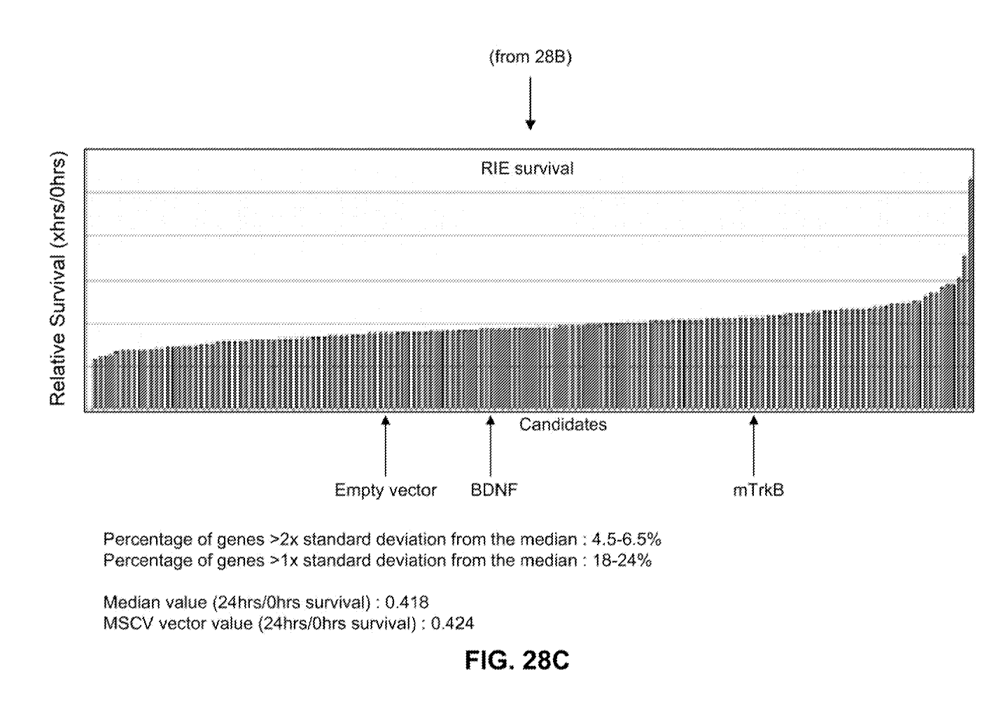

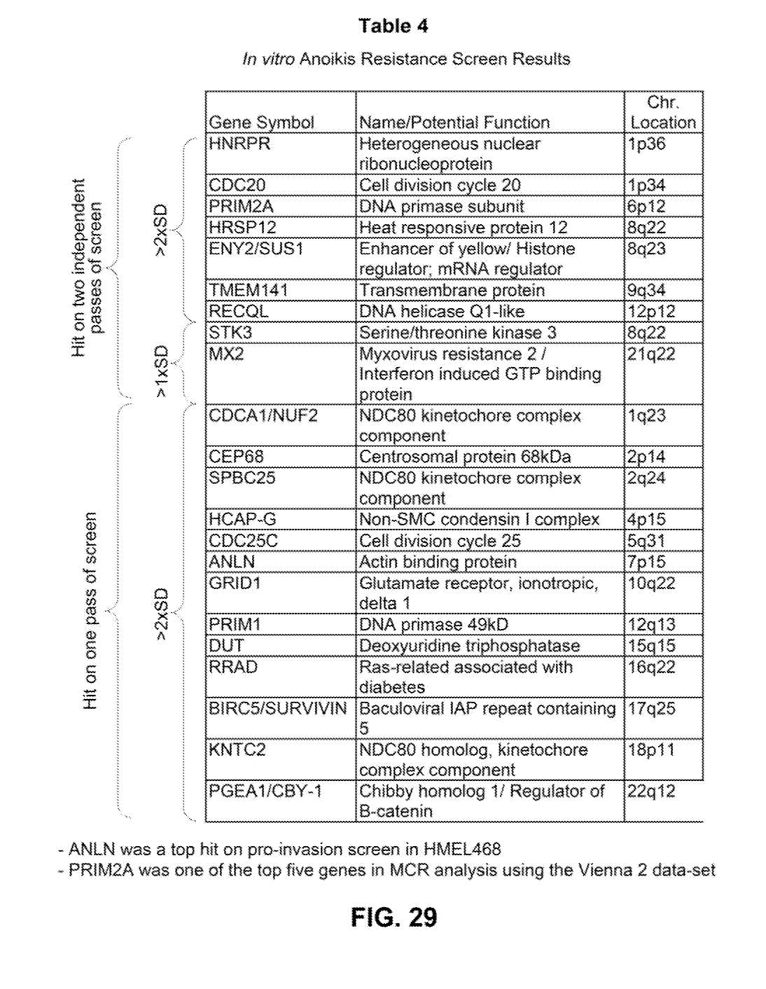

Metastasis is a complex, multi-step process (Gupta et al., Cell 127, 679-695 (2006)). In order for full metastasis to occur tumor cells must be able to proliferate at the primary tumor site, intravasate into the circulatory or lymphatic system, survive while in circulation, extravasate and form a secondary tumor. To accomplish this, circulating tumor cells must be able to overcome anoikis, or apoptosis induced by loss of matrix attachment (Simpson, C. D., Anyiwe, K., and Schimmer. A. D. (2008) Anoikis resistance and tumor metastasis. Cancer Lett 272, 177-185). In order to identify genes that confer anoikis resistance to anoikis sensitive cells, this example optimized an in vitro screen for anoikis sensitivity (FIG. 28B). It was hypothesized that cells seeded on a plate (ultra-low cluster) coated with a hydro-gel layer that prevented cell surface attachment would partially recapitulate in vitro the in vivo suspension of cells while in circulation.

In pilot studies, a cohort of melanoma cell lines was screen and it was found that all the cell lines, irrespective of melanoma stage (e.g. localized, invasive), were anoikis resistant. Instead, we and others found rat intestinal epithelial (RIE) cells to have reduced survival upon loss of adherence (Douma, S., Van Laar, T., Zevenhoven, J., Meuwissen, R., Van Garderen, E., and Peeper, D. S. (2004) Suppression of anoikis and induction of metastasis by the neurotrophic receptor TrkB. Nature 430, 1034-1039). RIE cells are immortalized but not transformed cell line. Cells undergoing anoikis initiate apoptotic pathways, while those that are viable upon loss of attachment demonstrate anoikis resistance. Therefore, we measured ATP generation, indicative of cellular metabolism, as a quantifiable and sensitive measure of cell viability.

Using the Gateway recombination system, 199 of the candidate ORFs identified through our cross-species oncogenomic analyses were cloned into the retroviral vector, MSCV/VS5. As analyzed by Western blot, mTrkB and a randomized sampling of clones of varying cDNA size expressed in RIE, thereby demonstrating the functionality of our expression system (FIG. 28B and data not shown).

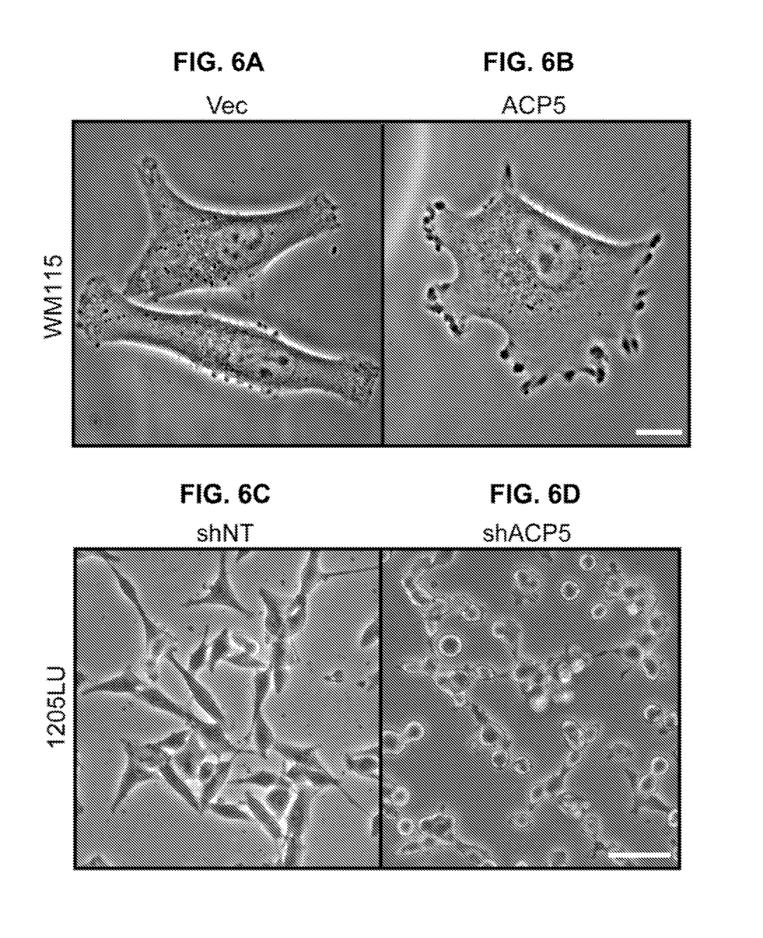

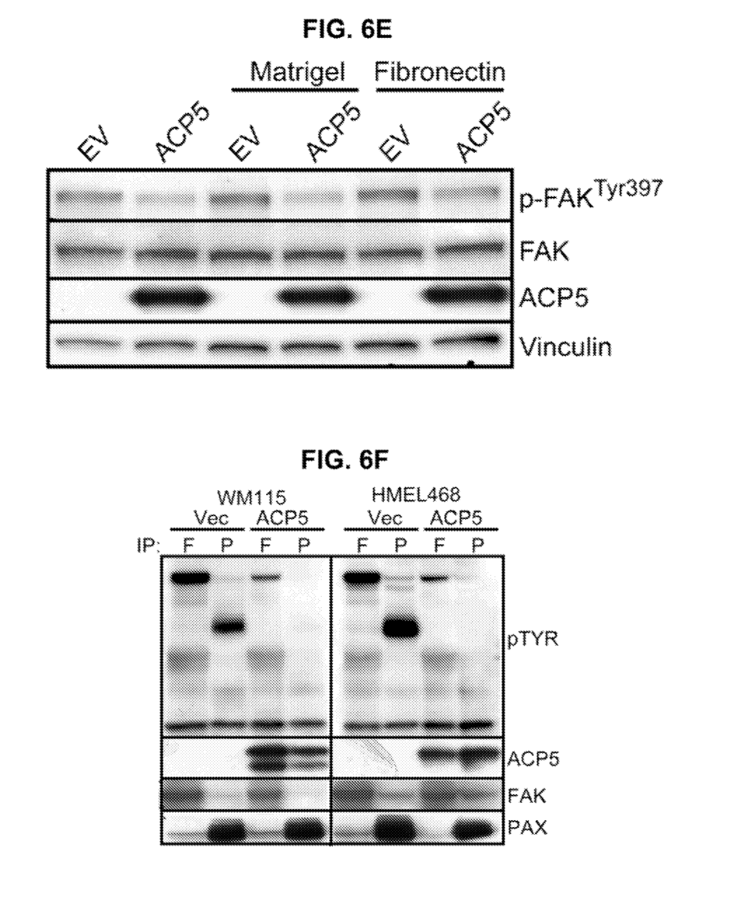

For the anoikis resistance screen, 293T cells were plated on 6-well plates and co-transfected with MSCV/V5 containing one ORF and the packaging vector, pCL-Eco (FIG. 6A). Cells were transfected with Lipofectamine 2000 (Invitrogen) and virus was harvested at multiple time points. RIE cells were plated on 6-well and 24 hr after plating were serially infected with 48 hr and 72 hr viral supernatant. RIE were harvested 24 hr after final infection and after generation of single-cell suspension, 7000 cells/well were plated in triplicate on 96-well ULC plates (time 0 hr). To determine baseline cell number, cells were lysed at 0 hr and ATP levels were measured (Cell Titer Glo, Promega). At 24 hr post-ULC plating, cells were lysed with Cell Titer Glo and lysate was transferred to 96-well opaque-welled luminometer plates for reading. In our analysis, ATP levels were compared at 24 hr relative to 0 hr thereby giving the fold change in ATP levels (FIG. 28C).

The neurotrophic receptor TrkB has been shown to confer anoikis resistance in vitro to anoikis sensitive cells and promote tumor formation and lung seeding in vivo. We have increased confidence in our screen since murine TrkB (mTrkB) and the human ligand to TrkB, BDNF, conferred anoikis resistance to RIE greater than vector alone (FIG. 28C).

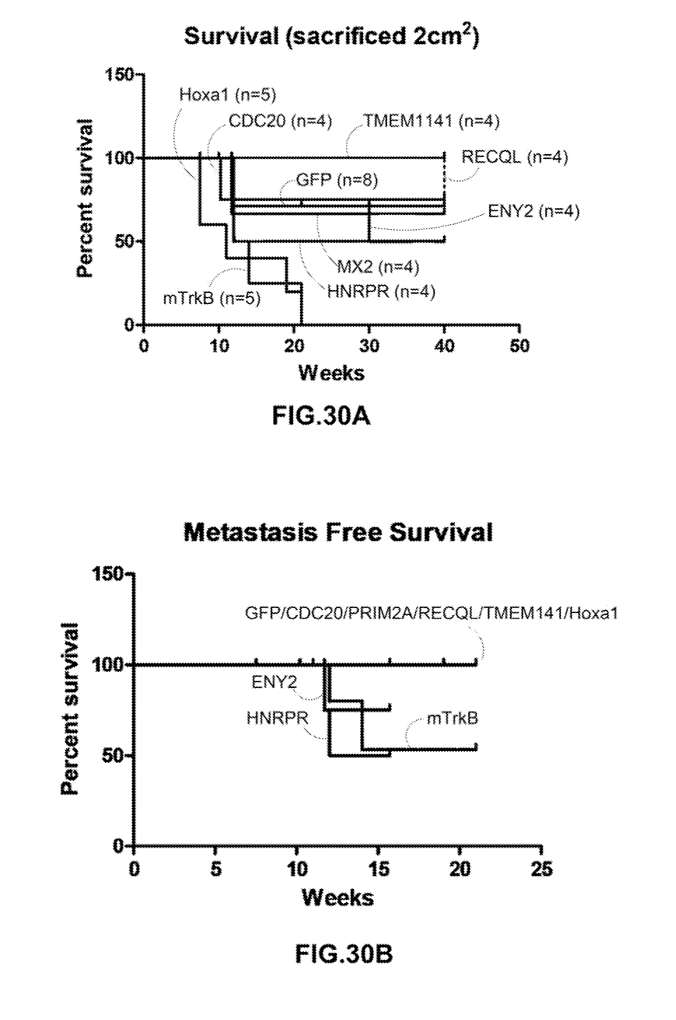

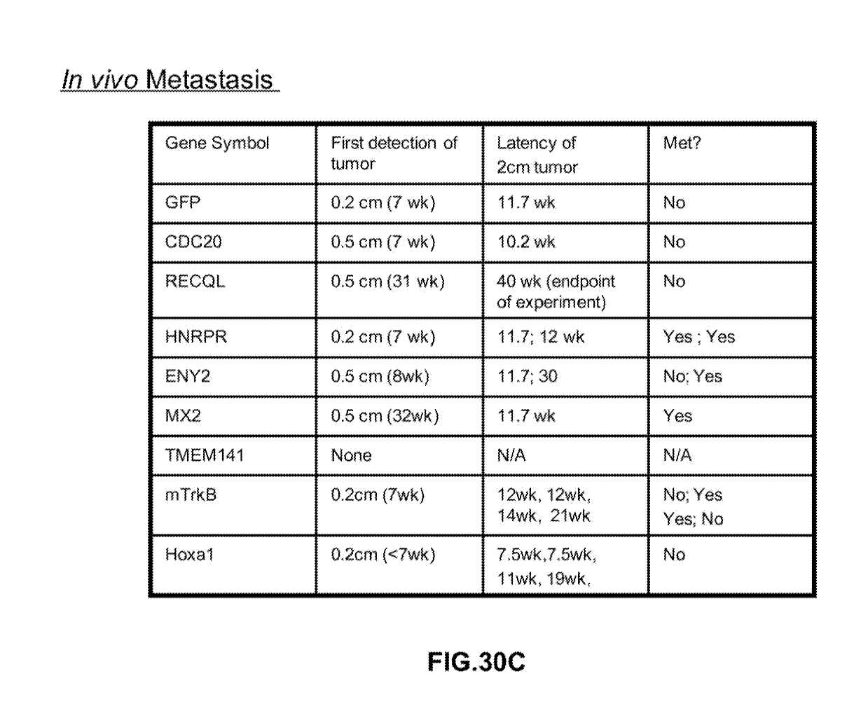

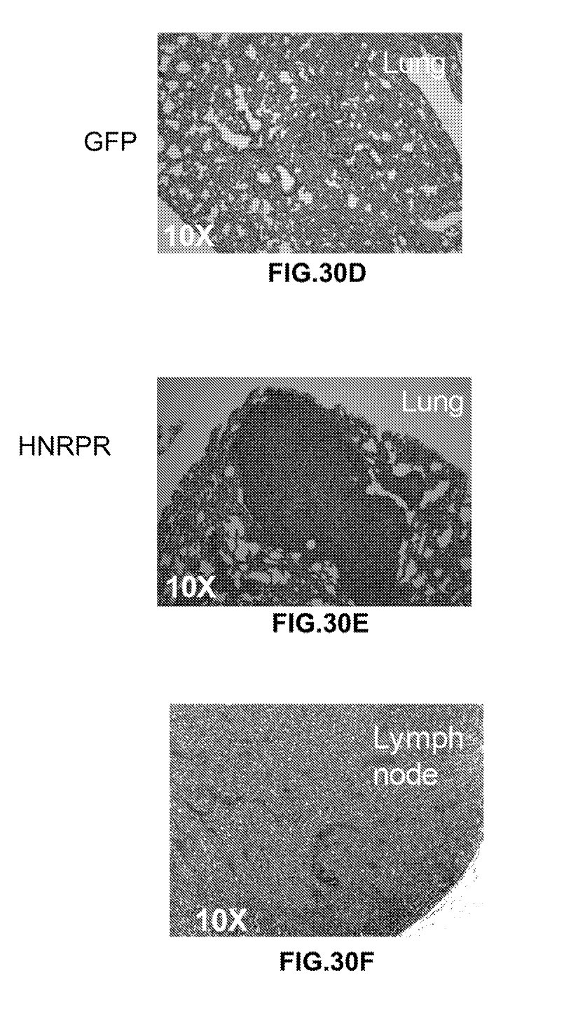

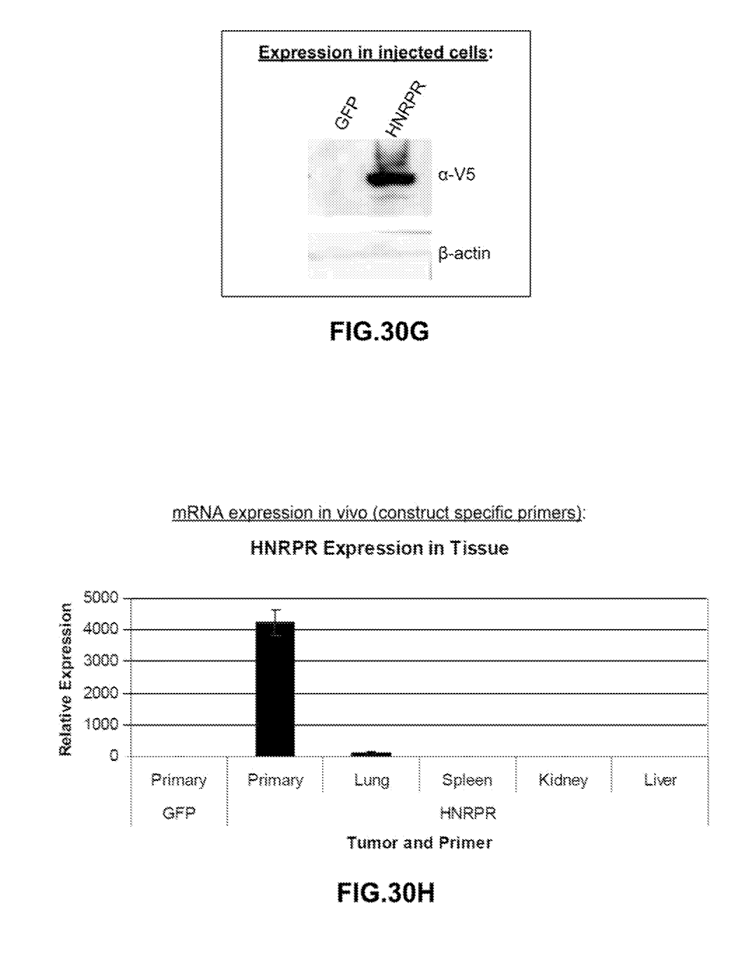

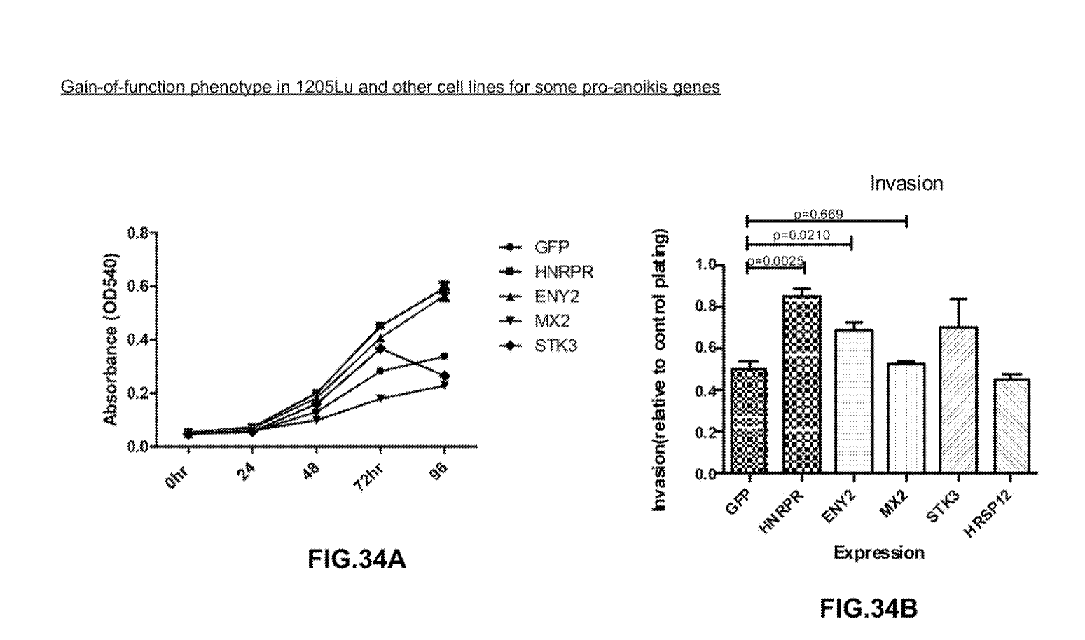

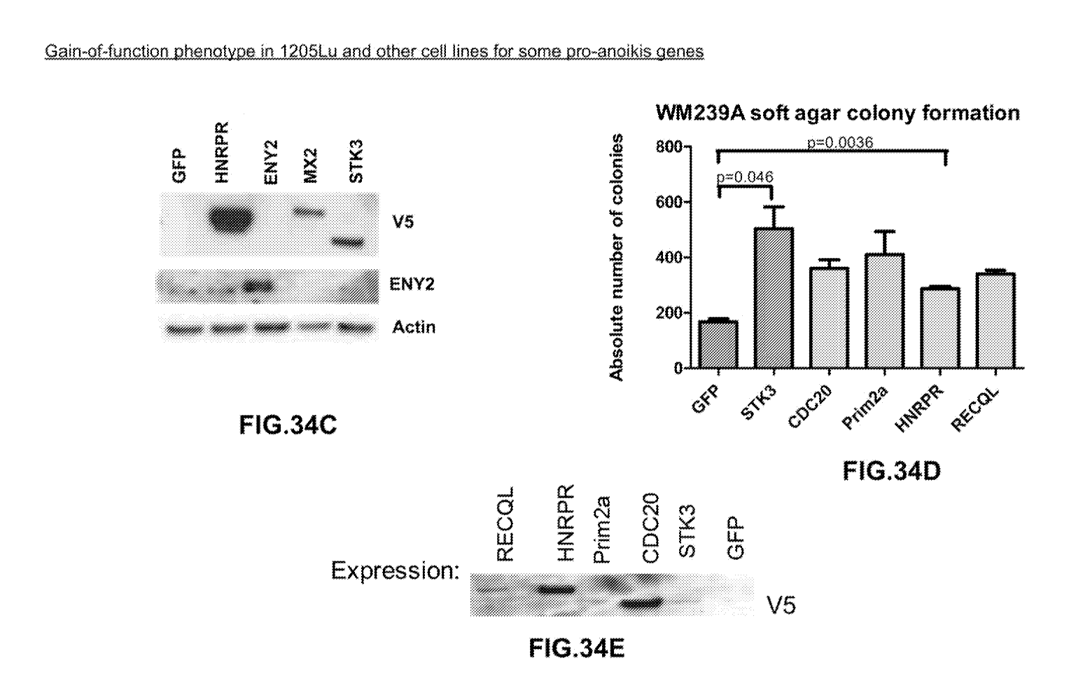

Twenty genes have greater than 2 standard deviations from the median in at least one pass of the screen (HNRPR, CDC20, PRIM2A, HRSP12, ENY2/sus1, TMEM141, RECQL, CDCA1/NUF2, CEP68, SPBC25, HCAP-G, CDC25C, ANLN, GRID1, PRIM1, DUT, RRAD, BIRC5/SURVIVIN, KNTC2, and PGEA1/CBY-1; see Table 4). Nine of these genes conferred greater than 1 standard deviation from the median in both screens HNRPR, CDC20, PRIM2A, HRSP12, ENY2/sus1, TMEM141, RECQL, STK3, and MX2; see Table 4). Seven of these nine gave greater than 2 standard deviations from the median in at least one pass of the screen (HNRPR. CDC20, PRIM2A HRSP12, ENY2, TMEM141, and RECQL: see Table 4). To further validate the relevance of the above-identified anoikis resistance associated biomarkers in tumorigenesis, functional studies were conducted on these biomarkers (see FIGS. 30A-38E)

Methods

In vivo injections: Nude mice were injected sub-cutaneously on one flank of the mouse with 0.6×10⁶ 1205Lu cells expressing a gene of interest. Mice were monitored for primary tumor formation and when tumor burden reached 2 cm² mice were euthanized. Various organs were collected for histological studies including H&E.

In vitro anoikis resistance screen and survival assays: For the anoikis resistance screen, 293T were co-transfected with one gene of interest (GOI) and the packaging vector, pCL-Eco. RIE were plated on adherent plates and serially infected with 48 hr and 72 hr viral supernatant. RIE were harvested 24 hr after the last infection and after trypsin mediated generation of single cell suspensions, 7000 cells/well were plated in triplicate on 96-well ULC plates (time 0 hr). At 24 hr post-ULC plating, cells were lysed with CellTiter Glo and lysate was transferred to 96-well opaque-welled luminometer plates for reading. ATP levels were compared at 24 hr to 0 hr ATP levels (e.g. 24 hr reading/0 hr reading) thereby giving the fold change in ATP levels.

Apoptosis assays: RIE stably expressing a GOI were plated in non-adherent conditions. At 0 hr and 24 hrs cells were stained with Annexin/PI and analyzed on a Gauva machine.

Soft Agar, Invasion, and Cell Proliferation assays: Cells stably expressing a GOI were plated on soft agar and monitored for growth up to two months. For cell proliferation, cells were plated 10,000/12-well. Cells were stained with crystal violet and absorbance was read in 10% acetic acid/PBS. For invasion assay, cells were plated on in a Boyden Invasion Chamber and cells were allowed to migrate for 24 hrs. Membranes were then stained with crystal violet.

Lentiviral production: 293T cells were transfected with either pL6, MSCV, pCDH-CMV-V5-T2A-GFP, or pLKO.1 vectors containing genes of interest with appropriate packaging constructs. Virus was harvested 48-72 hrs post transfection. Cells were infected with polybrene for 24 hrs. For some cells, a second round of infection was conducted after which cells were in some cases selected.

Results

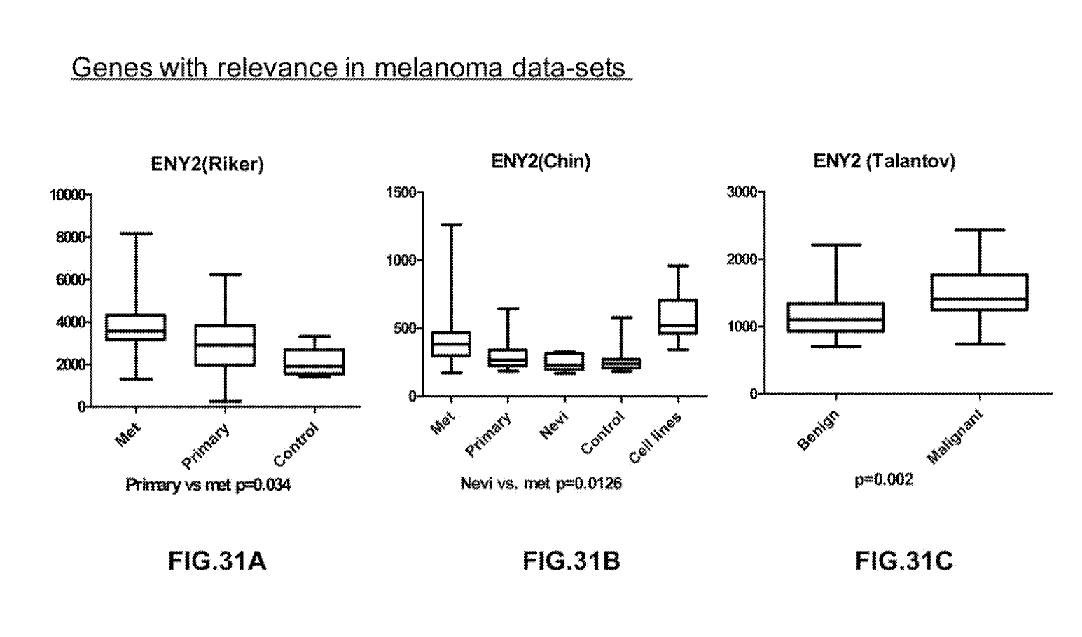

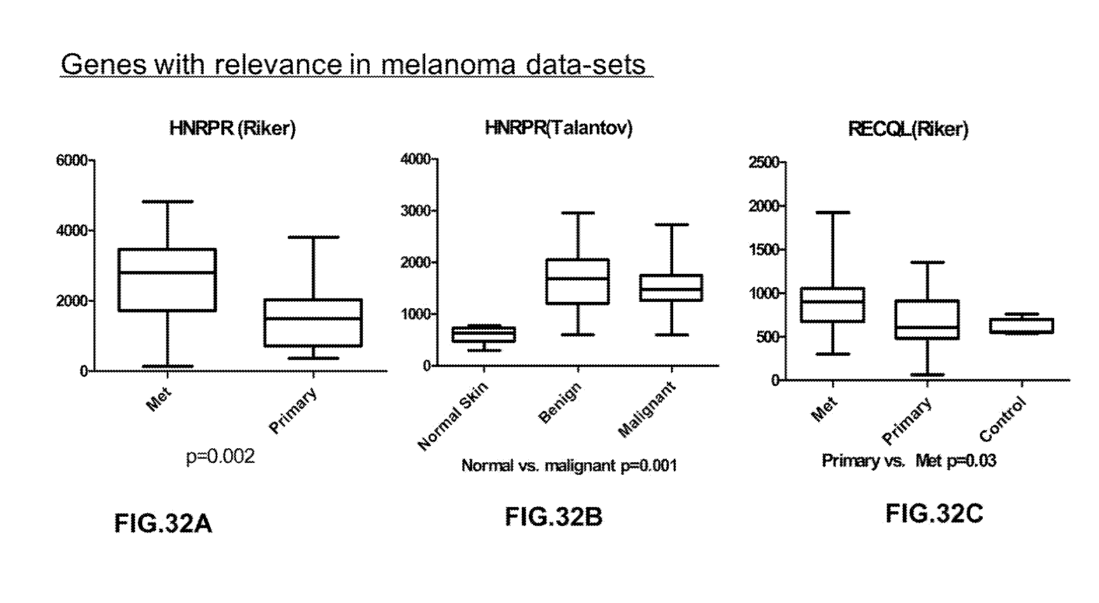

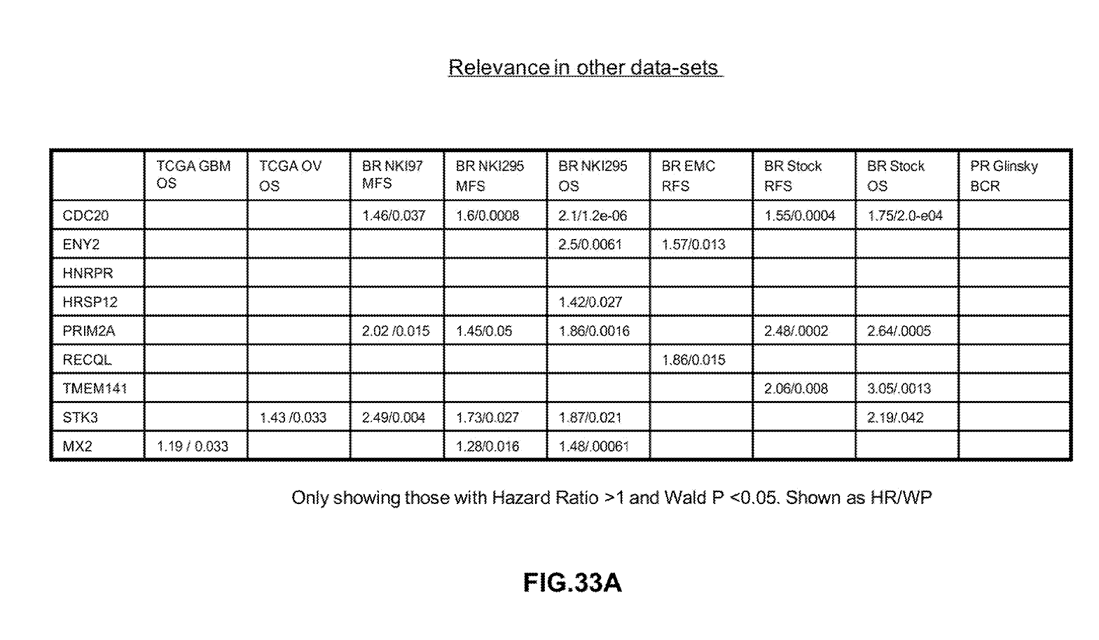

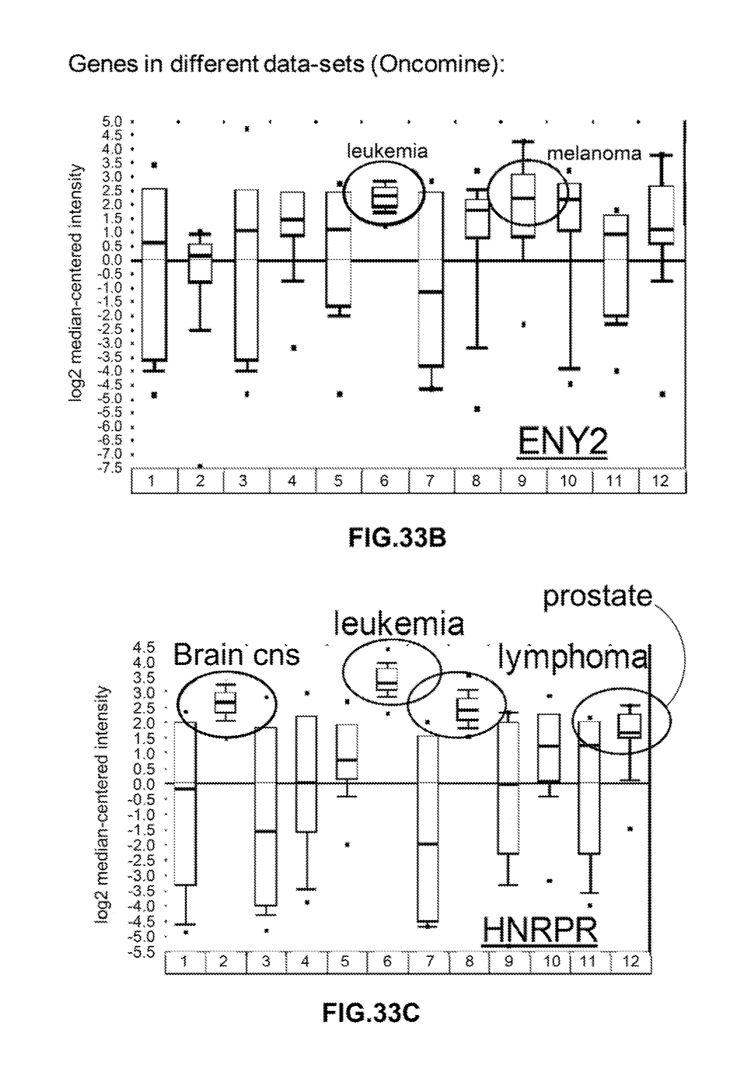

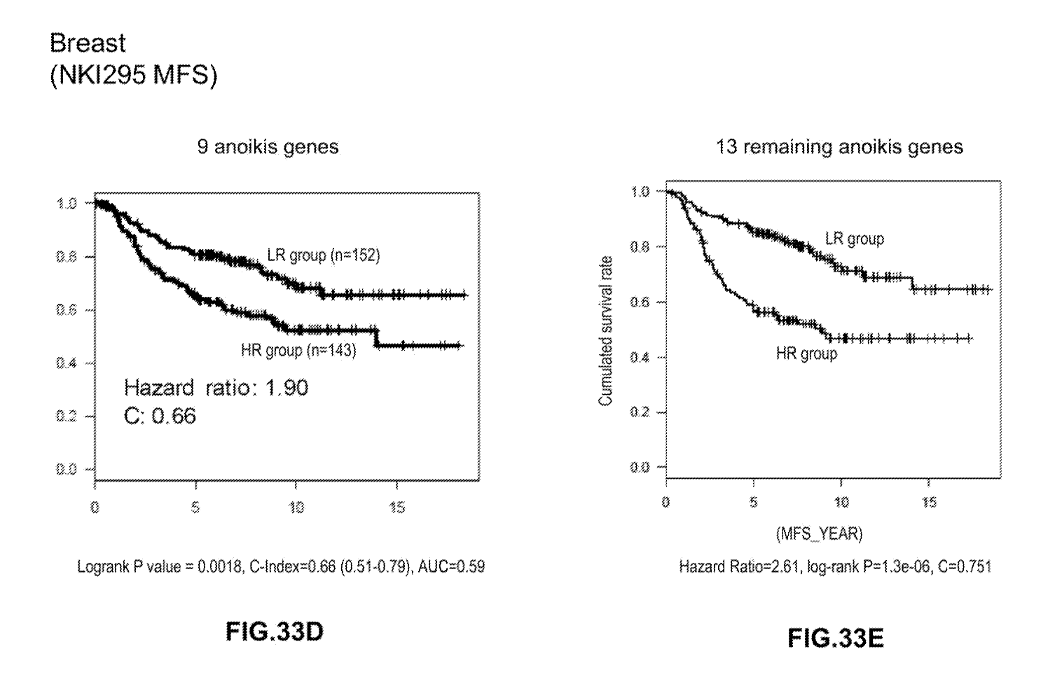

One of the identified anoikis resistant genes—CDC20—was shown to decrease tumor latency (FIGS. 30A-30C). Three of the identified anoikis resistance genes—HNRPR, ENY2, and MX2-promoted metastasis of a melanoma cell line from a sub-cutaneous injection (FIG. 30C). Of these three genes that promote metastasis in vivo, Eny2 and HNRPR also correlate with tumor progression in various melanoma data-sets (FIGS. 31A-31C and 32A-32B). Some of the identified anoikis resistance genes also show relevance in non-melanoma data-sets (FIGS. 33A-33E). Individual anoikis resistance genes show correlation with survival and expression in various tumor types, suggesting that these genes may have a broader role in tumor progression and may be relevant not only to melanoma.

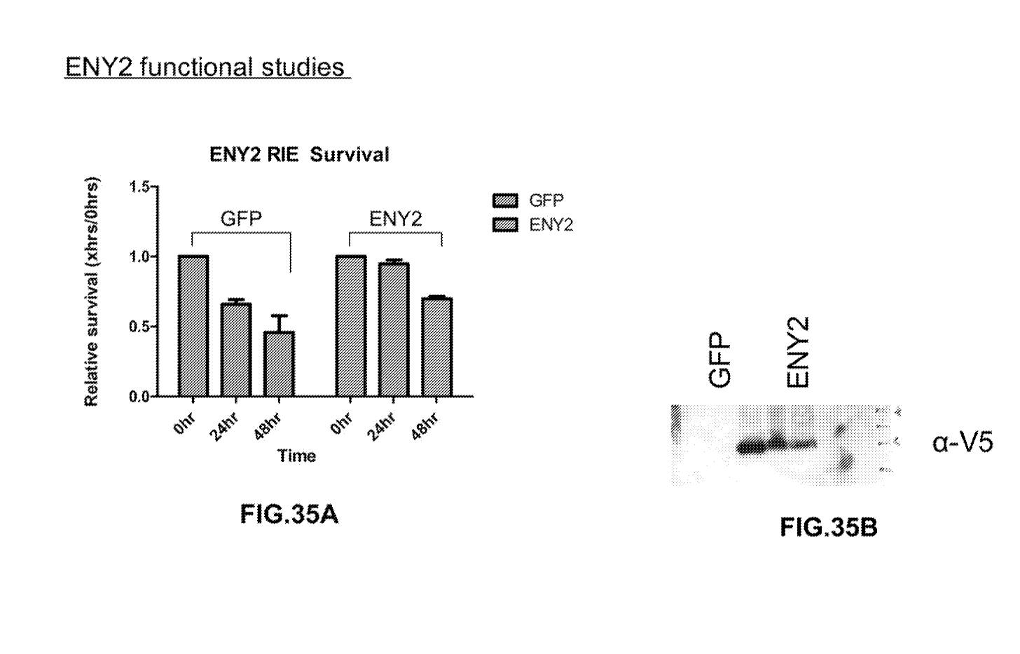

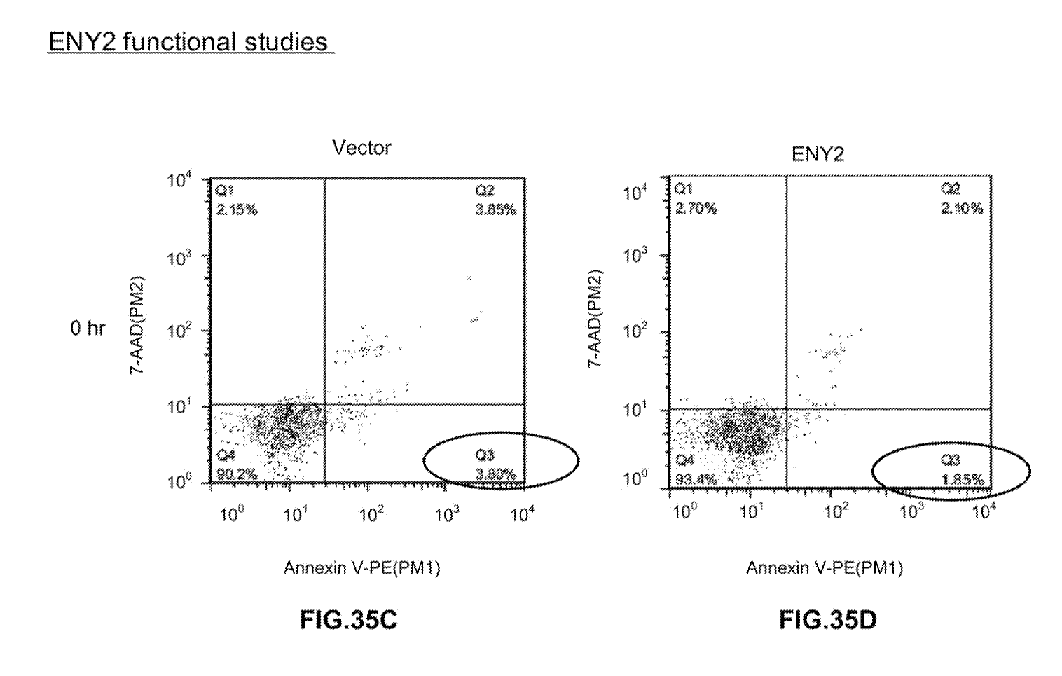

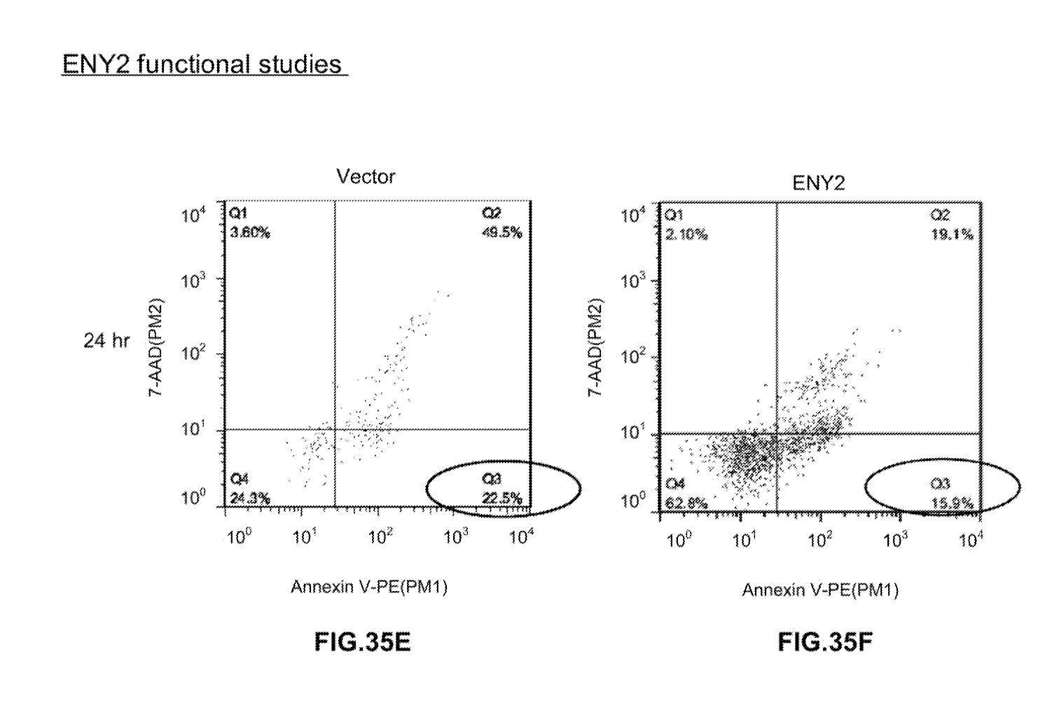

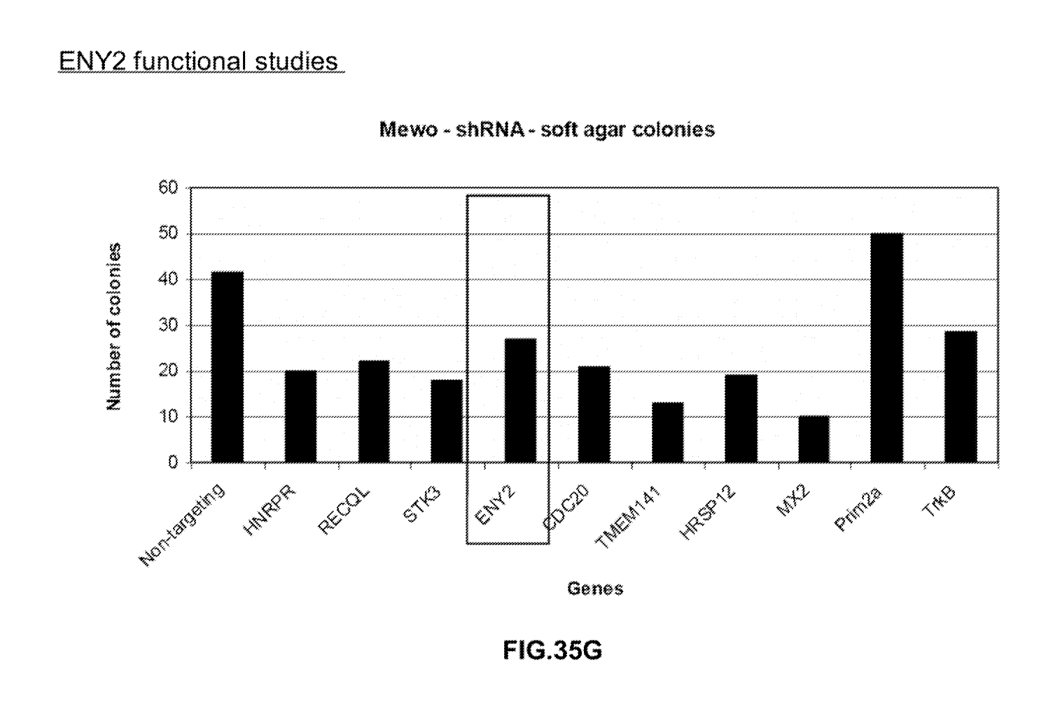

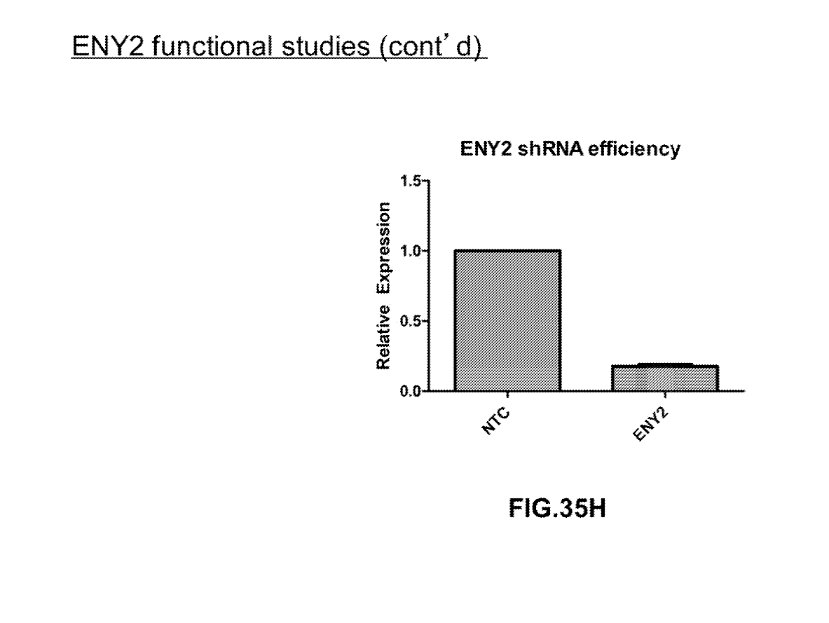

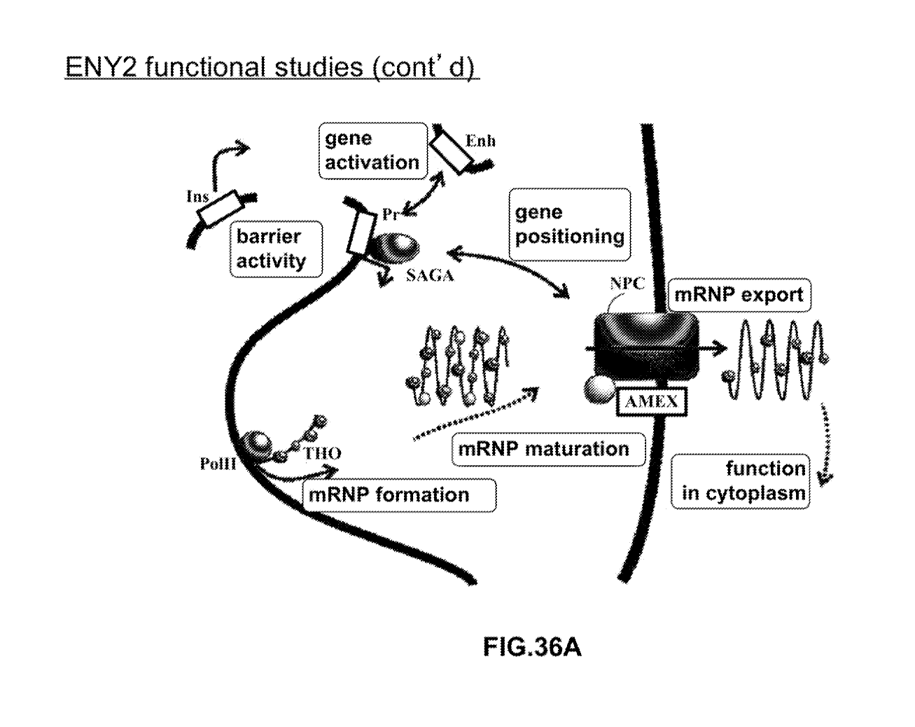

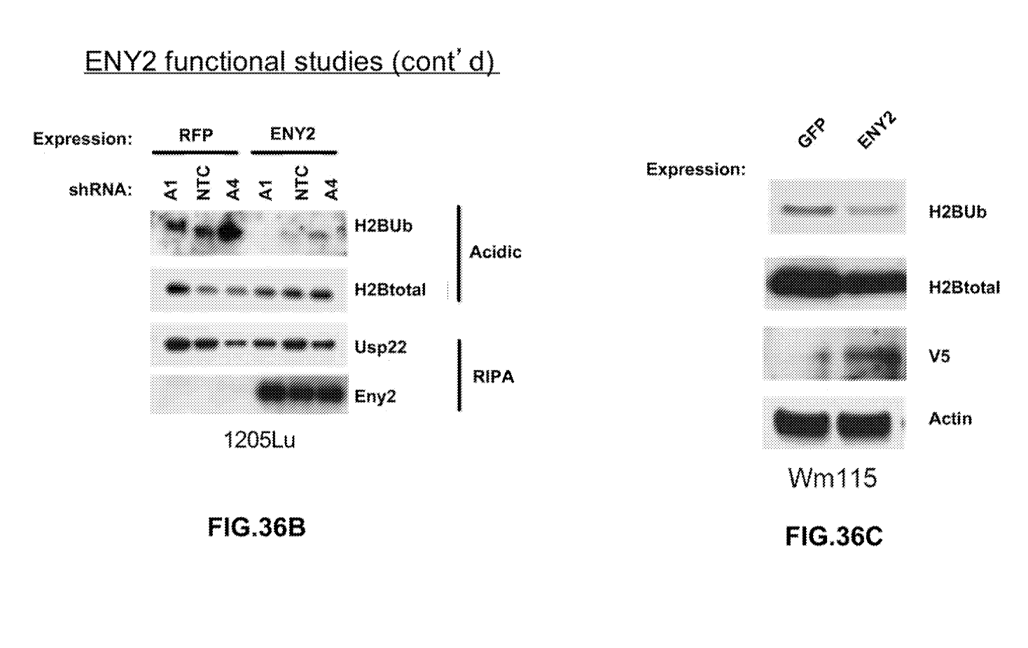

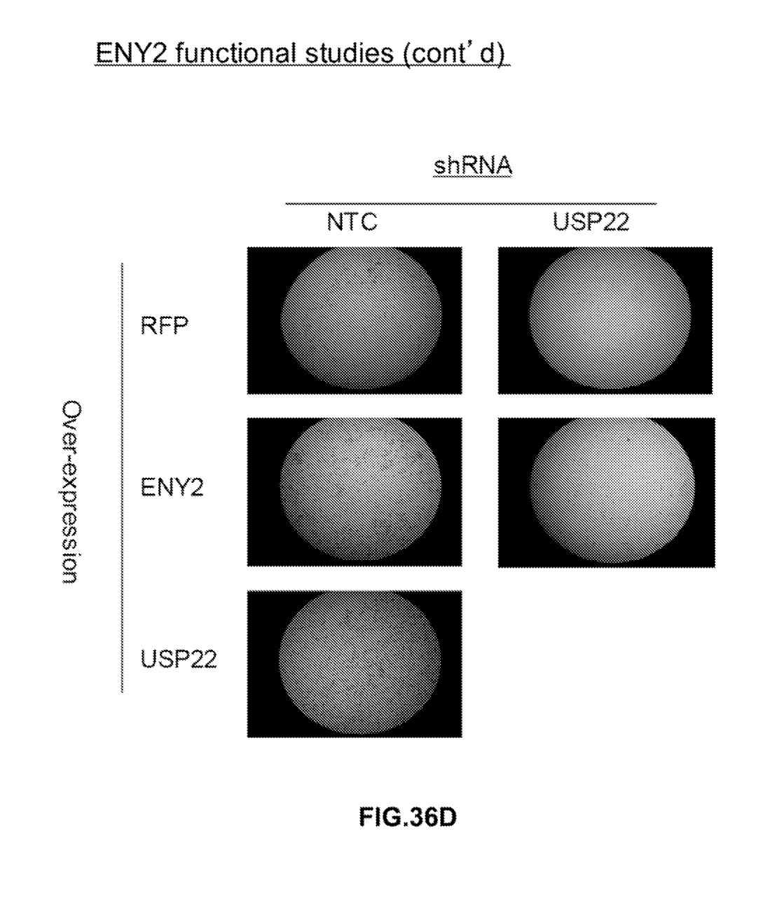

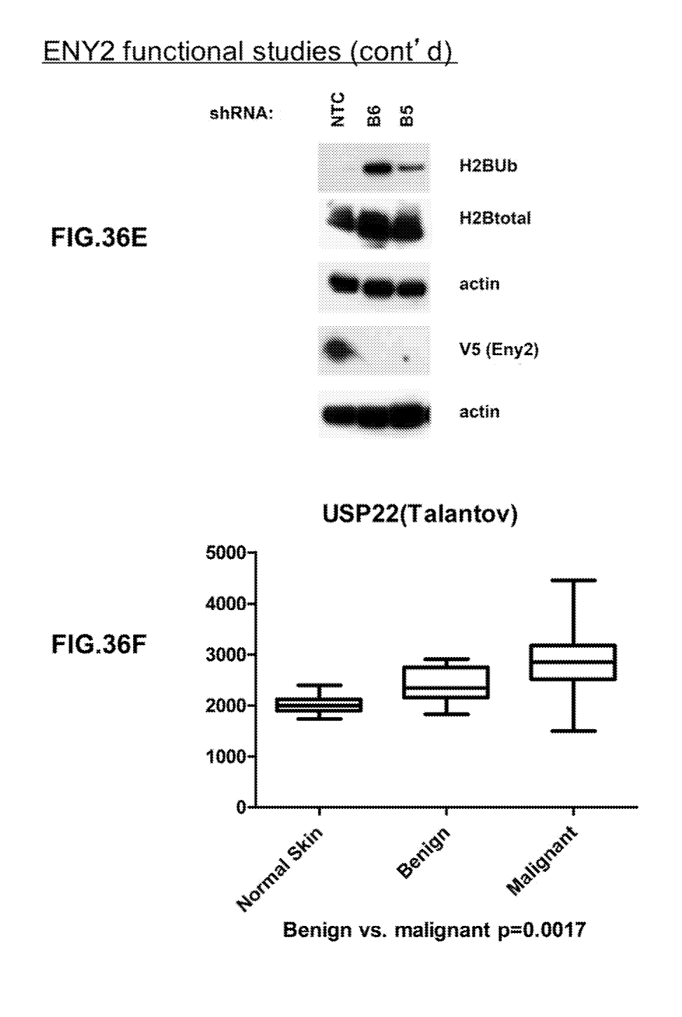

Eny2 functional studies: Eny2 over-expression not only increases over-all survival, but also reduces apoptosis of rat intestinal cells in non-adherent conditions. In addition, Eny2 promotes soft agar colony formation in Mewo, a cell line with low Eny2 levels. Eny2 also regulates H2Bub in some melanoma cells lines and this regulation may be dependent on the catalytic subunit of the SAGA-DUB complex, USP22. Furthermore, Eny2 promotion of invasion may also be dependent on USP22. Eny2 is necessary for inhibition of H2BUb in cells derived from metastatic lung nodules stably expressing Eny2. See FIGS. 35A-36C.

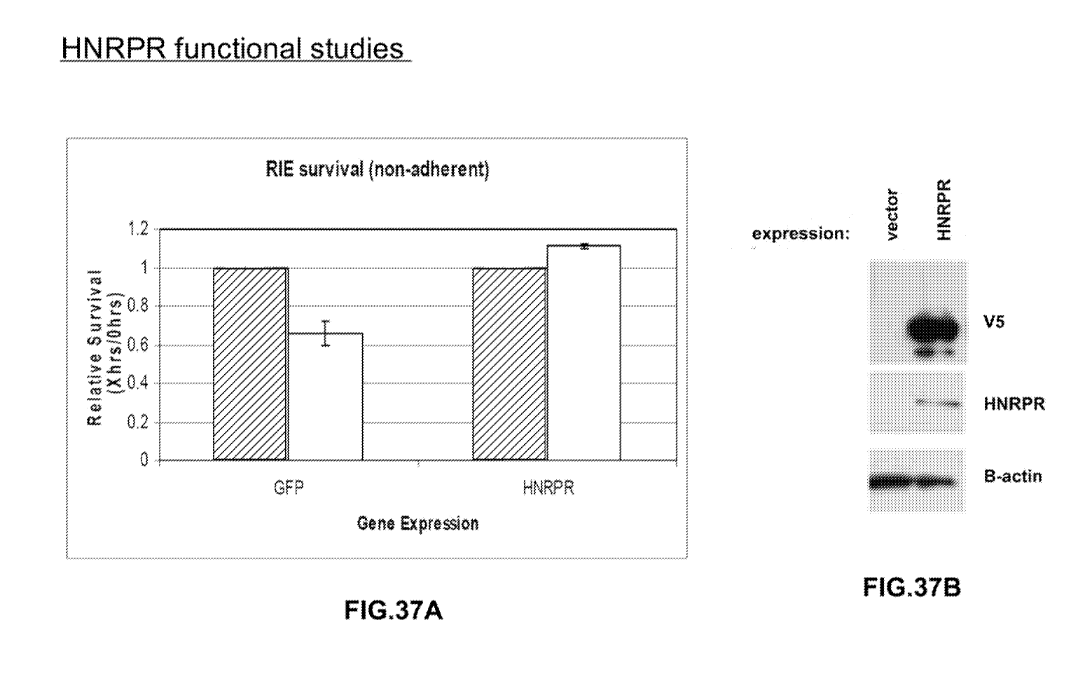

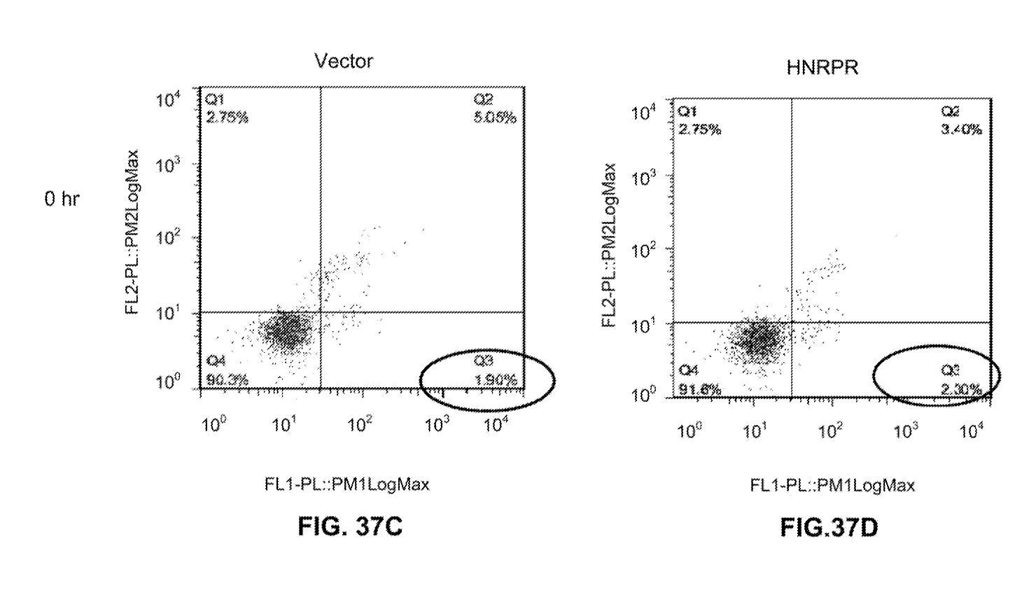

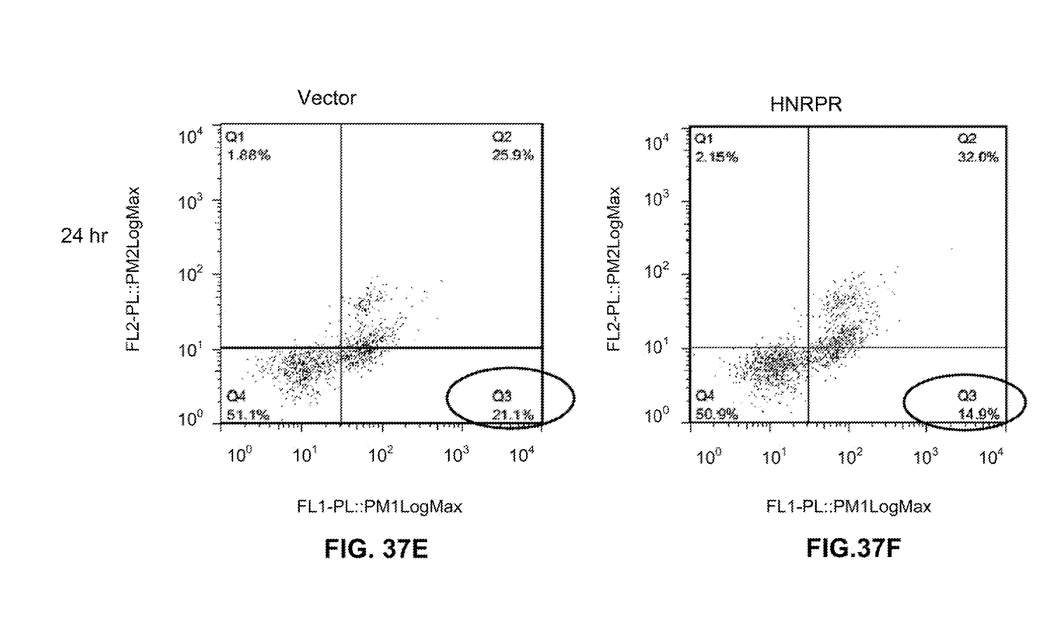

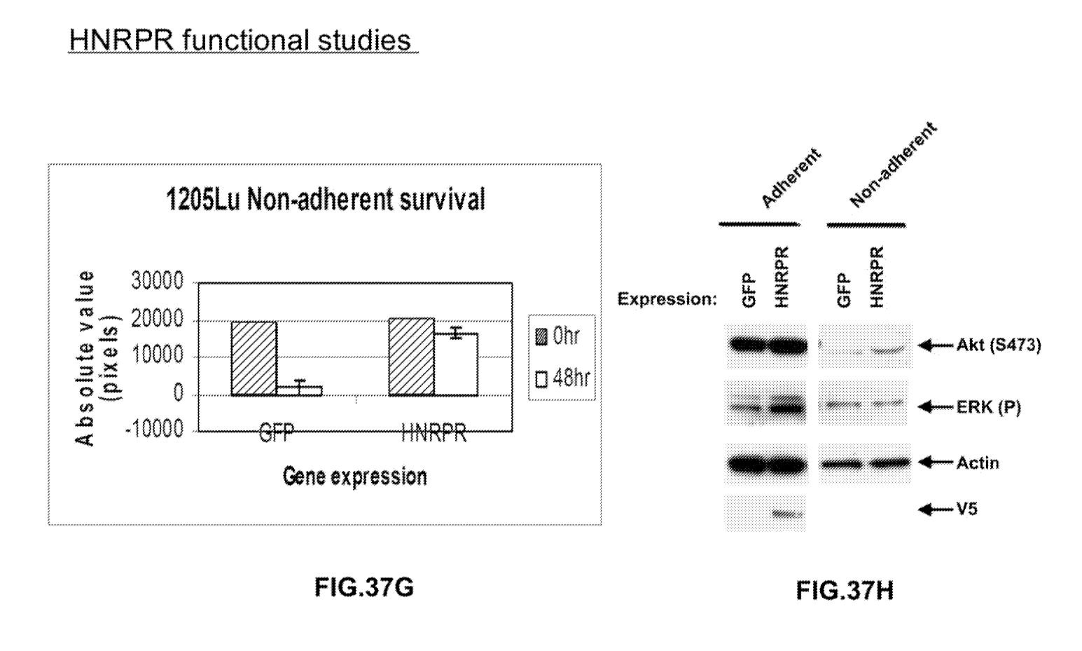

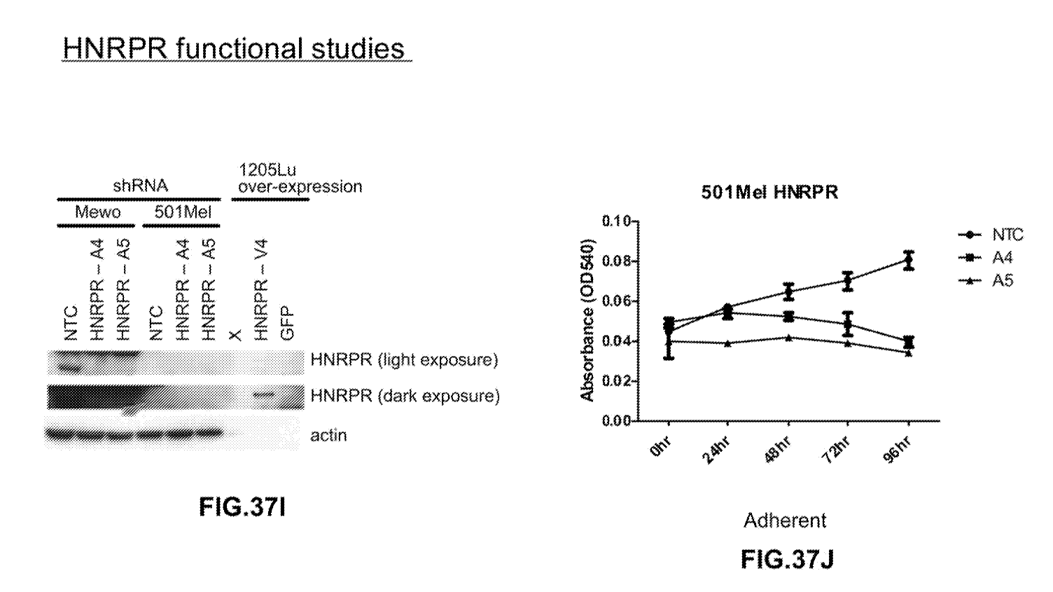

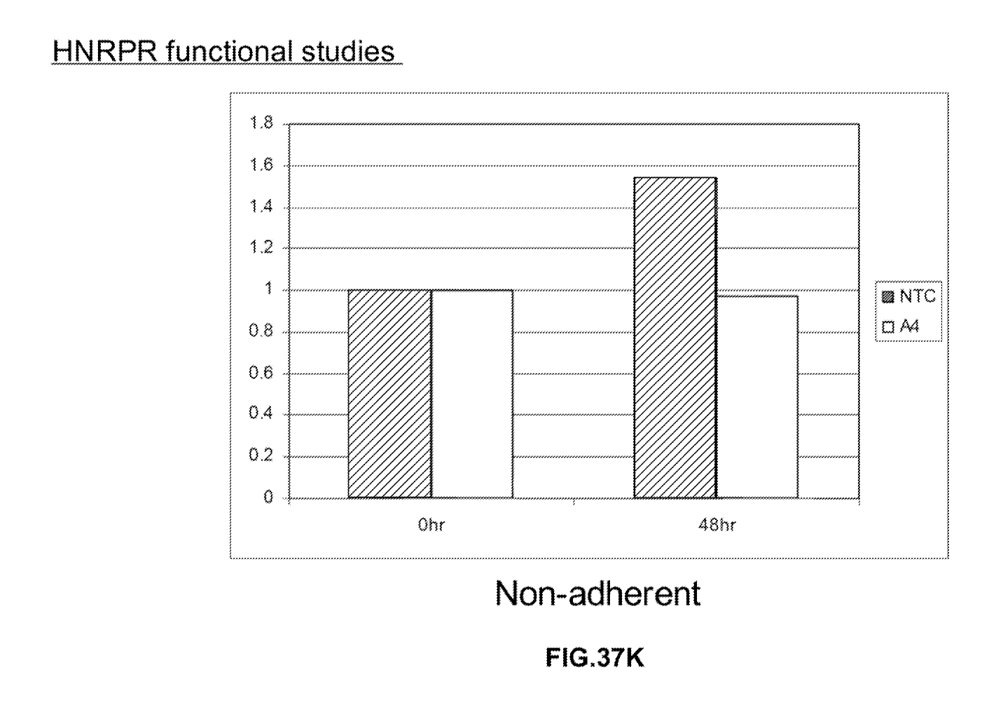

HNRPR functional studies: HNRPR over-expression increases survival of rat intestinal epithelial cells in non-adherent conditions. HNRPR over-expression also reduces apoptosis of rat intestinal epithelial cells in non-adherent conditions (Annexin/PI). shRNA-mediated loss of HNRPR in 501MeI decreases 501MeI cell proliferation and survival in non-adherent conditions. Loss of HNRPR in Mewo also has no effect on survival (data not shown). HNRPR over-expression increases survival of 1205Lu in non-adherent conditions and increases Akt (S473). See FIGS. 37A-37K.

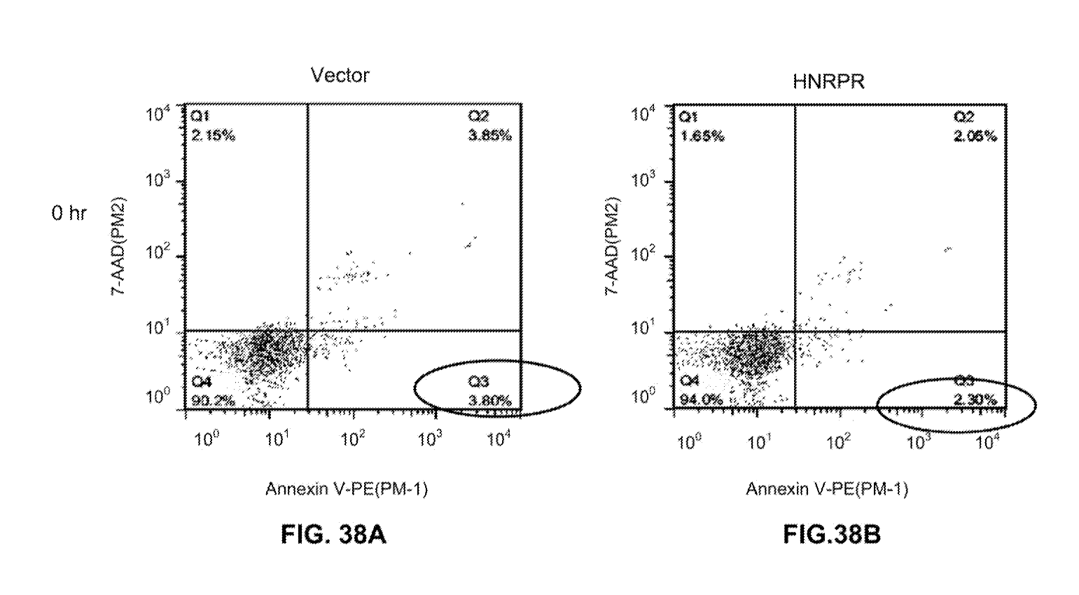

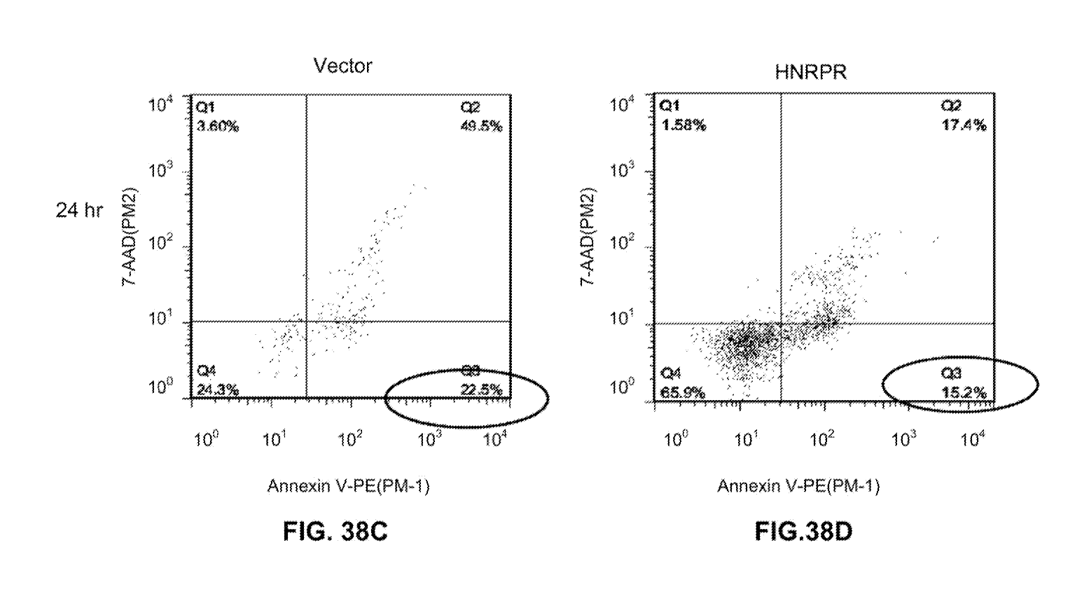

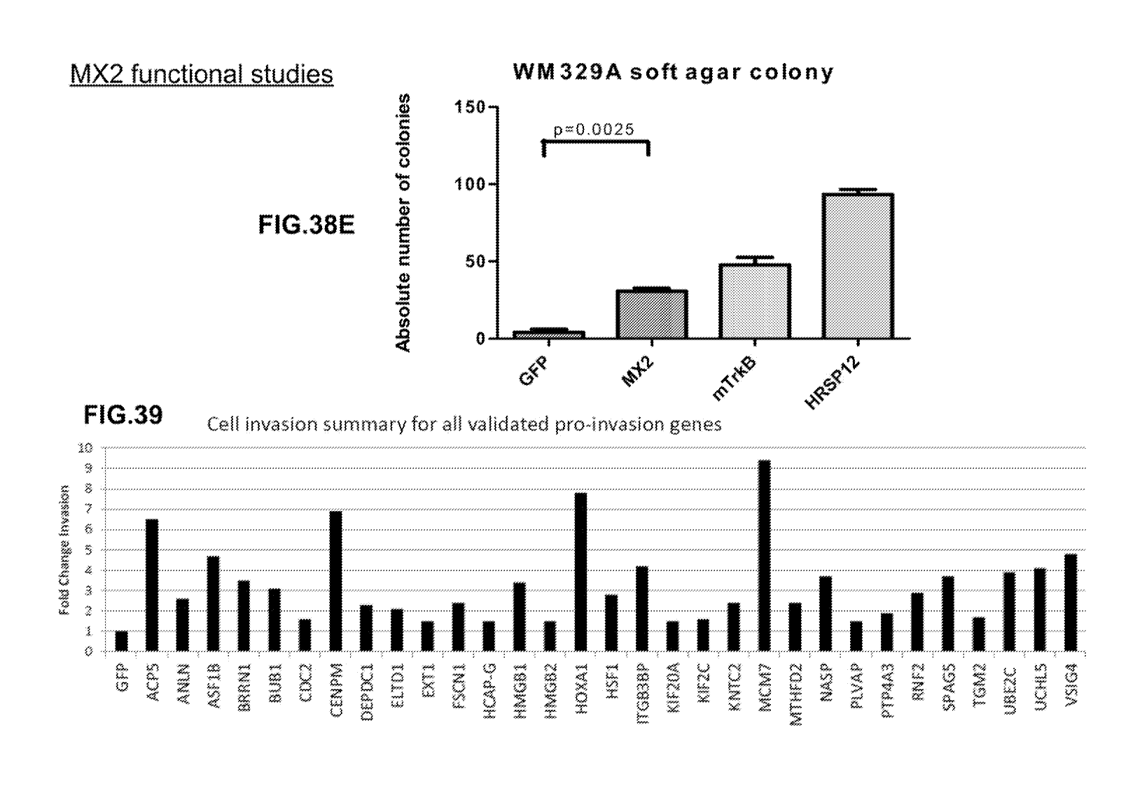

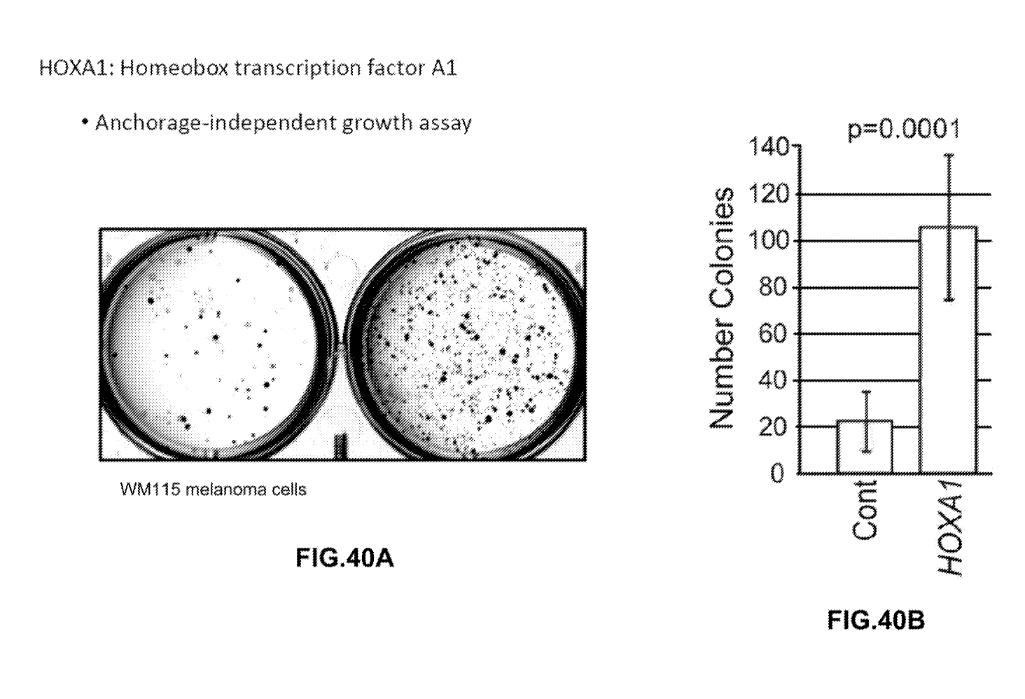

MX2 functional studies: Expression of MX2 increases survival and reduces apoptosis of rat intestinal epithelial cells in non-adherent conditions. See FIGS. 38A-38E.

### Example 3

### Functional and Clinical Validation Data for ACP5

Example 1 has identified ACP5 as one of the 6 pro-invasion oncogenes that can confer enhanced metastasis risk in vivo and therefore carry prognostic significance in patients diagnosed with primary melanomas. In this example, ACP5 was further examined as a proof-of-concept example based on the observations that (i) ACP5 was the only gene exhibiting significant expression correlation with transformation as well as progression (Table 1) and (ii) ACP5 has been used as a histochemical marker of osteoclastic activity, which is increased in conditions of bone diseases including bone metastases (Halleen et al., Clin Chem 47, 597-600 (2001); Capeller et al., Anticancer Res 23, 1011-1015 (2003); Lyubimova et al., Bull Exp Biol Med 138, 77-79 (2004)).

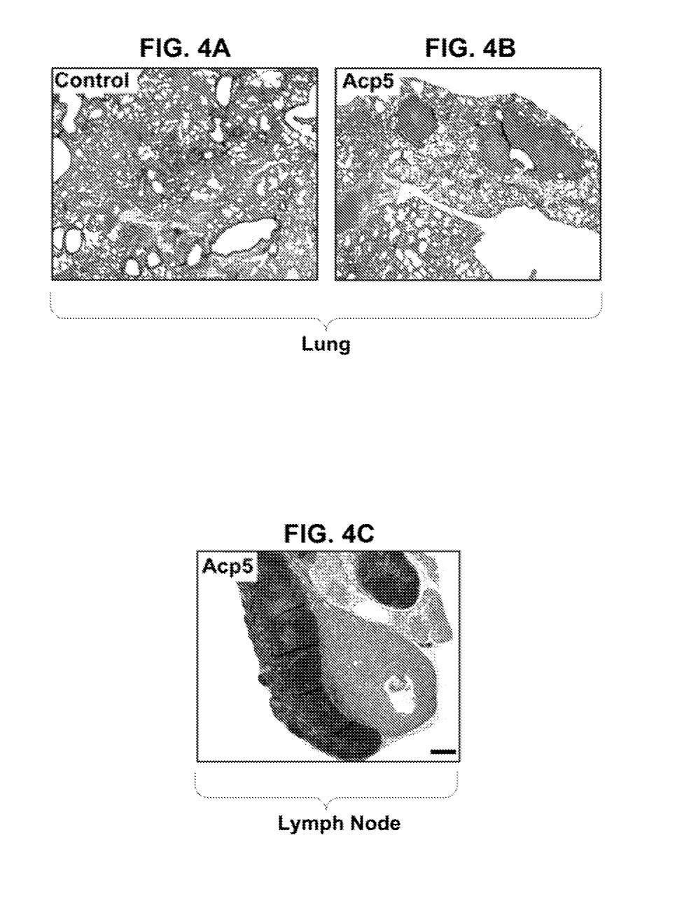

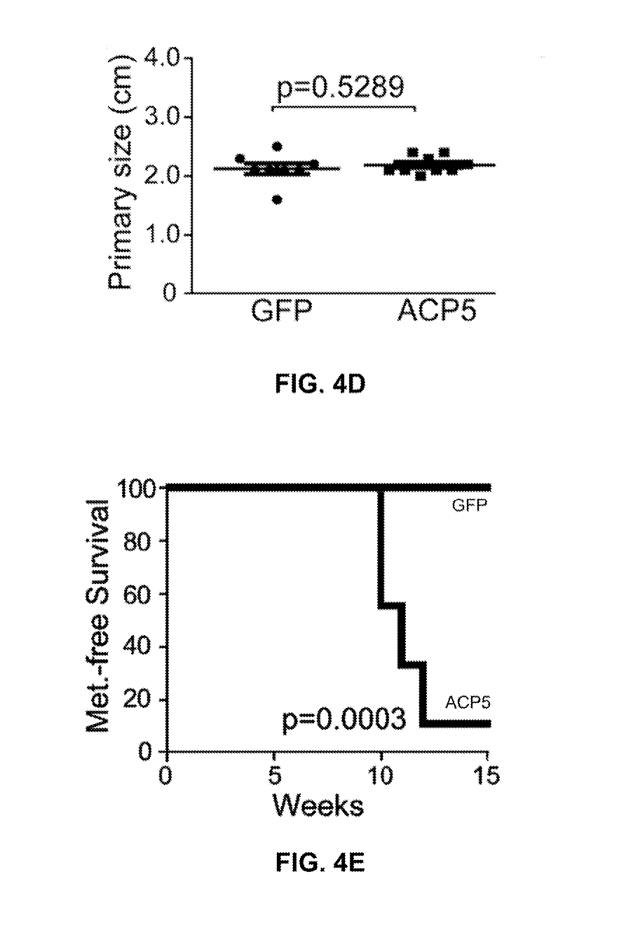

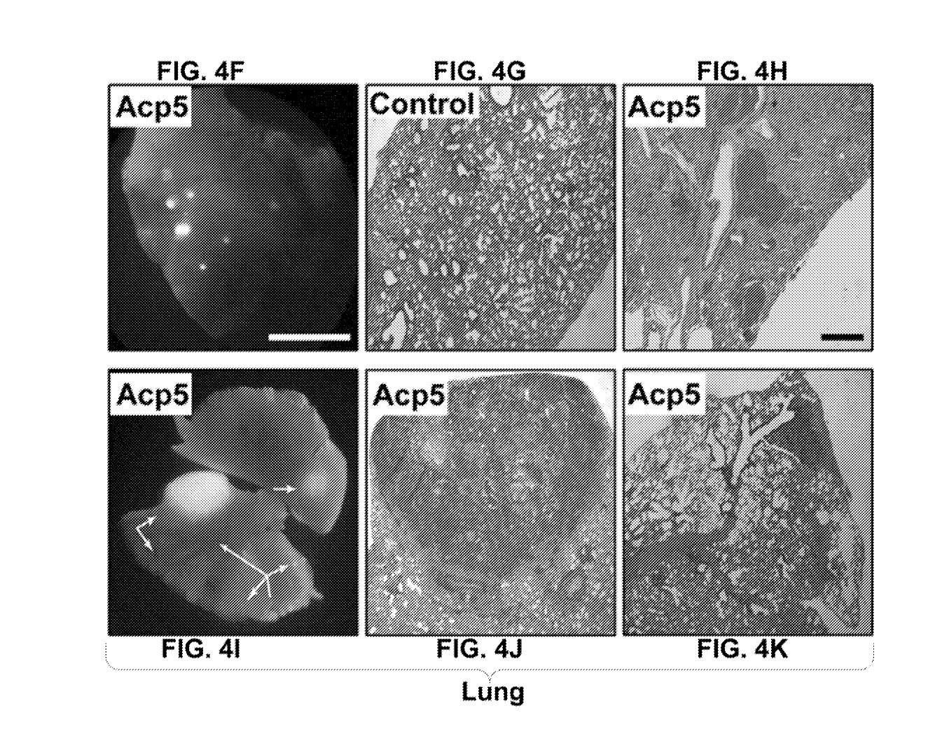

To demonstrate ACP5's ability to drive distal metastasis in vivo, ACP5 or GFP control was over-expressed in the human melanoma cell line 1205Lu, which shows minimal to no distal metastasis from skin orthotopic tumor sites. Briefly, cells (1×10⁶) were implanted into the subcutaneous orthotopic site in the skin on a single flank of athymic nude mice (n=5) and followed for primary tumor growth. When tumors reached 2 cm in one dimension, animals were sacrificed and examined for macro and micro metastasis in lymph nodes and distal organ systems. Consistent with its invasive activity, animals bearing ACP5-expressing melanomas in the subcutaneous sites developed spontaneous metastasis to the lung and lymph nodes (n=2; FIGS. 4A-4C) while none in the control cohort harbored any metastatic lesion despite similar tumor penetrance in both cohorts (n=5 each). Additionally, based on the prognostic significance of these genes in human breast cancers (see below), NB008 (mTerc−/−, p53+/−; mTerc), a well-characterized, non-metastatic cell line originating from a spontaneous murine breast adenocarcinoma (mTerc−/−, p53+/−) engineered to re-express mTerc, was utilized. Specifically, GFP-labeled NB008 cells stabling expressing ACP5 or vector control were orthotopically implanted into the right inguinal mammary fat pad of athymic nude mice. Macroscopic GFP-positive lesions in the lungs were scored at necropsy when primary mammary tumors reached 2 cm maximum size (FIGS. 4D-4E). As shown by Kaplan-Meier metastasis-free survival analysis, GFP-positive macro-metastasis was detected in the lungs of 89% (8/9) of mice bearing ACP5-expressing tumors, whereas none (0/8) of the animals injected with vector control tumor cells presented with lung nodules (p=0.0003; FIG. 4D-4E). Histopathological examination confirmed presence of macro- and micro-metastases (FIGS. 4F-4K). Together, these results show that ACP5 is a bona fide metastasis driver in vivo.

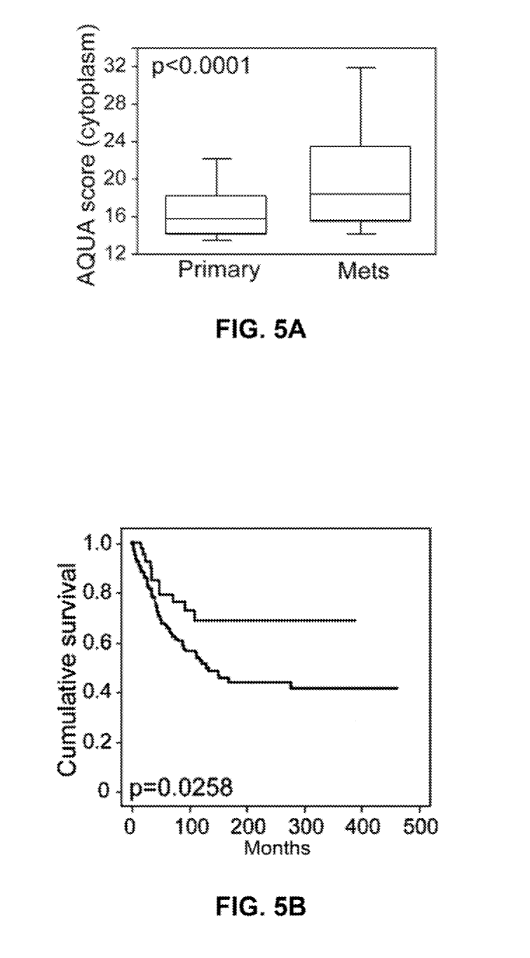

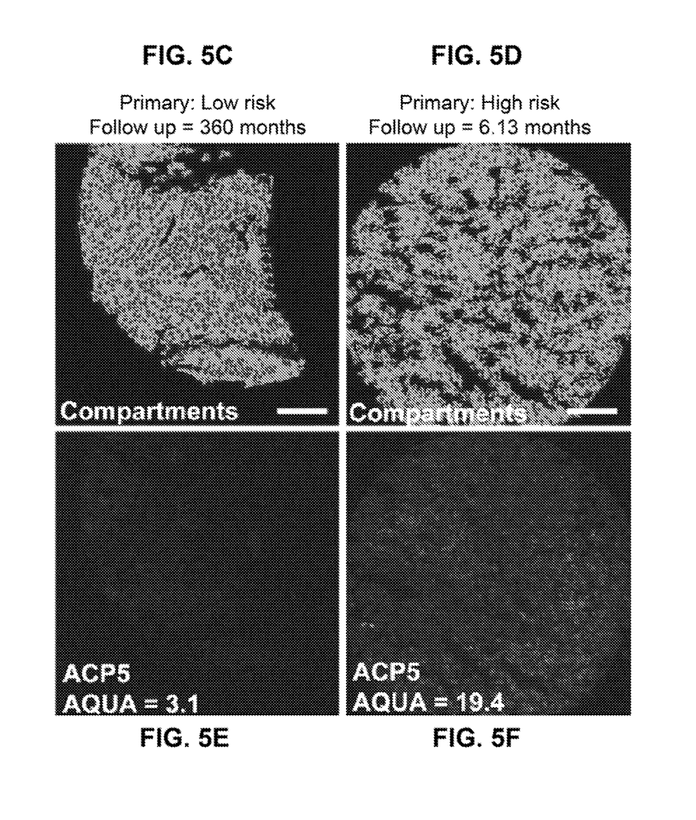

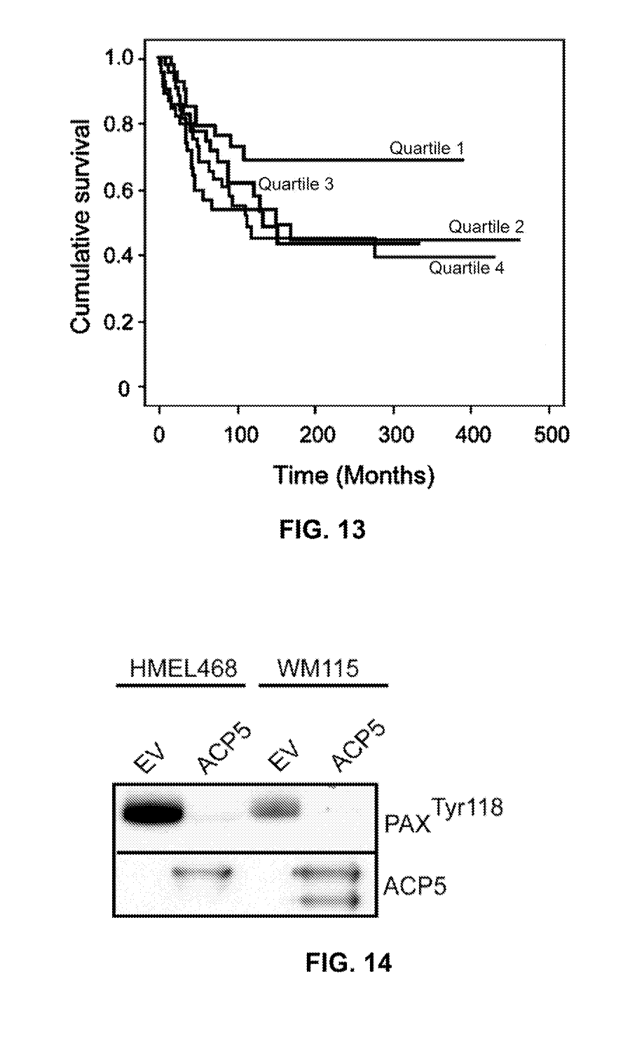

Next, to investigate the prognostic significance of ACP5 expression in human primary melanomas, the quantitative immunofluorescence platform AQUA® was employed to measure ACP5 protein expression on a tissue microarray (YTMA59) containing 196 cases of primary melanomas and 299 cases of metastatic melanomas annotated for survival outcome (Berger et al., Cancer Res 65, 11185-11192 (2005); Gould Rothberg et al., J Clin Oncol 27, 5772-5780 (2009)). As observed in the clinicopathological validation study (FIGS. 11A-11N), ACP5 staining was primarily cytoplasmic, and the differential distributions of staining intensity by AQUA were significantly up-regulated in the metastatic lesions compared to primary specimens (FIG. 5A; ANOVA P<0.0001). Importantly, ACP5 protein expression level in the primary melanoma cases is correlated with survival, for which a significantly shorter melanoma-specific survival was observed in cases with higher level of ACP5 cytoplasmic expression (log rank p=0.0258; FIGS. 5B-5F and FIG. 13). Collectively, the data therefore show that ACP5 is not only a pro-invasion oncogene but also a prognostic biomarker in human primary melanomas.

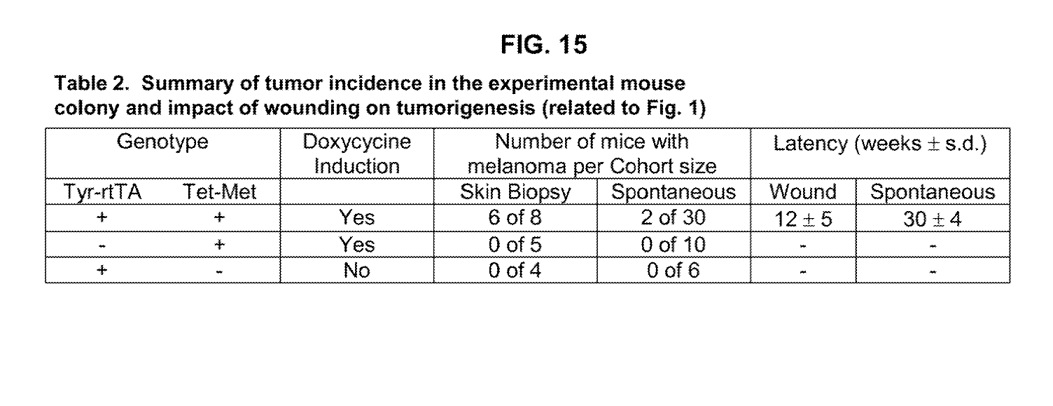

On the cell biological level, over-expression and RNAi-knockdown of ACP5 resulted in striking morphological changes such as cell spreading and cell rounding, respectively (FIGS. 6A-6D). Over-expression of ACP5 in WM115 melanoma cells led to a reproducible decrease in FAK auto-phosphorylation at Tyr397 in cells propagated with or without matrigel or fibronectin coatings (FIG. 6E). Phospho-tyrosine (pTyr) immunoblotting of FAK-immunoprecipitated (IP) WM115 and HMEL468 cell lysates revealed a global impact on FAK tyrosine phosphorylation beyond its autophosphorylation site (FIG. 6F). Similarly, anti-pTyr-IP analysis uncovered a more significant effect of ACP5 over-expression on tyrosine phosphorylation of Paxillin (PAX; FIG. 6F), including Tyr118 (FIG. 14), which is a critical residue thought to serve as docking sites for other signaling molecules. Live-cell imaging of ACP5 over-expressing cells translated these biochemical changes to increased cell movement, consistent with the data on ACP5's activity on cell invasion. Because the FAK complex activity has been implicated in metastasis (Zheng et al., Cell Cycle 8, 3474-3479 (2009)), this mechanistic link thus further substantiates the functional role of ACP5 in invasion and points to the FAK complex as a possible point of therapeutic intervention in high-risk primary melanoma with high ACP5 expression.

### Example 4

### Melanoma Tumorigenesis and Metastasis Requires Phosphatase Activity of ACP5

An improved ACP5 phosphatase activity assay (see Table 10) was used to examine whether the phosphatase activity of ACP5 is required for its function in cell invasion and in vivo metastasis. Molybdate was used as an acid phosphatase inhibitor. S. Perez-Amodio et al., Bone, (2005), 36: 1065-1077; and Pernilla Lang et al., the Journal of Histochemistry & Cytochemistry, (2001), 49(3): 379-396. 293T cells were transfected with GFP/pLenti6 and ACP5/pLenti6 lentiviral vectors using Lipofectamine 2000 for 48 h. Cell lysates and conditioned medium were collected and subjected to the acid phosphatase activity assay.

### Table 10

### Phosphatase Activity Assay for ACP5 (TRAP)

Lysis buffer: sodium acetate buffer (50 mM pH5.8) containing Triton X-100 (1% v/v) and a cocktail of proteinase inhibitors
Quantitate the lysates and add 1 ug lysates for the assay.

For Measurement in the Conditioned Media:
Add about 2-4 μL of media after normalization to the concentration of cell lysates.
TRAP enzyme activity was assayed in 96-well using 150 μl of the reaction buffer:

| p-nitrophenylphosphate (pNPP) | 10 | mM |
|---|---|---|
| Na-acetate (pH 5.8): | 0.1M | |
| KCl | 0.15M | |
| Triton X-100 | 0.1% (v/v) | |
| Na-tartrate | 10 | mM |
| ascorbic acid | 1 | mM |
| FeCl3 | 0.1 | mM |

Parallel incubation also contained 1000 μM molybdate as a TRAP inhibitor.
Then add 100 μL of NaOH (0.3M) to stop the reaction and read at OD 405 nm.

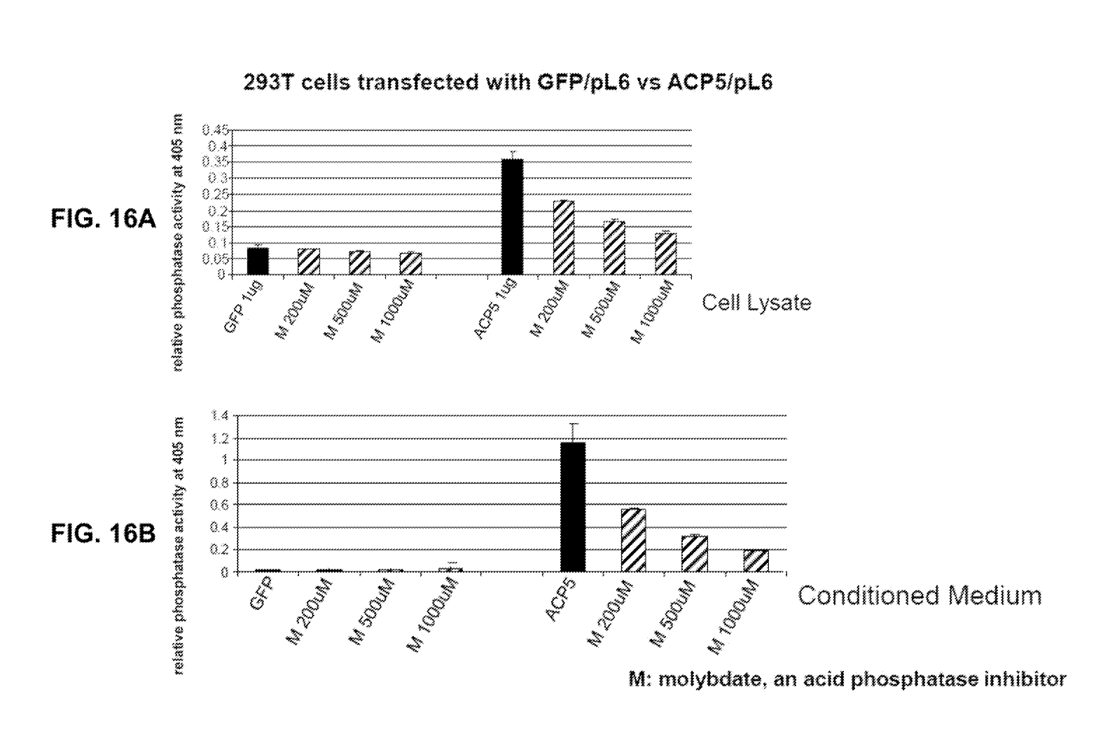

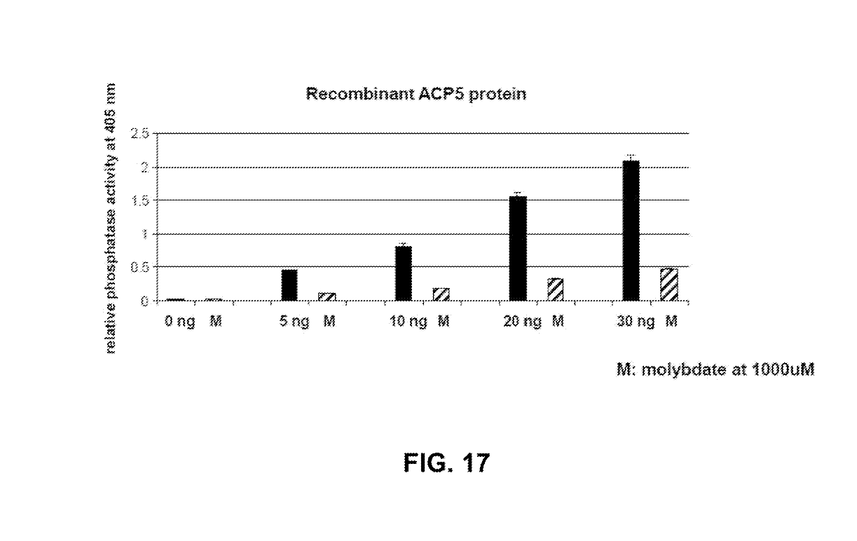

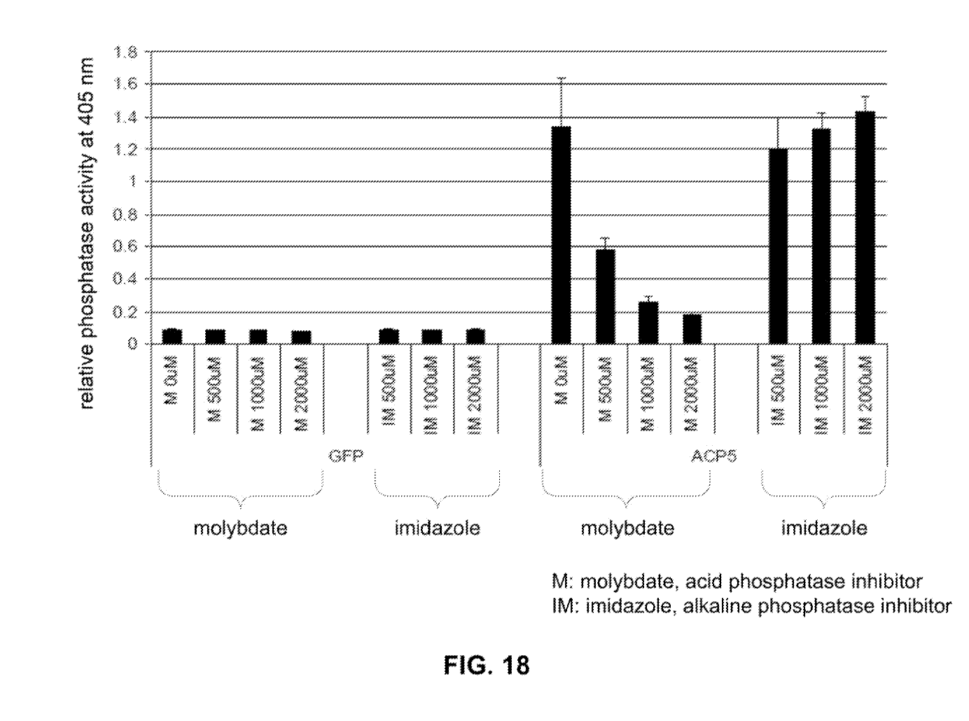

The improved acid phosphatase assay was used to measure the phosphatase activity of ACP5 in both cell lysates and conditioned medium (FIGS. 16A-16B). The increased phosphatase activity of ACP5 can be inhibited by increased concentrations of molybdate, an acid phosphatase inhibitor (FIGS. 16A-16B). Similar results were obtained with a recombinant ACP5 protein, when tested in a phosphatase activity assay using molybdate as an acid phosphatase inhibitor (FIG. 17). To confirm the specificity of the acid phosphatase activity induced by ACP5, the effect of molybdate, an acid phosphatase inhibitor, was compared to imidazole, an alkaline phosphatase inhibitor. See FIG. 18. HMEL cells stably expressing GFP and ACP5 were generated using lentiviral infection. Cell lysates were prepared and 1 μg lysates were subjected to acid phosphatase activity assay in the presence of increased concentrations of molybdate and imidazole. It was shown that the increased activity of ACP5 can be inhibited by molybdate, rather than imidazole.

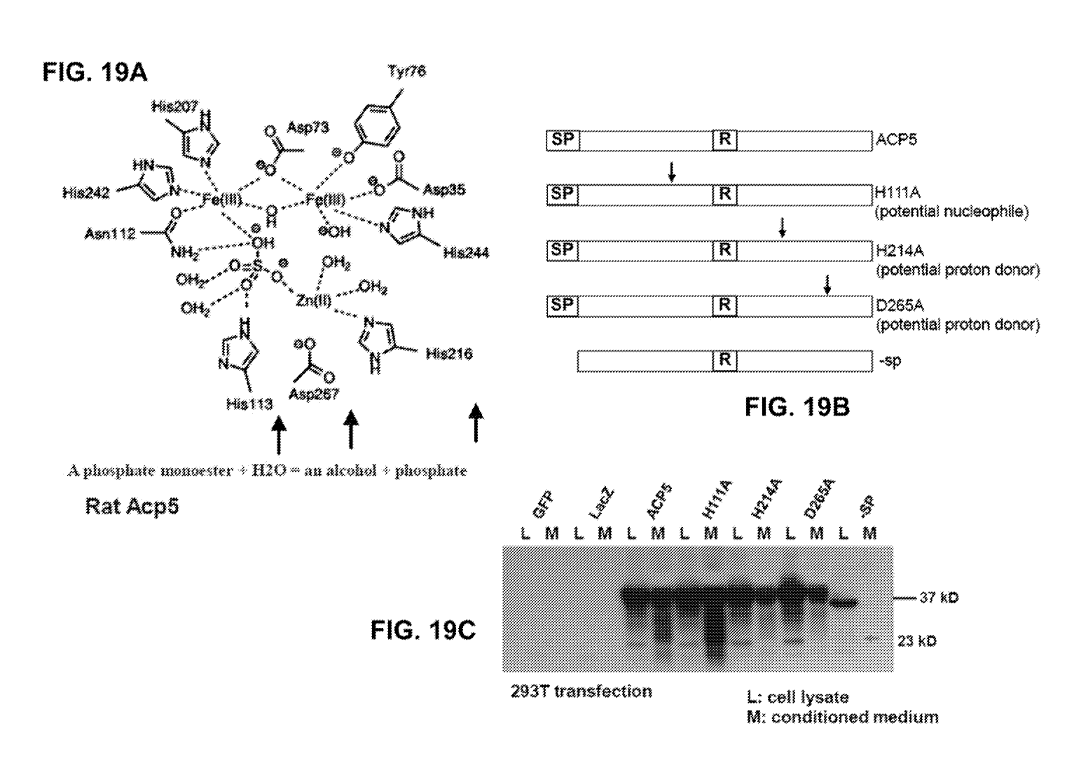

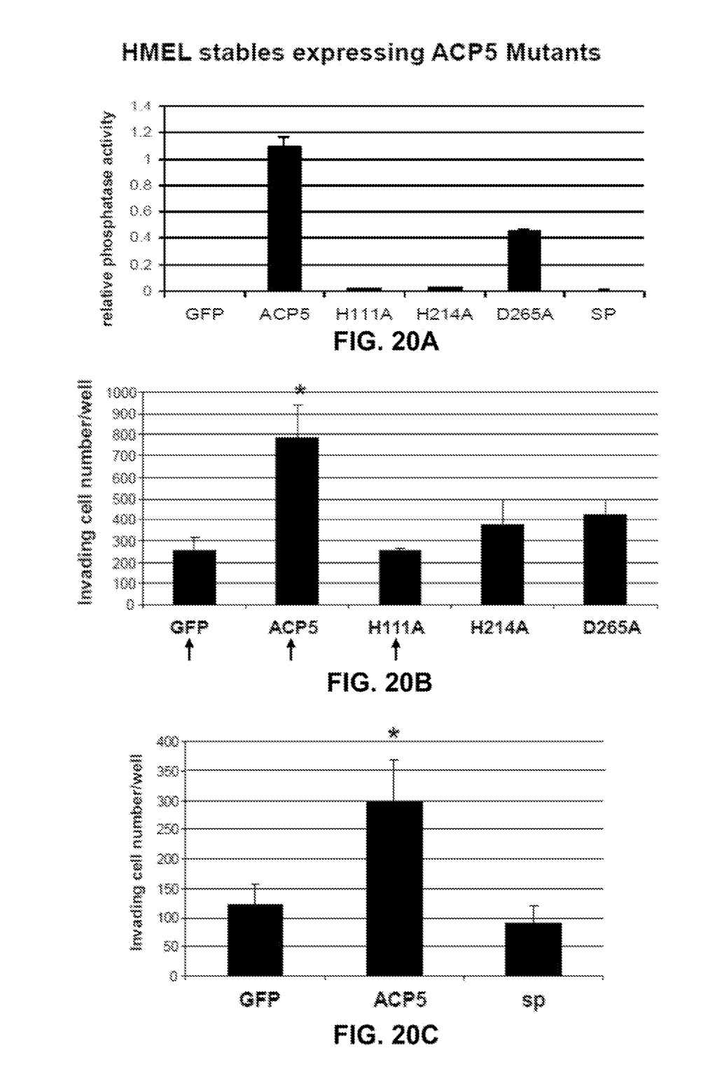

To confirm that ACP5 phosphatase activity is required for its function in cell invasion, three single amino acid mutants H111A, H214A and D265A were generated using Quikchange® site-directed mutagenesis kit (Strategen). These amino acid residues are important for the phosphatase activity of ACP5, based on the structural information on rat ACP5 protein (Lindqvist, et al., J. Mol. Biol. (1999) 291, 135-147). See FIGS. 19A-19B. The H111A and H214A mutants almost completely lost the phosphatase activity compared to wild type ACP5, while the D265A mutant still retained −40% of the activity (FIG. 20A).

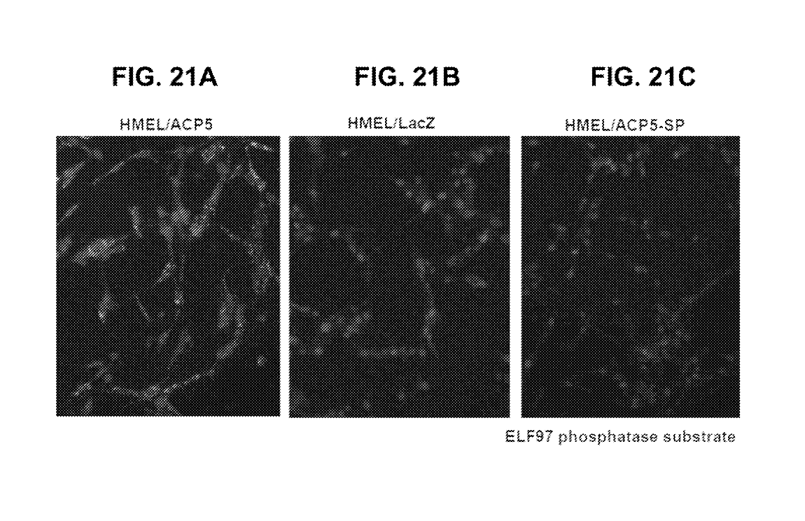

A deletion mutant (−sp) was also generated by deleting the signal peptide required for secretion of ACP5. In addition, the phosphatase activity was also confirmed by staining with ELF97 as the phosphatase substrate, based on the modified protocol reported by Filgueira, Histochem. Cytochem. (2004) 52(3): 411-414. The −sp deletion mutant, like mutant H111A, also lost phosphatase activity (FIG. 20A). Therefore, secretion is essential for ACP5 function. See FIGS. 21A-21C.

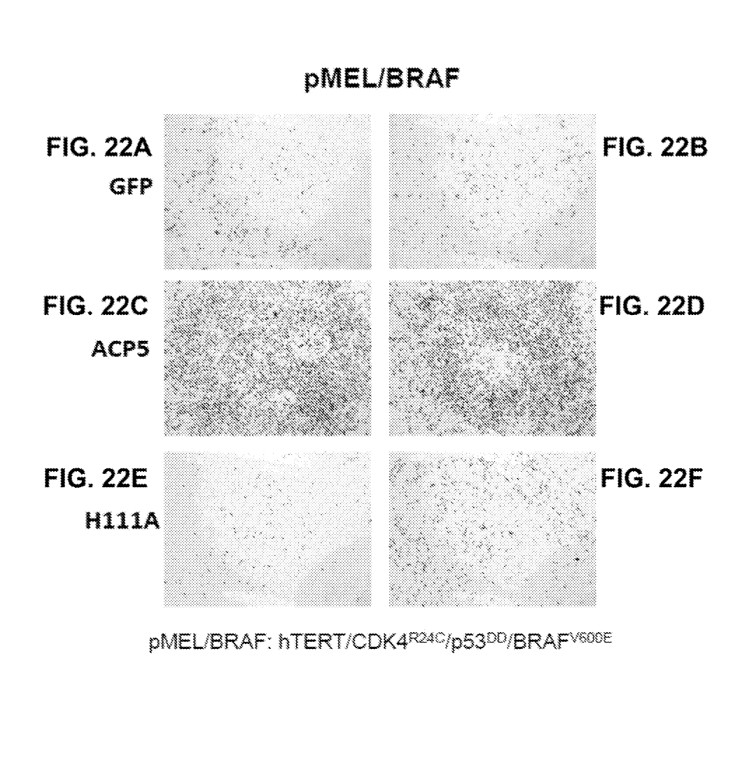

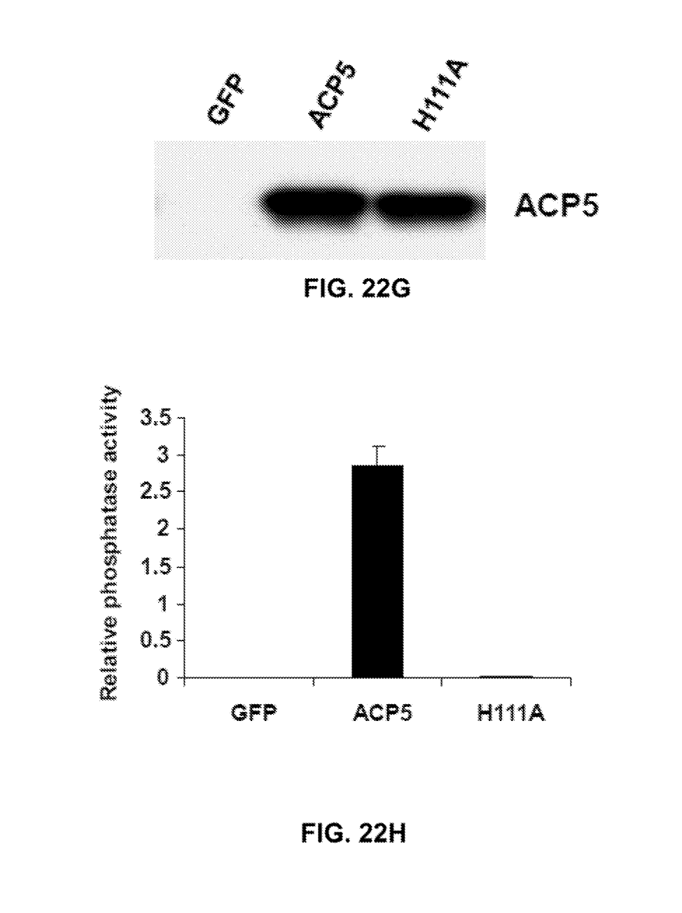

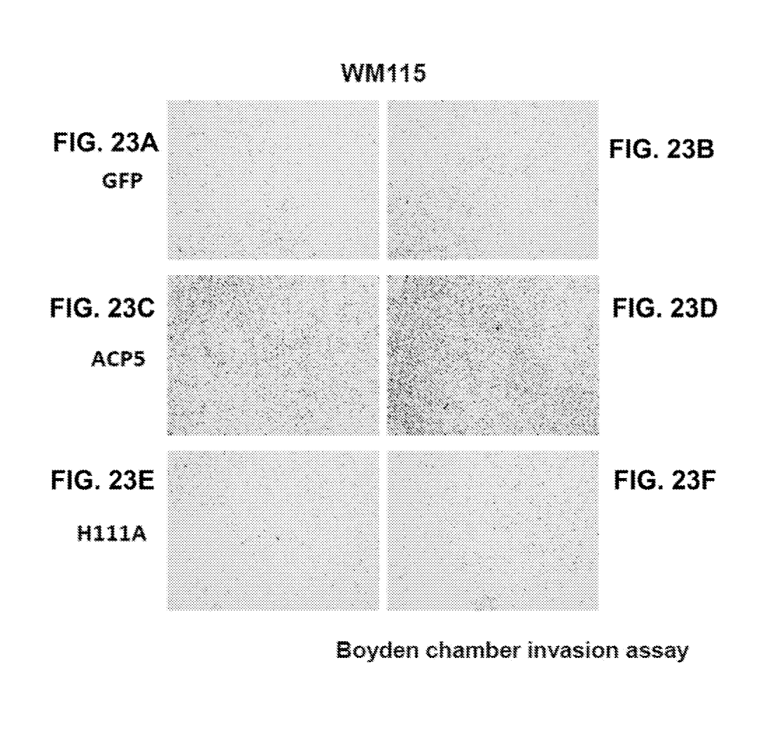

A Boyden Chamber Invasion assay was further used to confirm that phosphatase activity of ACP5 is required for its function in cell invasion. As shown in FIG. 20B-20C, only wild type ACP5 significantly induced invasion of HMEL cells, as compared to the H111A, H214A, D265A, and −sp deletion mutants. Moreover, ACP5 also significantly induced invasion of pMEL/BRAF and WM115 cells. In contrast, the H111A mutant has no effect on invasion. See FIGS. 22A-22F and 23A-23F.

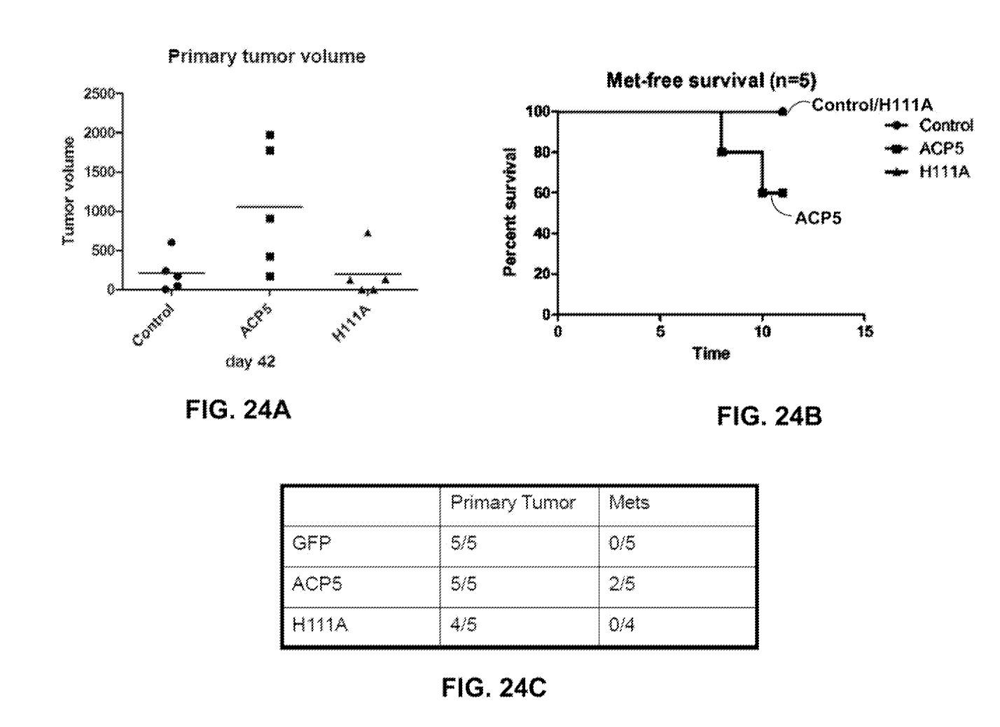

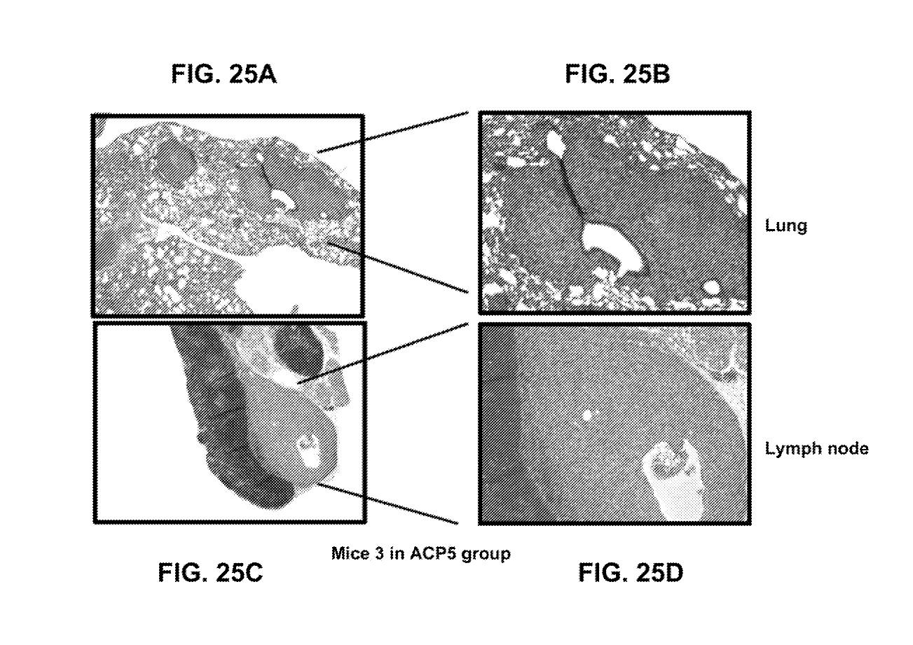

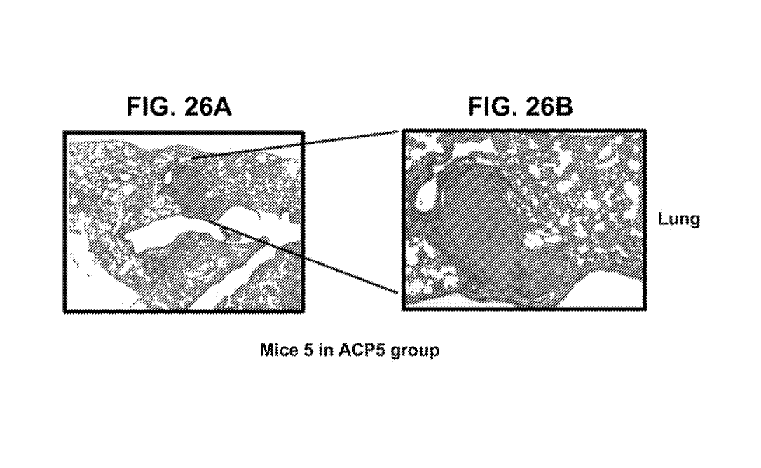

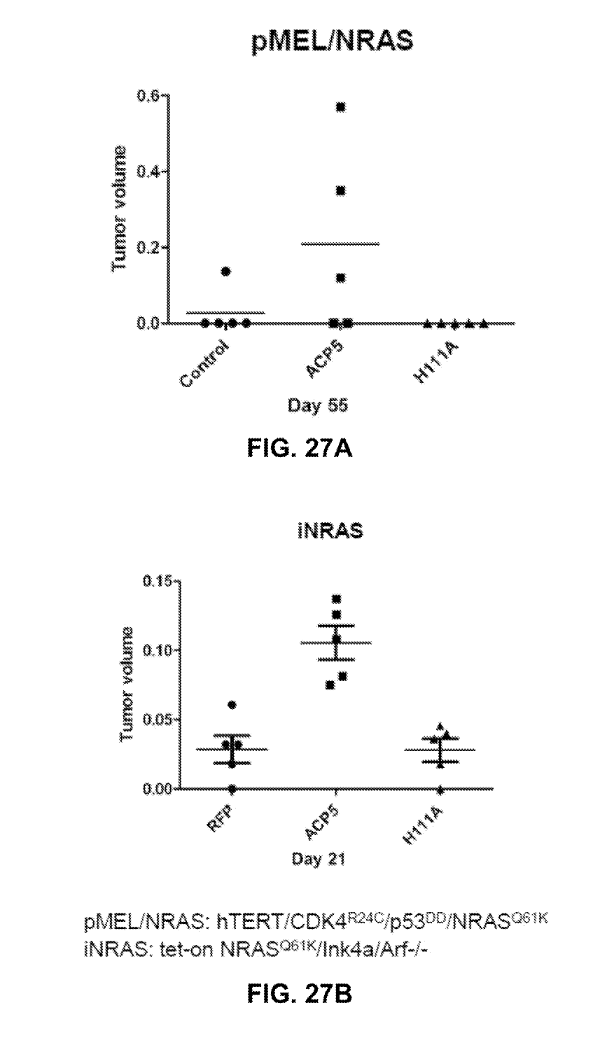

An in vivo metastasis assay was performed to confirm that phosphatase activity of ACP5 is required for its function in metastasis. Stable cell lines (1205Lu) expressing GFP, wild-type ACP5, and ACP5 H111A mutant were generated through lentiviral infection. Cells were injected subcutaneously into the right flank of nude mice at 1×10⁶ cells/site, 5 mice/group. Mice were monitored for tumor growth and sacrificed when tumors reached 2 cm in one dimension. Metastasis was confirmed by H&E. As shown in FIGS. 24A-24C, two out of the five mice in the ACP5 group had lung metastasis, while metastasis was not observed in those mice of the GFP control or H111A mutant group. These results indicate that ACP5 drives in vivo metastasis to lung and lymph node and the phosphatase activity of ACP5 is required for its function in melanoma metastasis. See FIGS. 24A-24C, 25A-25B, and 26A-26B.

Two additional in vivo metastasis assays were performed using pMEL/NRAS and iNRAS cell lines. The experiments were done in the same manner as the 1205Lu cell line experiment described above. The expression of ACP5 promoted primary tumor growth and this effect is dependent on the phosphatase activity of ACP5, consistent with the above-described observation in 1205Lu cell lines.

The data provided in this example can lead to new diagnostic methods and therapies and targeting the phosphatase activity of ACP5 to treat melanoma and other types of cancer. Examples of new therapeutics include, for example, neutralizing antibodies and chemical inhibitors.

### Example 4

### UBE2C

Example 1 has identified UBE2C as one of the 18 pro-invasion associated biomarkers. In this example, UBE2C was further shown to exhibit higher expression in melanomas versus nevi and cooperatively transforms primary fibroblasts.

RNA-Based Expression Assay by Panomics Technology:

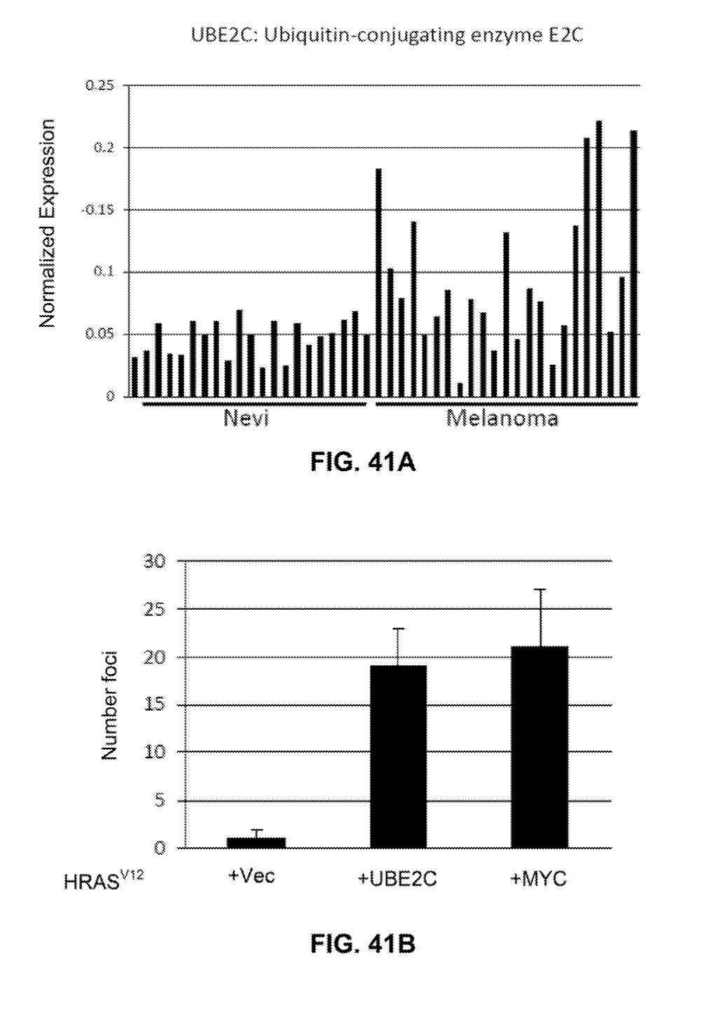

As an alternative to protein-based expression analysis, we also utilize QuantiGene® Plex technology (Panomics) to assess the RNA expression of biomarkers. The QuantiGene® platform is based on the branched DNA technology, a sandwich nucleic acid hybridization assay that provides a unique approach for RNA detection and quantification by amplifying the reporter signal rather than the sequence (Flagella et al., Analytical Biochemistry 352(1):50-60 (2006)). This technology can reliably measure quantitatively RNA expression in fresh, frozen or formalin-fixed, paraffin-embedded (FFPE) tissue homogenates (Knudsen et al., Journal of Molecular Diagnostics 10(2): 170-175 (2008)). As shown in FIG. 41A, a feasibility pilot has shown that we can reliably measure the RNA expression of UBE2C in 21 spitz nevi and 22 malignant melanoma specimens that are in FFPE blocks. Analysis of each gene achieved excellent reproducibility with Coefficient of Variation (CV) values in the 8-9% range, thus meeting maximum quality control standards. This methodology thus provides an ideal alternative to glean first insight into expression pattern of a candidate of interest without available antibody. Of note, the QuantiGene® Plex analysis of UBE2C corroborated results indicating oncogenic activity of UBE2C. Specifically, using the classical co-transformation assay we show that UBE2C cooperated with activated HRASV12 to increase transformed focus formation in Ink4a/Arf-deficient primary mouse embryonic fibroblasts (FIG. 41B)

### Example 5

### RNF2 and UCHL5

Methods

Boyden Chamber Assay: the assay is conducted as described above. Briefly, 100,000 cells were plated in matrigel coated Boyden Chamber (BD Biosciences) in serum free media and grown for 24-48 hrs. After incubation, cells were fixed, stained with crystal violet and pictured.

Mice injection: One million cells were injected subcutaneously in NCR/NUDE mice (5-10 mice per sample) and tumors were allowed to grow till they were 2 cm in one direction. Mice were sacrificed, dissected and lungs and tumor formaline fixed. These were then paraffin-embedded, sectioned and H&E stained.

Cell Culture: HMEL and WM115 Cells were grown in 37 degrees and 5% CO2 in standard cell-culture incubators in DMEM media.

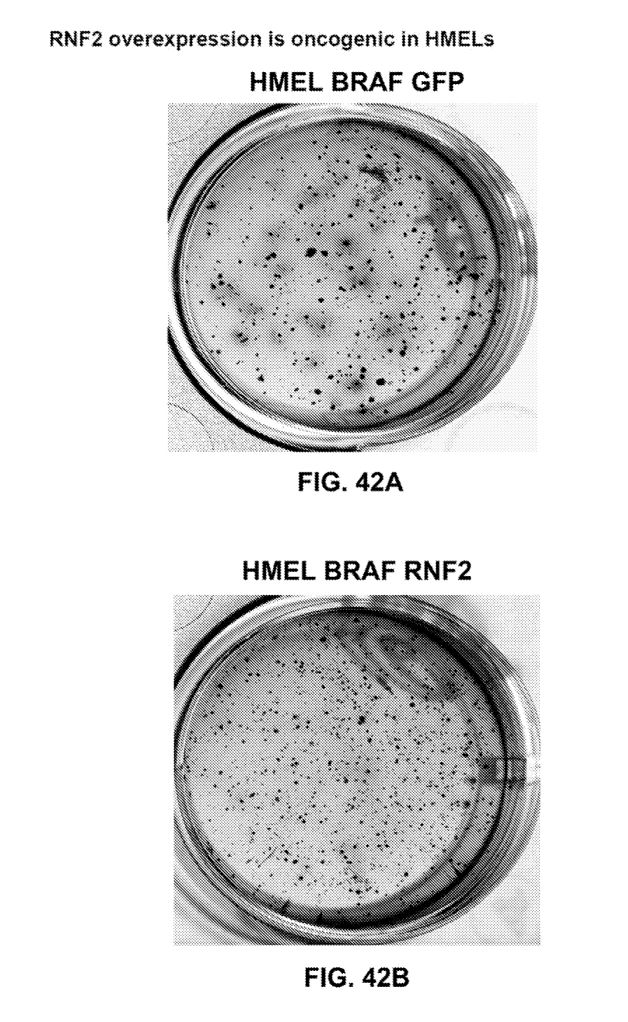

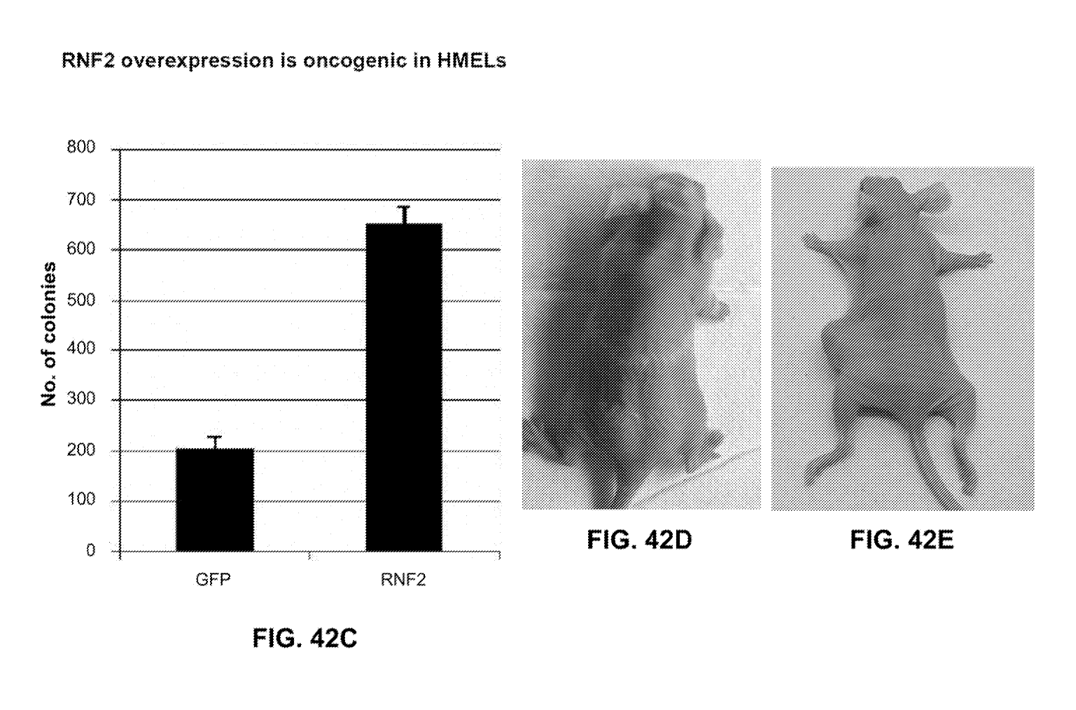

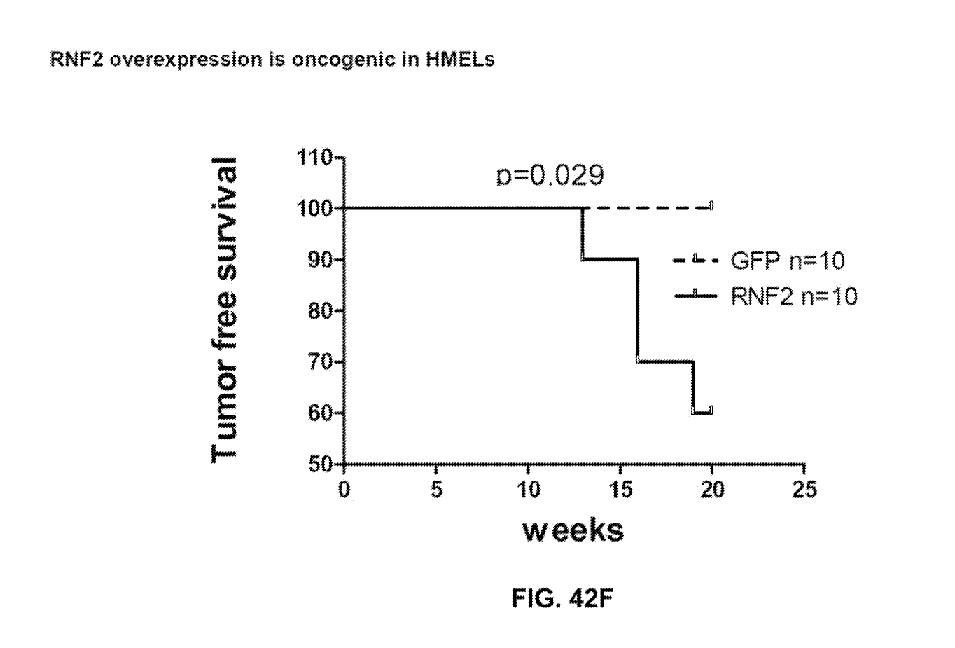

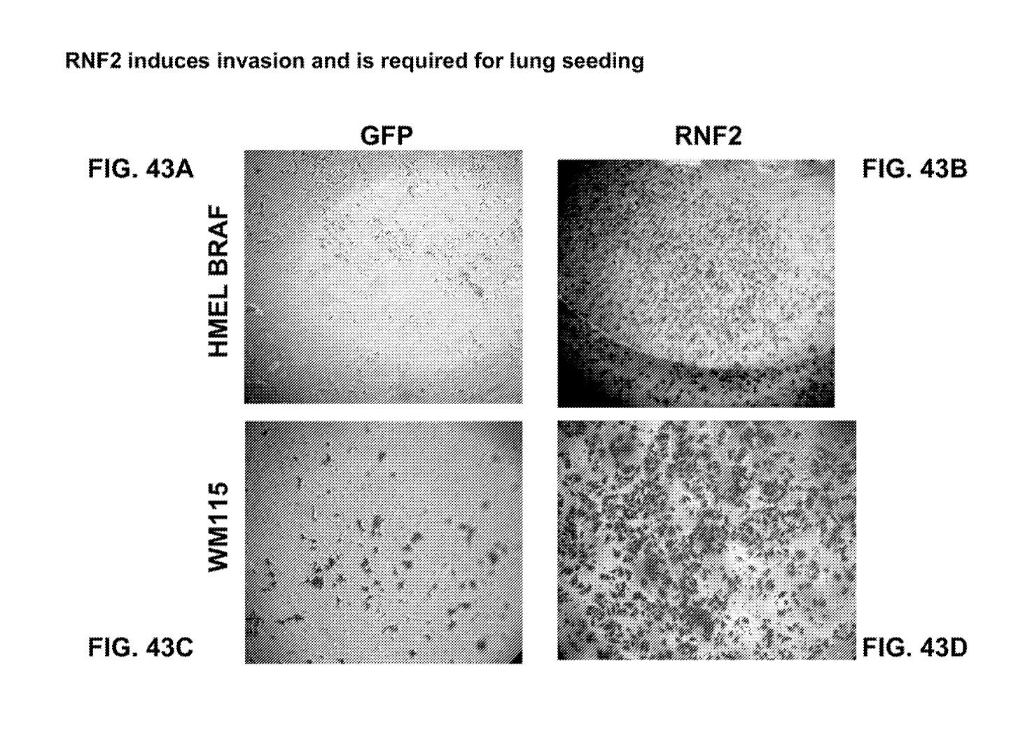

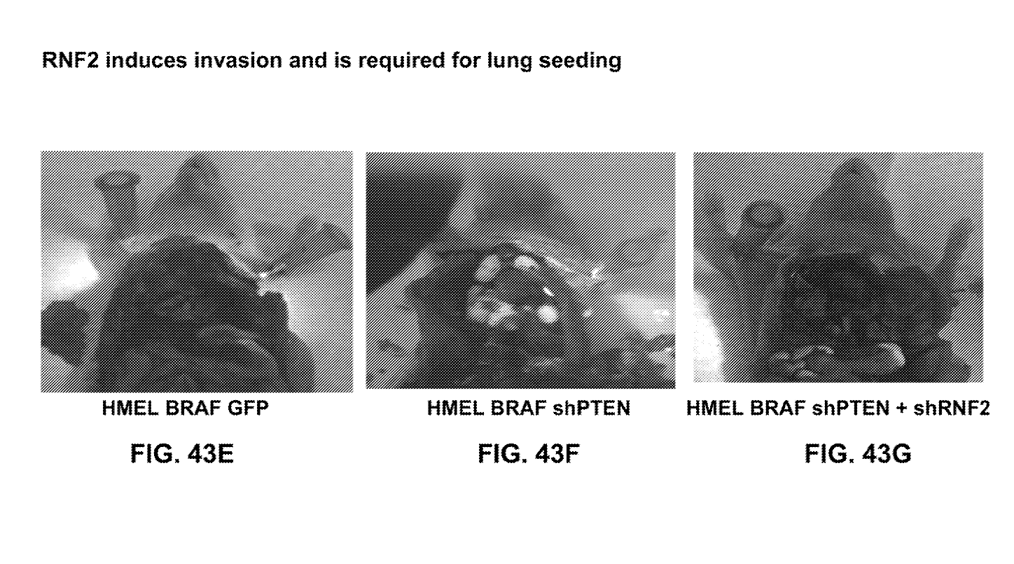

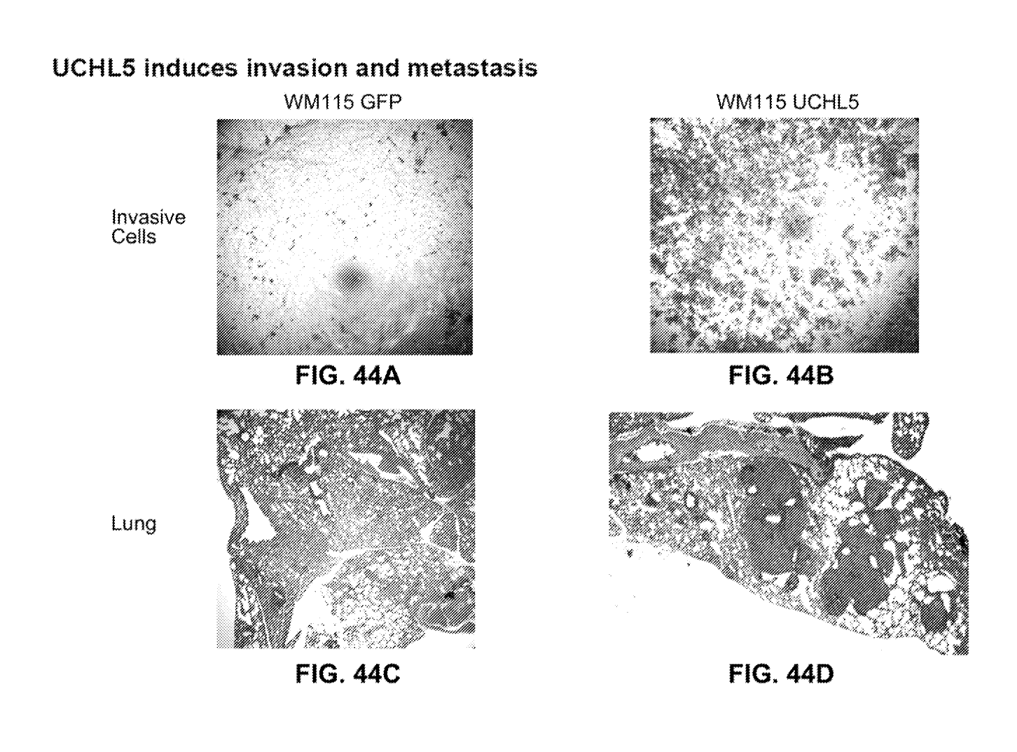

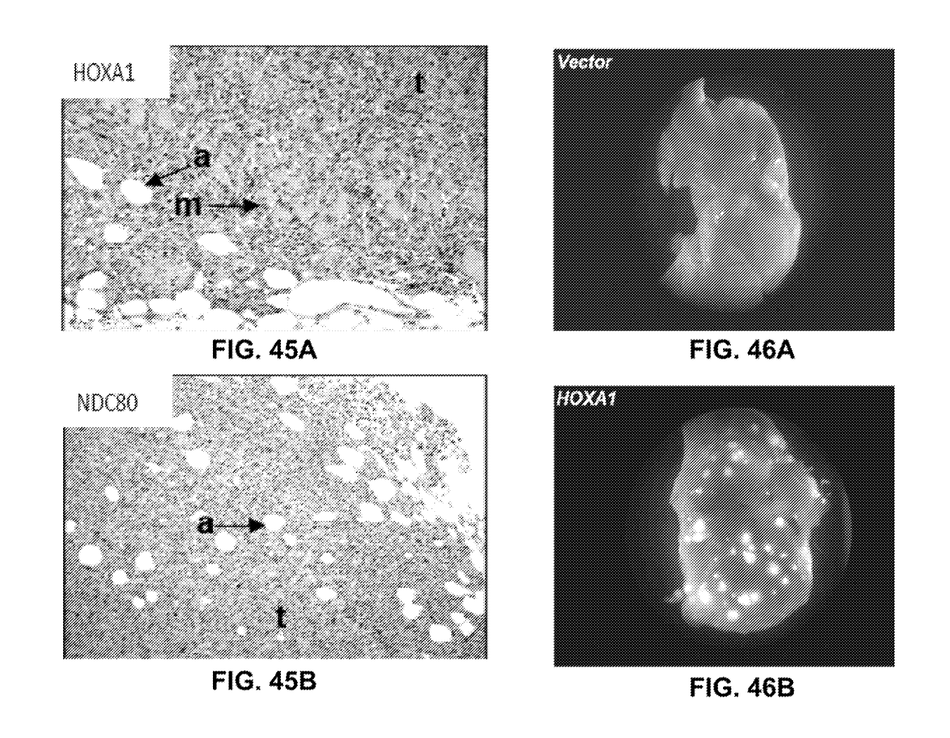

Results: As shown in FIGS. 42A-42F and 43A-43G, RNF2 promoted anchorage-independent growth and tumor formation of immortalized primary melanocytes in nude mice, indicating that RNF2 is oncogenic. Further, RNF2 promoted invasiveness of immortalized primary melanocytes and melanoma cells, suggesting its role in metastasis process. RNF2 is also essential for lung seeding of pro-invasive melanocytes establishing its requirement for metastasis process. As shown in FIGS. 44A-44D, UCHL5 promotes invasiveness of melanoma cells suggesting its role in metastasis process. UCHL5 over-expression leads to lung metastasis from subcutaneous site suggesting UCHL5 is sufficient to impart metastatic properties to non-metastatic melanoma cells.

TABLE 3
Summary of integrated dataset comprised of 360 potential metastasis genes.

| | | |
|---|---|---|
| Gene ID | Gene Symbol | Gene Name |

65 down-regulated/deleted candidates

| | | |
|---|---|---|
| 79026 | AHNAK | AHNAK nucleoprotein |
| 360 | AQP3 | aquaporin 3 (Gill blood group) |
| 622 | BDH1 | 3-hydroxybutyrate dehydrogenase, type 1 |
| 219738 | C10ORF35 | chromosome 10 open reading frame 35 |
| 726 | CAPN5 | calpain 5 |
| 999 | CDH1 | cadherin 1, type 1, E-cadherin (epithelial) |
| 51873 | CGI-38 | tubulin polymerization-promoting protein family member 3 |
| 1159 | CKMT1A | creatine kinase, mitochondrial 1A |
| 85445 | CNTNAP4 | contactin associated protein-like 4 |
| 1303 | COL12A1 | collagen, type XII, alpha 1 |
| 9244 | CRLF1 | cytokine receptor-like factor 1 |
| 1410 | CRYAB | crystallin, alpha B |
| 1428 | CRYM | crystallin, mu |
| 113878 | DTX2 | deltex homolog 2 (Drosophila) |
| 10278 | EFS | embryonal Fyn-associated substrate |
| 79993 | ELOVL7 | ELOVL family member 7 |
| 2041 | EPHA1 | EPH receptor A1 |
| 2045 | EPHA7 | EPH receptor A7 |
| 2051 | EPHB6 | EPH receptor B6 |
| 2125 | EVPL | envoplakin |
| 2159 | F10 | coagulation factor X |
| 375061 | FAM89A | family with sequence similarity 89, member A |
| 8857 | FCGBP | Fc fragment of IgG binding protein |
| 2261 | FGFR3 | fibroblast growth factor receptor 3 |
| 56776 | FMN2 | formin 2 |
| 2770 | GNAI1 | guanine nucleotide binding protein (G protein) |
| 7107 | GPR137B | G protein-coupled receptor 137B |
| 64388 | GREM2 | gremlin 2, cysteine knot superfamiiy |
| 3098 | HK1 | hexokinase 1 |
| 688 | KLF5 | Kruppel-like factor 5 (intestinal) |
| 5655 | KLK10 | kallikrein-related peptidase 10 |
| 11202 | KLK8 | kallikrein-related peptidase 8 |
| 10748 | KLRA1 | killer cell lectin-like receptor subfamily A, member 1 |
| 10219 | KLRG1 | killer cell lectin-like receptor subfamily G, member 1 |
| 4135 | MAP6 | microtubule-associated protein 6 |
| 5603 | MAPK13 | mitogen-activated protein kinase 13 |
| 4312 | MMP1 | matrix metaliopeptidase 1 (interstitial collagenase) |
| 10205 | MPZL2 | myelin protein zero-like 2 |
| 4486 | MST1R | macrophage stimulating 1 receptor |
| 4692 | NDN | necdin homolog (mouse) |
| 5092 | PCBD1 | pterin-4 alpha-carbinolamine dehydratase |
| 10158 | PDZK1IP1 | PDZK1 interacting protein 1 |
| 5317 | PKP1 | plakophilin 1 |
| 26499 | PLEK2 | pleckstrin 2 |
| 58473 | PLEKHB1 | pleckstrin homology domain containing |
| 5366 | PMAIP1 | phorbol-12-myristate-13-acetate-induced protein 1 |
| 79983 | POF1B | premature ovarian failure, 1B |
| 5453 | POU3F1 | POU class 3 homeobox 1 |
| 5579 | PRKCB1 | protein kinase C, beta |
| 5745 | PTHR1 | parathyroid hormone 1 receptor |
| 5792 | PTPRF | protein tyrosine phosphatase, receptor type, F |
| 57111 | RAB25 | RAB25, member RAS oncogene family |
| 6095 | RORA | RAR-related orphan receptor A |
| 6337 | SCNN1A | sodium channel, nonvoltage-gated 1 alpha |
| 6382 | SDC1 | syndecan 1 |
| 5268 | SERPINB5 | serpin peptidase inhibitor, clade B (ovalbumin), member 5 |
| 11254 | SLC6A14 | solute carrier family 6 (amino acid transporter), member 14 |
| 6578 | SLCO2A1 | solute carrier organic anion transporter family, member 2A1 |
| 6586 | SLIT3 | slit homolog 3 (Drosophila) |
| 10653 | SPINT2 | serine peptidase inhibitor, Kunitz type, 2 |
| 6768 | ST14 | suppression of tumorigenicity 14 (colon carcinoma) |
| 7070 | THY1 | Thy-1 cell surface antigen |
| 23650 | TRIM29 | tripartite motif-containing 29 |
| 23555 | TSPAN15 | tetraspanin 15 |
| 11197 | WIF1 | WNT inhibitory factor 1 |

295 up-regulated/amplified candidates

| | | |
|---|---|---|
| 79575 | ABHD8 | abhydrolase domain containing 8 |
| 1636 | ACE | angiotensin I converting enzyme (peptidyl-dipeptidase A) 1 |
| 54 | ACP5 | acid phosphatase 5, tartrate resistant |
| 8038 | ADAM12 | ADAM metallopeptidase domain 12 |
| 101 | ADAM8 | ADAM metallopeptidase domain 8 |
| 51327 | AHSP | erythroid associated factor |
| 23600 | AMACR | C1q and tumor necrosis factor related protein 3 |
| 54443 | ANLN | anillin, actin binding protein |
| 80833 | APOL3 | apolipoprotein L, 3 |
| 410 | ARSA | arylsulfatase A |
| 22901 | ARSG | arylsulfatase G |
| 55723 | ASF1B | ASF1 anti-silencing function 1 homolog B |
| 259266 | ASPM | asp (abnormal spindle) homolog, microcephaly associated |
| 477 | ATP1A2 | ATPase, Na+/K+ transporting, alpha 2 (+) polypeptide |
| 6790 | AURKA | aurora kinase A; aurora kinase A pseudogene 1 |
| 9212 | AURKB | aurora kinase B |
| 26053 | AUTS2 | autism susceptibility candidate 2 |
| 627 | BDNF | brain-derived neurotrophic factor |
| 638 | BIK | BCL2-interacting killer (apoptosis-inducing) |
| 332 | BIRC5 | baculoviral IAP repeat-containing 5 |
| 672 | BRCA1 | breast cancer 1, early onset |
| 699 | BUB1 | budding uninhibited by benzimidazoles 1 homolog |
| 701 | BUB1B | budding uninhibited by benzimidazoles 1 homolog beta |
| 51501 | C11orf73 | chromosome 11 open reading frame 73 |
| 29902 | C12ORF24 | chromosome 12 open reading frame 24 |
| 84935 | c13orf33 | chromosome 13 open reading frame 33 |
| 56942 | C16ORF61 | chromosome 16 open reading frame 61 |
| 719 | C3AR1 | complement component 3a receptor 1 |
| 57002 | C7ORF36 | chromosome 7 open reading frame 36 |
| 84933 | C8ORF76 | chromosome 8 open reading frame 76 |
| 781 | CACNA2D1 | calcium channel, voltage-dependent, alpha 2/delta subunit 1 |
| 4076 | Caprin1 | cell cycle associated protein 1 |
| 857 | CAV1 | caveolin 1, caveolae protein, 22 kDa |
| 25776 | CBY1 | chibby homolog 1 (Drosophila) |
| 54908 | CCDC99 | coiled-coil domain containing 99 |
| 6357 | CCL13 | chemokine (C-C motif) ligand 13 |
| 6347 | CCL2 | chemokine (C-C motif) ligand 2 |
| 8354 | CCL7 | chemokine (C-C motif) ligand 7 |
| 890 | CCNA2 | cyclin A2 |
| 947 | CD34 | CD34 molecule |
| 948 | CD36 | CD36 molecule (thrombospondin receptor) |
| 991 | CDC20 | cell division cycle 20 homolog |
| 995 | CDC25C | cell division cycle 25 homolog C |
| 990 | CDC6 | cell division cycle 6 homolog |
| 8317 | CDC7 | cell division cycle 7 homolog |
| 83461 | CDCA3 | cell division cycle associated 3 |
| 55536 | CDCA7L | cell division cycle associated 7-like |
| 983 | CDK1 | cell division cycle 2, G1 to S and G2 to M |
| 5218 | CDK14 | PFTAIRE protein kinase 1 |
| 81620 | CDT1 | chromatin licensing and DNA replication factor 1 |
| 1058 | CENPA | centromere protein A |
| 1062 | CENPE | centromere protein E, 312 kDa |
| 1063 | CENPF | centromere protein F, 350/400ka (mitosin) |
| 79019 | CENPM | centromere protein M |
| 55839 | CENPN | centromere protein N |
| 55165 | CEP55 | centrosomal protein 55 kDa |
| 23177 | CEP68 | centrosomal protein 68 kDa |
| 1070 | CETN3 | centrin, EF-hand protein, 3 |
| 1111 | CHEK1 | CHK1 checkpoint homolog (S. pombe) |
| 26586 | CKAP2 | cytoskeleton associated protein 2 |
| 1163 | CKS1B | CDC28 protein kinase regulatory subunit 1B |
| 1164 | CKS2 | CDC28 protein kinase regulatory subunit 2 |
| 1180 | CLCN1 | chloride channel 1, skeletal muscle |
| 7122 | CLDN5 | claudin 5 |
| 23601 | CLEC5A | C-type lectin domain family 5, member A |
| 10664 | CTCF | CCCTC-binding factor (zinc finger protein) |
| 1565 | CYP2D6 | cytochrome P450, family 2, subfamily D, polypeptide 6 |
| 9265 | CYTH3 | cytohesin 3 |
| 1601 | DAB2 | disabled homolog 2, mitogen-responsive phosphoprotein |
| 10928 | DBF4 | DBF4 homolog |
| 23564 | DDAH2 | dimethylarginine dimethylaminohydrolase 2 |
| 55635 | DEPDC1 | DEP domain containing 1 |
| 91614 | DEPDC7 | DEP domain containing 7 |
| 1719 | DHFR | dihydrofolate reductase |
| 27122 | DKK3 | dickkopf homolog 3 |
| 9787 | DLGAP5 | discs, large (Drosophila) homolog-associated protein 5 |
| 1769 | DNAH8 | dynein, axonemal, heavy chain 8 |
| 30836 | DNTTIP2 | deoxynucleotidyltransferase, terminal, interacting protein 2 |
| 51514 | DTL | denticleless homolog |
| 1854 | DUT | deoxyuridine triphosphatase |
| 1894 | ECT2 | epithelial cell transforming sequence 2 oncogene |
| 51162 | EGFL7 | EGF-like-domain, multiple 7 |
| 64123 | ELTD1 | EGF, latrophilin and seven transmembrane domain containing 1 |
| 56943 | ENY2 | enhancer of yellow 2 homolog |
| 54749 | EPDR1 | ependymin related protein 1 |
| 2115 | ETV1 | ets variant 1 |
| 2131 | EXT1 | exostoses (multiple) 1 |
| 2162 | F13A1 | coagulation factor XIII, A1 polypeptide |
| 116496 | FAM129A | family with sequence similarity 129, member A |
| 51647 | FAM96B | family with sequence similarity 96, member B |
| 2230 | FDX1 | ferredoxin 1 |
| 2235 | FECH | ferrochelatase (protoporphyria) |
| 63979 | FIGNL1 | fidgetin-like 1 |
| 51303 | FKBP11 | FK506 binding protein 11, 19 kDa |
| 2289 | FKBP5 | FK506 binding protein 5 |
| 2350 | FOLR2 | folate receptor 2 (fetal) |
| 2305 | FOXM1 | forkhead box M1 |
| 6624 | FSCN1 | fascin homolog 1, actin-bundling protein |
| 2530 | FUT8 | fucosyltransferase 8 (alpha (1,6) fucosyltransferase) |
| 51809 | GALNT7 | UDP-N-acetyl-alpha-D-galactosamine |
| 64096 | GFRA4 | GDNF family receptor alpha 4 |
| 152007 | GLIPR2 | GLI pathogenesis-related 2 |
| 2740 | GLP1R | glucagon-like peptide 1 receptor |
| 51053 | GMNN | geminin, DNA replication inhibitor |
| 2775 | GNAO1 | guanine nucleotide binding protein (G protein), polypeptide O |
| 2792 | GNGT1 | guanine nucleotide binding protein (G protein), polypeptide 1 |
| 2894 | GRID1 | glutamate receptor, ionotropic, delta 1 |
| 2936 | GSR | glutathione reductase |
| 2966 | GTF2H2 | general transcription factor IIH, polypeptide 2, polypeptide 2D |
| 51512 | GTSE1 | G-2 and S-phase expressed 1 |
| 3045 | HBD | hemoglobin, delta |
| 64151 | HCAP-G | non-SMC condensin I complex, subunit G |
| 50810 | HDGFRP3 | hepatoma-derived growth factor, related protein 3 |
| 3082 | HGF | hepatocyte growth factor (hepapoietin A; scatter factor) |
| 3012 | HIST1H2AB | histone cluster 1, H2ae; histone cluster 1, H2ab |
| 55355 | HJURP | Holliday junction recognition protein |
| 3142 | HLX1 | H2.0-like homeobox |
| 3146 | HMGB1 | high-mobility group box 1; high-mobility group box 1-like 10 |
| 3148 | HMGB2 | high-mobility group box 2 |
| 3161 | HMMR | hyaluronan-mediated motility receptor (RHAMM) |
| 10238 | HNRPR | heterogeneous nuclear ribonucleoprotein R |
| 3198 | HOXA1 | homeobox A1 |
| 10247 | HRSP12 | heat-responsive protein 12 |
| 3297 | HSF1 | heat shock transcription factor 1 |
| 3313 | HSPA9 | heat shock 70 kDa protein 9 (mortalin) |
| 10808 | HSPH1 | heat shock 105 kDa/110 kDa protein 1 |
| 25998 | IBTK | inhibitor of Bruton agammaglobulinemia tyrosine kinase |
| 3384 | ICAM2 | intercellular adhesion molecule 2 |
| 80173 | IFT74 | intraflagellar transport 74 homolog |
| 150084 | IGSF5 | immunoglobulin superfamily, member 5 |
| 3570 | IL6R | interleukin 6 receptor |
| 3684 | ITGAM | integrin, alpha M |
| 23421 | ITGB3BP | integrin beta 3 binding protein |
| 6453 | ITSN1 | intersectin 1 (SH3 domain protein) |
| 10008 | KCNE3 | potassium voltage-gated channel, Isk-related family, member 3 |
| 3776 | KCNK2 | potassium channel, subfamily K, member 2 |
| 9768 | KIAA0101 | KIAA0101 |
| 56243 | KIAA1217 | KIAA1217 |
| 85014 | KIAA1984 | KIAA1984; transmembrane protein 141 |
| 3832 | KIF11 | kinesin family member 11 |
| 81930 | KIF18A | kinesin family member 18A |
| 10112 | KIF20A | kinesin family member 20A |
| 11004 | KIF2C | kinesin family member 2C |
| 3833 | KIFC1 | kinesin family member C1 |
| 55220 | KLHDC8A | kelch domain containing 8A |
| 3912 | LAMB1 | laminin, beta 1 |
| 3915 | LAMC1 | laminin, gamma 1 (formerly LAMB2) |
| 55915 | LANCL2 | LanC lantibiotic synthetase component C-like 2 |
| 11025 | LILRB3 | leukocyte immunoglobulin-like receptor, subfamily B |
| 4005 | LMO2 | LIM domain only 2 (rhombotin-like 1) |
| 345711 | LOC345711 | similar to ankyrin repeat domain 33 |
| 26018 | LRIG1 | leucine-rich repeats and immunoglobulin-like domains 1 |
| 10894 | LYVE1 | lymphatic vessel endothelial hyaluronan receptor 1 |
| 4085 | MAD2L1 | MAD2 mitotic arrest deficient-like 1 |
| 55110 | MAGOHB | mago-nashi homolog B |
| 8300 | MAPK12 | mitogen-activated protein kinase 12 |
| 4147 | MATN2 | matrilin 2 |
| 4172 | MCM3 | minichromosome maintenance complex component 3 |
| 4174 | MCM5 | minichromosome maintenance complex component 5 |
| 4175 | MCM6 | minichromosome maintenance complex component 6 |
| 4178 | MCM7 | minichromosome maintenance complex component 7 |
| 90390 | MED30 | mediator complex subunit 30 |
| 9833 | MELK | maternal embryonic leucine zipper kinase |
| 4232 | MEST | mesoderm specific transcript homolog (mouse) |
| 4233 | MET | met proto-oncogene (hepatocyte growth factor receptor) |
| 4288 | MKI67 | antigen identified by monoclonal antibody Ki-67 |
| 8028 | MLLT10 | myeloid/lymphoid or mixed-lineage leukemia; translocated to, 10 |
| 4317 | MMP8 | matrix metallopeptidase 8 (neutrophil collagenase) |
| 4318 | MMP9 | matrix metallopeptidase 9 |
| 4353 | MPO | myeloperoxidase |
| 51878 | MPP6 | membrane protein, palmitoylated 6 |
| 116535 | MRGPRF | MRGPRF MAS-related GPR, member F |
| 64968 | MRPS6 | mitochondrial ribosomal protein S6 |
| 10335 | MRVI1 | murine retrovirus integration site 1 homolog |
| 10232 | MSLN | mesothelin |
| 10797 | MTHFD2 | methylenetetrahydrofolate dehydrogenase 2 |
| 4600 | MX2 | myxovirus (influenza virus) resistance 2 (mouse) |
| 4678 | NASP | nuclear autoantigenic sperm protein (histone-binding) |
| 9918 | NCAPD2 | non-SMC condensin I complex, subunit D2 |
| 54892 | NCAPG2 | non-SMC condensin II complex, subunit G2 |
| 23397 | NCAPH | non-SMC condensin I complex, subunit H |
| 10403 | NDC80 | NDC80 homolog, kinetochore complex component |
| 4751 | NEK2 | NIMA (never in mitosis gene a)-related kinase 2 |
| 23530 | NNT | nicotinamide nucleotide transhydrogenase |
| 4848 | NOS3 | nitric oxide synthase 3 (endothelial cell) |
| 4855 | NOTCH4 | Notch homolog 4 (Drosophila) |
| 84955 | NUDCD1 | NudC domain containing 1 |
| 11163 | NUDT4 | nudix (nucleoside diphosphate linked moiety X)-type motif 4 |
| 83540 | NUF2 | NUF2, NDC80 kinetochore complex component, homolog |
| 53371 | NUP54 | nucleoporin 54 kDa |
| 4928 | NUP98 | nucleoporin 98 kDa |
| 51203 | NUSAP1 | nucleolar and spindle associated protein 1 |
| 4999 | ORC2L | origin recognition complex, subunit 2-like |
| 116039 | OSR2 | odd-skipped related 2 (Drosophila) |
| 5019 | OXCT1 | 3-oxoacid CoA transferase 1 |
| 56288 | PARD3 | par-3 partitioning defective 3 homolog |
| 55872 | PBK | PDZ binding kinase |
| 11333 | PDAP1 | PDGFA associated protein 1 |
| 5138 | PDE2A | phosphodiesterase 2A, cGMP-stimulated |
| 5158 | PDGFRA | platelet-derived growth factor receptor, alpha polypeptide |
| 5175 | PECAM1 | platelet/endothelial cell adhesion molecule |
| 26227 | PHGDH | phosphoglycerate dehydrogenase |
| 83483 | PLVAP | plasmalemma vesicle associated protein |
| 57125 | PLXDC1 | plexin domain containing 1 |
| 5425 | POLD2 | polymerase (DNA directed), delta 2, regulatory subunit 50 kDa |
| 5427 | POLE2 | polymerase (DNA directed), epsilon 2 (p59 subunit) |
| 5448 | PON3 | paraoxonase 3 |
| 5557 | PRIM1 | primase, DNA, polypeptide 1 (49 kDa) |
| 5558 | PRIM2A | primase, DNA, polypeptide 2 (58 kDa) |
| 5578 | PRKCA | protein kinase C, alpha |
| 23627 | PRND | prion protein 2 (dublet) |
| 5743 | PTGS2 | prostaglandin-endoperoxide synthase 2 |
| 11156 | PTP4A3 | protein tyrosine phosphatase type IVA, member 3 |
| 5885 | RAD21 | RAD21 homolog (S. pombe) |
| 5888 | RAD51 | RAD51 homolog (RecA homolog, E. coli) |
| 5889 | RAD51C | RAD51 homolog C (S. cerevisiae) |
| 9584 | RBM39 | similar to RNA binding motif protein 39 |
| 3516 | RBPJ | recombination signal binding protein for immunoglobulin kappa J region |
| 5965 | RECQL | RecQ protein-like (DNA helicase Q1-like) |
| 5984 | RFC4 | replication factor C (activator 1) 4, 37 kDa |
| 5985 | RFC5 | replication factor C (activator 1) 5, 36.5 kDa |
| 23179 | RGL1 | ral guanine nucleotide dissociation stimulator-like 1 |
| 64407 | RGS18 | regulator of G-protein signaling 18 |
| 5997 | RGS2 | regulator of G-protein signaling 2, 24 kDa |
| 8490 | RGS5 | regulator of G-protein signaling 5 |
| 6045 | RNF2 | ring finger protein 2 |
| 6091 | ROBO1 | roundabout, axon guidance receptor, homolog 1 |
| 6118 | RPA2 | replication protein A2, 32 kDa |
| 6119 | RPA3 | replication protein A3, 14 kDa |
| 80135 | RPF1 | brix domain containing 5 |
| 6222 | RPS18 | ribosomal protein S18 pseudogene 12 |
| 6236 | RRAD | Ras-related associated with diabetes |
| 22800 | RRAS2 | related RAS viral (r-ras) oncogene homolog 2 |
| 6240 | RRM1 | ribonucleotide reductase M1 |
| 6241 | RRM2 | ribonucleotide reductase M2 polypeptide |
| 340419 | RSPO2 | R-spondin 2 homolog |
| 10371 | SEMA3A | sema domain, (semaphorin) 3A |
| 143686 | SESN3 | sestrin 3 |
| 85358 | SHANK3 | SH3 and multiple ankyrin repeat domains 3 |
| 79801 | SHCBP1 | SHC SH2-domain binding protein 1 |
| 8036 | SHOC2 | soc-2 suppressor of clear homolog |
| 23517 | SKIV2L2 | superkiller viralicidic activity 2-like 2 |
| 7884 | SLBP | stem-loop binding protein |
| 6509 | SLC1A4 | solute carrier family 1, member 4 |
| 115286 | SLC25A26 | solute carrier family 25, member 26 |
| 8526 | SLC5A3 | solute carrier family 5, member 3 |
| 8467 | SMARCA5 | SWI/SNF related, matrix associated, |
| 8243 | SMC1L1 | structural maintenance of chromosomes 1A |
| 10592 | SMC2 | structural maintenance of chromosomes 2 |
| 10051 | SMC4 | structural maintenance of chromosomes 4 |
| 6629 | SNRPB2 | small nuclear ribonucleoprotein polypeptide B″ |
| 64321 | SOX17 | SRY (sex determining region Y)-box 17 |
| 6662 | SOX9 | SRY (sex determining region Y)-box 9 |
| 10615 | SPAG5 | sperm associated antigen 5 |
| 57405 | SPBC25 | SPC25, NDC80 kinetochore complex component, homolog |
| 60559 | SPCS3 | signal peptidase complex subunit 3 homolog |
| 6741 | SSB | Sjogren syndrome antigen B (autoantigen La) |
| 6742 | SSBP1 | single-stranded DNA binding protein 1 |
| 26872 | STEAP1 | six transmembrane epithelial antigen of the prostate 1 |
| 6788 | STK3 | serine/threonine kinase 3 (STE20 homolog, yeast) |
| 10460 | TACC3 | transforming, acidic coiled-coil containing protein 3 |
| 23435 | TARDBP | TAR DNA binding protein |
| 25771 | TBC1D22A | TBC1 domain family, member 22A |
| 6899 | TBX1 | T-box 1 |
| 7052 | TGM2 | transglutaminase 2 |
| 8914 | TIMELESS | timeless homolog (Drosophila) |
| 7077 | TIMP2 | TIMP metallopeptidase inhibitor 2 |
| 54962 | TIPIN | TIMELESS interacting protein |
| 7083 | TK1 | thymidine kinase 1, soluble |
| 55273 | TMEM100 | transmembrane protein 100 |
| 55161 | TMEM33 | transmembrane protein 33 |
| 55706 | TMEM48 | transmembrane protein 48 |
| 84629 | TNRC18 | trinucleotide repeat containing 18 |
| 54543 | TOMM7 | translocase of outer mitochondrial membrane 7 homolog |
| 7153 | TOP2A | topoisomerase (DNA) II alpha 170 kDa |
| 22974 | TPX2 | TPX2, microtubule-associated, homolog |
| 54209 | TREM2 | triggering receptor expressed on myeloid cells 2 |
| 4591 | TRIM37 | tripartite motif-containing 37 |
| 9319 | TRIP13 | thyroid hormone receptor interactor 13 |
| 95681 | TSGA14 | testis specific, 14 |
| 9694 | TTC35 | tetratricopeptide repeat domain 35 |
| 11065 | UBE2C | ubiquitin-conjugating enzyme E2C |
| 51377 | UCHL5 | ubiquitin carboxyl-terminal hydrolase L5 |
| 7371 | UCK2 | uridine-cytidine kinase 2 |
| 83878 | USHBP1 | Usher syndrome 1C binding protein 1 |
| 79805 | VASH2 | vasohibin 2 |
| 11326 | VSIG4 | V-set and immunoglobulin domain containing 4 |
| 79971 | WLS | G protein-coupled receptor 177 |
| 51776 | ZAK | sterile alpha motif and leucine zipper containing kinase AZK |
| 221527 | ZBTB12 | zinc finger and BTB domain containing 12 |
| 346171 | ZFP57 | zinc finger protein 57 homolog |
| 23414 | ZFPM2 | zinc finger protein, multitype 2 |
| 79830 | ZMYM1 | zinc finger, MYM-type 1 |
| 7705 | ZNF146 | zinc finger protein 146 |
| 84858 | ZNF503 | zinc finger protein 503 |

TABLE 5
Functional annotation for each of the 18 pro-invasion oncogenes informed by the
DAVID Bioinformatics Resource (NIH NIAID, http://david.abcc.ncifcrf.gov/).

| ASF1 | Gene ID: 55723 | ASF1 anti-silencing function 1 homolog B |
|---|---|---|
| | GOTERM_BP_FAT | DNA packaging, chromatin organization, chromatin assembly or |
| | | disassembly, nucleosome assembly, transcription, gamete |
| | | generation, spermatogenesis, chromatin modification, sexual reproduction, chromatin |
| | | assembly, multicellular organism reproduction, cellular macromolecular complex subunit |
| | | organization, cellular macromolecular complex assembly, nucleosome |
| | | organization, macromolecular complex subunit organization, regulation of |
| | | transcription, male gamete generation, reproductive process in a multicellular |
| | | organism, chromosome organization, macromolecular complex assembly, protein-DNA |
| | | complex assembly, |
| | GOTERM_CC_FAT | chromatin, chromosome, non-membrane-bounded organelle, intracellular non- |
| | | membrane-bounded organelle, chromosomal part, |
| | GOTERM_MF_FAT | histone binding, |
| | INTERPRO | Histone chaperone, ASF1-like, |
| | SP_PIR_KEYWORDS | Chaperone, chromatin regulator, complete proteome, developmental |
| | | protein, differentiation, nucleus, phosphoprotein, spermatogenesis, Transcription, |
| | | transcription regulation, |
| | UP_SEQ_FEATURE | chain: Histone chaperone ASF1B, modified residue, region of interest: Interaction with |
| | | CHAF1B, region of interest: interaction with histone H3, sequence conflict, |
| DEPDC1 | Gene ID: 55635 | DEP domain containing 1 |
| | GOTERM_BP_FAT | intracellular signaling cascade, |
| | GOTERM_MF_FAT | GTPase activator activity, enzyme activator activity, GTPase regulator |
| | | activity, nucleoside-triphosphatase regulator activity, |
| | INTERPRO | RhoGAP, Pleckstrin/G-protein, interacting region, Winged helix repressor DNA-binding, |
| | SMART | DEP, RhoGAP, |
| | SP_PIR_KEYWORDS | 3d-structure, alternative splicing, complete proteome, GTPase |
| | | activation, nucleus, phosphoprotein, polymorphism, |
| | UP_SEQ_FEATURE | chain: DEP domain-containing protein 1A, domain: DEP, domain: Rho-GAP, helix, |
| | | modified residue, sequence variant, splice variant, strand, |
| NDC80 | Gene ID: 10403 | NDC80 homolog, kinetochore complex component |
| | COG_ONTOLOGY | Cell division and chromosome partitioning, |
| | GOTERM_BP_FAT | mitotic sister chromatid segregation, M phase of mitotic cell cycle, establishment of |
| | | mitotic spindle orientation, microtubule cytoskeleton organization, mitotic cell cycle, M |
| | | phase, nuclear division, sister chromatid segregation, cell morphogenesis, cytoskeleton |
| | | organization, microtubule-based process, cell cycle, spindle organization, mitotic spindle |
| | | organization, chromosome segregation, mitosis, establishment or maintenance of cell |
| | | polarity, intracellular signaling cascade, protein localization, attachment of spindle |
| | | microtubules to kinetochore, second-messenger-mediated signaling, cell cycle |
| | | process, cell cycle phase, establishment of cell polarity, maintenance of protein location |
| | | in cell, cellular component morphogenesis, microtubule anchoring, establishment of |
| | | mitotic spindle localization, maintenance of protein location, phosphoinositide-mediated |
| | | signaling, organelle fission, maintenance of location, chromosome |
| | | organization, establishment of spindle localization, establishment of spindle |
| | | orientation, cell division, attachment of spindle microtubules to chromosome, organelle |
| | | localization, maintenance of location in cell, spindle localization, establishment of |
| | | organelle localization, |
| | GOTERM_CC_FAT | chromosome, centromeric region, kinetochore, condensed chromosome |
| | | kinetochore, condensed chromosome, centromeric region, condensed |
| | | chromosome, chromosome, Ndc80 complex, non-membrane-bounded |
| | | organelle, intracellular non-membrane-bounded organelle, chromosomal part, |
| | INTERPRO | Kinetochore protein Ndc80, |
| | SP_PIR_KEYWORDS | 3d-structure, cell cycle, cell division, coiled coil, complete |
| | | proteome, kinetochore, mitosis, nucleus, phosphoprotein, polymorphism, |
| | UP_SEQ_FEATURE | chain: Kinetochore protein NDC80 homolog, helix, modified residue, mutagenesis site, |
| | | region of interest: Interaction with NEK2 and ZWINT, region of interest: Interaction with |
| | | PSMC2 and SMC1A, region of interest: Interaction with RB1, region of |
| | | interest: Interaction with SMC1A, region of interest: Interaction with the C-terminus of |
| | | CDCA1 and the SPBC24-SPBC25 subcomplex, region of interest: Interaction with the N- |
| | | terminus of CDCA1, region of interest: Nuclear localization, sequence variant, turn, |
| VSIG4 | Gene ID: 11326 | V-set and immunoglobulin domain containing 4 |
| | GOTERM_BP_FAT | regulation of cytokine production, negative regulation of cytokine production, immune |
| | | effector process, activation of immune response, acute inflammatory |
| | | response, activation of plasma proteins involved in acute inflammatory |
| | | response, negative regulation of immune system process, positive regulation of immune |
| | | system process, regulation of leukocyte activation, negative regulation of leukocyte |
| | | activation, proteolysis, defense response, inflammatory response, immune |
| | | response, complement activation, complement activation, alternative pathway, humoral |
| | | immune response, negative regulation of cell proliferation, response to wounding, protein |
| | | processing, regulation of interleukin-2 production, negative regulation of interleukin-2 |
| | | production, regulation of mononuclear cell proliferation, negative regulation of |
| | | mononuclear cell proliferation, regulation of cell proliferation, regulation of T cell |
| | | proliferation, negative regulation of T cell proliferation, innate immune response, positive |
| | | regulation of response to stimulus, regulation of lymphocyte proliferation, negative |
| | | regulation of lymphocyte proliferation, positive regulation of immune response, regulation |
| | | of T cell activation, regulation of cell activation, negative regulation of cell |
| | | activation, negative regulation of T cell activation, negative regulation of multicellular |
| | | organismal process, regulation of lymphocyte activation, negative regulation of |
| | | lymphocyte activation, protein maturation, protein maturation by peptide bond |
| | | cleavage, regulation of leukocyte proliferation, negative regulation of leukocyte |
| | | proliferation, |
| | GOTERM_CC_FAT | plasma membrane, integral to membrane, intrinsic to membrane, |
| | INTERPRO | Immunoglobulin subtype 2, Immunoglobulin subtype, Immunoglobulin- |
| | | like, Immunoglobulin V-set, Immunoglobulin, Immunoglobulin-like fold, |
| | SMART | IGc2, IG, |
| | SP_PIR_KEYWORDS | 3d-structure, alternative splicing, complement alternate pathway, complete |
| | | proteome, direct protein sequencing, disulfide bond, immune response, immunoglobulin |
| | | domain, innate immunity, membrane, polymorphism, repeat, signal, transmembrane, |
| | UP_SEQ_FEATURE | chain: V-set and immunoglobulin domain-containing protein 4, disulfide bond, domain: Ig- |
| | | like 1, domain: Ig-like 2, helix, sequence variant, signal peptide, splice variant, strand, |
| | | topological domain: Cytoplasmic, topological domain: Extracellular, transmembrane |
| | | region, turn, |
| ACP5 | Gene ID: 54 | acid phosphatase 5, tartrate resistant |
| | GOTERM_BP_FAT | skeletal system development, tissue homeostasis, phosphorus metabolic |
| | | process, phosphate metabolic process, immune response, response to organic |
| | | substance, dephosphorylation, response to cytokine stimulus, homeostatic |
| | | process, bone resorption, bone remodeling, skeletal system morphogenesis, tissue |
| | | remodeling, multicellular organismal homeostasis, anatomical structure |
| | | homeostasis, bone development, bone morphogenesis, |
| | GOTERM_CC_FAT | lytic vacuole, lysosome, vacuole, cytosol, integral to membrane, intrinsic to membrane, |
| | GOTERM_MF_FAT | acid phosphatase activity, iron ion binding, phosphatase activity, ion binding, cation |
| | | binding, metal ion binding, transition metal ion binding, |
| | INTERPRO | Metallophosphoesterase, |
| | KEGG_PATHWAY | Riboflavin metabolism, Lysosome, |
| | OMIM_DISEASE | Genome-wide association analysis of susceptibility and clinical phenotype in multiple |
| | | sclerosis, |
| | PIR_SUPERFAMILY | PIRSF000898: acid phosphatase, type 5, |
| | SP_PIR_KEYWORDS | 3d-structure, blocked amino end, complete proteome, direct protein |
| | | sequencing, disulfide bond, glycoprotein, hydrolase, iron, lysosome, metal- |
| | | binding, metalloprotein, phosphoric monoester hydrolase, polymorphism, signal, |
| | UP_SEQ_FEATURE | chain: Tartrate-resistant acid phosphatase type 5, disulfide bond, glycosylation site: N- |
| | | linked (GlcNAc . . .), helix, metal ion-binding site: Iron 1, metal ion-binding site: Iron 2, |
| | | sequence conflict, sequence variant, signal peptide, strand, turn, |
| ANLN | Gene ID: 54443 | anillin, actin binding protein |
| | GOTERM_BP_FAT | M phase of mitotic cell cycle, mitotic cell cycle, M phase, nuclear |
| | | division, cytokinesis, septin ring assembly, protein complex assembly, cytoskeleton |
| | | organization, cell cycle, mitosis, regulation of exit from mitosis, regulation of mitotic cell |
| | | cycle, regulation of cell cycle process, cell cycle process, cell cycle phase, septin ring |
| | | organization, septin cytoskeleton organization, cellular macromolecular complex subunit |
| | | organization, cellular macromolecular complex assembly, cellular protein complex |
| | | assembly, macromolecular complex subunit organization, organelle fission, cell |
| | | division, regulation of cell cycle, macromolecular complex assembly, protein complex |
| | | biogenesis, |
| | GOTERM_CC_FAT | contractile ring, cytoskeleton, cell cortex, actin cytoskeleton, cell division site, cell |
| | | division site part, non-membrane-bounded organelle, intracellular non-membrane- |
| | | bounded organelle, cytoskeletal part, cell cortex part, |
| | GOTERM_MF_FAT | actin binding, cytoskeletal protein binding, |
| | INTERPRO | Pleckstrin homology, Pleckstrin homology-type, |
| | SMART | PH, |
| | SP_PIR_KEYWORDS | acetylation, actin-binding, alternative splicing, cell cycle, cell division, coiled |
| | | coil, complete |
| | | proteome, cytoplasm, cytoskeleton, mitosis, nucleus, phosphoprotein, polymorphism, ubl |
| | | conjugation, |
| | UP_SEQ_FEATURE | chain: Actin-binding protein anillin, domain: PH, modified residue, mutagenesis site, |
| | | region of interest: Interaction with CD2AP, region of interest: Interaction with F-actin, |
| | | region of interest: Localization to the cleavage furrow, region of interest: Nuclear |
| | | localization, region of interest: Required for ubiquitination, sequence conflict, sequence |
| | | variant, splice variant, |
| FSCN1 | Gene ID: 6624 | fascin homolog 1, actin-bundling protein |
| | GOTERM_BP_FAT | cytoskeleton organization, actin filament organization, cell proliferation, actin filament- |
| | | based process, actin cytoskeleton organization, actin filament bundle formation, |
| | GOTERM_CC_FAT | cytoskeleton, plasma membrane, actin cytoskeleton, filopodium, cell projection, non- |
| | | membrane-bounded organelle, intracellular non-membrane-bounded organelle, |
| | GOTERM_MF_FAT | actin binding, cytoskeletal protein binding, protein binding, bridging, actin filament |
| | | binding, |
| | INTERPRO | Fascin, |
| | SP_PIR_KEYWORDS | 3d-structure, acetylation, actin-binding, complete proteome, cytoplasm, direct protein |
| | | sequencing, phosphoprotein, |
| | UP_SEQ_FEATURE | chain: Fascin, helix, modified residue, mutagenesis site, sequence conflict, strand, turn, |
| HSF1 | Gene ID: 3297 | heat shock transcription factor 1 |
| | GOTERM_BP_FAT | in utero embryonic development, regulation of cytokine production, negative regulation |
| | | of cytokine production, placenta development, embryonic placenta |
| | | development, response to molecule of bacterial origin, transcription, regulation of |
| | | transcription, DNA-dependent, protein amino acid phosphorylation, phosphorus |
| | | metabolic process, phosphate metabolic process, defense response, gamete |
| | | generation, spermatogenesis, female pregnancy, negative regulation of cell |
| | | proliferation, response to temperature stimulus, response to heat, response to |
| | | bacterium, response to abiotic stimulus, embryonic development ending in birth or egg |
| | | hatching, response to organic substance, phosphorylation, sexual |
| | | reproduction, response to lipopolysaccharide, multicellular organism |
| | | reproduction, regulation of tumor necrosis factor production, negative regulation of tumor |
| | | necrosis factor production, regulation of growth, regulation of multicellular organism |
| | | growth, positive regulation of multicellular organism growth, regulation of cell |
| | | proliferation, chordate embryonic development, regulation of transcription, positive |
| | | regulation of growth, male gamete generation, embryonic organ |
| | | development, reproductive process in a multicellular organism, positive regulation of |
| | | multicellular organismal process, negative regulation of multicellular organismal |
| | | process, regulation of RNA metabolic process, embryonic process involved in female |
| | | pregnancy, |
| | GOTERM_CC_FAT | nucleolus, membrane-enclosed lumen, nuclear lumen, non-membrane-bounded |
| | | organelle, intracellular non-membrane-bounded organelle, organelle |
| | | lumen, pronucleus, intracellular organelle lumen, |
| | GOTERM_MF_FAT | DNA binding, transcription factor activity, transcription regulator activity, sequence- |
| | | specific DNA binding, |
| | INTERPRO | Heat shock factor (HSF)-iype, DNA-binding, Vertebrate heat shock transcription |
| | | factor, Winged helix repressor DNA-binding, |
| | SMART | HSF, |
| | SP_PIR_KEYWORDS | acetylation, activator, alternative splicing, complete proteome, cytoplasm, direct protein |
| | | sequencing, dna-binding, isopeptide bond, nucleus, phosphoprotein, stress |
| | | response, Transcription, transcription regulation, ubl conjugation, |
| | UP_SEQ_FEATURE | chain: Heat shock factor protein 1, cross-link: Glycyl lysine isopeptide (Lys-Gly) |
| | | (interchain with G-Cter in SUMO), modified residue, mutagenesis site, region of |
| | | interest: Hydrophobic repeat HR-A/B, region of interest: Hydrophobic repeat HR-C, region |
| | | of interest: Regulatory domain, region of interest: Transactivation domain, splice variant, |
| HMGB1 | Gene ID: 3146 | high-mobility group box 1 |
| | BIOCARTA | Apoptotic DNA fragmentation and tissue homeostasis, The information-processing |
| | | pathway at the IFN-beta enhancer, |
| | GOTERM_BP_FAT | negative regulation of transcription from RNA polymerase II promoter, DNA metabolic |
| | | process, DNA replication, DNA-dependent DNA replication, DNA ligation, DNA |
| | | unwinding during replication, DNA repair, base-excision repair, base-excision repair, |
| | | DNA ligation, DNA recombination, chromatin organization, regulation of transcription, |
| | | DNA-dependent, regulation of transcription from RNA polymerase II promoter, anti- |
| | | apoptosis, response to DNA damage stimulus, negative regulation of biosyntheiic |
| | | process, negative regulation of macromolecule biosynthetic process, negative regulation |
| | | of macromolecule metabolic process, negative regulation of gene expression, regulation |
| | | of cell death, negative regulation of transcription, negative regulation of transcriptional |
| | | preinitiation complex assembly, negative regulation of cellular biosynthetic process, DNA |
| | | geometric change, DNA duplex unwinding, cellular response to stress, regulation of |
| | | apoptosis, negative regulation of apoptosis, regulation of programmed cell |
| | | death, negative regulation of programmed cell death, regulation of protein complex |
| | | assembly, regulation of cellular component biogenesis, regulation of |
| | | transcription, negative regulation of transcription, DNA-dependent, regulation of |
| | | transcriptional preinitiation complex assembly, negative regulation of nucleobase, |
| | | nucleoside, nucleotide and nucleic acid metabolic process, DNA ligation during DNA |
| | | repair, negative regulation of cellular component organization, negative regulation of |
| | | nitrogen compound metabolic process, regulation of RNA metabolic process, negative |
| | | regulation of RNA metabolic process, chromosome organization, regulation of |
| | | transcription initiation from RNA polymerase II promoter, negative regulation of cell |
| | | death, |
| | GOTERM_CC_FAT | condensed chromosome, nucleoplasm, chromosome, nucleolus, membrane-enclosed |
| | | lumen, nuclear lumen, non-membrane-bounded organelle, intracellular non-membrane- |
| | | bounded organelle, organelle lumen, intracellular organelle lumen, |
| | GOTERM_MF_FAT | DNA binding, transcription factor binding, DNA bending activity, |
| | INTERPRO | High mobility group, HMG1/HMG2, subgroup, High mobility group, HMG1/HMG2, HMG |
| | | box A DNA-binding domain, conserved site, |
| | KEGG_PATHWAY | Base excision repair, |
| | PIR_SUPERFAMILY | PIRSF002054: nonhistone chromosomal protein HMG-2, |
| | SMART | HMG, |
| | SP_PIR_KEYWORDS | 3d-structure, acetylation, chromosomal protein, complete proteome, direct protein |
| | | sequencing, DNA binding, dna-binding, isopeptide |
| | | bond, nucleus, phosphoprotein, polymorphism, repeat, ubl conjugation, |
| | UP_SEQ_FEATURE | chain: High mobility group protein 1-like 10, chain: High mobility group protein B1, |
| | | compositionally biased region: Asp/Glu-rich (acidic), cross-link: Glycyl, lysine isopeptide |
| | | (Lys-Gly) (interchain with G-Cter in ubiquitin), DNA-binding region: HMG box 1, DNA- |
| | | binding region: HMG box 2, helix, modified residue, sequence conflict, sequence variant, |
| | | strand, turn, |
| HOXA1 | Gene ID: 3198 | homeobox A1 |
| | COG ONTOLOGY | Transcription, |
| | GOTERM_BP_FAT | cell morphogenesis, cell morphogenesis involved in |
| | | differentiation, regionalization, transcription, regulation of transcription, DNA- |
| | | dependent, regulation of transcription from RNA polymerase II promoter, cell |
| | | motion, pattern specification process, axonogenesis, axon guidance, sensory organ |
| | | development, motor axon guidance, positive regulation of biosynthetic |
| | | process, anterior/posterior pattern formation, positive regulation of macromolecule |
| | | biosynthetic process, positive regulation of macromolecule metabolic process, positive |
| | | regulation of gene expression, cranial nerve development, rhombomere |
| | | development, pons development, facial nerve development, rhombomere 3 |
| | | development, rhombomere 4 development, rhombomere 5 development, cranial nerve |
| | | morphogenesis, cranial nerve structural organization, facial nerve morphogenesis, facial |
| | | nerve structural organization, nerve development, facial nucleus |
| | | development, preganglionic parasympathetic nervous system development, central |
| | | nervous system neuron differentiation, metencephalon development, cell projection |
| | | organization, neuron differentiation, hindbrain development, neuron projection |
| | | development, positive regulation of cellular biosynthetic process, cellular component |
| | | morphogenesis, cell part morphogenesis, ear morphogenesis, inner ear |
| | | morphogenesis, ear development, regulation of transcription, positive regulation of |
| | | transcription, DNA-dependent, positive regulation of nucleobase, nucleoside, nucleotide |
| | | and nucleic acid metabolic process, positive regulation of transcription, positive |
| | | regulation of transcription from RNA polymerase II promoter, autonomic nervous system |
| | | development, parasympathetic nervous system development, anatomical structure |
| | | arrangement, embryonic organ morphogenesis, embryonic organ |
| | | development, embryonic morphogenesis, neuron development, cell morphogenesis |
| | | involved in neuron differentiation, neuron projection morphogenesis, inner ear |
| | | development, neural nucleus development, cell projection morphogenesis, positive |
| | | regulation of nitrogen compound metabolic process, regulation of RNA metabolic |
| | | process, positive regulation of RNA metabolic process, |
| | GOTERM_MF_FAT | DNA binding, transcription factor activity, RNA polymerase II transcription factor |
| | | activity, transcription regulator activity, sequence-specific DNA binding, |
| | INTERPRO | Homeobox, Homeobox protein, antennapedia type, conserved site, Homeodomain- |
| | | related, Homeobox, conserved site, |
| | OMIM_DISEASE | Athabaskan brainstem dysgenesis syndrome, Bosley-Salih-Alorainy syndrome, |
| | PIR_SUPERFAMILY | PIRSF002608: homeotic protein Hox B1, |
| | SMART | HOX, |
| | SP_PIR_KEYWORDS | alternative splicing, complete proteome, developmental protein, DNA binding, dna - |
| | | binding, Homeobox, nucleus, polymorphism, Transcription, transcription regulation, |
| | UP_SEQ_FEATURE | chain: Homeobox protein Hox-A1, compositionally biased region: Poly-His, |
| | | compositionally biased region: Poly-Ser, DNA-binding region: Homeobox, sequence |
| | | variant, short sequence motif: Antp-type hexapeptide, splice variant, |
| ITGB3BP | Gene ID: 23421 | integrin beta 3 binding protein |
| | GOTERM_BP_FAT | transcription, apoptosis, induction of apoptosis, cell adhesion, cell death, induction of |
| | | apoptosis by extracellular signals, regulation of cell death, positive regulation of cell |
| | | death, programmed cell death, induction of programmed cell death, death, biological |
| | | adhesion, regulation of apoptosis, positive regulation of apoptosis, regulation of |
| | | programmed cell death, positive regulation of programmed cell death, regulation of |
| | | transcription, |
| | GOTERM_CC_FAT | cell fraction, chromosome, centromeric region, membrane fraction, insoluble |
| | | fraction, nucleoplasm, chromosome, cytosol, membrane-enclosed lumen, nuclear |
| | | lumen, non-membrane-bounded organelle, intracellular non-membrane-bounded |
| | | organelle, organelle lumen, chromosomal part, intracellular organelle lumen, |
| | GOTERM_MF_FAT | protein C-terminus binding, |
| | INTERPRO | Nuclear receptor co-activator NRIF3, |
| | PIR_SUPERFAMILY | PIRSF011860: NRIF3_coact_rcpt, PIRSF011860: nuclear receptor-interacting factor 3, |
| | SP_PIR_KEYWORDS | activator, alternative splicing, Apoptosis, centromere, chromosomal protein, coiled |
| | | coil, complete |
| | | proteome, cytoplasm, nucleus, phosphoprotein, polymorphism, repressor, Transcription, |
| | | transcription regulation, |
| | UP_SEQ_FEATURE | chain: Centromere protein R, modified residue, mutagenesis site, region of interest: DD1, |
| | | sequence variant, short sequence motif: LXXIL motif, short sequence motif: LXXLL motif, |
| | | short sequence motif: Nuclear localization signal, splice variant, |
| MTHFD2 | Gene ID: 10797 | methylenetetrahydrofolate dehydrogenase |
| | COG_ONTOLOGY | Coenzyme metabolism, |
| | GOTERM_BP_FAT | one-carbon metabolic process, coenzyme metabolic process, folic acid and derivative |
| | | metabolic process, coenzyme biosynthetic orocess, folic acid and derivative biosynthetic |
| | | process, pteridine and derivative metabolic process, tetrahydrofolate metabolic |
| | | process, cofactor metabolic process, cofactor biosynthetic process, oxidation reduction, |
| | GOTERM_CC_FAT | mitochondrion, |
| | GOTERM_MF_FAT | magnesium ion binding, methenyltetrahydrofolate cyclohydrolase |
| | | activity, methylenetetrahydrofolate dehydrogenase activity, methylenetetrahydrofolate |
| | | dehydrogenase (NAD+) activity, methylenetetrahydrofolate dehydrogenase (NADP+) |
| | | activity, oxidoreductase activity, acting on the CH—NH group of donors, oxidoreductase |
| | | activity, acting on the CH—NH group of donors, NAD or NADP as acceptor, hydrolase |
| | | activity, acting on carbon-nitrogen (but not peptide) bonds, in cyclic amidines, phosphate |
| | | binding, ion binding, anion binding, cation binding, metal ion binding, |
| | INTERPRO | Tetrahydrofolate dehydrogenase/cyclohydrolase, NAD(P)-binding domain, |
| | KEGG_PATHWAY | Glyoxylate and dicarboxylate metabolism, One carbon pool by folate, |
| | SP_PIR_KEYWORDS | 3d-structure, acetylation, complete |
| | | proteome, hydrolase, magnesium, mitochondrion, multifunctional enzyme, nad, one- |
| | | carbon metabolism, oxidoreductase, transit peptide, |
| | UP_SEQ_FEATURE | chain: Bifunctional methylenetetrahydrofolate dehydrogenase/cyclohydrolase, |
| | | mitochondrial, modified residue, sequence conflict, transit peptide: Mitochondrion, |
| MCM7 | Gene ID: 4176 | minichromosome maintenance complex component 7 |
| | GOTERM_BP_FAT | DNA metabolic process, DNA replication, DNA-dependent DNA replication, DNA |
| | | unwinding during replication, DNA replication initiation, transcription, response to DNA |
| | | damage stimulus, cell cycle, cell proliferation, regulation of phosphate metabolic |
| | | process, DNA geometric change, DNA duplex unwinding, cellular response to |
| | | stress, regulation of phosphorylation, regulation of transcription, regulation of |
| | | phosphorus metabolic process, |
| | GOTERM_CC_FAT | nuclear chromosome, chromatin, nucleoplasm, chromosome, membrane-enclosed |
| | | lumen, nuclear lumen, MCM complex, non-membrane-bounded organelle, intracellular |
| | | non-membrane-bounded organelle, organelle lumen, chromosomal part, nucleoplasm |
| | | part, nuclear chromosome part, intracellular organelle lumen, |
| | GOTERM_MF_FAT | nucleotide binding, nucleoside binding, purine nucleoside binding, DNA binding, DNA |
| | | helicase activity, single-stranded DNA binding, helicase activity, ATP binding, purine |
| | | nucleotide binding, adenyl nucleotide binding, ribonucleotide binding, purine |
| | | ribonucleotide binding, adenyl ribonucleotide binding, structure-specific DNA binding, |
| | INTERPRO | DNA-dependent ATPase MCM, ATPase, AAA+ type, core, MCM protein 7, Nucleic acid- |
| | | binding, OB-foid, DNA-dependent ATPase MCM, conserved site, |
| | KEGG_PATHWAY | DNA replication, Cell cycle, |
| | SMART | MCM, AAA, |
| | SP_PIR_KEYWORDS | acetylation, alternative splicing, alt-binding, cell cycle, complete proteome, direct protein |
| | | sequencing, dna replication, dna-binding, nucleotide- |
| | | binding, nucleus, phosphoprotein, polymorphism, Transcription, transcription regulation, |
| | UP_SEQ_FEATURE | chain: DNA replication licensing factor mcm7, domain: MCM, modified residue, nucleotide |
| | | phosphate-binding region: ATP, region of interest: Interaction with ATRIP, region of |
| | | interest: interaction with RAD17, sequence conflict, sequence variant, splice variant, |
| NCAPH | Gene ID: 23397 | non-SMC condensin I complex, subunit H |
| | GOTERM_BP_FAT | mitotic sister chromatid segregation, M phase of mitotic cell cycle, mitotic cell cycle, M |
| | | phase, nuclear division, sister chromatid segregation, DNA packaging, cell |
| | | cycle, chromosome segregation, mitosis, mitotic chromosome condensation, cell cycle |
| | | process, cell cycle phase, chromosome condensation, organelle fission, chromosome |
| | | organization, cell division, |
| | GOTERM_CC_FAT | condensed chromosome, condensin complex, chromosome, non-membrane-bounded |
| | | organelle, intracellular non-membrane-bounded organelle, chromosomal part, |
| | INTERPRO | Barren, |
| | PIR_SUPERFAMILY | PIRSF017126: chromosome condensation complex condensin, subunit H, |
| | | PIRSF017126: Condensin_H, |
| | SP_PIR_KEYWORDS | acelylation, cell cycle, cell division, complete proteome, cytoplasm, dna |
| | | condensation, mitosis, nucleus, phosphoprotein, polymorphism, |
| | UP_SEQ_FEATURE | chain: Condensin complex subunit 2, modified residue, sequence variant, |
| RNF2 | Gene ID: 6045 | ring finger protein 2 |
| | COG_ONTOLOGY | General function prediction only, |
| | GOTERM_BP_FAT | negative regulation of transcription from RNA polymerase II promoter, mitotic cell |
| | | cycle, gastrulation with mouth forming second, regionalization, chromatin |
| | | organization, transcription, regulation of transcription, DNA-dependent, regulation of |
| | | transcription from RNA polymerase II promoter, proteolysis, cell |
| | | cycle gastrulation, pattern specification process macromolecule catabolic process, axis |
| | | specification, negative regulation of biosynthetic process, anterior/posterior axis |
| | | specification, anterior/posterior pattern formation, negative regulation of macromolecule |
| | | biosynthetic process, negative regulation of macromolecule metabolic process, negative |
| | | regulation of gene expression, negative regulation of transcription, protein |
| | | ubiquitination, chromatin modification, covalent chromatin modification, histone |
| | | modification, histone ubiquitination, modification-dependent protein catabolic |
| | | process, protein catabolic process, negative regulation of cellular biosynthetic |
| | | process, protein modification by small protein conjugation, modification-dependent |
| | | macromolecule catabolic process, cellular protein catabolic process, cellular |
| | | macromolecule catabolic process, regulation of transcription, negative regulation of |
| | | transcription, DNA-dependent, negative regulation of nucleobase, nucleoside, nucleotide |
| | | and nucleic acid metabolic process, embryonic morphogenesis, negative regulation of |
| | | nitrogen compound metabolic process, regulation of RNA metabolic process, negative |
| | | regulation of RNA metabolic process, chromosome organization, proteolysis involved in |
| | | cellular protein catabolic process, protein modification by small protein conjugation or |
| | | removal, |
| | GOTERM_CC_FAT | ubiquitin ligase complex, nuclear chromosome, chromatin, nuclear |
| | | chromatin, heterochromatin, sex chromosome, sex |
| | | chromatin, nucleoplasm, chromosome, nuclear heterochromatin, nuclear body, PcG |
| | | protein complex, membrane-enclosed lumen, nuclear lumen, non-membrane-bounded |
| | | organelle, intracellular non-membrane-bounded organelle, organelle |
| | | lumen, chromosomal part, nucleoplasm part, nuclear chromosome part, intracellular |
| | | organelle lumen, |
| | GOTERM_MF_FAT | chromatin binding, ubiquitin-protein ligase activity, zinc ion binding, transcription |
| | | repressor activity, ligase activity, forming carbon-nitrogen bonds, acid-amino acid ligase |
| | | activity, small conjugating protein ligase activity, transcription regulator activity, ion |
| | | binding, cation binding, metal ion binding, transition metal ion binding, |
| | INTERPRO | Zinc finger, RING-type, Zinc finger, RING-type, conserved site, Zinc finger, C3HC4 |
| | | RING-type, |
| | SMART | RING, |
| | SP_PIR_KEYWORDS | 3d-structure, chromosomal protein, complete proteome, ligase, metal- |
| | | binding, nucleus, phosphoprotein, repressor, Transcription, transcription regulation, ubl |
| | | conjugation pathway, zinc, zinc-finger, |
| | UP_SEQ_FEATURE | chain: E3 ubiquitin-protein ligase RING2, helix, modified residue, mutagenesis site, |
| | | region of interest: Interaction with HIP2, strand, turn, zinc finger region: RING-type, |
| SPAG5 | Gene ID: 10615 | sperm associated antigen 5 |
| | GOTERM_BP_FAT | M phase of mitotic cell cycle, microtubule cytoskeleton organization, mitotic cell cycle, M |
| | | phase, nuclear division, cytoskeleton organization, microtubule-based process, cell |
| | | cycle, spindle organization, mitosis, intracellular signaling cascade, second-messenger- |
| | | mediated signaling, cell cycle process, cell cycle phase, phosphoinositide-mediated |
| | | signaling, organelle fission, cell division, |
| | GOTERM_CC_FAT | chromosome, centromeric region, kinetochore, condensed chromosome |
| | | kinetochore, condensed chromosome, centromeric region, condensed |
| | | chromosome, chromosome spindle, cytoskeleton microtubule, spindle |
| | | microtubule, microtubule cytoskeleton, non-membrane-bounded organelle, intracellular |
| | | non-membrane-bounded organelle, chromosomal part, cytoskeletal part, |
| | SP_PIR_KEYWORDS | cell cycle, cell division, coiled coil, complete |
| | | proteome, cytoplasm, cytoskeleton, kinetochore, microtubule, mitosis, phosphoprotein, |
| | UP_SEQ_FEATURE | chain: Sperm-associated antigen 5, compositionally biased region: Gln-rich, modified |
| | | residue, sequence conflict, |
| UCHL5 | Gene ID: 51377 | ubiquitin carboxyl-terminal hydrolase L5 |
| | GOTERM_BP_FAT | proteolysis, ubiquitin-dependent protein catabolic process, macromolecule catabolic |
| | | process, modification-dependent protein catabolic process, protein catabolic |
| | | process, modification-dependent macromolecule catabolic process, cellular protein |
| | | catabolic process, cellular macromolecule catabolic process, proteolysis involved in |
| | | cellular protein catabolic process, |
| | GOTERM_CC_FAT | proteasome complex, |
| | GOTERM_MF_FAT | ubiquitin thiolesterase activity, peptidase activity, cysteine-type peptidase |
| | | activity, thiolester hydrolase activity, peptidase activity, acting on L-amino acid peptides, |
| | INTERPRO | Peptidase C12, ubiquitin carboxyl-terminal hydrolase 1, Ubiquitinyl hydrolase, UCH37 |
| | | type, |
| | PIR_SUPERFAMILY | PIRSF038120: ubiquitin carboxyl-terminal hydrolase, UCH37type, |
| | | PIRSF038120: Ubiquitinyl_hydrolase_UCH37, |
| | SP_PIR_KEYWORDS | 3d-structure, acetylation, alternative splicing, complete |
| | | proteome, hydrolase, polymorphism, Protease, proteasome, thiol protease, ubl |
| | | conjugation pathway, |
| | UP_SEQ_FEATURE | chain: Ubiquitin carboxyl-terminal hydrolase isozyme L5, helix, modified residue, |
| | | mutagenesis site, region of interest: Interaction with ADRM1, sequence conflict, |
| | | sequence variant, splice variant, strand, turn, |
| UBE2C | Gene ID: 11065 | ubiquitin-conjugating enzyme E2C |
| | GOTERM_BP_FAT | M phase of mitotic cell cycle, microtubule cytoskeleton organization, mitotic cell cycle, M |
| | | phase, nuclear division, proteolysis, ubiquitin-dependent protein catabolic |
| | | process, ubiquitin cycle, cytoskeleton organization, microtubule-based process, cell |
| | | cycle, spindle organization, mitosis, regulation of exit from mitosis, intracellular signaling |
| | | cascade, regulation of mitotic cell cycle, cyclin catabolic process, macromolecule |
| | | catabolic process, proteasomal protein catabolic process, regulation of cell cycle |
| | | process, positive regulation of macromolecule metabolic process, negative regulation of |
| | | macromolecule metabolic process, protein ubiquitination, second-messenger-mediated |
| | | signaling, modification-dependent protein catabolic process, cell cycle process, cell cycle |
| | | phase, protein catabolic process, anaphase-promoting complex-dependent proteasomal |
| | | ubiquitin-dependent protein catabolic process, regulation of protein |
| | | ubiquitination, negative regulation of protein ubiquitination, positive regulation of protein |
| | | ubiquitination, regulation of protein modification process, negative regulation of protein |
| | | modification process, positive regulation of protein modification process, positive |
| | | regulation of exit from mitosis, regulation of cellular protein metabolic process, negative |
| | | regulation of cellular protein metabolic process, positive regulation of cellular protein |
| | | metabolic process, protein modification by small protein conjugation, positive regulation |
| | | of catalytic activity, negative regulation of catalytic activity, proteasomal ubiquitin- |
| | | dependent protein catabolic process, modification-dependent macromolecule catabolic |
| | | process, negative regulation of molecular function, positive regulation of molecular |
| | | function, cellular protein catabolic process, cellular macromolecule catabolic |
| | | process, phosphoinositide-mediated signaling, organelle fission, positive regulation of |
| | | protein metabolic process, negative regulation of protein metabolic process, cell |
| | | division, regulation of ligase activity, positive regulation of ligase activity, negative |
| | | regulation of ligase activity, negative regulation of ubiquitin-protein ligase activity during |
| | | mitotic cell cycle, positive regulation of ubiquitin-protein ligase activity during mitotic cell |
| | | cycle, regulation of ubiquitin-protein ligase activity, regulation of ubiquitin-protein ligase |
| | | activity during mitotic cell cycle, positive regulation of ubiquitin-protein ligase |
| | | activity, negative regulation of ubiquitin-protein ligase activity, proteolysis involved in |
| | | cellular protein catabolic process, regulation of cell cycle, protein modification by small |
| | | protein conjugation or removal, |
| | GOTERM_CC_FAT | nucleoplasm, cytosol, membrane-enclosed lumen, nuclear lumen, organelle |
| | | lumen, intracellular organelle lumen, |
| | GOTERM_MF_FAT | nucleotide binding, nucleoside binding, purine nucleoside binding, ubiquitin-protein |
| | | ligase activity, ATP binding, ligase activity, forming carbon-nitrogen bonds, acid-amino |
| | | acid ligase activity, purine nucleotide binding, small conjugating protein ligase |
| | | activity, adenyl nucleotide binding, ribonucleotide binding, purine ribonucleotide |
| | | binding, adenyl ribonucleotide binding, |
| | INTERPRO | Ubiquitin-conjugating enzyme, E2, Ubiquitin-conjugating enzyme E2 H10, Ubiquitin- |
| | | conjugating enzyme/RWD-like, |
| | KEGG_PATHWAY | Ubiquitin mediated proteolysis, |
| | PIR_SUPERFAMILY | PIRSF001567: ubiquitin-protein ligase E2, |
| | SMART | UBCc, |
| | SP_PIR_KEYWORDS | 3d-structure, acetylation, atp-binding, cell cycle, cell division, complete |
| | | proteome, ligase, mitosis, nucleotide-binding, polymorphism, ubl conjugation, ubl |
| | | conjugation pathway, |
| | UP_SEQ_FEATURE | active site: Glycyl thioester intermediate, chain: Ubiquitin-conjugating enzyme E2 C, helix, |
| | | modified residue, mutagenesis site, sequence variant, strand, turn, |

TABLE 6

| | | | | | | GOF soft agar | LOF soft agar |
|---|---|---|---|---|---|---|---|
| | | | | | Anoth is | (WM115 or | (M619, C918, |
| | Invasion hit | | | | hit | WM239A or | UACC257, |
| Gene name | HMEL | WM115 | WM3211 | 1205LU | RIE | HMEL) | 501Met) |
| ACP5 | + | + | | NT | − | − | + |
| ANLN | + | | + | NT | + | − | NT |
| ASF1B | + | + | + | NT | − | − | NT |
| BRRN1 | + | + | + | NT | − | − | NT |
| BUB1 | + | | | NT | − | NT | NT |
| CDC2 | + | | | NT | − | NT | NT |
| CENPM | + | | | NT | − | − | NT |
| DEPDC1 | + | | | NT | − | − | NT |
| ELTD1 | + | + | | NT | − | − | NT |
| EXT1 | + | | | NT | − | NT | NT |
| FSCN1 | + | + | + | NT | − | − | + |
| HCAP-G | + | | | NT | + | NT | NT |
| HMGB1 | + | | | NT | − | NT | NT |
| HMGB2 | + | | | NT | − | NT | NT |
| HOXA1 | + | + | + | NT | − | + | + |
| HSF1 | + | + | | NT | − | − | + |
| ITGB3BP | + | | | NT | − | − | NT |
| KIF20A | + | | | NT | − | NT | NT |
| KIF2C | + | | | NT | − | NT | NT |
| KNTC2/NDC80 | + | + | + | NT | + | − | + |
| MCM7 | + | | | NT | − | − | NT |
| MTHFD2 | + | + | | NT | − | − | NT |
| NASP | + | | | NT | − | NT | NT |
| PLVAP | + | | | NT | − | NT | NT |
| PTP4A3 | + | + | | NT | − | NT | NT |
| RNF2 | + | + | + | NT | − | + | NT |
| SPAG5 | + | + | + | NT | − | − | NT |
| TGM2 | + | | | NT | − | NT | NT |
| UBE2C | + | | | NT | − | − | NT |
| UCHL5 | + | + | + | NT | − | − | NT |
| VSIG4 | + | + | | NT | − | − | + |
| HNRPR | − | NT | NT | + | + | + | + |
| CDC20 | − | NT | NT | NT | + | + | NT |
| PRIM2A | − | NT | NT | NT | + | + | NT |
| HRSP12 | − | NT | NT | − | + | NT | NT |
| ENY2/SUS1 | − | NT | NT | + | + | NT | + |
| TMEM141 | − | NT | NT | NT | + | NT | NT |
| RECQL | − | NT | NT | NT | + | + | NT |
| STK3 | − | NT | NT | + | + | + | NT |
| MX2 | − | NT | NT | − | + | NT | NT |
| CDCA1/NUF2 | − | NT | NT | NT | + | NT | NT |
| CEPS8/ | − | NT | NT | NT | + | NT | NT |
| KIAA0582 | | | | | | | |
| SPBC25 | − | NT | NT | NT | + | NT | NT |
| CDC25C | − | NT | NT | NT | + | NT | NT |
| GRID1 | − | NT | NT | NT | + | NT | NT |
| PRIM1 | − | NT | NT | NT | + | NT | NT |
| DUT | − | NT | NT | NT | + | NT | NT |
| RRAD | − | NT | NT | NT | + | NT | NT |
| BIRC5/ | − | NT | NT | NT | + | NT | NT |
| SURVIVIN | | | | | | | |
| PGEA1/ | − | NT | NT | NT | + | NT | NT |
| CBY-1 | | | | | | | |

| | | Tail-Vein | | | |
|---|---|---|---|---|---|
| | Oncogenic | lung | In vivo metastasis | In vivo | |
| | in vivo | seeding | (distal met to LN | metastasis | Tail Vein |
| | (sq tumor: | (M3HRAS | or to: 1205LU or | (distal met: | (knock- |
| Gene name | HMEL) | or WM115) | WM115) | NB008 model) | down) |
| ACP5 | + | NT | + | + | |
| ANLN | − | NT | NT | NT | |
| ASF1B | − | NT | − | NT | |
| BRRN1 | + | NT | NT | NT | |
| BUB1 | − | NT | NT | NT | |
| CDC2 | NT | NT | NT | NT | |
| CENPM | NT | NT | NT | NT | |
| DEPDC1 | − | NT | NT | NT | |
| ELTD1 | − | NT | NT | NT | |
| EXT1 | NT | NT | NT | NT | |
| FSCN1 | + | + | NT | NT | |
| HCAP-G | NT | NT | NT | NT | |
| HMGB1 | − | NT | NT | NT | |
| HMGB2 | NT | NT | NT | NT | |
| HOXA1 | + | + | NT | + | |
| HSF1 | + | NT | NT | NT | |
| ITGB3BP | − | NT | NT | NT | |
| KIF20A | NT | NT | NT | NT | |
| KIF2C | NT | NT | NT | NT | |
| KNTC2/NDC80 | + | NT | NT | NT | |
| MCM7 | NT | NT | NT | NT | |
| MTHFD2 | − | NT | NT | NT | |
| NASP | NT | NT | NT | NT | |
| PLVAP | NT | NT | NT | NT | |
| PTP4A3 | NT | NT | NT | NT | |
| RNF2 | + | NT | − | NT | + |
| SPAG5 | − | NT | NT | NT | |
| TGM2 | NT | NT | NT | NT | |
| UBE2C | − | NT | NT | NT | |
| UCHL5 | − | NT | + | NT | |
| VSIG4 | + | NT | NT | NT | |
| HNRPR | NT | NT | + | NT | |
| CDC20 | NT | NT | − | NT | |
| PRIM2A | NT | NT | + | NT | |
| HRSP12 | NT | NT | + | NT | |
| ENY2/SUS1 | NT | NT | + | NT | |
| TMEM141 | NT | NT | − | NT | |
| RECQL | NT | NT | − | NT | |
| STK3 | NT | NT | − | NT | |
| MX2 | NT | NT | + | NT | |
| CDCA1/NUF2 | NT | NT | NT | NT | |
| CEPS8/ | NT | NT | NT | NT | |
| KIAA0582 | | | | | |
| SPBC25 | NT | NT | NT | NT | |
| CDC25C | NT | NT | NT | NT | |
| GRID1 | NT | NT | NT | NT | |
| PRIM1 | NT | NT | NT | NT | |
| DUT | NT | NT | NT | NT | |
| RRAD | NT | NT | NT | NT | |
| BIRC5/ | NT | NT | NT | NT | |
| SURVIVIN | | | | | |
| PGEA1/ | NT | NT | NT | NT | |
| CBY-1 | | | | | |

TABLE 11
Candidate cDNAs screened and primary hits identified
in the genetic screen for pro-invasion genes.

| | | primary screen 45 hits |
|---|---|---|
| 199 candidates screened | | (2xSD in 2 screens) |
| Gene ID | Gene Symbol | Gene Symbol |
| 54 | ACP5 | ACP5 |
| 54443 | ANLN | ANLN |
| 410 | ARSA | ARSA |
| 55723 | ASF1B | ASF1B |
| 9212 | AURKB | AURKB |
| 23397 | NCAPH | NCAPH |
| 80135 | BXDC5 | BXDC5 |
| 947 | CD34 | CD34 |
| 55536 | CDCA7L | CDCA7L |
| 79019 | CENPM | CENPM |
| 55635 | DEPDC1 | DEPDC1 |
| 51514 | DTL | DTL |
| 64123 | ELTD1 | ELTD1 |
| 2131 | EXT1 | EXT1 |
| 63979 | FIGNL1 | FIGNL1 |
| 55220 | KLHDC8A | FLJ10748 |
| 6624 | FSCN1 | FSCN1 |
| 2775 | GNAO1 | GNAO1 |
| 2792 | GNGT1 | GNGT1 |
| 2894 | GRID1 | GRID1 |
| 50810 | HDGFRP3 | HDGFRP3 |
| 3146 | HMGB1 | HMGB1 |
| 3148 | HMGB2 | HMGB2 |
| 3198 | HOXA1 | HOXA1 |
| 3297 | HSF1 | HSF1 |
| 10808 | HSPH1 | HSPH1 |
| 23421 | ITGB3BP | ITGB3BP |
| 10403 | NDCC80 | NDC80 |
| 4174 | MCM5 | MCM5 |
| 4176 | MCM7 | MCM7 |
| 10797 | MTHFD2 | MTHFD2 |
| 4855 | NOTCH4 | NOTCH4 |
| 53371 | NUP54 | NUP54 |
| 4999 | ORC2L | ORC2L |
| 83483 | PLVAP | PLVAP |
| 9265 | PSCD3 | PSCD3 |
| 6045 | RNF2 | RNF2 |
| 10615 | SPAG5 | SPAG5 |
| 26872 | STEAP1 | STEAP1 |
| 7052 | TGM2 | TGM2 |
| 54543 | TOMM7 | TOMM7 |
| 22974 | TPX2 | TPX2 |
| 11065 | UBE2C | UBE2C |
| 51377 | UCHL5 | UCHL5 |
| 11326 | VSIG4 | VSIG4 |
| 23600 | AMACR | |
| 10928 | DBF4 | |
| 259268 | ASPM | |
| 477 | ATP1A2 | |
| 627 | BDNF | |
| 638 | BIK | |
| 332 | BIRC5 | |
| 55839 | C16ORF60 | |
| 672 | BRCA1 | |
| 699 | BUB1 | |
| 701 | BUB1B | |
| 55165 | CEP55 | |
| 79971 | MIER1 | |
| 116496 | C1ORF24 | |
| 719 | C3AR1 | |
| 57002 | C7ORF36 | |
| 84933 | C8ORF76 | |
| 152007 | C9ORF19 | |
| 857 | CAV1 | |
| 6357 | CCL13 | |
| 6347 | CCL2 | |
| 948 | CD36 | |
| 983 | CDC2 | |
| 991 | CDC20 | |
| 995 | CDC25C | |
| 83540 | CDCA1 | |
| 83461 | CDCA3 | |
| 1058 | CENPA | |
| 1070 | CETN3 | |
| 26586 | CKAP2 | |
| 1163 | CKS1B | |
| 1164 | CKS2 | |
| 9918 | CNAP1 | |
| 10664 | CTCF | |
| 1601 | DAB2 | |
| 56942 | C16ORF61 | |
| 23564 | DDAH2 | |
| 1719 | DHFR | |
| 55355 | DKFZP762E1312 | |
| 27122 | DKK3 | |
| 9787 | DLG7 | |
| 30836 | DNTTIP2 | |
| 1854 | DUT | |
| 51162 | EGFL7 | |
| 56943 | ENY2 | |
| 54749 | EPDR1 | |
| 2162 | F13A1 | |
| 51303 | FKBP11 | |
| 55110 | FLJ10292 | |
| 55273 | TMEM100 | |
| 79805 | FLJ12505 | |
| 84935 | FLJ14834 | |
| 54962 | FLJ20516 | |
| 2305 | FOXM1 | |
| 51809 | GALNT7 | |
| 51053 | GMNN | |
| 2936 | GSR | |
| 2966 | GTF2H2 | |
| 51512 | GTSE1 | |
| 3045 | HBD | |
| 64151 | HCAP-G | |
| 3082 | HGF | |
| 3142 | HLX1 | |
| 10236 | HNRPR | |
| 10247 | HRSP12 | |
| 3313 | HSPA9B | |
| 51501 | HSPC138 | |
| 29902 | C12ORF24 | |
| 3384 | ICAM2 | |
| 10008 | KCNE3 | |
| 9768 | K1AA0101 | |
| 9694 | KIAA0103 | |
| 23177 | CEP68 | |
| 22901 | ARSG | |
| 56243 | KIAA1217 | |
| 10112 | KIF20A | |
| 11004 | KIF2C | |
| 3915 | LAMC1 | |
| 55915 | LANCL2 | |
| 4005 | LMO2 | |
| 91614 | LOC91614 | |
| 4076 | GPIAP1 | |
| 4085 | MAD2L1 | |
| 6300 | MAPK12 | |
| 4172 | MCM3 | |
| 4175 | MCM6 | |
| 4232 | MEST | |
| 85014 | MGC14141 | |
| 4318 | MMP9 | |
| 219928 | MRGPRF | |
| 64968 | MRPS6 | |
| 10232 | MSLN | |
| 4600 | MX2 | |
| 4678 | NASP | |
| 4751 | NEK2 | |
| 23530 | NNT | |
| 4846 | NOS3 | |
| 11163 | NUDT4 | |
| 51203 | NUSAP1 | |
| 116039 | OSR2 | |
| 5019 | OXCT1 | |
| 56288 | PARD3 | |
| 55872 | PBK | |
| 11333 | PDAP1 | |
| 5156 | PDGFRA | |
| 25776 | PGEA1 | |
| 57125 | PLXDC1 | |
| 5425 | POLD2 | |
| 5446 | PON3 | |
| 5557 | PRIM1 | |
| 5558 | PRIM2A | |
| 23627 | PRND | |
| 5743 | PTGS2 | |
| 11156 | PTP4A3 | |
| 5885 | RAD21 | |
| 5889 | RAD51C | |
| 3516 | RBPSUH | |
| 5965 | RECQL | |
| 5984 | RFC4 | |
| 5985 | RFC5 | |
| 64407 | RGS18 | |
| 5997 | RGS2 | |
| 8490 | RGS5 | |
| 6118 | RPA2 | |
| 6119 | RPA3 | |
| 6236 | RRAD | |
| 22800 | RRAS2 | |
| 6240 | RRM1 | |
| 6241 | RRM2 | |
| 340419 | RSPO2 | |
| 79801 | SHCBP1 | |
| 8036 | SHOC2 | |
| 7884 | SLBP | |
| 115288 | SLC25A26 | |
| 8467 | SMARCA5 | |
| 8829 | SNRPB2 | |
| 57405 | SPBC25 | |
| 80559 | SPCS3 | |
| 8742 | SSBP1 | |
| 8788 | STK3 | |
| 23435 | TARDBP | |
| 25771 | TBC1D22A | |
| 90390 | THRAP6 | |
| 8914 | TIMELESS | |
| 7077 | TIMP2 | |
| 7083 | TK1 | |
| 4591 | TRIM37 | |
| 9319 | TRIP13 | |
| 7371 | UCK2 | |
| 83878 | USHBP1 | |
| 10894 | XLKD1 | |
| 51776 | ZAK | |
| 79830 | ZMYM1 | |
| 84858 | ZNF503 | |

SD = standard deviations

TABLE 12
Two Biomarkers Combinations

| | ACP5 | ANLN | ASF1B | BRRN1 | BUB1 | CDC2 | CENPM | DEPDC1 | ELTD1 | EXT1 | FSCN1 | HCAP-G | HMGB1 |
|---|---|---|---|---|---|---|---|---|---|---|---|---|---|
| ACP5 | | + | + | + | + | + | + | + | + | + | + | + | + |
| ANLN | | | + | + | + | + | + | + | + | + | + | + | + |
| ASF1B | | | | + | + | + | + | + | + | + | + | + | + |
| BRRN1 | | | | | + | + | + | + | + | + | + | + | + |
| BUB1 | | | | | | + | + | + | + | + | + | + | + |
| CDC2 | | | | | | | + | + | + | + | + | + | + |
| CENPM | | | | | | | | + | + | + | + | + | + |
| DEPDC1 | | | | | | | | | + | + | + | + | + |
| ELTD1 | | | | | | | | | | + | + | + | + |
| EXT1 | | | | | | | | | | | + | + | + |
| FSCN1 | | | | | | | | | | | | + | + |
| HCAP-G | | | | | | | | | | | | | + |
| HMGB1 | | | | | | | | | | | | | |
| HMGB2 | | | | | | | | | | | | | |
| HOXA1 | | | | | | | | | | | | | |
| HSF1 | | | | | | | | | | | | | |
| ITGB3BP | | | | | | | | | | | | | |
| KIF20A | | | | | | | | | | | | | |
| KIF2C | | | | | | | | | | | | | |
| KNTC2 | | | | | | | | | | | | | |
| MCM7 | | | | | | | | | | | | | |
| MTHFD2 | | | | | | | | | | | | | |
| NASP | | | | | | | | | | | | | |
| PLVAP | | | | | | | | | | | | | |
| PTP4A3 | | | | | | | | | | | | | |
| RNF2 | | | | | | | | | | | | | |
| SPAG5 | | | | | | | | | | | | | |
| TGM2 | | | | | | | | | | | | | |
| UBE2C | | | | | | | | | | | | | |
| UCHL5 | | | | | | | | | | | | | |
| VSIG4 | | | | | | | | | | | | | |
| HNRPR | | | | | | | | | | | | | |
| CDC20 | | | | | | | | | | | | | |
| PRIM2A | | | | | | | | | | | | | |
| HRSP12 | | | | | | | | | | | | | |
| ENY2 | | | | | | | | | | | | | |
| TMEM141 | | | | | | | | | | | | | |
| RECQL | | | | | | | | | | | | | |
| STK3 | | | | | | | | | | | | | |
| MX2 | | | | | | | | | | | | | |
| CDCA1 | | | | | | | | | | | | | |
| CEP68 | | | | | | | | | | | | | |
| SPBC25 | | | | | | | | | | | | | |
| CDC25C | | | | | | | | | | | | | |
| GRID1 | | | | | | | | | | | | | |
| PRIM1 | | | | | | | | | | | | | |
| DUT | | | | | | | | | | | | | |
| RRAD | | | | | | | | | | | | | |
| BIRC5 | | | | | | | | | | | | | |
| PGEA1 | | | | | | | | | | | | | |

| | HMGB2 | HOXA1 | HSF1 | ITGB3BP | KIF20A | KIF2C | KNTC2 | MCM7 | MTHFD2 | NASP | PLVAP | PTP4A3 | RNF2 |
|---|---|---|---|---|---|---|---|---|---|---|---|---|---|
| ACP5 | + | + | + | + | + | + | + | + | + | + | + | + | + |
| ANLN | + | + | + | + | + | + | + | + | + | + | + | + | + |
| ASF1B | + | + | + | + | + | + | + | + | + | + | + | + | + |
| BRRN1 | + | + | + | + | + | + | + | + | + | + | + | + | + |
| BUB1 | + | + | + | + | + | + | + | + | + | + | + | + | + |
| CDC2 | + | + | + | + | + | + | + | + | + | + | + | + | + |
| CENPM | + | + | + | + | + | + | + | + | + | + | + | + | + |
| DEPDC1 | + | + | + | + | + | + | + | + | + | + | + | + | + |
| ELTD1 | + | + | + | + | + | + | + | + | + | + | + | + | + |
| EXT1 | + | + | + | + | + | + | + | + | + | + | + | + | + |
| FSCN1 | + | + | + | + | + | + | + | + | + | + | + | + | + |
| HCAP-G | + | + | + | + | + | + | + | + | + | + | + | + | + |
| HMGB1 | + | + | + | + | + | + | + | + | + | + | + | + | + |
| HMGB2 | | + | + | + | + | + | + | + | + | + | + | + | + |
| HOXA1 | | | + | + | + | + | + | + | + | + | + | + | + |
| HSF1 | | | | + | + | + | + | + | + | + | + | + | + |
| ITGB3BP | | | | | + | + | + | + | + | + | + | + | + |
| KIF20A | | | | | | + | + | + | + | + | + | + | + |
| KIF2C | | | | | | | + | + | + | + | + | + | + |
| KNTC2 | | | | | | | | + | + | + | + | + | + |
| MCM7 | | | | | | | | | + | + | + | + | + |
| MTHFD2 | | | | | | | | | | + | + | + | + |
| NASP | | | | | | | | | | | + | + | + |
| PLVAP | | | | | | | | | | | | + | + |
| PTP4A3 | | | | | | | | | | | | | + |
| RNF2 | | | | | | | | | | | | | |
| SPAG5 | | | | | | | | | | | | | |
| TGM2 | | | | | | | | | | | | | |
| UBE2C | | | | | | | | | | | | | |
| UCHL5 | | | | | | | | | | | | | |
| VSIG4 | | | | | | | | | | | | | |
| HNRPR | | | | | | | | | | | | | |
| CDC20 | | | | | | | | | | | | | |
| PRIM2A | | | | | | | | | | | | | |
| HRSP12 | | | | | | | | | | | | | |
| ENY2 | | | | | | | | | | | | | |
| TMEM141 | | | | | | | | | | | | | |
| RECQL | | | | | | | | | | | | | |
| STK3 | | | | | | | | | | | | | |
| MX2 | | | | | | | | | | | | | |
| CDCA1 | | | | | | | | | | | | | |
| CEP68 | | | | | | | | | | | | | |
| SPBC25 | | | | | | | | | | | | | |
| CDC25C | | | | | | | | | | | | | |
| GRID1 | | | | | | | | | | | | | |
| PRIM1 | | | | | | | | | | | | | |
| DUT | | | | | | | | | | | | | |
| RRAD | | | | | | | | | | | | | |
| BIRC5 | | | | | | | | | | | | | |
| PGEA1 | | | | | | | | | | | | | |

| | SPAG5 | TGM2 | UBE2C | UCHL5 | VSIG4 | HNRPR | CDC20 | PRIM2A | HRSP12 | ENY2 | TMEM141 | RECQL | STK3 |
|---|---|---|---|---|---|---|---|---|---|---|---|---|---|
| ACP5 | + | + | + | + | + | + | + | + | + | + | + | + | + |
| ANLN | + | + | + | + | + | + | + | + | + | + | + | + | + |
| ASF1B | + | + | + | + | + | + | + | + | + | + | + | + | + |
| BRRN1 | + | + | + | + | + | + | + | + | + | + | + | + | + |
| BUB1 | + | + | + | + | + | + | + | + | + | + | + | + | + |
| CDC2 | + | + | + | + | + | + | + | + | + | + | + | + | + |
| CENPM | + | + | + | + | + | + | + | + | + | + | + | + | + |
| DEPDC1 | + | + | + | + | + | + | + | + | + | + | + | + | + |
| ELTD1 | + | + | + | + | + | + | + | + | + | + | + | + | + |
| EXT1 | + | + | + | + | + | + | + | + | + | + | + | + | + |
| FSCN1 | + | + | + | + | + | + | + | + | + | + | + | + | + |
| HCAP-G | + | + | + | + | + | + | + | + | + | + | + | + | + |
| HMGB1 | + | + | + | + | + | + | + | + | + | + | + | + | + |
| HMGB2 | + | + | + | + | + | + | + | + | + | + | + | + | + |
| HOXA1 | + | + | + | + | + | + | + | + | + | + | + | + | + |
| HSF1 | + | + | + | + | + | + | + | + | + | + | + | + | + |
| ITGB3BP | + | + | + | + | + | + | + | + | + | + | + | + | + |
| KIF20A | + | + | + | + | + | + | + | + | + | + | + | + | + |
| KIF2C | + | + | + | + | + | + | + | + | + | + | + | + | + |
| KNTC2 | + | + | + | + | + | + | + | + | + | + | + | + | + |
| MCM7 | + | + | + | + | + | + | + | + | + | + | + | + | + |
| MTHFD2 | + | + | + | + | + | + | + | + | + | + | + | + | + |
| NASP | + | + | + | + | + | + | + | + | + | + | + | + | + |
| PLVAP | + | + | + | + | + | + | + | + | + | + | + | + | + |
| PTP4A3 | + | + | + | + | + | + | + | + | + | + | + | + | + |
| RNF2 | + | + | + | + | + | + | + | + | + | + | + | + | + |
| SPAG5 | | + | + | + | + | + | + | + | + | + | + | + | + |
| TGM2 | | | + | + | + | + | + | + | + | + | + | + | + |
| UBE2C | | | | + | + | + | + | + | + | + | + | + | + |
| UCHL5 | | | | | + | + | + | + | + | + | + | + | + |
| VSIG4 | | | | | | + | + | + | + | + | + | + | + |
| HNRPR | | | | | | | + | + | + | + | + | + | + |
| CDC20 | | | | | | | | + | + | + | + | + | + |
| PRIM2A | | | | | | | | | + | + | + | + | + |
| HRSP12 | | | | | | | | | | + | + | + | + |
| ENY2 | | | | | | | | | | | + | + | + |
| TMEM141 | | | | | | | | | | | | + | + |
| RECQL | | | | | | | | | | | | | + |
| STK3 | | | | | | | | | | | | | |
| MX2 | | | | | | | | | | | | | |
| CDCA1 | | | | | | | | | | | | | |
| CEP68 | | | | | | | | | | | | | |
| SPBC25 | | | | | | | | | | | | | |
| CDC25C | | | | | | | | | | | | | |
| GRID1 | | | | | | | | | | | | | |
| PRIM1 | | | | | | | | | | | | | |
| DUT | | | | | | | | | | | | | |
| RRAD | | | | | | | | | | | | | |
| BIRC5 | | | | | | | | | | | | | |
| PGEA1 | | | | | | | | | | | | | |

| | MX2 | CDCA1 | CEP68 | SPBC25 | CDC25C | GRID1 | PRIM1 | DUT | RRAD | BIRC5 | PGEA1 |
|---|---|---|---|---|---|---|---|---|---|---|---|
| ACP5 | + | + | + | + | + | + | + | + | + | + | + |
| ANLN | + | + | + | + | + | + | + | + | + | + | + |
| ASF1B | + | + | + | + | + | + | + | + | + | + | + |
| BRRN1 | + | + | + | + | + | + | + | + | + | + | + |
| BUB1 | + | + | + | + | + | + | + | + | + | + | + |
| CDC2 | + | + | + | + | + | + | + | + | + | + | + |
| CENPM | + | + | + | + | + | + | + | + | + | + | + |
| DEPDC1 | + | + | + | + | + | + | + | + | + | + | + |
| ELTD1 | + | + | + | + | + | + | + | + | + | + | + |
| EXT1 | + | + | + | + | + | + | + | + | + | + | + |
| FSCN1 | + | + | + | + | + | + | + | + | + | + | + |
| HCAP-G | + | + | + | + | + | + | + | + | + | + | + |
| HMGB1 | + | + | + | + | + | + | + | + | + | + | + |
| HMGB2 | + | + | + | + | + | + | + | + | + | + | + |
| HOXA1 | + | + | + | + | + | + | + | + | + | + | + |
| HSF1 | + | + | + | + | + | + | + | + | + | + | + |
| ITGB3BP | + | + | + | + | + | + | + | + | + | + | + |
| KIF20A | + | + | + | + | + | + | + | + | + | + | + |
| KIF2C | + | + | + | + | + | + | + | + | + | + | + |
| KNTC2 | + | + | + | + | + | + | + | + | + | + | + |
| MCM7 | + | + | + | + | + | + | + | + | + | + | + |
| MTHFD2 | + | + | + | + | + | + | + | + | + | + | + |
| NASP | + | + | + | + | + | + | + | + | + | + | + |
| PLVAP | + | + | + | + | + | + | + | + | + | + | + |
| PTP4A3 | + | + | + | + | + | + | + | + | + | + | + |
| RNF2 | + | + | + | + | + | + | + | + | + | + | + |
| SPAG5 | + | + | + | + | + | + | + | + | + | + | + |
| TGM2 | + | + | + | + | + | + | + | + | + | + | + |
| UBE2C | + | + | + | + | + | + | + | + | + | + | + |
| UCHL5 | + | + | + | + | + | + | + | + | + | + | + |
| VSIG4 | + | + | + | + | + | + | + | + | + | + | + |
| HNRPR | + | + | + | + | + | + | + | + | + | + | + |
| CDC20 | + | + | + | + | + | + | + | + | + | + | + |
| PRIM2A | + | + | + | + | + | + | + | + | + | + | + |
| HRSP12 | + | + | + | + | + | + | + | + | + | + | + |
| ENY2 | + | + | + | + | + | + | + | + | + | + | + |
| TMEM141 | + | + | + | + | + | + | + | + | + | + | + |
| RECQL | + | + | + | + | + | + | + | + | + | + | + |
| STK3 | + | + | + | + | + | + | + | + | + | + | + |
| MX2 | | + | + | + | + | + | + | + | + | + | + |
| CDCA1 | | | + | + | + | + | + | + | + | + | + |
| CEP68 | | | | + | + | + | + | + | + | + | + |
| SPBC25 | | | | | + | + | + | + | + | + | + |
| CDC25C | | | | | | + | + | + | + | + | + |
| GRID1 | | | | | | | + | + | + | + | + |
| PRIM1 | | | | | | | | + | + | + | + |
| DUT | | | | | | | | | + | + | + |
| RRAD | | | | | | | | | | + | + |
| BIRC5 | | | | | | | | | | | + |
| PGEA1 | | | | | | | | | | | |

1. A method for predicting prognosis of a cancer patient, or for identifying a cancer patient in need of adjuvant therapy, or for monitoring the progression of a tumor in a patient, comprising:
obtaining a tissue sample from the patient; and
measuring the levels of two or more biomarkers in the sample or determining the nucleotide or amino acid sequence of one or more biomarkers in the sample, wherein the biomarkers are selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1,
wherein the measured levels, or a mutation in the determined sequence as compared to a reference sequence, is indicative of the prognosis of the cancer patient, or indicates that the patient is in need of adjuvant therapy, or is indicative of the progression of the tumor in the patient.

2. A method for predicting prognosis of a cancer patient, comprising:
obtaining a tissue sample from the patient; and
measuring the levels or determining the nucleotide or amino acid sequences of two or more biomarkers in the sample,
a) wherein at least one of the two or more biomarkers is associated with anoikis resistance; and at least one of the two or more biomarkers is associated with invasion; or
b) wherein at least one of the two or more biomarkers is associated with tumorigenesis; and at least one of the two or more biomarkers is associated with invasion; or
c) wherein at least one of the two or more biomarkers is associated with tumorigenesis; and at least one of the two or more biomarkers is associated with anoikis resistance; and
wherein the measured levels, or a mutation in the determined sequences as compared to a reference sequence, is indicative of the prognosis of the cancer patient.

3. The method of claim 2, wherein
a) the biomarkers associated with anoikis resistance are selected from the group consisting of HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA1;
b) the biomarkers associated with invasion are selected from the group consisting of ACP5, FSCN1, HOXA1, HSF1, NDC80, VSIG4, NCAPH, ASF1B, MTHFD2, RNF2, SPAG5, ANLN, DEPDC1, HMGB1, ITGB3BP, MCM7, UBE2C, and UCHL5;
c) the biomarkers associated with invasion are selected from the group consisting of ACP5, ANLN, ASF1B, BRRN1, BUB1, CDC2, CENPM, DEPDC1, ELTD1, EXT1, FSCN1, HCAP-G, HMGB1, HMGB2, HOXA1, HSF1, ITGB3BP, KIF20A, KIF2C, KNTC2, MCM7, MTHFD2, NASP, PLVAP, PTP4A3, RNF2, SPAG5, TGM2, UBE2C, UCHL5, and VSIG4; or
d) the biomarkers associated with tumorigenesis are selected from the group consisting of: ACP5, FSCN1, HOXA1, HSF1. NDC80, VSIG4, BRRN1, RNF2, UCHL5, HNRPR, PRIM2A, HRSP12, ENY2, and MX2.

4-6. (canceled)

7. The method of claim 1, wherein the prognosis is that the patient is at a low risk of having metastatic cancer or recurrence of cancer.

8. The method of claim 1, wherein the prognosis is that the patient is at a high risk of having metastatic cancer or recurrence of cancer.

9. A method for analyzing a tissue sample from a cancer patient, comprising:
obtaining the tissue sample from the patient; and
measuring the levels of two or more biomarkers in the sample or determining the nucleotide or amino acid sequence of one or more biomarkers in the sample,
wherein the biomarkers are selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1.

10. (canceled)

11. The method of claim 1, wherein the adjuvant therapy is selected from the group consisting of radiation therapy, chemotherapy, immunotherapy, hormone therapy, and targeted therapy.

12-13. (canceled)

14. A method for treating a cancer patient, comprising:
a) measuring the levels of two or more biomarkers, or determining the nucleotide or amino acid sequence of one or more biomarkers, in a tissue sample from the patient, wherein the biomarkers are selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1, and treating the patient with adjuvant therapy if the measured levels, or a mutation in the determined sequence as compared to a reference sequence, indicates that the patient is at a high risk of having metastatic cancer or recurrence of cancer, or
b) measuring the level of a biomarker selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1, and administering an agent that modulates the level of the selected biomarker.

15. (canceled)

16. The method of claim 1, wherein the patient has melanoma or breast cancer.

17-18. (canceled)

19. The method of claim 8, further comprising performing sentinel lymph node biopsy on the patient.

20. The method of claim 7, further comprising not performing sentinel lymph node biopsy on the patient.

21. The method of claim 1, wherein
a) the selected biomarkers comprise one or more of ACP5, FSCN1, HOXA1, HSF1, NDC80, and VSIG4;
b) the selected biomarkers comprise one or more of ACP5, FSCN1, HOXA1, HSF1, NDC80, and VSIG4 and further comprise one or more of ASF1B, MTHFD2, RNF2, and SPAG5;
c) the selected biomarkers comprise one or more of HNRPR, CDC20, PRIM2A, HRSP12, ENY2, TMEM141, RECQL, STK3, and MX2;
d) the selected biomarkers comprise one or more of ACP5, FSCN1, HOXA1, HSF1, NDC80, VSIG4, NCAPH, ASF1B, MTHFD2, RNF2, SPAG5, ANLN, DEPDC1, HMGB1, ITGB3BP, MCM7, UBE2C, and UCHL5;
e) the selected biomarkers comprise one or more of HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA1; or
f) the selected biomarkers comprise at least one or more of ACP5, FSCN1 HOXA1, HSF1, NDC80, VSIG4, NCAPH, ASF1B, MTHFD2, RNF2, SPAG5, ANLN, DEPDC1, HMGB1, ITGB3BP, MCM7, UBE2C, and UCHL5; and at least one or more of HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA1.

22-26. (canceled)

27. The method of claim 1, wherein the measuring step comprises (a detecting the DNA copy number alteration of the selected biomarkers, (b) measuring the RNA transcript levels of the selected biomarkers, or (c) measuring the protein levels of the selected biomarkers.

28-29. (canceled)

30. The method of claim 1, wherein the nucleotide sequence or amino acid sequence is determined by sequencing.

31-48. (canceled)

49. The method of claim 1, further comprising measuring at least one standard parameter associated with the cancer.

50. (canceled)

51. A kit for measuring the levels of two or more biomarkers, or for determining the nucleotide or amino acid sequence of one or more biomarkers in the sample, wherein the biomarkers are selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1, wherein the kit comprises reagents for specifically measuring the levels of the selected biomarkers, or reagents for specifically determining the sequences of the selected biomarkers.

52. (canceled)

53. The kit of claim 51, wherein the reagents are nucleic acid molecules or antibodies.

54-55. (canceled)

56. A method for predicting prognosis of a cancer patient, comprising measuring the level of ACP5 or determining the nucleotide or amino acid sequence of ACP5 in a tissue sample from the patient, wherein the measured level of ACP5, or a mutation in the determined sequence of ACP5 as compared to a reference sequence of ACP5, is indicative of the prognosis of the cancer patient.

57. The method of claim 56, wherein the measuring step comprising measuring the level of the catalytic activity of ACP5, or measuring the level of the phosphatase activity of ACP5.

58. (canceled)

59. The method of claim 56, further comprising measuring the levels of or determining the nucleotide or amino acid sequence of one or more biomarkers selected from the group consisting of ANLN, ASF1B, BRRN1, BUB1, CDC2, CENPM, DEPDC1, ELTD1, EXT1, FSCN1, HCAP-G, HMGB1, HMGB2, HOXA1, HSF1, ITGB3BP, KIF20A, KIF2C, KNTC2, MCM7, MTHFD2, NASP, PLVAP, PTP4A3, RNF2, SPAG5, TGM2, UBE2C, UCHL5, and VSIG4.

60. The method of claim 56, further comprising measuring the levels or determining the nucleotide or amino acid sequence of one or more biomarkers selected from the group consisting of HNRPR, CDC20, PRIM2A, HRSP12, ENY2, TMEM141, RECQL, STK3, MX2, CDCA1, CEP68, SPBC25, HCAP-G, CDC25C, ANLN, GRID1, PRIM1, DUT, RRAD, BIRC5, KNTC2, and PGEA1.

61-63. (canceled)

64. The method of claim 14, wherein the administered agent is a small molecule modulator, a small molecule inhibitor, an siRNA, or an antibody.

65-67. (canceled)

68. The method of claim 14, wherein the selected biomarker in b) is ACP5, RNF2, UCHL5, HOXA1, UBE2C, FSCN1, HSF1, NDC80, VSIG4, BRRN1, HNRPR, PRIM2A, HRSP12, ENY2, or MX2.

69. The method of claim 68, wherein the selected biomarker is ACP5 and wherein the administered agent
a) causes a conformational change of ACP5, thereby preventing the biological activity of ACP5;
b) causes disruption of the interaction between ACP5 and a substrate of ACP5;
c) targets the catalytic activity of ACP5;
d) targets the phosphatase activity of ACP5;
e) targets one or more residues of ACP5, wherein the residues are selected from the histidine residue at position 111, the histidine residue at position 214, and the aspartic acid residue at position 265 of ACP5;
f) inhibits the secretion of ACP5; or
g) inhibits the secreted ACP5.

70-82. (canceled)

83. A method of identifying a compound capable of reducing the risk of cancer recurrence or development of metastatic cancer, or identifying a compound capable of treating cancer, or identifying a compound capable of reducing the risk of cancer occurrence or development of cancer, comprising:
(a) providing a cell expressing a biomarker selected from the group consisting of FSCN1, KIF2C, DEPDC1, ACP5, ANLN, ASF1B, BRRN1, BUB1, CDC2, CENPM, ELTD1, EXT1, HCAP-G, HMGB1, HMGB2, HOXA1, HSF1, ITGB3BP, KIF20A, KNTC2, MCM7, MTHFD2, NASP, PLVAP, PTP4A3, RNF2, SPAG5, TGM2, UBE2C, UCHL5, VSIG4, HNRPR, CDC20, PRIM2A, HRSP12, ENY2, TMEM141, RECQL, STK3, MX2, CDCA1, CEP68, SPBC25, CDC25C, GRID1, PRIM1, DUT, RRAD, BIRC5, and PGEA1;
(b) contacting the cell with a candidate compound; and
(c) determining whether the candidate compound alters the expression or activity of the selected biomarker,
whereby the alteration observed in the presence of the compound indicates that the compound is capable of reducing the risk of cancer recurrence or development of metastatic cancer, or that the compound is capable of treating cancer, or that the compound is capable of reducing the risk of cancer occurrence or development of cancer.

84-96. (canceled)
